(19) United States
(12) Patent Application Publication
(10) Pub. No.: US 2004/0067512 A1
(43) Pub. Date: Apr. 8, 2004

(54) Single nucleotide polymorphisms and mutations on Alpha-2-Macroglobulin

(75) Inventors: Kenneth David Becker, San Diego, CA (US); Gonul Velicelebi, San Diego, CA (US); Xin Wang, San Diego, CA (US); Lars Bertram, Brighton, MA (US); Aleister J. Saunders, Elkins Park, PA (US); Rudolph E. Tanzi, Hull, MA (US)

Correspondence Address: Stephanie Seidman, Heller Ehrman White & McAuliffe LLP, 7th Floor, 4350 La Jolla Village Drive, San Diego, CA, 92122-1246, US

(73) Assignees: Neurogenetics, Inc.; The General Hospital Corporation

(21) Appl. No.: 10/608,397

(22) Filed: Jun. 26, 2003

Related U.S. Application Data
(63) Continuation-in-part of application No. 10/292,081, filed on Nov. 8, 2002.
(60) Provisional application No. 60/337,434, filed on Nov. 9, 2001.

(51) Int. Cl. C12Q 1/68

(52) U.S. Cl. 435/6

(57) ABSTRACT

The present invention is related to the discovery of several single nucleotide polymorphisms (SNPs) and/or mutations in the Alpha-2-Macroglobulin gene (A2M), which are risk factors for Alzheimer's Disease (AD). More specifically, aspects of the invention concern nucleic acids corresponding to the A2M gene or fragments thereof, which contain one or more of the SNPs and/or mutations described herein, peptides or proteins encoded by said nucleic acids, antibodies to said peptides or proteins and methods of making said compositions, diagnostic methods, methods of data analysis, and pharmaceutical discovery and preparation methods.

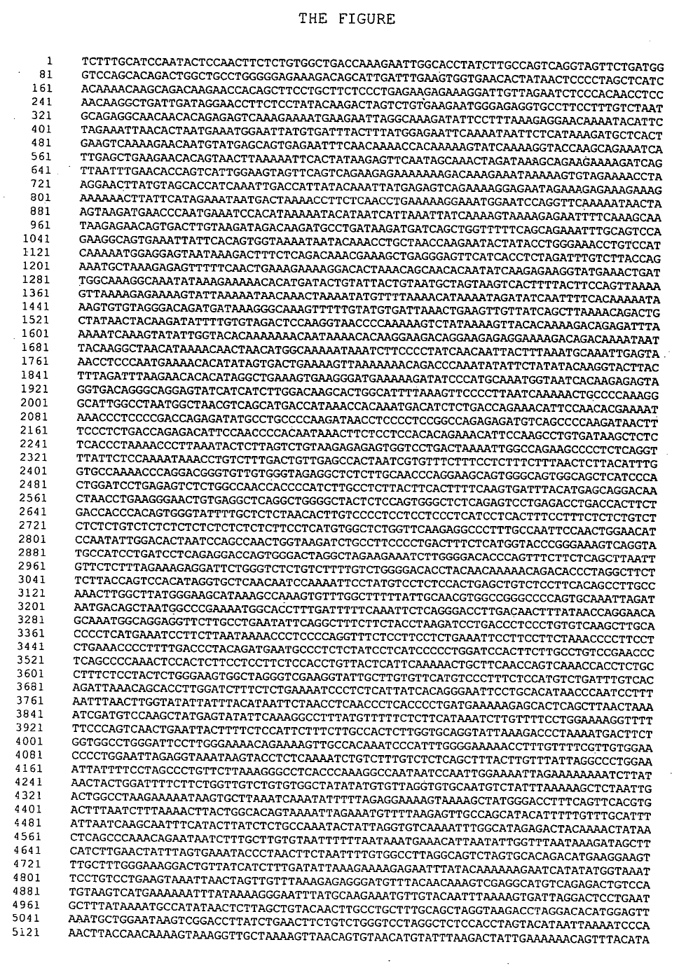

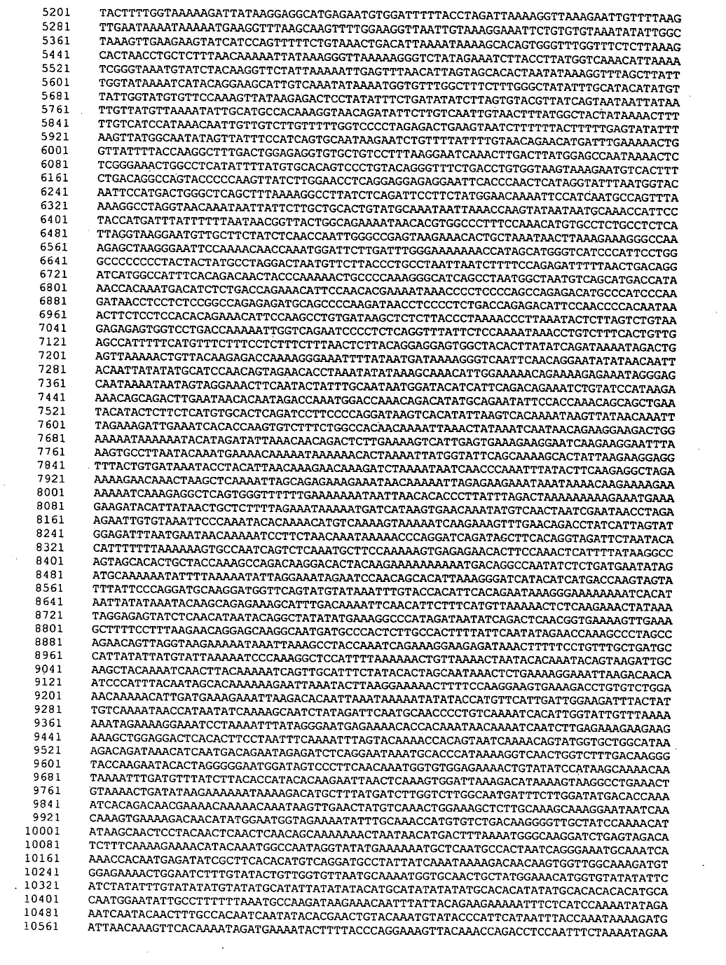

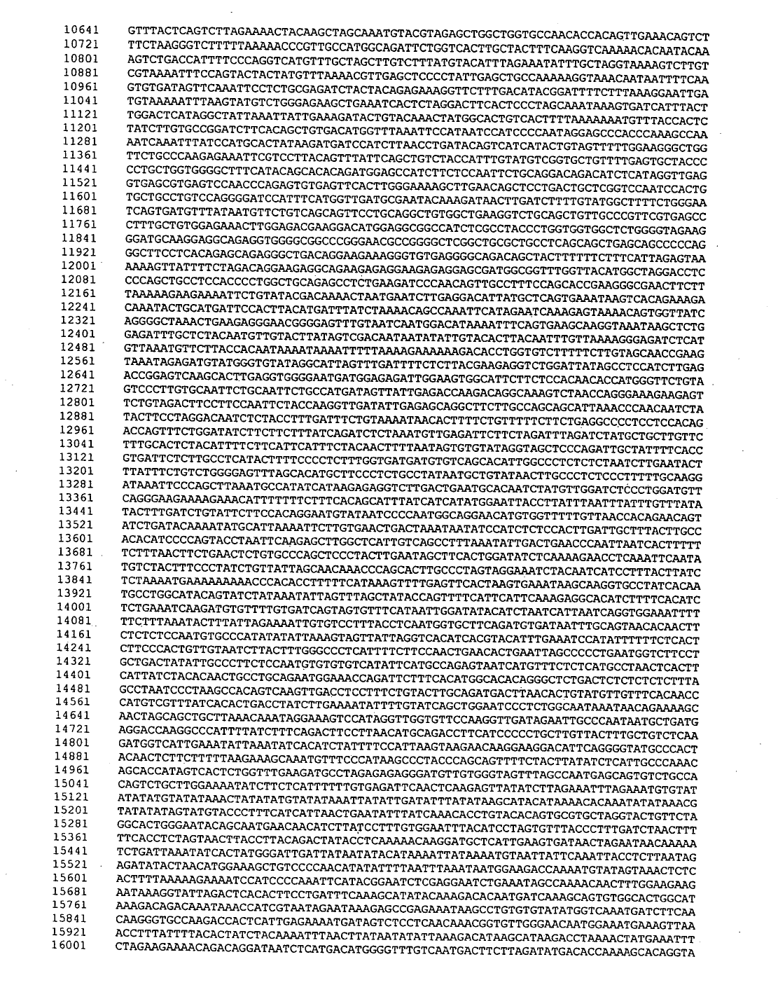

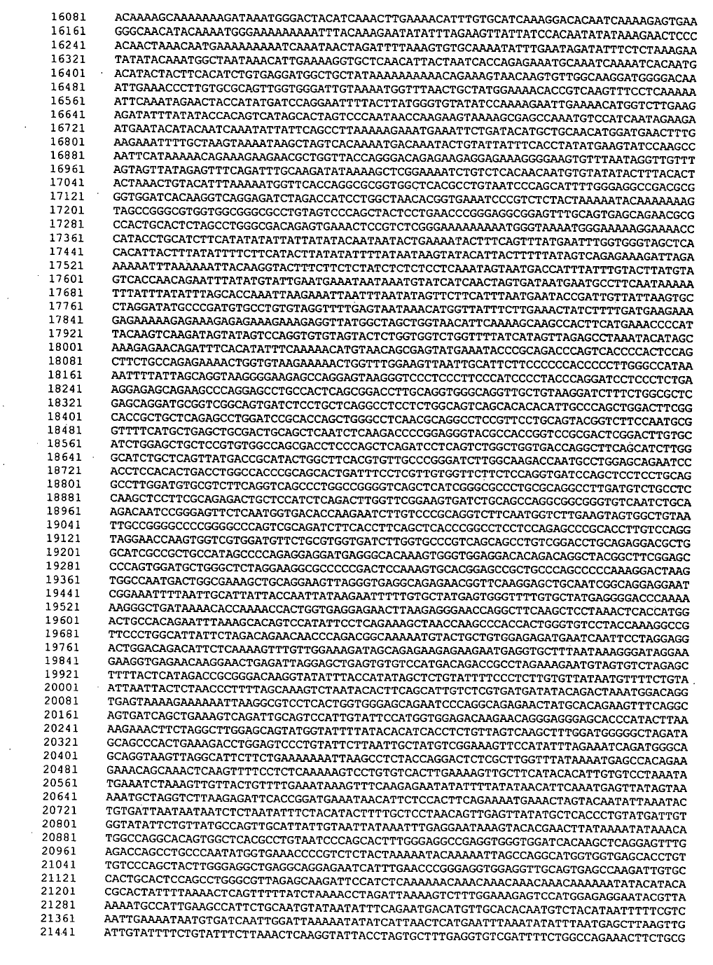

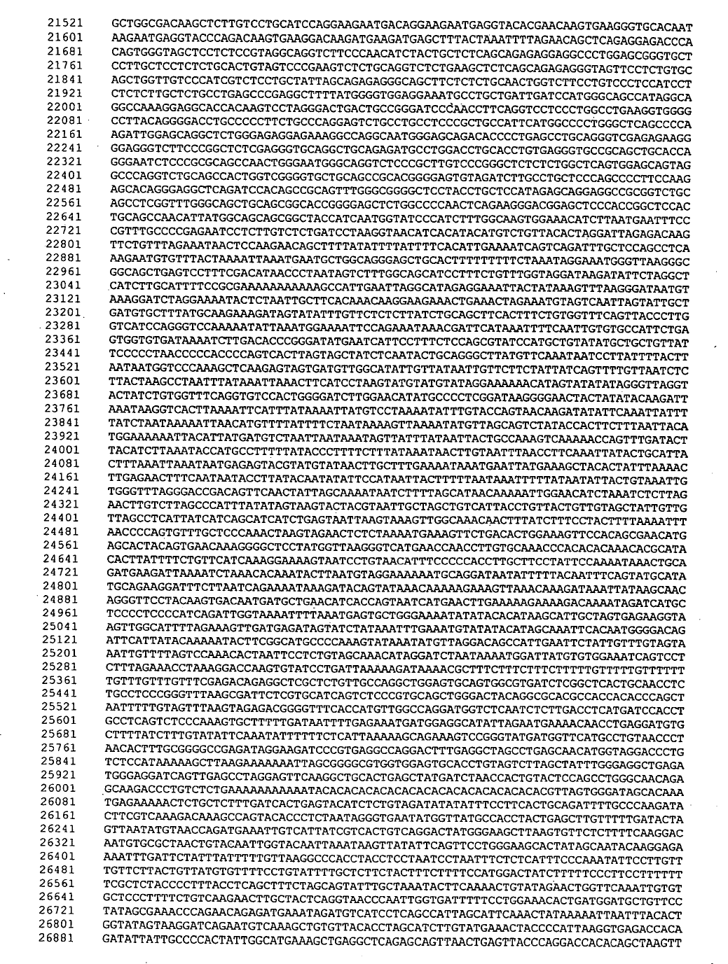

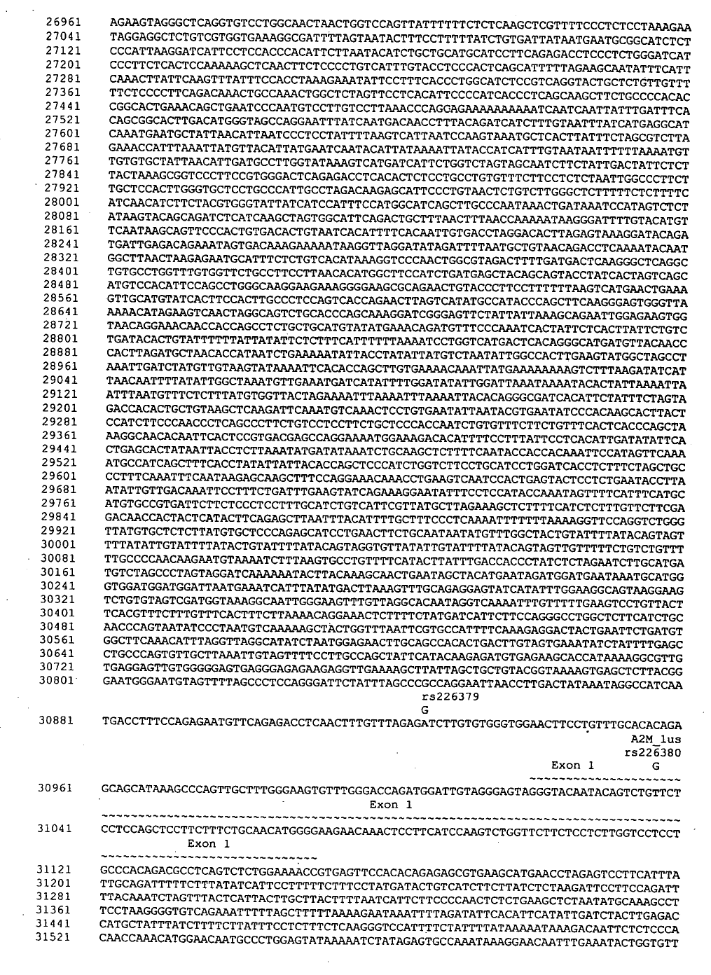

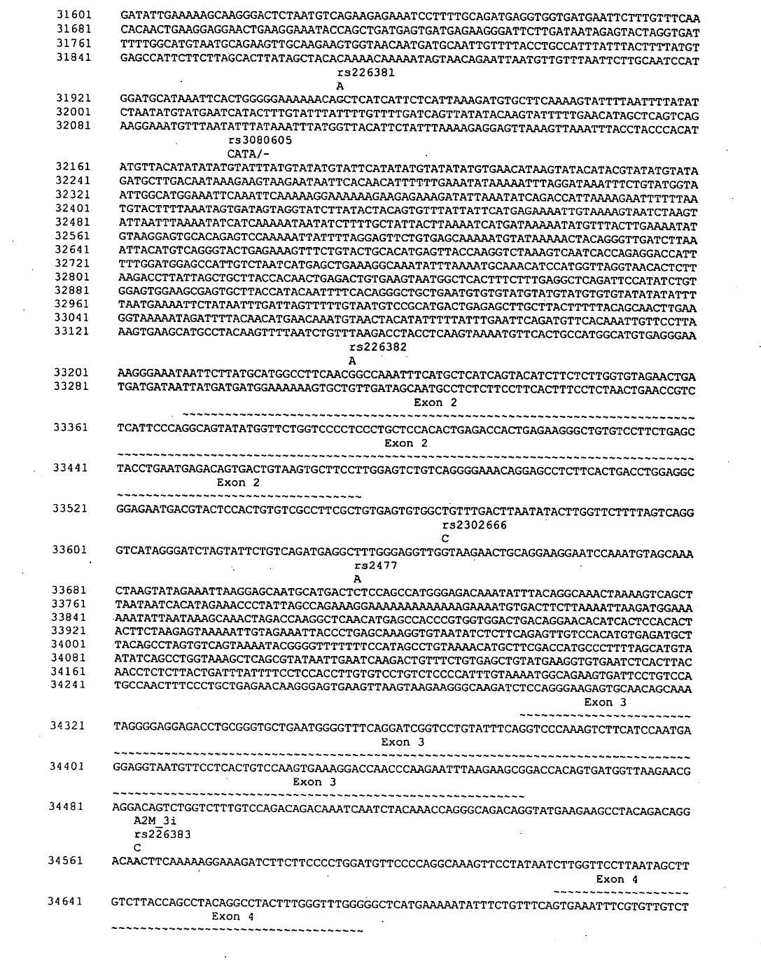

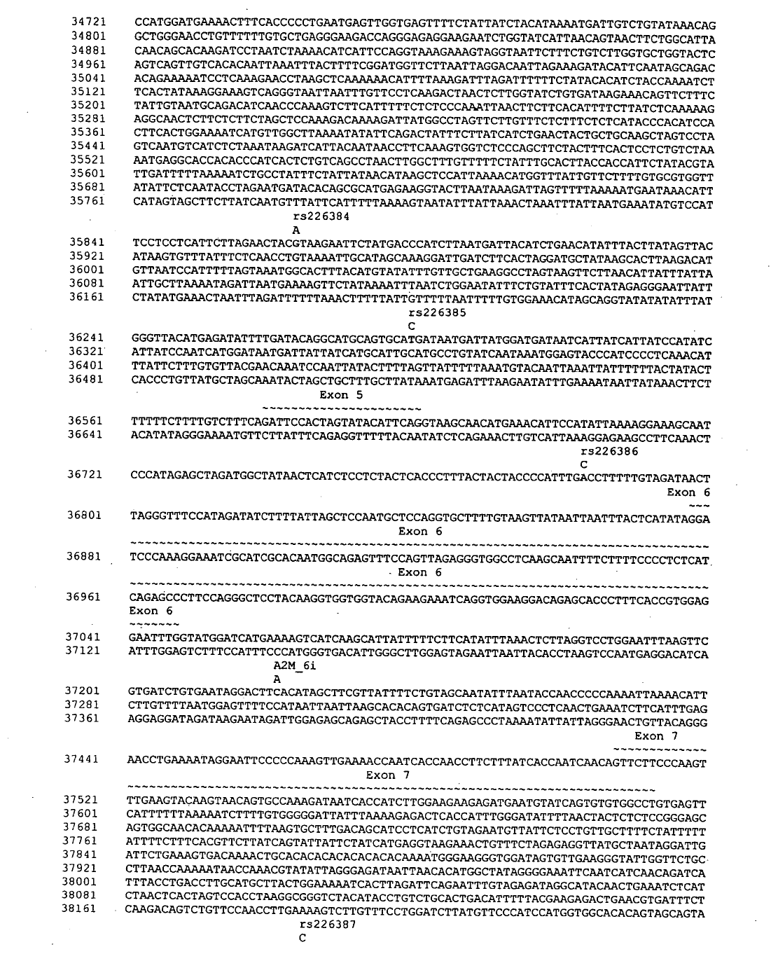

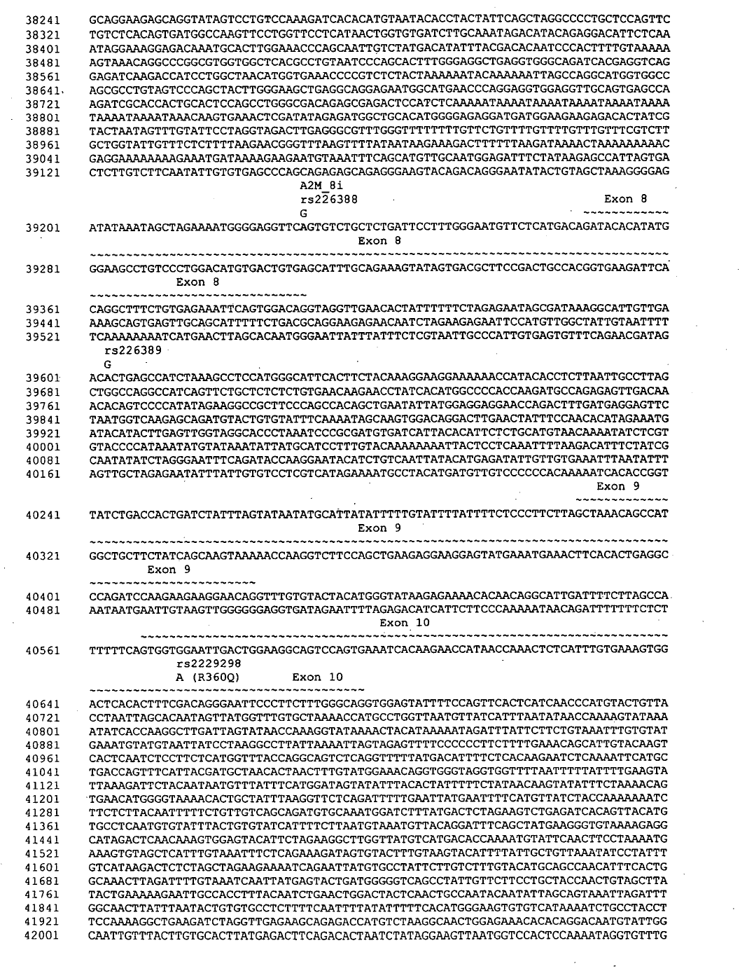

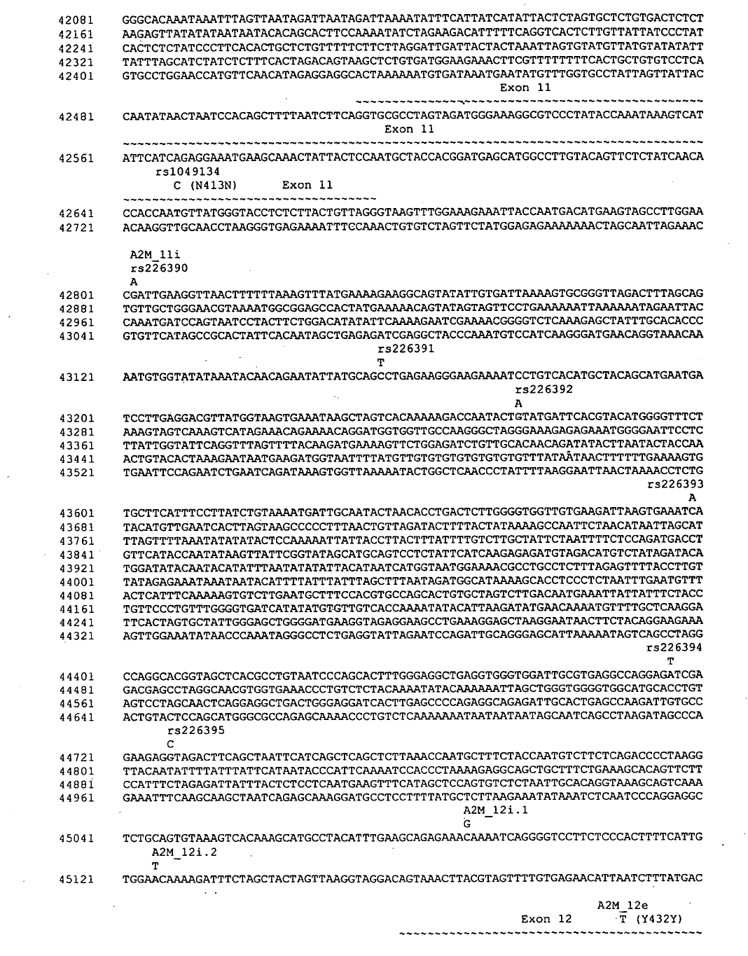

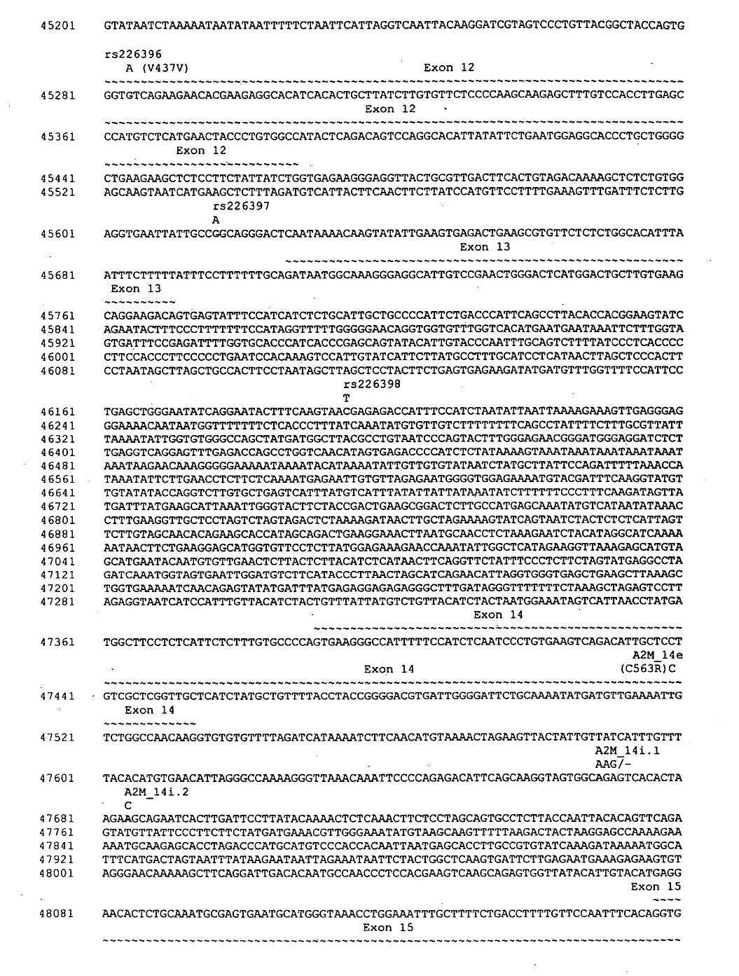

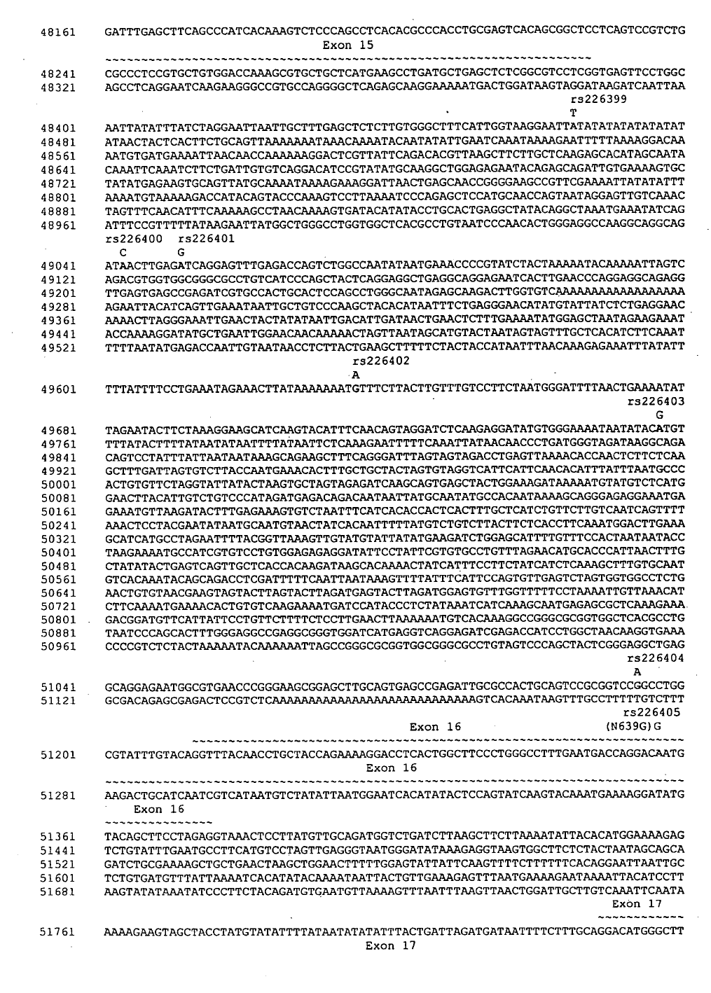

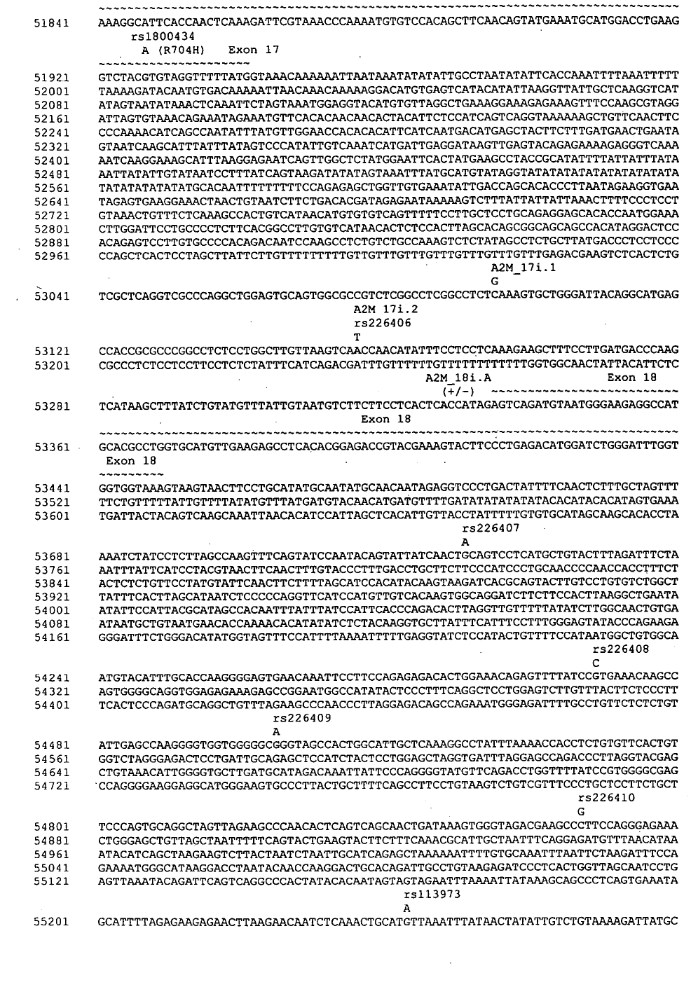

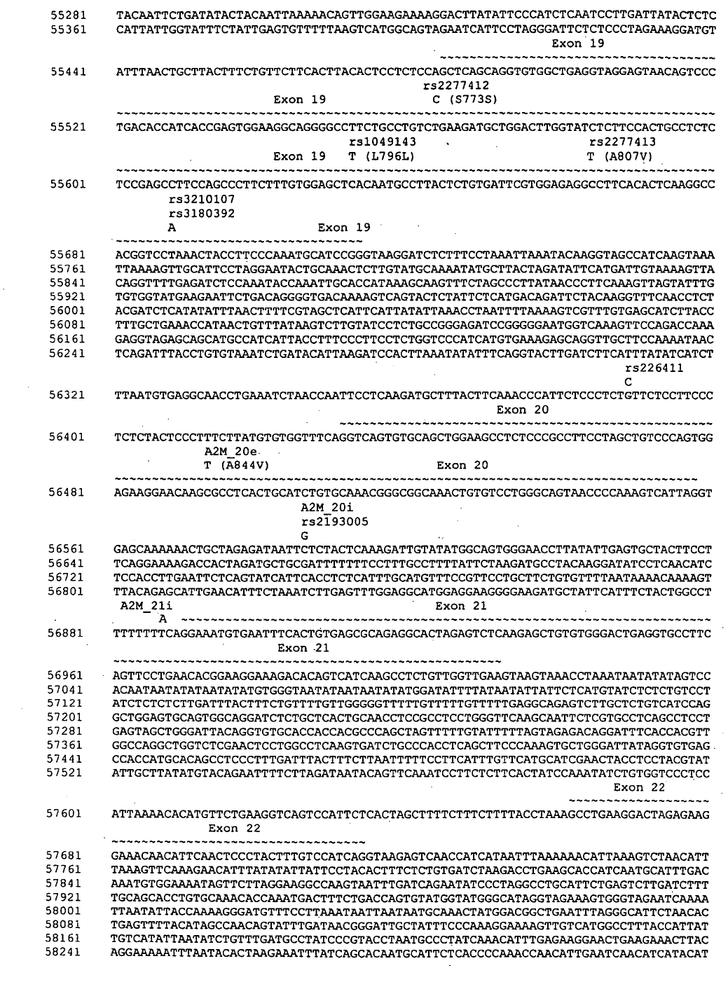

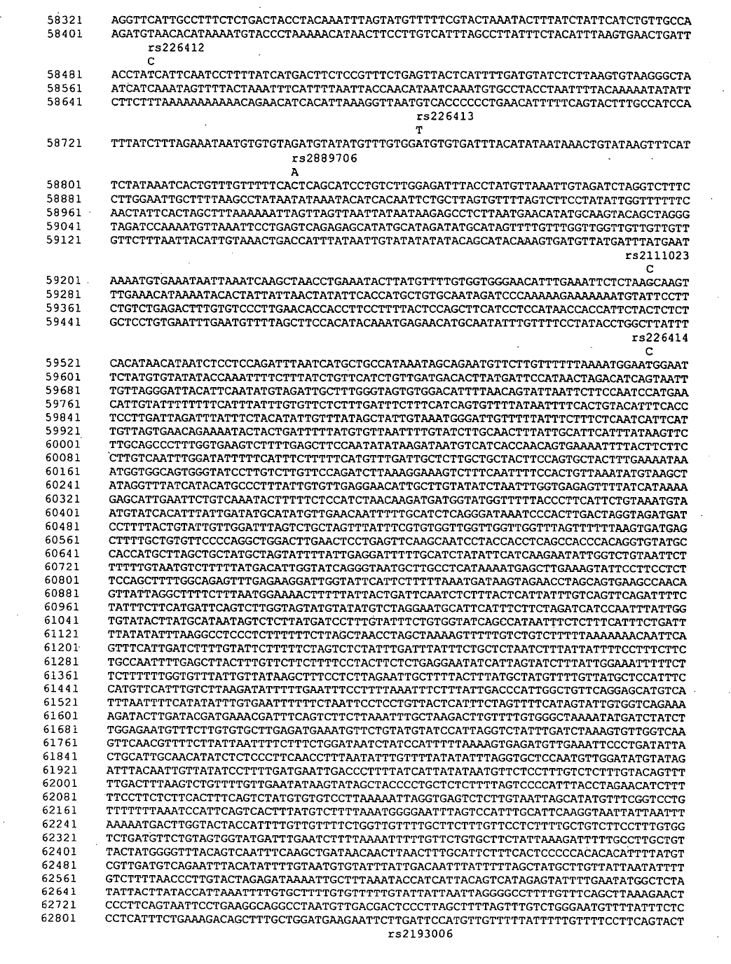

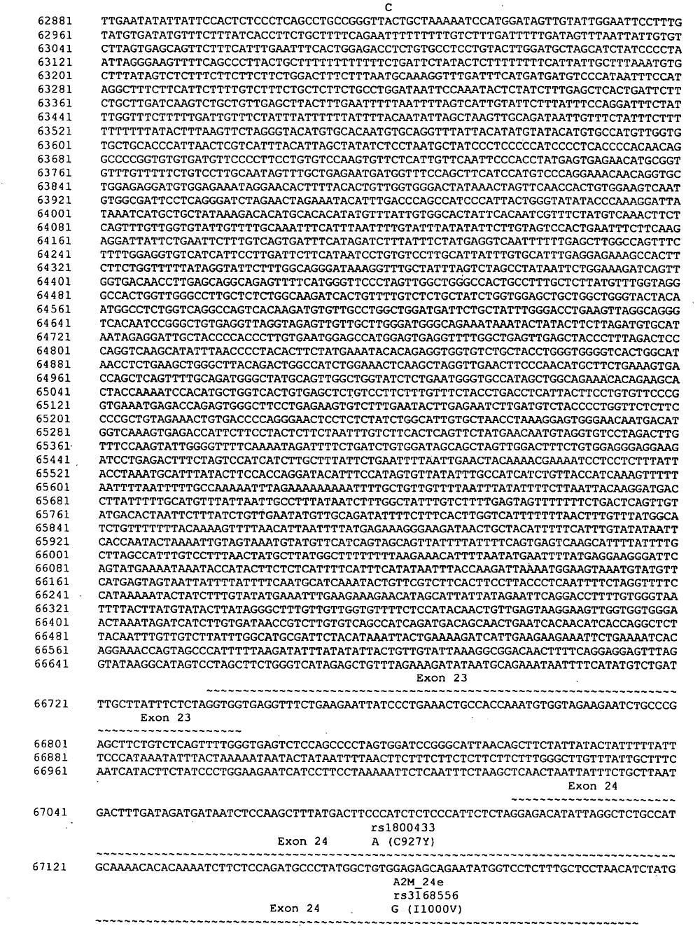

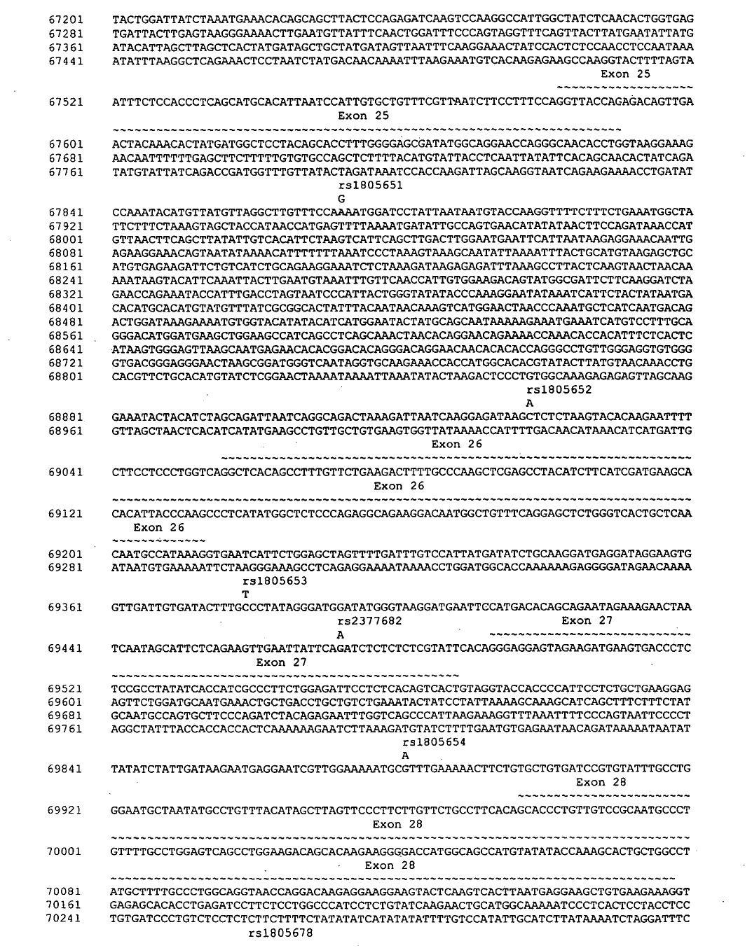

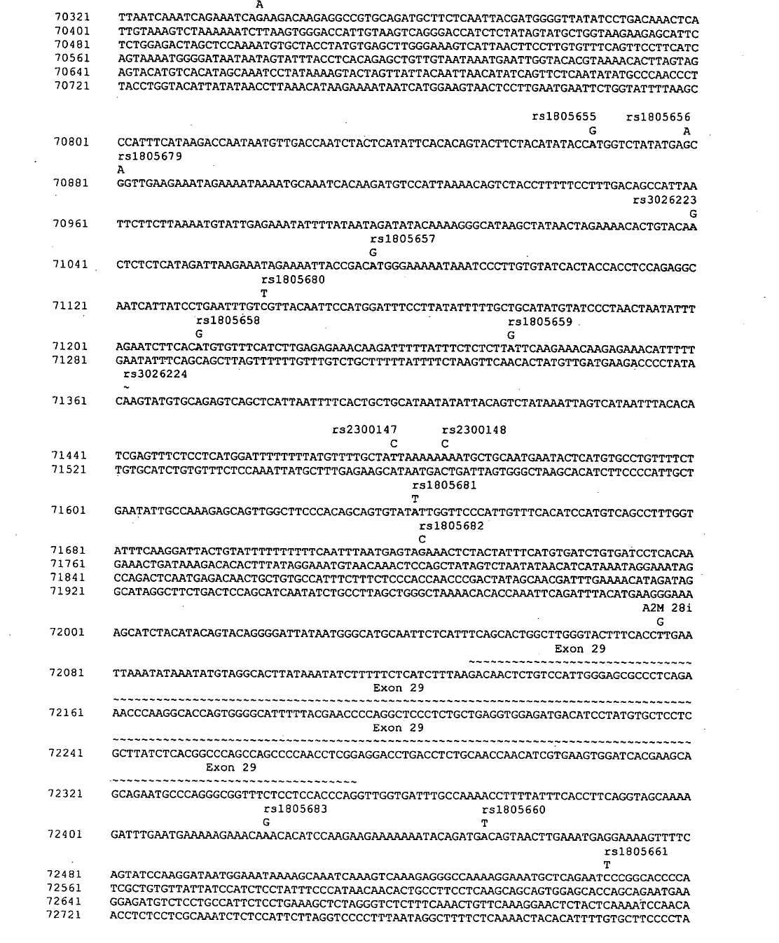

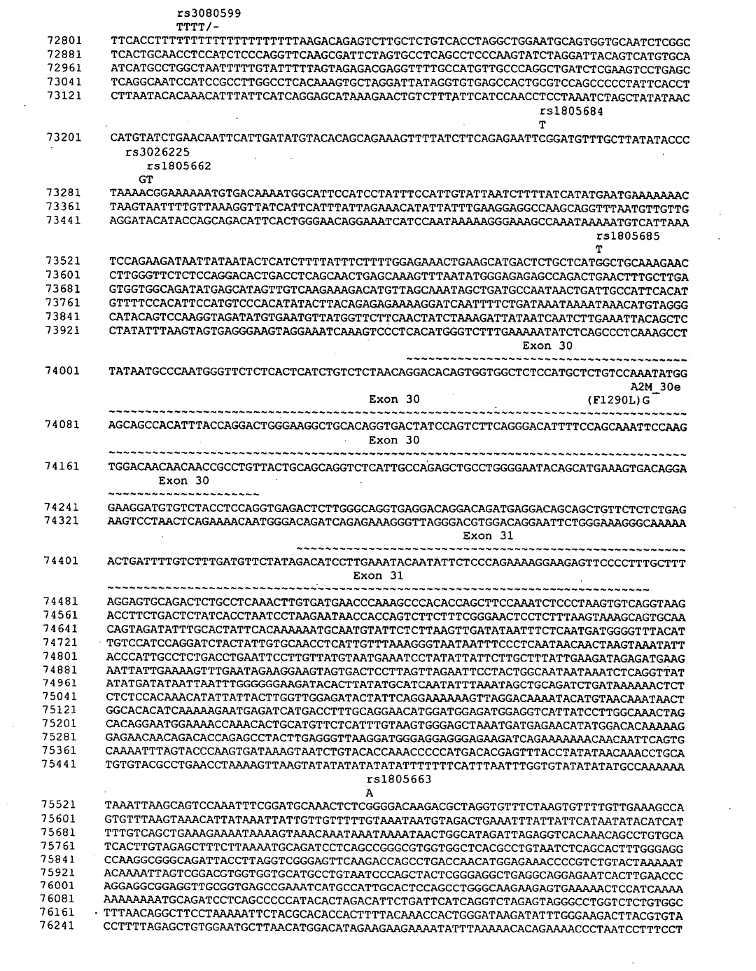

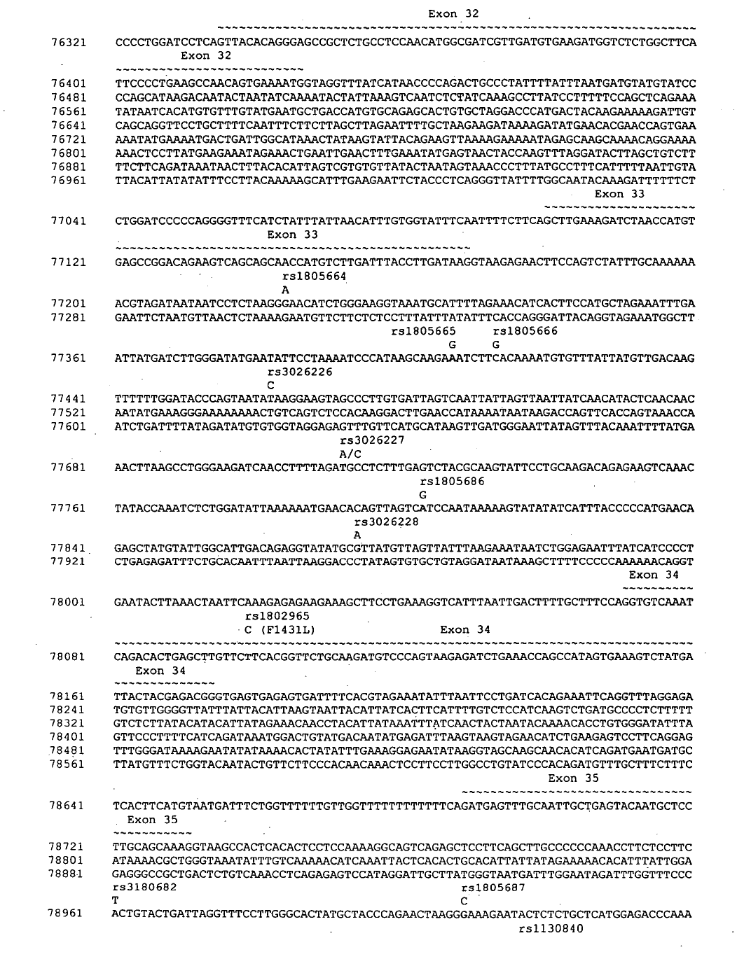

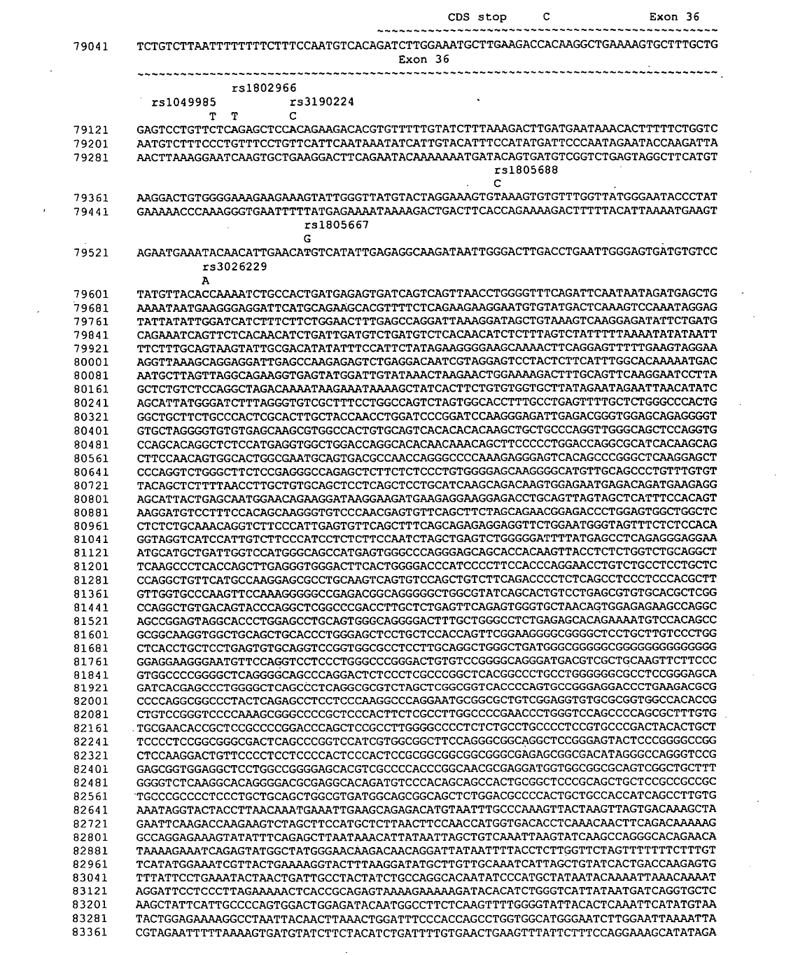

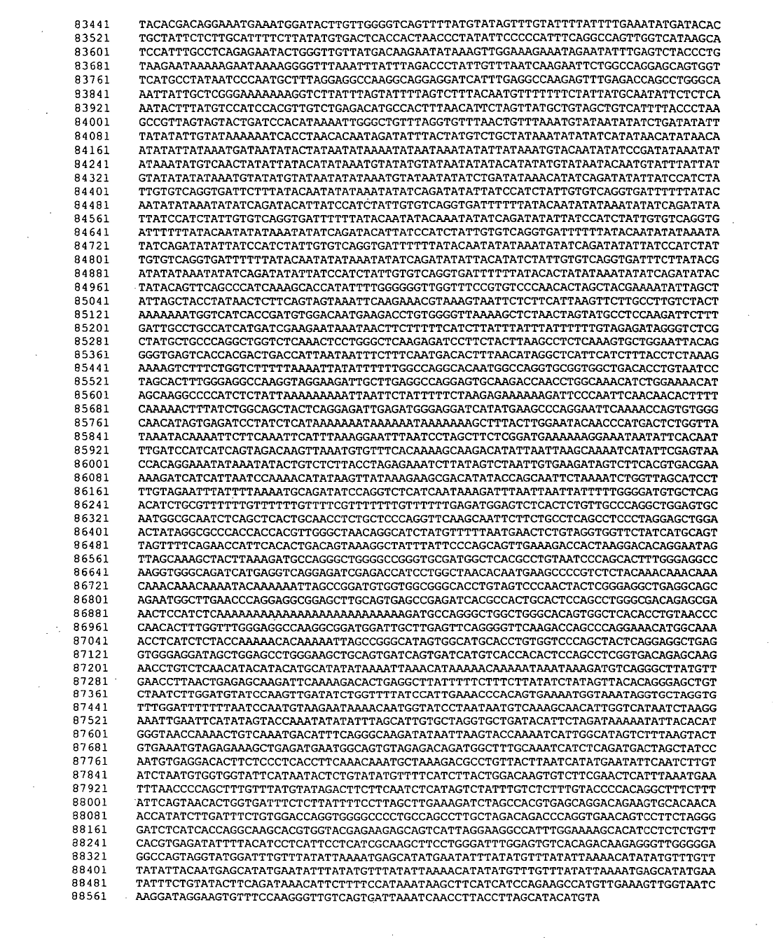

### RELATED APPLICATIONS

This application is a continuation-in-part of U.S. patent application Ser. No. 10/292,081, entitled SINGLE NUCLEOTIDE POLYMORPHISMS AND MUTATIONS ON ALPHA-2 MACROGLOBULIN, filed Nov. 8, 2002, and also a continuation-in-part of International PCT Application US 02/36095, entitled SINGLE NUCLEOTIDE POLYMORPHISMS AND MUTATIONS ON ALPHA-2 MACROGLOBULIN, filed Nov. 8, 2002; each of which are incorporated herein by reference in their entirety.

U.S. application Ser. No. 10/292,081 claims benefit under 119(e) of priority to U.S. Provisional Patent Application No. 60/337,434, entitled SINGLE NUCLEOTIDE POLYMORPHISMS AND MUTATIONS ON ALPHA-2 MACROGLOBULIN, filed Nov. 9, 2001, the disclosure of which is incorporated herein by reference in its entirety.

### GOVERNMENTAL INTERESTS

Subject matter of this application was made in part with government support. The United States Government may retain certain rights in this subject matter.

### FIELD OF THE INVENTION

The present invention is related to the field of disease diagnosis and treatment. More specifically, the invention is related to the discovery of single nucleotide polymorphisms (SNPs) and/or mutations in the Alpha-2-Macroglobulin gene (A2M). Included among the A2M polymorphisms and/or mutations are those that can be indicative of an altered risk for Alzheimer's Disease (AD).

### BACKGROUND OF THE INVENTION

Alpha-2-Macroglobulin (A2M) is an abundant plasma protein similar in structure and function to a group of proteins called α-macroglobulins. A2M is also produced in the brain where it binds multiple extracellular ligands and is internalized by neurons and astrocytes. In the brain of Alzheimer's disease (AD) patients, A2M has been localized to diffuse amyloid plaques. A2M also binds soluble β-amyloid and mediates its degradation. An excess of A2M, however, can have neurotoxic effects. Kovacs, Experimental Gerontology, 35:473-479 (2000). Based on genetic evidence, A2M is now recognized as one of the two confirmed late onset AD genes. As for the three early onset genes (the amyloid β-protein precursor and the two presenilins) and for the other late onset gene (ApoE), DNA polymorphisms in the A2M gene associated with AD result in significantly increased accumulation of amyloid plaques in AD brains. These data support an important role for A2M in AD etiopathology.

Human A2M is a 720 kDa soluble glycoprotein composed of four identical 180 kDa (1451 amino acid) subunits, each of which is encoded by a single-copy gene on chromosome 12. Disulfide bonds and noncovalent interactions connect the subunits within the tetramer. A2M is often referred to as a panprotease inhibitor, because it entraps and isolates virtually any protease from the extracellular environment followed by its degradation. Activation of A2M involves a complex conformational change of the tetramer, triggered either by protease cleavage of A2M or by methylamine treatment. Activation of A2M results in the entrapment of proteases and the exposure of the four receptor binding domains to the extracellular environment.

In the human A2M tetramer, each subunit contains at least five binding sites: the bait region, the internal thiol ester, the receptor binding site, the Aβ binding site, and the zinc binding site. The bait region, the internal thiol ester and the receptor binding site have a pivotal role in the activation and internalization of A2M. The bait region in each monomer is located between amino acids 666 to 706, at the center of each molecule, and it binds any known protease. The four bait regions in the tetramer are in close contact and are cleaved by the bound proteases, which triggers activation of A2M. This conformational change results in a sudden exposure of the four thiol esters between Cys949 and Glu952, and of the four receptor binding sites, to the extracellular environment.

The A2M region of chromosome 12 has first been associated with AD in genetic linkage analyses. (See e.g., Scott et al., JAMA, 281:513-514 (1999)). Two specific AD-associated polymorphisms have been reported in the A2M gene: an intronic deletion at exon 18 (18i; see e.g., Matthijs and Marynen, Nucleic Acids Res., 19:5102 (1991)) and a single amino acid substitution at position 1000 (1000 V/I; see e.g., Liao et al., Hum. Mol. Genet., 7:1953-1956 (1998)). Both of these polymorphisms were found to be associated with increased β-amyloid deposition (Myllykangas et al., Ann. Neurol., 46:382-390 (1999)).

Alzheimer's disease is a devastating neurodegenerative disorder that affects more than 4 million people per year in the US (Döbeli, H., Nat. Biotech. 15: 223-24 (1997)). It is the major form of dementia occurring in mid to late life: approximately 10% of individuals over 65 years of age, and approximately 40% of individuals over 80 years of age, are symptomatic of AD (Price, D. L., and Sisodia, S. S., Ann. Rev. Neurosci. 21:479-505 (1998)). The need for diagnostics and therapeutics for AD is manifest.

### SUMMARY OF THE INVENTION

Some aspects of the present invention are described in the numbered paragraphs below.

1. A method for identifying a polymorphism or combination of polymorphisms associated with an A2M-mediated disease or disorder, comprising testing one or more polymorphisms in an A2M gene individually and/or in combinations for genetic association with an A2M-mediated disease or disorder, wherein the one or more polymorphisms is/are selected from the group consisting of 6i, 12i.1, 12i.2, 12e, 14e, 14i.1, 14i.2, 17i.1, 20e, 20i, 21i, 28i and 30e.

2. A method for identifying a polymorphism or combination of polymorphisms associated with a neurodegenerative disease or disorder, comprising testing one or more polymorphisms in an A2M gene individually and/or in combinations for genetic association with a neurodegenerative disease or disorder, wherein the one or more polymorphisms is/are selected from the group consisting of 6i, 12i.1, 12i.2, 12e, 14e, 14i.1, 14i.2, 17i.1, 20e, 20i, 21i, 28i and 30e.

3. The method of Paragraph 1, wherein the nucleotide at 6i is C or A, the nucleotide at 12i.1 is C or G, the nucleotide at 12i.2 is A or T, the nucleotide at 12e is C or T, the nucleotide at 14e is T or C, the nucleotide at 14i.1 is no insertion or insertion of AAG, the nucleotide at 14i.2 is A or C, the nucleotide at 17i.1 is C or G, the nucleotide at 20e is C or T, the nucleotide at 20i is C or G, the nucleotide at 21i is T or A, the nucleotide at 28i is T or G and the nucleotide at 30e is T or C, or the complementary nucleotide thereof.

4. The method of Paragraph 2, wherein the nucleotide at 6i is C or A, the nucleotide at 12i.1 is C or G, the nucleotide at 12i.2 is A or T, the nucleotide at 12e is C or T, the nucleotide at 14e is T or C, the nucleotide at 14i.1 is no insertion or insertion of AAG, the nucleotide at 14i.2 is A or C, the nucleotide at 17i.1 is C or G, the nucleotide at 20e is C or T, the nucleotide at 20i is C or G, the nucleotide at 21i is T or A, the nucleotide at 28i is T or G and the nucleotide at 30e is T or C, or the complementary nucleotide thereof.

5. The method of Paragraph 2, wherein the disease is Alzheimer's disease.

6. A method of genotyping a cell comprising:

obtaining from an individual a biological sample containing an alpha-2-macroglobulin nucleic acid or portion thereof; and

determining the identity of one or more nucleotides in said alpha-2-macroglobulin nucleic acid or portion thereof wherein said one or more nucleotides are located at a position selected from the group consisting of 6i, 12i.1, 12i.2, 12e, 14e, 14i.1, 14i.2, 17i.1, 20e, 20i, 21i, 28i and 30e.

7. The method of Paragraph 6, wherein said alpha-2-macroglobulin nucleic acid is genomic DNA.

8. The method of Paragraph 6, wherein said alpha-2-macroglobulin nucleic acid is RNA.

9. The method of Paragraph 6, comprising determining the identity of one or more nucleotides at a position selected from the group consisting of 6i, 12e, and 14i.1.

10. The method of Paragraph 9, further comprising determining the identity of one or more nucleotides at a position selected from the group consisting of 18i and 1us.

11. The method of Paragraph 10, comprising determining the identity of one or more nucleotides at each of positions 1us, 6i, 12e, 14i.1 and 18i.

12. The method of Paragraph 6, comprising determining the identity of one or more nucleotides at a position selected from the group consisting of 6i, 12e, 14i.1 and 20e.

13. The method of Paragraph 12, further comprising determining the identity of one or more nucleotides at position 18i.

14. The method of Paragraph 6, comprising determining the identity of one or more nucleotides at a position selected from the group consisting of 6i, 12e, 14i.1 and 21i.

15. The method of Paragraph 14, further comprising determining the identity of one or more nucleotides at a position selected from the group consisting of 18i and 24e.

16. The method of Paragraph 15, comprising determining the identity of one or more nucleotides at each of positions 6i, 12e, 14i.1, 18i and 21i.

17. The method of Paragraph 6, comprising determining the identity of one or more nucleotides at a position selected from the group consisting of 12e, 14i.1 and 21i.

18. The method of Paragraph 17, further comprising determining the identity of one or more nucleotides at a position selected from the group consisting of 18i and 24e.

19. The method of Paragraph 18, comprising determining the identity of one or more nucleotides at each of positions 12e, 14i.1, 18i, 21i and 24e.

20. The method of Paragraph 6, comprising determining the identity of one or more nucleotides at a position selected from the group consisting of 14i.1, 20e and 21i.

21. The method of Paragraph 20, further comprising determining the identity of one or more nucleotides at a position selected from the group consisting of 18i and 24e.

22. The method of Paragraph 6, comprising determining the identity of one or more nucleotides at a position selected from the group consisting of 20e and 21i.

23. The method of Paragraph 22, further comprising determining the identity of one or more nucleotides at a position selected from the group consisting of 18i, 24e and rs1805654.

24. The method of Paragraph 6, comprising determining the identity of one or more nucleotides at a position selected from the group consisting of 14i.1 and 21i.

25. The method of Paragraph 24, further comprising determining the identity of one or more nucleotides at a position selected from the group consisting of 18i, 24e and rs1805654.

26. The method of Paragraph 25, comprising determining the identity of one or more nucleotides at each of positions 14i.1, 18i, 21i, 24e and rs1805654.

27. A method of genotyping a cell comprising:

obtaining from an individual a biological sample containing an alpha-2-macroglobulin polypeptide or portion thereof; and

determining the identity of one or more amino acids in said alpha-2-macroglobulin polypeptide or portion thereof wherein said one or more amino acids are located at a position selected from the group consisting of 14e, 20e and 30e.

28. A method of identifying a subject at risk for Alzheimer's Disease, said method comprising:

obtaining from said subject a biological sample containing an alpha-2-macroglobulin nucleic acid or portion thereof; and

determining the presence or absence of one or more polymorphisms or mutations in said alpha-2-macroglobulin nucleic acid or portion thereof wherein said one or more polymorphisms or mutations occur at a position selected from the group consisting of 6i, 12i.1, 12i.2, 12e, 14e, 14i.1, 14i.2, 17i.1, 20e, 20i, 21i, 28i and 30e.

29. The method of Paragraph 28, wherein said alpha-2-macroglobulin nucleic acid is genomic DNA.

30. The method of Paragraph 28, wherein said alpha-2-macroglobulin nucleic acid is RNA.

31. The method of Paragraph 28, wherein the nucleotide at 6i is C or A, the nucleotide at 12i.1 is C or G, the nucleotide at 12i.2 is A or T, the nucleotide at 12e is C or T, the nucleotide at 14e is T or C, the nucleotide at 14i.1 is no insertion or insertion of AAG, the nucleotide at 14i.2 is A or C, the nucleotide at 17i.1 is C or G, the nucleotide at 20e is C or T, the nucleotide at 20i is C or G, the nucleotide at 21i is T or A, the nucleotide at 28i is T or G and the nucleotide at 30e is T or C, or the complementary nucleotide thereof.

32. The method of Paragraph 28, comprising determining the presence or absence of one or more polymorphisms at a position selected from the group consisting of 6i, 12e, and 14i.1.

33. The method of Paragraph 32, further comprising determining the presence or absence of one or more polymorphisms at a position selected from the group consisting of 18i and 1us.

34. The method of Paragraph 33, comprising determining the presence or absence of one or more polymorphisms at each of positions 1us, 6i, 12e, 14i.1 and 18i.

35. The method of Paragraph 34, wherein the nucleotide at position 1us is G, the nucleotide at position 6i is C, the nucleotide at position 12e is C, the nucleotide at position 14i.1 is insertion of AAG, the nucleotide at position 18i is a pentanucleotide deletion, or the complementary nucleotide thereof.

36. The method of Paragraph 35, wherein the nucleotide at position 1us is G, the nucleotide at position 6i is C, the nucleotide at position 12e is T, the nucleotide at position 14i.1 is insertion of AAG, the nucleotide at position 18i is a pentanucleotide deletion, or the complementary nucleotide thereof.

37. The method of Paragraph 35, wherein the nucleotide at position 1us is T, the nucleotide at position 6i is C, the nucleotide at position 12e is T, the nucleotide at position 14i.1 is insertion of AAG, the nucleotide at position 18i is no deletion, or the complementary nucleotide thereof.

38. The method of Paragraph 28, comprising determining the presence or absence of one or more polymorphisms at a position selected from the group consisting of 6i, 12e, 14i.1 and 20e.

39. The method of Paragraph 38, further comprising determining the presence or absence of one or more polymorphisms at position 18i.

40. The method of Paragraph 28, comprising determining the presence or absence of one or more polymorphisms at a position selected from the group consisting of 6i, 12e, 14i.1 and 21i.

41. The method of Paragraph 40, further comprising determining the presence or absence of one or more polymorphisms at a position selected from the group consisting of 18i and 24e.

42. The method of Paragraph 41, comprising determining the presence or absence of one or more polymorphisms at each of positions 6i, 12e, 14i.1, 18i and 21i.

43. The method of Paragraph 42, wherein the nucleotide at position 6i is C, the nucleotide at position 12e is T, the nucleotide at position 14i.1 is insertion of AAG, the nucleotide at position 18i is no deletion, and the nucleotide at position 21i is A, or the complementary nucleotide thereof.

44. The method of Paragraph 28, comprising determining the presence or absence of one or more polymorphisms at a position selected from the group consisting of 12e, 14i.1 and 21i.

45. The method of Paragraph 44, further comprising determining the presence or absence of one or more polymorphisms at a position selected from the group consisting of 18i and 24e.

46. The method of Paragraph 45, comprising determining the presence or absence of one or more polymorphisms at each of positions 12e, 14i.1, 18i, 21i and 24e.

47. The method of Paragraph 46, wherein the nucleotide at position 12e is T, the nucleotide at position 14i.1 is insertion of AAG, the nucleotide at position 18i is no deletion, the nucleotide at position 21i is A, and the nucleotide at position 24e is A, or the complementary nucleotide thereof.

48. The method of Paragraph 28, comprising determining the presence or absence of one or more polymorphisms at a position selected from the group consisting of 14i.1, 20e and 21i.

49. The method of Paragraph 48, further comprising determining the presence or absence of one or more polymorphisms at a position selected from the group consisting of 18i and 24e.

50. The method of Paragraph 28, comprising determining the presence or absence of one or more polymorphisms at a position selected from the group consisting of 20e and 21i.

51. The method of Paragraph 50, further comprising determining the presence or absence of one or more polymorphisms at a position selected from the group consisting of 18i, 24e and rs1805654.

52. The method of Paragraph 28, comprising determining the presence or absence of one or more polymorphisms at a position selected from the group consisting of 14i.1 and 21i.

53. The method of Paragraph 52, further comprising determining the presence or absence of one or more polymorphisms at a position selected from the group consisting of 18i, 24e and rs1805654.

54. The method of Paragraph 53, comprising determining the identity of one or more nucleotides at each of positions 14i.1, 18i, 21i, 24e and rs1805654.

55. The method of Paragraph 54, wherein the nucleotide at position 14i.1 is insertion of AAG, the nucleotide at position 18i is a pentanucleotide deletion, the nucleotide at position 21i is T, the nucleotide at position 24e is A, and the nucleotide at position rs1805654 is G, or the complementary nucleotide thereof.

56. The method of Paragraph 28, comprising determining the presence or absence of one or more polymorphisms at a position selected from the group consisting of 12e, 14i.1, and 21i.

57. The method of Paragraph 56, wherein the nucleotide at position 12e is T, or the complement thereof, the nucleotide at position 14i.1 is AAG insertion, or the complement thereof, and the nucleotide at position 21i is T.

58. A method of identifying a subject at risk for Alzheimer's Disease, said method comprising:

obtaining from said subject a biological sample containing an alpha-2-macroglobulin polypeptide or portion thereof; and

determining the presence or absence of one or more polymorphisms or mutations in said alpha-2-macroglobulin polypeptide or portion thereof wherein said one or more polymorphisms or mutations occur at a position selected from the group consisting of 14e, 20e and 30e.

59. A method of identifying a compound that modulates an alpha-2-macroglobulin activity comprising:

providing a plurality of cells that express the LRP receptor;

contacting said cells with a candidate compound;

contacting said cells with an alpha-2-macroglobulin polypeptide comprising at least one polymorphism or mutation having a position selected from the group consisting of 14e, 20e, and 30e; and

identifying a compound that modulates an alpha-2-macroglobulin activity.

60. The method of Paragraph 59, wherein said alpha-2-macroglobulin activity is an interaction of said alpha-2-macroglobulin polypeptide with the LRP receptor.

61. The method of Paragraph 59, wherein said alpha-2-macroglobulin activity is the degradation of said alpha-2-macroglobulin polypeptide.

62. The method of Paragraph 59, wherein said alpha-2-macroglobulin activity is a protease inhibitor activity.

63. The method of Paragraph 59, wherein said alpha-2-macroglobulin activity is the clearance of said alpha-2-macroglobulin polypeptide.

64. The method of Paragraph 59, wherein said cells are contacted with an alpha-2-macroglobulin polypeptide in the presence of amyloid β.

65. The method of Paragraph 64, wherein said alpha-2-macroglobulin activity is an interaction of amyloid β or said alpha-2-macroglobulin polypeptide with the LRP receptor.

66. The method of Paragraph 65, wherein said alpha-2-macroglobulin mediates clearance of amyloid β.

67. A method of identifying a compound that modulates an alpha-2-macroglobulin activity comprising:

providing an alpha-2-macroglobulin polypeptide comprising at least one of the polymorphisms or mutations having a position selected from the group consisting of 14e, 20e, and 30e;

contacting said alpha-2-macroglobulin polypeptide with said compound;

contacting said alpha-2-macroglobulin polypeptide with methylamine; and

identifying a compound that modulates an alpha-2-macroglobulin activity by detecting a modulation in the activation of said alpha-2-macroglobulin polypeptide.

68. A method of identifying a compound that modulates an alpha-2-macroglobulin activity comprising:

providing an alpha-2-macroglobulin polypeptide comprising at least one of the polymorphisms or mutations having a position selected from the group consisting of 14e, 20e, and 30e;

contacting said alpha-2-macroglobulin polypeptide with said compound;

contacting said alpha-2-macroglobulin polypeptide with amyloid β; and

identifying a compound that modulates an alpha-2-macroglobulin activity by detecting a modulation in the formation of a complex of amyloid β and said alpha-2-macroglobulin polypeptide.

69. A method of making a pharmaceutical comprising:

identifying a compound by a method of any one of Paragraphs 59, 67 and 68

incorporating said compound into a pharmaceutical.

70. A purified or isolated nucleic acid comprising an alpha-2-macroglobulin sequence having a polymorphism or mutation at a position selected from the group consisting of 6i, 12i.1, 12i.2, 12e, 14e, 14i.1, 14i.2, 17i.1, 20e, 20i, 21i, 28i and 30e, wherein the nucleotide or nucleotide sequence at said position is other than an A2M-1.

71. The purified or isolated nucleic acid of Paragraph 70, wherein said alpha-2-macroglobulin sequence is SEQ ID NO: 1 or a sequence complementary thereto.

72. The purified or isolated nucleic acid of Paragraph 71, wherein the nucleotide or nucleotide sequence at said position is A2M-2.

73. The purified or isolated nucleic acid of Paragraph 70, wherein said alpha-2-macroglobulin sequence is selected from the group consisting of SEQ ID NOS: 2-8 and said polymorphism or mutation is at a position selected from the group consisting of 14e, 20e and 30e.

74. The purified or isolated nucleic acid of Paragraph 73, wherein the nucleotide or nucleotide sequence at said position is A2M-2.

75. The purified or isolated nucleic acid comprising a fragment of at least 16 consecutive nucleotides of SEQ ID NO: 1 having a polymorphism or mutation at a position selected from the group consisting of 6i, 12i.1, 12i.2, 12e, 14e, 14i.1, 14i.2, 17i.1, 20e, 20i, 21i, 28i and 30e, wherein the nucleotide or nucleotide at said position is other than an A2M-1 or a sequence complementary thereto.

76. The purified or isolated nucleic acid of Paragraph 75, wherein the nucleotide or nucleotide sequence at said position is A2M-2.

77. A purified or isolated polypeptide comprising an amino acid sequence selected from the group consisting of SEQ ID NOS: 9-15 having a polymorphism or mutation at a position selected from the group consisting of 14e, 20e and 30e, wherein the amino acid at said position is other than A2M-1.

78. The purified or isolated polypeptide of Paragraph 77, wherein the amino acid at said position is A2M-2.

79. A purified or isolated polypeptide comprising a fragment of an amino acid sequence selected from the group consisting of SEQ ID NOS: 9-15 having a polymorphism or mutation at a position selected from the group consisting of 14e, 20e and 30e, wherein the amino acid mutation at said position is other than A2M-1.

80. The purified or isolated polypeptide of Paragraph 79, wherein the amino acid at said position is A2M-2.

81. A recombinant vector comprising the nucleic acid of any one of Paragraphs 70-76.

82. A cultured cell comprising the nucleic acid of any one of Paragraphs 70-76 or the polypeptide of any one of Paragraphs 77-80.

83. A cultured cell comprising the recombinant vector of Paragraph 81.

84. An isolated or purified antibody that specifically binds to the polypeptide of any one of Paragraphs 77-80.

85. The antibody of Paragraph 84, wherein said antibody is monoclonal.

86. A method of expressing an alpha-2-macroglobulin polypeptide comprising:

providing a construct comprising a promoter operably linked to an alpha-2-macroglobulin nucleic acid having a polymorphism or mutation at a position selected from the group consisting of 14e, 20e and 30e, wherein the nucleotide at said position is other than an A2M-1; and

expressing said alpha-2-macroglobulin from said construct.

87. The method of Paragraph 86, wherein said nucleotide at said position is A2M-2.

### BRIEF DESCRIPTION OF THE DRAWINGS

The FIGURE shows a nucleotide sequence of a portion of chromosome 12 that includes the genomic sequence of A2M that has been annotated to include the locations of exons as well as the names and locations of the polymorphisms and/or mutations described herein. The name of the polymorphism and/or mutation as well as the corresponding nucleotide change(s) are indicated at positions above the A2M gene sequence. The nucleotide sequence provided in the FIGURE is from the University of California at Santa Cruz draft human genome sequence build 12 for chromosome positions 9007566-8918942 as is available at www.genome.ucsc.edu. The sequence presented is that of the “minus” strand in the sense that it is the complement of the strand that extends 5′→3′ from the p terminus to the centromere of chromosome 12. The sequence is, however, presented as the “sense” strand for the A2M gene. The sense strand refers to that strand of a double stranded nucleic acid molecule associated with a gene that has the sequence of the mRNA that encodes the amino acid sequence. This sequence also corresponds to nucleotides 1-88624 of NCBI Accession Number AC007436 (SEQ ID NO: 1).

### DETAILED DESCRIPTION OF THE INVENTION

Several single nucleotide polymorphisms (SNPs) and/or mutations of A2M gene have been discovered. Specifically, several novel SNPs and/or mutations were found in patients suffering from Alzheimer's Disease (AD). These SNPs and/or mutations are referred to as: 6i, 12i.1, 12i.2, 12e, 14e, 14i.1, 14i.2, 17i.1, 20e, 20i, 21i, 28i and 30e. The location of each of these SNPs and/or mutations on the A2M gene (Human Genome Project Gene Locus chr12: 9007566-8918942 (minus strand); including a section of human chromosome 12 the sequence of which is provided in National Center for Biotechnology Information (NCBI) Accession Number NT009702, incorporated herein by reference, and also present as nucleotides 1-88624 of NCBI Accession Number AC007436, incorporated herein by reference) (SEQ ID NO: 1) is identified in Table 1 and the FIGURE. Provided herein are polymorphisms in the region of chromosome 12 surrounding and including the A2M gene. Thus, the polymorphisms provided herein include polymorphisms in exons, introns or intervening sequences, intergenic regions and gene upstream and downstream regions, such as, for example, gene expression regulatory regions.

A particular polymorphism, depending on the nature and location of the polymorphism(s) in a gene allele, can play various roles in the manifestation of a disease condition or disorder. A polymorphism that gives rise to a particular variant phenotype can produce its effect(s), for example, at the level of RNA or protein. Effects on RNA include altered splicing, stability, editing and expression. Effects on the protein include altered protein function, folding, transport, localization, stability and expression. Polymorphisms located in the 5′ untranslated region of the gene may alter the activity of an element of the gene promoter and change the expression of the mRNA (e.g., level, pattern and/or timing of expression). Polymorphisms located in introns may alter RNA stability, editing, splicing, etc. Polymorphisms located in the 3′ untranslated region may influence polyadenylation, transcription and/or mRNA stability. Silent alterations in the coding region of a gene may affect codon usage and/or splicing. Changes in an encoded amino acid sequence, e.g., deletions and insertions, may affect protein function by increasing or decreasing a native function or bringing about an altered function.

The first column of Table 1 provides a name for each of the novel SNPs or mutations described herein. The name of the SNP or mutation (i.e., the polymorphism designation) corresponds to its general location in the A2M gene. For example, 14e refers to a SNP present in exon 14 of the A2M gene whereas 12i.1 refers to a SNP present in intron 12 of the A2M gene. The number to the right of the decimal point in 12i.1 indicates that this SNP is one of multiple SNPs found in intron 12. Table 1 also provides the location of each SNP with reference to SEQ ID NO: 1 (SEQ ID NO: 1 is the sequence of nucleotides 1-88624 of NCBI Accession Number AC007436, which contains the sequence of an A2M gene) and the nucleotide change(s) caused by each SNP or mutation. In particular, for each of the polymorphisms and/or mutations set out in Table 1, except for the 14i.1 mutation, the nucleotide to the left of the arrow in column 4 represents the nucleotide present in SEQ ID NO: 1 at the position indicated in column 2 of Table 1 (A2M-1). The nucleotide to the right of the arrow represents the nucleotide substitution that occurs at this position (A2M-2). For example, the A2M-1 allele of SNP 6i comprises a C at nucleotide position 37221 of NCBI Accession Number AC007436. The A2M-2 allele of SNP 6i comprises an A at nucleotide position 37221 of NCBI Accession Number AC007436. For the 14i.1 mutation, the A2M-2 allele comprises an insertion of the nucleotides “AAG” immediately following the nucleotide position indicated in column 2 of Table 1.

When reference is made herein to a SNP or mutation (as designated in column 1) with respect to a cDNA or any other contiguous nucleic acid sequence which encodes A2M, the location of the SNP or mutation with respect to a specific cDNA or A2M coding sequence is set out in column 3 of Table 1. Accordingly, the location of a SNP and or mutation in a particular cDNA or A2M coding sequence can be determined with reference to Table 1, column 3.

In cases where the SNP or mutation results in an amino acid change, the amino acid change and position are noted. The amino acid to the left of the arrow in column 5 represents the A2M-1 amino acid at the position indicated. The amino acid to the right of the arrow represents the A2M-2 amino acid at the position indicated. The FIGURE provides an annotated A2M gene sequence which shows each of the SNPs and/or mutations listed in Table 1, including both the A2M-1 alleles, represented by the nucleotides of SEQ ID NO: 1, and the A2M-2 alleles, represented by the nucleotides listed immediately above SEQ ID NO: 1. Accordingly, the locations of nucleotide or amino acid sequence polymorphisms set forth in Table 1 are referred to by the polymorphism designation (i.e., as set forth in column 1 of Table 1) with reference to a location corresponding to the nucleotide or amino acid position as set forth in columns 2 and 5 of Table 1, respectively.

Generally, when a polymorphism designation, for example, 6i, is referred to herein, it is used to specify a position or location within an A2M gene, cDNA, mRNA, mRNA or protein sequence, without regard to the particular nucleotide or amino acid that may be present at the position. The nucleotide or amino acid at the specified location of the A2M gene or A2M protein can be any nucleotide or amino acid unless a particular nucleotide or amino acid is specified.

TABLE 1
Novel SNPs and Mutations Associated with Alzheimer's Disease

| | Location with reference to NCBI | Location with | | |
|---|---|---|---|---|
| SNP/ | Accession Number AC007436 | reference to coding nucleotide | Nucleotide | Amino Acid Change (with |
| Mutation | (SEQ ID NO: 1) | sequences (e.g. cDNAs) | Change(s) | reference to SEQ ID NO: 9) |
| 6i | 174 bp downstream of exon 6 | | C→A | |
| | nucleotide position 37221 | | | |
| 12e | exon 12 | Nucleotide positions: 1339 of SEQ ID NOs: 3 | C→T | Y→Y |
| | nucleotide position 45269 | and 5; and 1338 of SEQ ID NO: 7 | | Silent effect |
| 12i.1 | 152 bp upstream of exon 12 | | C→G | |
| | nucleotide position 45088 | | | |
| 12i.2 | 115 bp upstream of exon 12 | | A→T | |
| | nucleotide position 45125 | | | |
| 14e | exon 14 | Nucleotide positions: 1730 of SEQ ID NOs: 3 | T→C | C→R |
| | nucleotide position 47519 | and 5; and 1729 of SEQ ID NO: 7 | | Amino acid position 563 |
| 14i.1 | 136 bp downstream of exon 14 | | No | |
| | nucleotide position 47669 | | insertion→ | |
| | | | insertion of | |
| | | | AAG | |
| 14i.2 | 151 bp downstream of exon 14 | | A→C | |
| | nucleotide position 47684 | | | |
| 17i.1 | 240 bp upstream of exon 18 | | C→G | |
| | nucleotide position 53095 | | | |
| 20e | exon 20 | Nucleotide positions: 2574 of SEQ ID NOs: 3 | C→T | A→V |
| | nucleotide position 56493 | and 5; 2573 of SEQ ID NO: 7; and 38 of SEQ | | Amino acid position 844 |
| | | ID NO: 4 | | |
| 20i | 27 bp downstream of exon 20 | | C→G | |
| | nucleotide position 56586 | | | |
| 21i | 2 bp upstream of exon 21 | | T→A | |
| | nucleotide position 56887 | | | |
| 28i | 55 upstream of exon 29 | | T→G | |
| | nucleotide position 72076 | | | |
| 30e | exon 30 | Nucleotide positions: 3912 of SEQ ID NOs: 3 | T→C | F→L |
| | nucleotide position 74154 | and 5; 3911 of SEQ ID NO: 7; and 1376 of | | Amino acid position 1290 |
| | | SEQ ID NO: 4 | | |

Table 2 provides a list of additional SNPs and mutations and their position on the A2M gene. The FIGURE also shows the positions of each of the SNPs and mutations listed in Table 2 as well as the nucleotide change (A2M-2) that is associated with the SNP and/or mutation.

TABLE 2
Additional SNPs and Mutations Associated with Alzheimer's Disease

| | | A2M Gene Sequence |
|---|---|---|
| Database | Chromosome 12 | Coordinate NCBI |
| SNP Identifier | Coordinate | Accession AC007436 |
| rs226379 | 8976642 | 30925 |
| rs226380 | 8976530 | 31037 |
| rs226381 | 8975616 | 31951 |
| rs3080605 | 8975391 | 32176 |
| rs226382 | 8974334 | 33233 |
| rs2302666 | 8973921 | 33646 |
| rs2477 | 8973853 | 33714 |
| rs226383 | 8973003 | 34564 |
| rs226384 | 8971704 | 35863 |
| rs226385 | 8971288 | 36279 |
| rs226386 | 8970784 | 36783 |
| rs226387 | 8969302 | 38265 |
| rs226388 | 8968337 | 39230 |
| rs226389 | 8967964 | 39603 |
| rs1049134 | 8964919 | 42648 |
| rs226390 | 8964765 | 42802 |
| rs226391 | 8964411 | 43156 |
| rs226392 | 8964312 | 43255 |
| rs226393 | 8963888 | 43679 |
| rs226394 | 8963091 | 44476 |
| rs226395 | 8962840 | 44727 |
| rs226396 | 8962283 | 45284 |
| rs226397 | 8961951 | 45616 |
| rs226398 | 8961373 | 46194 |
| rs226399 | 8959102 | 48465 |
| rs226400 | 8958524 | 49043 |
| rs226401 | 8958516 | 49051 |
| rs226402 | 8957932 | 49635 |
| rs226403 | 8957810 | 49757 |
| rs226404 | 8956453 | 51114 |
| rs226405 | 8956290 | 51277 |
| rs1800434 | 8955640 | 51927 |
| rs226406 | 8954411 | 53156 |
| rs226407 | 8953836 | 53731 |
| rs226408 | 8953258 | 54309 |
| rs226409 | 8953062 | 54505 |
| rs226410 | 8952700 | 54867 |
| rs113973 | 8952324 | 55243 |
| rs2277412 | 8952004 | 55563 |
| rs1049143 | 8951935 | 55632 |
| rs2277413 | 8951903 | 55664 |
| rs3180392 | 8951879 | 55688 |
| rs3210107 | 8951879 | 55688 |
| rs226411 | 8951178 | 56389 |
| rs226412 | 8949081 | 58486 |
| rs226413 | 8948804 | 58763 |
| rs2889706 | 8948741 | 58826 |
| rs2111023 | 8948292 | 59275 |
| rs226414 | 8947972 | 59595 |
| rs2193006 | 8944647 | 62920 |
| rs1800433 | 8940408 | 67159 |
| rs3168556 | 8940325 | 67242 |
| rs1805651 | 8939695 | 67872 |
| rs1805652 | 8938629 | 68938 |
| rs1805653 | 8938188 | 69379 |
| rs2377682 | 8938095 | 69472 |
| rs1805654 | 8937686 | 69881 |
| rs1805678 | 8937227 | 70340 |
| rs1805655 | 8936701 | 70866 |
| rs1805656 | 8936688 | 70879 |
| rs1805679 | 8936686 | 70881 |
| rs3026223 | 8936527 | 71040 |
| rs1805657 | 8936491 | 71076 |
| rs1805680 | 8936426 | 71141 |
| rs1805658 | 8936355 | 71212 |
| rs1805659 | 8936312 | 71255 |
| rs3026224 | 8936205 | 71362 |
| rs2300147 | 8936088 | 71479 |
| rs2300148 | 8936081 | 71486 |
| rs1805681 | 8935925 | 71642 |
| rs1805682 | 8935844 | 71723 |
| rs1805683 | 8935145 | 72422 |
| rs1805660 | 8935115 | 72452 |
| rs1805661 | 8935018 | 72549 |
| rs3080599 | 8934757 | 72810 |
| rs1805684 | 8934307 | 73260 |
| rs3026225 | 8934282 | 73285 |
| rs1805662 | 8934281 | 73286 |
| rs1805685 | 8933979 | 73588 |
| rs1805663 | 8932010 | 75557 |
| rs1805664 | 8930343 | 77224 |
| rs1805665 | 8930160 | 77407 |
| rs1805666 | 8930154 | 77413 |
| rs3026226 | 8930105 | 77462 |
| rs3026227 | 8929855 | 77712 |
| rs1805686 | 8929764 | 77803 |
| rs3026228 | 8929693 | 77874 |
| rs3180682 | 8928606 | 78961 |
| rs1805687 | 8928558 | 79009 |
| rs1049985 | 8928436 | 79131 |
| rs3190224 | 8928425 | 79142 |
| rs1805688 | 8928157 | 79410 |
| rs1805667 | 8928023 | 79544 |
| rs3026229 | 8927957 | 79610 |

It will be appreciated that the nomenclature for the polymorphisms and/or mutations used in the FIGURE and in Tables 1 and 2 refers to the location of the polymorphism and/or mutation disclosed herein. Accordingly, the use of a polymorphism or mutation name (or designation), such as 6i, 14e, or rs226381 indicates a polymorphic position in the reference nucleotide or amino acid sequence and not necessarily the identity of the nucleotide or amino acid change. The nucleotide and amino acid changes indicated in the FIGURE and in Table 1 correspond to one of many changes which can occur at the location of the polymorphism and/or mutation.

The reference nucleic acid sequence is provided by SEQ ID NO: 1 which corresponds to nucleotides 1-88624 of NCBI Accession Number AC007436. It will be appreciated that a nucleic acid corresponding to an A2M coding sequence (SEQ ID NO: 2) can be constructed by joining the exons at the splice sites listed for nucleotide sequence region 1-88624 as provided in the header section of NCBI Accession Number AC007436. Additionally, a number of cDNA variants of A2M are also available. These cDNAs, some of which encode variant polypeptides, are provided as SEQ ID NOs: 3-8. Variant A2M polypeptide sequences are provided as SEQ ID NOS: 9-15.

In view of the above, it will be appreciated that, although each of the novel SNPs and/or mutations disclosed herein are described with reference to SEQ ID NO: 1 (as well as SEQ ID NOS:2-15), each of these SNPs and/or mutations can occur in the context of nucleic acid sequence variants. For example, in addition to one or more of the SNPs disclosed herein, SNPs and/or mutations previously described for A2M (e.g. SNPs and/or mutations described in Table 2) may occur within SEQ ID NO: 1 (as well as SEQ ID NOS:2-15). Such nucleic acids having both one or more of the SNPs and/or mutations described herein and one or more known or previously described SNPs and/or mutations for A2M are contemplated by the present invention. Furthermore, A2M genes that have one or more of the SNPs and/or mutations described herein and which are altered from SEQ ID NO: 1 (as well as SEQ ID NOS:2-15) or known variants thereof as result from one or more sequencing errors are also contemplated by the present invention. As used herein, the term “mutation” means nucleotide variations that are not limited to single nucleotide substitution. For example, a mutation includes, but is not limited to, the insertion of one or more bases, the deletion of one or more bases, or an inversion of multiple bases.

In view of the above, as used herein, “A2M”, “A2M gene” or “A2M genomic nucleic acid”, when used with reference to SEQ ID NO: 1, means the nucleic acid sequence of SEQ ID NO: 1 or portions thereof as well as any nucleic acid variants which include one or more SNPs and/or mutations, such as those described in Table 2 and the FIGURE. Similarly, “A2M cDNA”, “A2M coding sequence” or “A2M coding nucleic acid”, when used with reference to SEQ ID NOS: 2-8, means the nucleic acid sequences of SEQ ID NOS: 2-8 or portions thereof as well as nucleic acid variants which include one or more SNPs and/or mutations, such as those described in Table 2 and the FIGURE. With respect to polypeptides “A2M”, “A2M polypeptide” or “A2M protein”, when used with reference to SEQ ID NOS: 9-15, means the amino acid sequence of SEQ ID NOS: 9-15 or portions thereof as well as amino acid sequence variants which are encoded by nucleic acids which include one or more SNPs and/or mutations, such as those described in Table 2, and the FIGURE and which effect the polypeptide encoded by the A2M coding sequence.

According to some aspects of the present invention, A2M includes nucleotide sequences having at least 99%, 98%, 97%, 96%, 95%, 94%, 93%, 92%, 91%, 90%, 89%, 88%, 87%, 86%, or 85% sequence identity to SEQ ID NO: 1 as determined by BLASTN with default parameters (Altschul et al, (1990) J. Mol. Biol. 215: 403, incorporated herein by reference in its entirety). In other aspects of the present invention, A2M coding sequence includes nucleotides sequences having at least 99%, 98%, 97%, 96%, 95%, 94%, 93%, 92%, 91%, 90%, 89%, 88%, 87%, 86%, or 85% sequence identity to any one of SEQ ID NOS: 2-8 as determined by BLASTN version 2.0 with default parameters (Altschul et al, (1990) J. Mol. Biol. 215: 403, incorporated herein by reference in its entirety). In still other aspects of the present invention, A2M includes polypeptide sequences having at least 95%, 94%, 93%, 92%, 91%, 90%, 89%, 88%, 87%, 86%, 85%, 84%, 83%, 82%, 81%, or 80% sequence identity or similarity to any one of SEQ ID NOS: 9-15 as determined by FASTA version 3.0t78 with default parameters (Pearson and Lipman, (1988) Proc. Natl. Acad. Sci. USA, 85: 2444, incorporated herein by reference in its entirety).

As used in connection with any one of the polymorphisms and/or mutations disclosed herein, A2M-1 refers to the nucleotide or nucleotide sequence of SEQ ID NO: 1 which is present at the location of the polymorphism or mutation. As used in connection with any one of the polymorphisms and/or mutations disclosed herein, A2M-2 refers to the nucleotide change, nucleotide insertion or nucleotide deletion indicated in the FIGURE and/or in Table 1 which is present at the location of the polymorphism or mutation. As used in connection with any one of the polymorphisms and/or mutations disclosed herein, A2M-1 refers to the amino acid of SEQ ID NO: 9 which is present at the location of the polymorphism or mutation. As used in connection with any one of the polymorphisms and/or mutations disclosed herein, A2M-2 refers to the amino acid change indicated in the FIGURE and/or in Table 1 which is present at the location of the polymorphism or mutation.

Polymorphisms can serve as genetic markers. A genetic marker is a DNA segment with an identifiable location in a chromosome. Genetic markers may be used in a variety of genetic studies such as, for example, locating the chromosomal position or locus of a DNA sequence of interest, identifying genetic associations of a disease, and determining if a subject is predisposed to or has a particular disease. Because DNA sequences that are relatively close together on a chromosome tend to be inherited together, tracking of a genetic marker through generations in a family and comparing its inheritance to the inheritance of another DNA sequence of interest can provide information useful in determining the relative position of the DNA sequence of interest on a chromosome. Genetic markers particularly useful in such genetic studies are polymorphic. Such markers also may have an adequate level of heterozygosity to allow a reasonable probability that a randomly selected person will be heterozygous.

The polymorphisms provided herein in the region of chromosome 12 surrounding and including the A2M gene include single nucleotide polymorphisms (SNPs). SNPs have use as genetic markers, for example, in fine genetic mapping and genetic association analysis, as well as linkage analysis [see, e.g., Kruglyak (1997) Nature Genetics 17:21-24]. Combinations of SNPs (which individually occur about every 100-300 bases) can also yield informative haplotypes. Also provided herein, are polymorphisms of the A2M gene and surrounding region of chromosome 12 that are associated, individually and/or in combination, with a neurodegenerative disease, such as, for example, Alzheimer's disease.

Based on the discovery of association between SNPs described herein, individually and/or in combinations (haplotypes), with AD, additional markers associated with AD may now be identified using methods as described herein and known in the art. The availability of additional markers is of particular interest in that it will increase the density of markers for this chromosomal region and can provide a basis for identification of an AD DNA segment or gene in the region of chromosome 12. An AD DNA segment or gene may be found in the vicinity of the marker or set of markers showing the highest correlation with AD. Furthermore, the availability of markers associated with AD makes possible genetic analysis-based methods of determining a predisposition to or the occurrence of AD in an individual by detection of a particular allele.

Polymorphisms of the A2M gene region of chromosome 12 provided herein may be analyzed individually and in combinations, e.g., haplotypes, for genetic association with any disease or disorder. In a particular example, the disease is a neurodegenerative disease, such as, for example, AD. Thus, also provided herein are methods of identifying polymorphisms associated with diseases and disorders. The methods involve a step of testing polymorphisms of the A2M gene, and/or surrounding region of chromosome 12, and in particular the polymorphisms provided herein, individually or in combination, e.g., haplotypes, for association with a disease or disorder. For example, the polymorphisms provided herein can be tested individually, in combinations of the provided polymorphisms, or in combinations with other previously described polymorphisms (e.g., polymorphisms listed in Table 2). The analysis or testing may involve genotyping DNA from individuals affected with the disease or disorder, and possibly also from related or unrelated individuals, with respect to the polymorphic marker and analyzing the genotyping data for association with the disease or disorder using methods described herein and/or known to those of skill in the art. For example, statistical analysis of the data may involve a chi-squared or Fisher's exact test and may be conducted in conjunction with a number of programs, such as the transmission disequilibrium test (TDT), affected family based control test (AFBAC) and the haplotype relative risk test (HRR). Case-control strategies can be applied to the testing, as can, for example, TDT approaches.

Several embodiments of the invention have biotechnological, diagnostic, and therapeutic use. For example, the nucleic acids and proteins described herein can be used as probes to isolate more polymorphic and/or mutant A2M genes, to detect the presence or absence of wild type or polymorphic and/or mutant A2M proteins in an individual, and these molecules can be incorporated into constructs for preparing recombinant polymorphic and/or mutant A2M proteins or used in methods of searching or identifying agents that modulate A2M levels and/or activity, for example, candidate therapeutic agents. The sequences of the nucleic acids and/or proteins described herein can also be incorporated into computer systems, used with modeling software so as to enable rational drug design. Information obtained from genotyping methods provided herein can be used, for example, in computer systems, in pharmacogenomic profiling of therapeutic agents to predict effectiveness of an agent in treating an individual for a neurodegenerative disease such as AD. The nucleic acids and/or proteins described herein can also be incorporated into pharmaceuticals and used for the treatment of neuropathies, such as Alzheimer's Disease (AD).

Accordingly, some embodiments of the invention include isolated or purified nucleic acids comprising, consisting essentially of, or consisting of an A2M gene, cDNA or mRNA with one or more of the SNPs and/or mutations described in Table 1 or a fragment of said A2M gene, cDNA or mRNA, wherein said fragment contains at least 9, at least 16 or at least 18 consecutive nucleotides of the polymorphic or mutant A2M gene, cDNA or mRNA but including at least one of the SNPs and/or mutations in Table 1. Isolated or purified nucleic acids that are complementary to said A2M nucleic acids and fragments thereof are also embodiments.

Some nucleic acid embodiments for example, include genomic DNA, RNA, and cDNA encoding the polymorphic and/or mutant A2M proteins or fragments thereof. Methods for obtaining such nucleic acid sequences are also embodiments. The nucleic acid embodiments can be altered, mutated, or changed such that the alteration, mutation, or change results in a conservative amino acid replacement. These altered or changed nucleic acids are equivalent to the nucleic acids described herein. In some contexts, the term “consisting essentially of” is used to include nucleic acids having the changes or alterations above.

Vectors having the nucleic acids above, including expression vectors, and cells containing said nucleic acids and vectors are also embodiments. Methods of making these constructs and cells are aspects of the invention, as well. Other embodiments of the invention include genetically altered organisms that express the polymorphic and/or mutant A2M transgenes or polymorphic portions thereof (e.g., mutant A2M transgenic or knockout animals). Methods of making such organisms are also aspects of the invention. Transgenic animals that are contemplated (particularly non-human animals) can be used, for example, in elucidating disease processes and/or identifying therapeutic agents.

Some polypeptide embodiments of the invention include isolated, enriched, recombinant or purified polypeptides consisting of, consisting essentially of, or comprising the complete amino acid sequences (or portions thereof containing the polymorphic amino acid change) of the polymorphic and/or mutant A2M proteins described herein. (See Table 1, which includes the nucleotide polymorphisms of the A2M gene coding sequence that result in corresponding amino acid changes in the A2M polypeptide sequence. Additionally, Table 1 sets out the identity and location of the amino acid substitution with respect to a reference A2M polypeptide sequence). Other polypeptide embodiments are equivalents to the polymorphic and/or mutant A2M proteins described herein in that said equivalent molecules have conservative amino acid substitutions. In some contexts, the term “consisting essentially of” is used to include polypeptides having such conservative amino acid substitutions. Embodiments also include isolated, enriched, recombinant or purified fragments of the polymorphic and/or mutant A2M proteins at least 3 amino acids in length so long as said fragments contain at least one of the amino acid polymorphisms and/or mutants described herein (See Table 1). Additional embodiments concern methods of preparing the polypeptides and peptides described herein and, in some preparative methods, chemical synthesis and/or recombinant techniques are used.

Embodiments of the invention also include antibodies directed to the mutant and/or polymorphic A2M proteins. Preferably, said antibodies specifically interact with the mutant and/or polymorphic A2M proteins and can be used to differentiate wild-type A2M proteins (e.g., A2M proteins having a reference sequence of amino acids and/or that are most prevalent in the population or in a particular study) from polymorphic and/or mutant A2M proteins. The antibody embodiments can be monoclonal or polyclonal and approaches to manufacture both types of antibodies, which are specific for the polymorphic and/or mutant A2M proteins are disclosed.

Approaches to rational drug design are also provided in this disclosure, and these methods can be used to identify molecules that interact with the polymorphic and/or mutant A2M proteins or fragments thereof. Molecules that interact with the polymorphic and/or mutant A2M proteins or fragments thereof are referred to as “binding partners”. Preferred binding partners modulate (e.g., increase or decrease) the activity of the polymorphic and/or mutant A2M proteins or fragments thereof. The various activities of the polymorphic and/or mutant A2M proteins or fragments thereof can include, but are not limited to, the ability to bind proteases, bind amyloid-P, bind a receptor (e.g., the LRP receptor), bind zinc, and the ability to form a tetramer. Several computer-based methodologies are discussed, which involve three-dimensional modeling of the polymorphic and/or mutant A2M proteins or fragments thereof and suspected binding partners (e.g., antibodies, proteases, amyloid-β, zinc, and the LRP receptor).

Several A2M characterization assays are also described. These assays test the functionality of a polymorphic and/or mutant A2M protein or fragment thereof and can identify agents that modulate the activity and/or expression of such proteins, including, for example, binding partners that interact with said molecules. Agents that modulate the activity of a wild-type or polymorphic or mutant A2M, for example, can be identified using an A2M characterization assay and molecules identified using these methods can be incorporated into medicaments and pharmaceuticals, which can be provided to subjects in need of treatment or prevention of neuropathies, including AD.

Some functional assays involve the use of multimeric polymorphic and/or mutant A2M proteins or fragments thereof and/or binding partners, which are disposed on a support, such as a resin, bead, lipid vesicle or cell membrane. These multimeric agents are contacted with candidate binding partners and the association of the binding partner with the multimeric agent is determined. Successful binding agents can be further analyzed for their effect on A2M function in other types of cell based assays. One such assay evaluates internalization of a protease or amyloid β. Other types of characterization assays involve molecular biology techniques designed to identify protein-protein interactions (e.g., two-hybrid systems).

The diagnostic embodiments of the invention (including diagnostic kits) are designed to identify individuals at risk of acquiring AD or individuals that have a predilection for AD. Nucleic acid and protein based diagnostics are provided. Some of these diagnostics identify individuals at risk for acquiring AD by detecting a particular nucleotide or amino acid polymorphism and/or mutation or combinations of polymorphisms and/or mutations, for example a haplotype, in an A2M gene or A2M protein. Other diagnostic approaches are concerned with the detection of aberrant amounts or levels of expression of polymorphic or mutant A2M RNA or A2M protein. The polymorphisms and/or mutations, levels of expression of polymorphic or mutant A2M RNAs or proteins can be recorded in a database, which can be accessed to identify a type of AD, a suitable treatment., and subjects for which further genotyping should be investigated. It is contemplated that many other SNPs and/or mutations, which are predictive of AD, can be found in subjects identified as already having at least one SNP and/or mutation described herein.

Accordingly, a method of identifying an individual having an altered risk for AD is provided, wherein a biological sample containing nucleic acid is obtained from an individual, and the sample is analyzed to determine the nucleotide identity of at least one novel SNP and/or mutation, such as at least one SNP and/or mutation selected from the group consisting of 6i, 12i.1, 12i.2, 12e, 14e, 14i.1, 14i.2, 17i.1, 20e, 20i, 21i, 28i and 30e. The presence or absence of a particular nucleotide or nucleotide sequence at the location of any one of these SNPs and/or mutations can indicate an altered risk of AD. Additionally, the nucleotide identity information obtained from the analysis of combinations of SNPs and/or mutations can further indicate an altered risk of AD. The biological sample can also be analyzed to determine the nucleotide identity of publicly available SNPs and/or mutations. Nucleotide identity information obtained from the analysis of publicly available SNPs and/or mutations in combination with novel SNPs disclosed herein, such as at least one SNP and/or mutation selected from the group consisting of 6i, 12i.1, 12i.2, 12e, 14e, 14i.1, 14i.2, 17i.1, 20e, 20i, 21i, 28i and 30e, can indicate an altered risk for AD. The analysis can include an association study (e.g., a family study) and/or haplotype analysis.

Also provided are methods of identifying polymorphisms associated with a disease or disorder. The novel SNPs and/or mutations described herein, such as a SNP and/or mutation selected from the group consisting of 6i, 12i.1, 12i.2, 12e, 14e, 14i.1, 14i.2, 17i.1, 20e, 20i, 21i, 28i and 30e, can be analyzed separately or in combinations to identify association with any A2M-mediated disease or disorder. The polymorphisms can be analyzed to identify association with neurodegenerative diseases. For example, a single or combinations of novel SNPs and/or mutations can be checked for association with neurodegenerative disorders or other diseases having a relationship to the A2M gene using methods well known in the art, such as those described herein.

For example, the genotype of individuals with respect to one or more polymorphisms and/or mutations selected from the group consisting of 6i, 12i.1, 12i.2, 12e, 14e, 14i.1, 14i.2, 17i.1, 20e, 20i, 21i, 28i and 30e can be compared between individuals that have AD or a particular disease or a family history of the disease and individuals that do not have the disease or a family history of the disease so as to identify a polymorphism or combination of polymorphisms that associate with a disease or disorder, such as a neurodegenerative disease or disorder, for example AD. Additionally, since there are many different genotypes that can be associated with AD, individuals with AD having one genotype can be compared with individuals with AD having another genotype to identify the presence of a novel SNP and/or mutation. In one embodiment of the invention, the information and analysis above can be recorded on a database and the comparisons can be performed by a computer system accessing said database. Thus, by virtue of the fact that at least one SNP and/or mutation selected from the group consisting of 6i, 12i.1, 12i.2, 12e, 14e, 14i.1, 14i.2, 17i.1, 20e, 20i, 21i, 28i and 30e has been identified in an individual or a family, the nucleic acids and proteins isolated or purified from said individuals becomes a novel tool with which more SNPs and mutations associated with AD can be identified.

In yet another aspect of the present invention, the information gained from analyzing biological samples obtained from one or more individuals to determine the nucleotide identity of at least one novel SNP and/or mutation described herein, such as the SNPs and/or mutations selected from the group consisting of 6i, 12i.1, 12i.2, 12e, 14e, 14i.1, 14i.2, 17i.1, 20e, 20i, 21i, 28i and 30e, can be used in fine chromosome mapping of chromosome 12, in genetic association studies, in pharmacogenetic profiling and pharmacogenetic-based treatment programs and in the search for a gene responsible for AD or other AD-associated genes.

Also provided herein are methods of genotyping an individual comprising obtaining a nucleic acid sample from an individual and determining the nucleotide identity of at least one novel SNP and/or mutation described herein, such as at least one SNPs and/or mutations selected from the group consisting of 6i, 12i.1, 12i.2, 12e, 14e, 14i.1, 14i.2, 17i.1, 20e, 20i, 21i, 28i and 30e. In a particular embodiment, the nucleotide identity of more than one novel polymorphism and/or mutation is determined. Accordingly, a set of novel polymorphisms and/or mutations can be analyzed to determine the nucleotide identity for each polymorphism and/or mutation in the entire set. The set of polymorphisms and/or mutations can also include polymorphisms and/or mutations that are publicly available as well as novel polymorphisms and/or mutations. Determination of the nucleotide identities for sets of polymorphisms and/or mutations as described above provides a method for determining the haplotype of an individual.

Also provided herein are methods of confirming a phenotypic diagnosis of a disease or disorder which include a step of detecting in nucleic acid obtained from a subject diagnosed with a disease or disorder the presence or absence of one or more polymorphisms and/or selected from the group consisting of 6i, 12i.1, 12i.2, 12e, 14e, 14i.1, 14i.2, 17i.1, 20e, 20i, 2 1i, 28i and 30e, wherein the presence of the one or more polymorphisms, individually and/or in combination, confirms a phenotypic diagnosis of the disease or disorder. In a particular embodiment of these methods, the disease or disorder is an A2M-mediated disease disorder. In one embodiment, the disease or disorder is a neurodegenerative disease or disorder, such as, for example, AD. For example, the disease may be Alzheimer's disease with an onset age of greater than or equal to about 50 years, or greater than or equal to about 60 years, or greater than or equal to about 65 years. In another embodiment of the methods of confirming a phenotypic diagnosis of a neurodegenerative disease or disorder, the method further includes a step of detecting in nucleic acid obtained from the subject the presence or absence of one or more polymorphisms of at least one different gene allele associated with neurodegenerative disease. In a particular embodiment, the at least one different gene allele is an APOE4 allele.

Further provided are methods of treating a subject manifesting an Alzheimer's disease phenotype. Certain ambiguous phenotypes, e.g., dementia, manifested in AD also occur in connection with other diseases and conditions which may be treated using drugs and other treatments that are different from drugs and methods used to treat AD.

Genotyping of polymorphisms of the A2M gene region described herein, and optionally other AD-associated markers, in subjects manifesting such an AD phenotype(s) permits confirmation of AD phenotypic diagnoses and assists in distinguishing between AD and other possible diseases or disorders. Once an individual is genotyped as having or being predisposed to AD, he or she may be treated with any known methods effective in treating AD.

Accordingly, methods of treating a subject manifesting an Alzheimer's disease phenotype provided herein include steps of

(a) determining the nucleotide identity, in a nucleic acid obtained from the subject, of one or more polymorphisms selected from the group consisting of 6i, 12i.1, 12i.2, 12e, 14e, 14i.1, 14i.2, 17i.1, 20e, 20i, 21i, 28i and 30e, wherein the presence of a particular nucleotide or nucleotides at the one or more polymorphisms, individually and/or in combination, is indicative of the occurrence of Alzheimer's disease in a subject; and

(b) selecting and/or administering a treatment that is effective for treatment of Alzheimer's disease.

The pharmaceutical embodiments of the invention include medicaments containing an agent, for example, a binding partners that modulates the activity of wild-type or polymorphic or mutant A2M. These medicaments can be prepared in accordance with conventional methods of galenic pharmacy for administration to organisms in need of treatment. A therapeutically effective amount of agent, for example, a binding partner (e.g., an amount sufficient to modulate the function of a wild-type or polymorphic or mutant A2M) can be incorporated into a pharmaceutical composition with or without a carrier. Routes of administration of the pharmaceuticals of the invention include, but are not limited to, topical, transdermal, parenteral, gastrointestinal, transbronchial, and transalveolar. These pharmaceuticals can be provided to subjects in need of treatment for neurodegenerative diseases, in particular AD. The section below describes several of the nucleic acid embodiments of the invention.

A2M Nucleic Acids

The A2M nucleotide sequences of the invention include: (a) the nucleotide sequence provided in NCBI Accession Number AC007436 nucleotide positions 1-88624, incorporated herein by reference in its entirety (SEQ ID NO: 1), or a portion thereof, as modified by a nucleotide(s) change at least one SNP and/or mutation selected from the group consisting of 6i, 12i.1, 12i.2, 12e, 14e, 14i.1, 14i.2, 17i.1, 20e, 20i, 21i, 28i and 30e as indicated in the FIGURE and/or in Table 1; (b) nucleotide sequences encoding amino acid sequences (a sequence formed by the joining the exons of the genomic sequence provided in NCBI Accession Number AC007436 between nucleotide positions 31033 and 79197 (SEQ ID NO: 2), or A2M, mRNA or cDNA sequences (e.g., SEQ ID NOS: 3-8) as modified by a nucleotide(s) change at least one SNP and/or mutation selected from the group consisting of 12e, 14e, 20e, and 30e as indicated in the FIGURE and/or in Table 1; (c) the nucleotide sequence provided in SEQ ID NO: 1, or a portion(s) thereof, wherein the nucleotide at a position corresponding to 37221 is A, T or G, the nucleotide at a position corresponding to 45269 is T, A or G, the nucleotide at a position corresponding to 45088 is G, A or T, the nucleotide at a position corresponding to 45125 is T, C or G, the nucleotide at a position corresponding to 47519 is C, A or G, the nucleotide at a position corresponding to 47684 is C, G or T, the nucleotide at a position corresponding to 53095 is G, A or T, the nucleotide at a position corresponding to 56493 is T, A or G, the nucleotide at a position corresponding to 56586 is G, A or T, the nucleotide at a position corresponding to 56887 is C, G or A, the nucleotide at a position corresponding to 72076 is T, A or C, the nucleotide at a position corresponding to 74154 is C, A or G, and/or the sequence of AAG occurs between nucleotides at positions corresponding to positions 47669 and 47670; and (d) the nucleotide sequence provided in SEQ ID NO: 1, or a portion(s) thereof, wherein the nucleotide at a position corresponding to 37221 is A, the nucleotide at a position corresponding to 45269 is T, the nucleotide at a position corresponding to 45088 is G, the nucleotide at a position corresponding to 45125 is T, the nucleotide at a position corresponding to 47519 is C, the nucleotide at a position corresponding to 47684 is C, the nucleotide at a position corresponding to 53095 is G, the nucleotide at a position corresponding to 56493 is T, the nucleotide at a position corresponding to 56586 is G, the nucleotide at a position corresponding to 56887 is C, the nucleotide at a position corresponding to 72076 is T, the nucleotide at a position corresponding to 74154 is C, and/or the sequence of AAG occurs between nucleotides corresponding to positions 47669 and 47670.

Additionally, aspects of the present invention include the A2M coding sequences and cDNAs of SEQ ID NOS: 2-8 as modified by a nucleotide(s) change at least one SNP and/or mutation selected from the group consisting of 12e, 14e, 20e, and 30e. More embodiments concern the nucleic acids of SEQ ID NOS: 1-8 having nucleotide(s) variations at one or more previously described SNPs and/or mutations for A2M (e.g. SNPs and/or mutations provided in Table 2) in addition to a nucleotide(s) change at least one SNP and/or mutation selected from the group consisting of 6i, 12i.1, 12i.2, 12e, 14e, 14i.1, 14i.2, 17i.1, 20e, 20i, 21i, 28i and 30e.

In this regard, the nucleic acid embodiments described herein can have from 9 to approximately 88,624 consecutive nucleotides so long as the sequence contains nucleotide(s) variation at a SNP and/or mutation selected from the group consisting of 6i, 12i.1, 12i.2, 12e, 14e, 14i.1, 14i.2, 17i.1, 20e, 20i, 21i, 28i and 30e, for example, or the nucleotides specified for the particular locations within SEQ ID NO: 1 as set forth in (c) and (d) immediately above. Some of these compositions, for example, include nucleic acids having any number between 9-50, 16-50, 17-50, 18-50, 19-50, 50-100, 100-500, 500-1000, 1000-10,000, 10,000-50,000, or 50-88,634 consecutive nucleotides of SEQ. ID. NO. 1, wherein said nucleic acid contains a SNP and/or mutation selected from the group consisting of 6i, 12i.1, 12i.2, 12e, 14e, 14i.1, 14i.2, 17i.1, 20e, 20i, 21i, 28i and 30e (e.g., greater than or equal to 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 500, 600, 700, 800, 900, 1000, 1300, 1400, 1500, 1600, 1700, 1800, 1900, 2000, 3000, 5000, 10,000, 25,000, 50,000, 75,000 and 88,624 consecutive nucleotides of a sequence of SEQ ID NO: 1 or portions of the above nucleotide list for SEQ ID NOS: 2-8, wherein said nucleic acid contains a nucleotide(s) variation at a SNP and/or mutation selected from the group consisting of 6i, 12i.1, 12i.2, 12e, 14e, 14i.1, 14i.2, 17i.1, 20e, 20i, 21i, 28i and 30e). In one embodiment, the nucleic acids comprise at least 12, 13, 14, 15, 16, 17, 18, 19, 20 consecutive nucleotides of a sequence of SEQ ID NO: 1 or SEQ ID NOS: 2-8, wherein said nucleic acid contains a nucleotide(s) variation SNP and/or mutation selected from the group consisting of 6i, 12i.1, 12i.2, 12e, 14e, 14i.1, 14i.2, 17i.1, 20e, 20i, 21i, 28i and 30e, for example, or the nucleotides specified for the particular locations within SEQ ID NO: 1 as set forth in (c) and (d) immediately above, or a complement thereof. In another embodiment, the nucleic acid embodiments comprise at least 20-30 consecutive nucleotides of a sequence of SEQ ID NO: 1 or SEQ ID NOS: 2-8, wherein said nucleic acid contains a nucleotide(s) variation at a SNP and/or mutation selected from the group consisting of 6i, 12i.1, 12i.2, 12e, 14e, 14i.1, 14i.2, 17i.1, 20e, 20i, 21i, 28i and 30e, for example, or the nucleotides specified for the particular locations within SEQ ID NO: 1 as set forth in (c) and (d) immediately above, or complement thereof.

Several embodiments also include the above-described fragments of the nucleic acids of SEQ ID NOS: 1-8 having a nucleotide(s) variation at one or more previously described SNPs and/or mutations for A2M (e.g. SNPs and/or mutations provided in Table 2) in addition to a nucleotide(s) variation at least one SNP and/or mutation selected from the group consisting of 6i, 12i.1, 12i.2, 12e, 14e, 14i.1, 14i.2, 17i.1, 20e, 20i, 21i, 28i and 30e, for example, the nucleotides specified for the particular locations within SEQ ID NO: 1 as set forth in (c) and (d) immediately above.

The nucleic acid embodiments described herein can also be altered by mutation such as substitutions, additions, or deletions that provide for sequences encoding equivalent molecules. Due to the degeneracy of nucleotide coding sequences, other DNA sequences that encode substantially the same polymorphic/mutant A2M amino acid sequence can be made. These include, but are not limited to, nucleic acid sequences comprising all or portions of SEQ ID NO: 1 or SEQ ID NOS: 2-8, wherein said nucleic acid sequences contain a nucleotide(s) variation at a SNP and/or mutation selected from the group consisting of 6i, 12i.1, 12i.2, 12e, 14e, 14i.1, 14i.2, 17i.1, 20e, 20i, 21i, 28i and 30e, or complements thereof, which have been altered by the substitution of different codons that encode a functionally equivalent amino acid residue within the sequence, thus producing a silent change.

The nucleic acid sequences described above have biotechnological and diagnostic use, e.g., in nucleic acid hybridization assays, Southern and Northern Blot analysis, etc. and the prognosis of neuropathies, such as Alzheimer's Disease (AD). By using the nucleic acid sequences described herein, for example, probes that complement the polymorphic and/or mutant A2M genes or cDNAs can be designed and manufactured by oligonucleotide synthesis. Desirable probes comprise a nucleic acid sequence that is unique to the polymorphic and/or mutant A2M genes or cDNAs. These probes can be used to screen nucleic acids isolated from tested individuals so as to identify the presence or absence of a polymorphism or combination of polymorphisms indicative of an altered, for example increased, risk of AD. Analysis can involve denaturing gradient gel electrophoresis or denaturing HPLC methods, for example. For guidance regarding probe design and denaturing gradient gel electrophoresis or denaturing HPLC methods see, e.g., Ausubel et al., 1989, Current Protocols in Molecular Biology, Green Publishing Associates and Wiley Interscience, N.Y., including updated materials, U.S. Pat. Nos. 5,795,976; 5,585,236; 6,024,878; 6,210,885; Huber, et al., Chromatographia 37:653 (1993); Huber, et al., Anal. Biochem. 212:351 (1993); Huber, et al., Anal. Chem. 67:578 (1995); O'Donovan et al., Genomics 52:44 (1998), Am J Hum Genet. December;67(6):1428-36 (2000); Ann Hum Genet. September:63 (Pt 5):383-91 (1999); Biotechniques, April;28(4):740-5 (2000); Biotechniques. November;29(5):1084-90, 1092 (2000); Clin Chem. August;45(8 Pt 1):1133-40 (1999); Clin Chem. April;47(4):635-44 (2001); Genomics. August 15;52(1):44-9 (1998); Genomics. March 15;56(3):247-53 (1999); Genet Test. ;1(4):237-42 (1997-98); Genet Test.:4(2):125-9 (2000); Hum Genet. June; 106(6):663-8 (2000); Hum Genet. November; 107(5):483-7 (2000); Hum Genet. November;107(5):488-93 (2000); Hum Mutat. December;16(6):518-26 (2000); Hum Mutat. 15(6):556-64 (2000); Hum Mutat. March; 17(3):210-9 (2001); J Biochem Biophys Methods. November 20;46(1-2):83-93 (2000); J Biochem Biophys Methods. January 30;47(1-2):5-19 (2001); Mutat Res. November 29;430(1):13-21(1999); Nucleic Acids Res. March 1;28(5):E13 (2000); and Nucleic Acids Res. October 15;28(20):E89 (2000), all of which, including the references contained therein, are hereby expressly incorporated by reference in their entireties.

Also provided herein are oligonucleotides that can serve as primers. Such oligonucleotides can be made, for example, by conventional oligonucleotide synthesis for use in isolation and diagnostic procedures that employ the Polymerase Chain Reaction (PCR) or other enzyme-mediated nucleic acid amplification techniques or primer extension techniques. For a review of PCR technology, see Molecular Cloning to Genetic Engineering White, B. A. Ed. in Methods in Molecular Biology 67: Humana Press, Totowa (1997), the disclosure of which is incorporated herein by reference in its entirety and the publication entitled “PCR Methods and Applications” (1991, Cold Spring Harbor Laboratory Press), the disclosure of which is incorporated herein by reference in its entirety.

Oligonucleotide primers provided herein can contain a sequence of nucleotides that specifically hybridizes adjacent to or at a polymorphic region of the A2M gene spanning a nucleotide position corresponding to any of the following nucleotide positions of SEQ ID NO: 1: 37221, 45269, 45088, 45125, 47519, 47684, 53095, 56493, 56586, 56887, 72076, 74154 and 47669, or the complementary positions thereof adjacent to or at a polymorphic region of an A2M cDNA spanning a nucleotide position corresponding to any of the following positions: 1339, 1730, 2574 and 3912 of SEQ ID NOs: 3 and 5; 1338, 1729, 2573 and 3911 of SEQ ID NO: 7; and 38 and 1376 of SEQ ID NO: 4. In particular embodiments, the oligonucleotides hybridize to a polymorphic region of the A2M gene under conditions of moderate or high stringency. Also provided are oligonucleotides, such as primers and probes that are the complements of these primers and probes. In particular embodiments, the probes or primers contain a number of nucleotides sufficient to allow specific hybridization to the target nucleotide sequence. In particular embodiments of the probes and primers provided herein, the molecules are of sufficient length to specifically hybridize to portions of an A2M gene at polymorphic sites. Typically such lengths depend upon the complexity of the source organism genome. For humans such lengths generally are at least 14, 15, 16, 17, 18 or 19 nucleotides, and typically may be at least 20, 30, 40, 50, 60, 70, 80, 90, 100, 200, 300, 400 or 500 or more nucleotides. In other embodiments, such lengths of the probes and primers provided are not more than 14, 15, 16, 17, 18 or 19 nucleotides, and further may be not more than 20, 30, 40, 50, 60, 70, 80, 90, 100, 200, 300, 400, 500, 600, 700, 800, 900 or 1000 nucleotides in length.

For amplification of mRNAs, it is within the scope of the invention to reverse transcribe mRNA into cDNA followed by PCR (RT-PCR); or, to use a single enzyme for both steps as described in U.S. Pat. No. 5,322,770, the disclosure of which is incorporated herein by reference in its entirety. Another technique involves the use of Reverse Transcriptase Asymmetric Gap Ligase Chain Reaction (RT-AGLCR), as described by Marshall R. L. et al. (PCR Methods and Applications 4:80-84, 1994), the disclosure of which is incorporated herein by reference in its entirety. In each of these amplification procedures, primers on either side of the sequence to be amplified are added to a suitably prepared nucleic acid sample along with dNTPs and a thermostable polymerase, such as Taq polymerase, Pfu polymerase, or Vent polymerase. The nucleic acid in the sample is denatured and the primers are specifically hybridized to complementary nucleic acid sequences in the sample. The hybridized primers are then extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188, the disclosure of which is incorporated herein by reference in their entirety.

The primers are selected to be substantially complementary to a portion of the nucleic acid sequence of SEQ ID NO: 1 or SEQ ID NOS: 2-8 that is downstream and upstream of the SNP and/or mutation to be detected such that the fragment produced by the amplification or extension reaction contains the SNP and/or mutant. Preferably, primers are designed to be downstream and upstream of at least one of 6i, 12i.1, 12i.2, 12e, 14e, 14i.1, 14i.2, 17i.1, 20e, 20i, 21i, 28i and 30e, for example downstream or upstream of a nucleotide position corresponding to any of the following positions: 1339, 1730, 2574 and 3912 of SEQ ID NOS: 3 and 5; 1338, 1729, 2573 and 3911 of SEQ ID NO: 7; and 38 and 1376 of SEQ ID NO: 4, thereby allowing the sequences between the primers to be amplified or extended. Primers are desirably 16, 17, 18, 19, 20, 21, 22, 23, 24, 25, 26, 27, 28, 29 and 30 nucleotides in length. The formation of stable hybrids depends on the melting temperature (Tm) of the DNA. The Tm depends on the length of the primer, the ionic strength of the solution and the G+C content. The higher the G+C content of the primer, the higher is the melting temperature because G:C pairs are held by three H bonds whereas A:T pairs have only two. The G+C content of the amplification primers of the present invention preferably ranges between 10 and 75%, more preferably between 35 and 60%, and most preferably between 40 and 55%. The appropriate length for primers under a particular set of assay conditions can be empirically determined by one of skill in the art.

The spacing of the primers relates to the length of the segment to be amplified. In the context of the present invention, amplified segments carrying nucleotides corresponding to a nucleotide location of 6i, 12i.1, 12i.2, 12e, 14e, 14i.1, 14i.2, 17i.1, 20e, 20i, 21i, 28i and/or 30e can range in size from at least about 25 bp to 35 kb. Amplification fragments that are any number from 25-1000 bp, 50-1000 bp, and fragments that are any number from 100-600 bp are common. It will be appreciated that amplification primers can be of any sequence that allows for specific amplification of a region of a polymorphic and/or mutant A2M gene and can, for example, include modifications such as restriction sites to facilitate cloning.

The PCR product can be subcloned and sequenced to ensure that the amplified sequences represent the sequences of polymorphic and/or mutant A2M gene. The PCR fragment can then be used to isolate a full length cDNA clone by a variety of methods. For example, the amplified fragment can be labeled and used to screen a cDNA library, such as a bacteriophage cDNA library. Alternatively, the labeled fragment can be used to isolate genomic clones via the screening of a genomic library.

Aspects of the invention also encompass (a) DNA vectors that contain any of the foregoing nucleic acid sequences; (b) DNA expression vectors that contain any of the foregoing nucleic acid sequences operatively associated with a regulatory element that directs the expression of the coding sequences; and (c) genetically engineered host cells that contain any of the foregoing nucleic acid sequences operatively associated with a regulatory element that directs the expression of the coding sequences in the host cell. These recombinant constructs are capable of replicating autonomously in a host cell. Alternatively, the recombinant constructs can become integrated into the chromosomal DNA of a host cell.

As used herein, regulatory elements include, but are not limited to, inducible and non-inducible promoters, enhancers, operators and other elements known to those skilled in the art that drive and regulate expression. Such regulatory elements include, but are not limited to, the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

In addition, recombinant polymorphic and/or mutant A2M-encoding nucleic acid sequences can be engineered so as to modify processing or expression of the protein. For example, and not by way of limitation, the polymorphic and/or mutant A2M genes can be combined with a promoter sequence and/or ribosome binding site, or a signal sequence can be inserted upstream of A2M-encoding sequences to permit secretion of the A2M protein and thereby facilitate harvesting or bioavailability. Additionally, a given polymorphic and/or mutant A2M nucleic acid can be mutated in vitro or in vivo, to create and/or destroy translation, initiation, and/or termination sequences, or to create variations in coding regions and/or form new restriction sites or destroy preexisting ones, or to facilitate further in vitro modification. Any technique for mutagenesis known in the art can be used, including but not limited to, in vitro site-directed mutagenesis. (Hutchinson et al., J. Biol. Chem., 253:6551 (1978), herein incorporated by reference).

Further, nucleic acids encoding other proteins or domains of other proteins can be joined to nucleic acids encoding polymorphic and/or mutant A2M proteins or fragments thereof so as to create a fusion protein. Nucleotides encoding fusion proteins can include, but are not limited to, a full length polymorphic and/or mutant A2M protein, a truncated polymorphic and/or mutant A2M protein or a peptide fragment of a polymorphic and/or mutant A2M protein fused to an unrelated protein or peptide, such as for example, a transmembrane sequence, which anchors the A2M peptide fragment to the cell membrane; an Ig Fc domain which increases the stability and half life of the resulting fusion protein (e.g., A2M-Ig); or an enzyme, fluorescent protein, luminescent protein which can be used as a marker (e.g., an A2M-Green Fluorescent Protein (“A2M-GFP”) fusion protein). The fusion proteins are useful as biotechnological tools or pharmaceuticals or both, as will be discussed infra. The section below describes several of the polypeptides of the invention and methods of making these molecules.

The disclosed nucleic acids and others that can be obtained using methods described herein may be transferred into a host cell such as bacteria, yeast, insect, mammalian, or plant cell for recombinant expression therein. Thus, provided herein are recombinant cells containing an A2M gene or a portion or portions thereof, such as, for example, a transcriptional control region (including, for example, a promoter and 3′ untranslated (UTR) sequences) and/or a coding sequence of an A2M gene. The A2M gene or portion(s) thereof contains at least one polymorphic region and is thus referred to as a polymorphic A2M gene or portion(s) thereof. An “A2M gene or a portion or portions thereof” includes an A2M cDNA or portion(s) thereof.

Cells containing nucleic acids encoding polymorphic A2M proteins, and vectors and cells containing the nucleic acids as provided herein permit production of the polymorphic proteins, as well as antibodies to the proteins. This provides a means to prepare synthetic or recombinant polymorphic proteins and fragments thereof that are substantially free of contamination from other proteins, the presence of which can interfere with analysis of the polymorphic proteins. In addition, the polymorphic proteins may be expressed in combination with selected other proteins that the protein of interest may associate with in cells. The ability to selectively express the polymorphic proteins alone or in combination with other selected proteins makes it possible to observe the functioning of the recombinant polymorphic proteins within the environment of a cell.

Recombinant cells provided herein may be used for numerous purposes. For example, the cells may be used in testing polymorphic A2M genes or portion(s) thereof for characterization of phenotypic outcomes correlated with the particular polymorphisms. The cells may also be used in the production of recombinant A2M protein. Such protein may be used, for example, in assays for molecules that bind to, and in particular affect the activity of, A2M. The proteins may also be used in the production of antibodies specific for the protein. Additionally, the recombinant A2M protein may be used as a source of a protease inhibitor. Recombinant cells containing polymorphic A2M genes or portion(s) thereof may also be used in methods of identifying agents that modulate A2M gene and protein expression and/or activity or that modulate a biological event characteristic of a disease or disorder involving altered A2M gene and/or protein expression or function which may be candidate treatments for a disease or disorder.

Also provided herein are methods of producing recombinant cells by introducing nucleic acid containing a polymorphic A2M gene or portion(s) as described herein thereof into a cell. The cell may be any transfectable cell. Such cells, and methods of introducing heterologous nucleic acids into the cells, are known to those of skill in the art.

The exogenous nucleic acid containing a polymorphic A2M gene or portion(s) thereof that is used in the generation of recombinant cells provided herein contains, in particular embodiments, a sequence of nucleotides that ultimately provides for a product upon transcription of the A2M gene or portion(s) thereof. The product can be, for instance, RNA and/or a protein translated from a transcript. For example, the product can be A2M mRNA and/or an A2M protein or a reporter molecule such as a reporter protein. If the polymorphic A2M gene or portion(s) thereof being used in the generation of recombinant cells provided herein does not contain sequences that provide for transciption of the A2M gene or portion(s) thereof, any appropriate transcription control sequences, such as a promoter, from any appropriate source which will provide for transciption of the A2M gene or portion(s) thereof in the cell can be used. If the polymorphism(s) occur in a transcription control region of an A2M gene, the polymorphic control region of the gene can be isolated or synthesized and operatively linked to nucleic acid encoding a reporter molecule, e.g.,. -galactosidase, a fluorescent protein such as green fluorescent protein, or some other readily detectable molecule, or nucleic acid encoding an A2M protein. The resultant fusion gene can be used as the transgene that is introduced into a host cell for use in development of recombinant cells therefrom. The patterns and levels of expression of the reporter or other molecule in the recombinant cells can be analyzed and compared to those in cells containing a fusion gene in which a wild-type or reference A2M transcription control region sequence is operatively linked to nucleic acid encoding a reporter or other molecule.

Polymorphic and/or Mutant A2M Polypeptides

Isolated or purified polymorphic and/or mutant A2M polypeptides and fragments of these molecules at least 3 amino acids in length, which contain at least one of the mutations identified in Table 1, are embodiments of the invention. In some contexts, the term “polymorphic and/or mutant A2M polypeptides” refers not only to the full-length polymorphic and/or mutant A2M proteins but also to fragments of these molecules at least 3 amino acids in length but containing at least one of the mutations identified in Table 1.

The nucleic acids encoding the A2M polypeptides or fragments thereof, described in the previous section, can be manipulated using conventional techniques in molecular biology so as to create recombinant constructs that express polymorphic and/or mutant A2M polypeptides. The polymorphic and/or mutant A2M polypeptides or fragments thereof of the invention, include but are not limited to, those containing as a primary amino acid sequence all or part of the amino acid sequence encoded by SEQ ID NO: 1, SEQ ID NO: 2 (encoding SEQ ID NO: 9) or SEQ ID NOS: 3-8 (encoding SEQ ID NOS: 10-15), as modified by a SNP and/or mutation described in Table 1 (for example, 14e, 20e and 30e), and fragments of these proteins at least three amino acids in length but including at least one of the mutations listed in Table 1, including altered sequences in which functionally equivalent amino acid residues are substituted for residues within the sequence resulting in a silent change. The A2M peptide fragments of the invention can be, for example, any number of between 4-20, 20-50, 50-100, 100-300, 300-600, 600-1000, 1000-1450 consecutive amino acids of SEQ. ID NOs. 9-15 (e.g., less than or equal to 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41, 42, 43, 44, 45, 46, 47, 48, 49, 50, 51, 52, 53, 54, 55, 56, 57, 58, 59, 60, 61, 62, 63,64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 500, 600, 700, 800, 900, 1000, and 1450 amino acids in length of SEQ ID NOS: 9-15). Polypeptides of the present invention also contemplate the polypeptides of SEQ ID NOS: 9-15 or fragments thereof encoded by the nucleic acids of SEQ ID NOS: 2-8 having one or more previously described SNPs and/or mutations for A2M which affect the A2M polypeptide (e.g. some SNPs and/or mutations provided in Table 2) in addition to at least one SNP and/or mutation selected from the group consisting of 14e, 20e and 30e.

Embodiments also include isolated or purified polymorphic and/or mutant A2M polypeptides that have one or more amino acid residues within the polypeptide that are substituted by another amino acid of a similar polarity that acts as a functional equivalent, resulting in a silent alteration. Substitutes for an amino acid within the sequence can be selected from other members of the class to which the amino acid belongs. For example, the non-polar (hydrophobic) amino acids include alanine, leucine, isoleucine, valine, proline, phenylalanine, tryptophan, and methionine. The polar neutral amino acids include glycine, serine, threonine, cysteine, tyrosine, asparagine and glutamine. The positively charged (basic) amino acids include arginine, lysine, and histidine. The negatively charged (acidic) amino acids include aspartic acid and glutamic acid. The aromatic amino acids include phenylalanine, tryptophan, and tyrosine.

The sequences, constructs, vectors, clones, and other materials comprising the embodiments of the present invention can be in enriched or isolated form. As used herein, “enriched” means that the concentration of the material is at least about 2, 5, 10, 100, or 1000 times its natural concentration (for example), advantageously 0.01%, by weight, preferably at least about 0. 1% by weight. Enriched preparations from about 0.5%, 1%, 5%, 10%, and 20% by weight are also contemplated. The term “isolated” requires that the material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated. It is also advantageous that the sequences be in purified form. The term “purified” does not require absolute purity; rather, it is intended as a relative definition. Isolated proteins have been conventionally purified to electrophoretic homogeneity by Coomassie staining, for example. Purification of starting material or natural material to at least one order of magnitude, preferably two or three orders, and more preferably four or five orders of magnitude is expressly contemplated.

The polymorphic and/or mutant A2M polypeptides described herein can be prepared by chemical synthesis methods (such as solid phase peptide synthesis) using techniques known in the art such as those set forth by Merrifield et al., J. Am. Chem. Soc. 85:2149 (1964), Houghten et al., Proc. Natl. Acad. Sci. USA, 82:51:32 (1985), Stewart and Young (Solid phase peptide synthesis, Pierce Chem Co., Rockford, Ill. (1984), and Creighton, 1983, Proteins: Structures and Molecular Principles, W. H. Freeman & Co., N.Y., all of which are hereby incorporated by reference in their entireties. Such polypeptides can be synthesized with or without a methionine on the amino terminus. Chemically synthesized polypeptides can be oxidized using methods set forth in these references to form disulfide bridges.

While the polymorphic and/or mutant A2M polypeptides and fragments thereof can be chemically synthesized, it can be more effective to produce these molecules by recombinant DNA technology using techniques well known in the art. Such methods can be used to construct expression vectors containing the polymorphic and/or mutant A2M nucleotide sequences, for example, and appropriate transcriptional and translational control signals. These methods include, for example, in vitro recombinant DNA techniques, synthetic techniques, and in vivo genetic recombination. Alternatively, RNA capable of encoding an polymorphic and/or mutant A2M polypeptide sequences and fragments thereof can be chemically synthesized using, for example, synthesizers. See, for example, the techniques described in Oligonucleotide Synthesis, 1984, Gait, M. J. ed., IRL Press, Oxford, which is incorporated by reference herein in its entirety.

In several embodiments, polymorphic and/or mutant A2M nucleic acids and polypeptides are expressed in a cell line. For example, some cells are made to express the a polymorphic and/or mutant A2M polypeptide having the sequence encoded by SEQ ID NOs: 2-8 or such nucleic acids having one or more previously described SNPs and/or mutations for A2M which affect the A2M polypeptide in addition to at least one SNP and/or mutation selected from the group consisting of 14e, 20e and 30e. A variety of host-expression vector systems can be utilized to express the polymorphic and/or mutant A2M nucleic acids and polypeptides of the invention. The expression systems that can be used include, but are not limited to, microorganisms such as bacteria (e.g., E. coli or B. subtilis) transformed with recombinant bacteriophage DNA, plasmid DNA or cosmid DNA expression vectors containing polymorphic and/or mutant A2M nucleotide sequences; yeast (e.g., Saccharomyces, Pichia) transformed with recombinant yeast expression vectors containing the polymorphic and/or mutant A2M nucleotide sequences; insect cell systems infected with recombinant virus expression vectors (e.g., Baculovirus) containing the polymorphic and/or mutant A2M sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing polymorphic and/or mutant A2M nucleotide sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors can be advantageously selected depending upon the use intended for the polymorphic and/or mutant A2M gene product being expressed. For example, when a large quantity of such a protein is to be produced, for the generation of pharmaceutical compositions of polymorphic and/or mutant A2M polypeptide or for raising antibodies to the polymorphic and/or mutant A2M polypeptide, for example, vectors which direct the expression of high levels of fusion protein products that are readily purified can be desirable. Such vectors include, but are not limited, to the E. coli expression vector pUR278 (Ruther et al., EMBO J., 2:1791 (1983), in which the polymorphic and/or mutant A2M nucleic acids can be ligated individually into the vector in frame with the lacZ coding region so that a fusion protein is produced; pIN vectors (Inouye & Inouye, Nucleic Acids Res., 13:3101-3109 (1985); Van Heeke & Schuster, J. Biol. Chem., 264:5503-5509 (1989)); and the like, herein expressly incorporated by reference. pGEX vectors can also be used to express foreign polypeptides as fusion proteins with glutathione S-transferase (GST). In general, such fusion proteins are soluble and can be purified from lysed cells by adsorption to glutathione-agarose beads followed by elution in the presence of free glutathione. The PGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The polymorphic and/or mutant A2M nucleic acid sequences can be cloned individually into non-essential regions (for example the polyhedrin gene) of the virus and placed under control of an AcNPV promoter (for example the polyhedrin promoter). Successful insertion of polymorphic and/or mutant A2M nucleic acid sequence will result in inactivation of the polyhedrin gene and production of non-occluded recombinant virus, (i.e., virus lacking the proteinaceous coat coded for by the polyhedrin gene). These recombinant viruses are then used to infect Spodoptera frugiperda cells in which the inserted gene is expressed. (E.g., see Smith et al., J. Virol. 46: 584 (1983); and Smith, U.S. Pat. No. 4,215,051, all of which are hereby expressly incorporated by reference in their entireties).

In mammalian host cells, a number of viral-based expression systems can be utilized. In cases where an adenovirus is used as an expression vector, the polymorphic and/or mutant A2M nucleotide sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the polymorphic and/or mutant A2M gene product in infected hosts. (E.g., See Logan & Shenk, Proc. Natl. Acad. Sci. USA 81:3655-3659 (1984), herein expressly incorporated by reference in its entirety). Specific initiation signals can also be required for efficient translation of inserted nucleotide sequences. These signals include the ATG initiation codon and adjacent sequences. In cases where an entire polymorphic and/or mutant A2M gene or cDNA, including its own initiation codon and adjacent sequences, is inserted into the appropriate expression vector, no additional translational control signals are needed.

However, in cases where only a portion of the polymorphic and/or mutant A2M coding sequence is inserted, exogenous translational control signals, including, perhaps, the ATG initiation codon, may be provided. Furthermore, the initiation codon is desirably in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (See Bittner et al., Methods in Enzymol., 153:516-544 (1987)).

In addition, a host cell strain can be chosen which modulates the expression of the inserted sequences, or modifies and processes the gene product in the specific fashion desired. Such modifications (e.g., glycosylation) and processing (e.g., cleavage) of protein products are important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include, but are not limited to, CHO, VERO, BHK, HeLa, COS, MDCK, 293, 3T3, and WI38.

For long-term, high-yield production of recombinant proteins, stable expression is preferred. For example, cell lines that stably express the polymorphic and/or mutant A2M sequences described herein can be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells are allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn are cloned and expanded into cell lines. This method is advantageously used to engineer cell lines which express the polymorphic and/or mutant A2M gene product. Such engineered cell lines are particularly useful in screening and evaluation of compounds that affect the endogenous activity of the polymorphic and/or mutant A2M gene product.

A number of selection systems can be used, including but not limited to the herpes simplex virus thymidine kinase (Wigler, et al., Cell 11:223 (1977), hypoxanthine-guanine phosphoribosyltransferase (Szybalska & Szybalski, Proc. Natl. Acad. Sci. USA 48:2026 (1962), and adenine phosphoribosyltransferase (Lowy, et al., Cell 22:817 (1980) genes can be employed in tk⁻, hgprt⁻ or aprt⁻ cells, respectively. Also, antimetabolite resistance can be used as the basis of selection for the following genes: dhfr, which confers resistance to methotrexate (Wigler, et al., Proc. Natl. Acad. Sci. USA 77:3567 (1980); O'Hare, et al., Proc. Natl. Acad. Sci. USA 78:1527 (1981); gpt, which confers resistance to mycophenolic acid (Mulligan & Berg, Proc. Natl. Acad. Sci. USA 78:2072 (1981); neo, which confers resistance to the aminoglycoside G-418 (Colberre-Garapin, et al., J. Mol. Biol. 150:1 (1981); and hygro, which confers resistance to hygromycin (Santerre, et al., Gene 30:147 (1984)).

Alternatively, any fusion protein can be readily purified by utilizing an antibody specific for the fusion protein being expressed. For example, a system described by Janknecht et al. allows for the ready purification of non-denatured fusion proteins expressed in human cell lines. (Janknecht, et al., Proc. Natl. Acad. Sci. USA 88: 8972-8976 (1991)). In this system, the gene of interest is subcloned into a Vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

The polymorphic and/or mutant A2M nucleic acids and polypeptides can also be expressed in plants, insects, and animals so as to create a transgenic organism. Plants and insects of almost any species can be made to express the polymorphic and/or mutant A2M nucleic acids and/or polypeptides, described herein. Desirable transgenic plant systems having one or more of these sequences include Arabadopsis, Maize, and Chlamydomonas. Desirable insect systems having one or more of the polymorphic and/or mutant A2M nucleic acids and/or polypeptides include, for example, D. melanogaster and C. elegans. Animals of any species, including, but not limited to, amphibians, reptiles, birds, mice, rats, rabbits, guinea pigs, pigs, micro-pigs, goats, dogs, cats, and non-human primates, e.g., baboons, monkeys, and chimpanzees can be used to generate polymorphic and/or mutant A2M containing transgenic animals. Transgenic organisms of the invention desirably exhibit germline transfer of polymorphic and/or mutant A2M nucleic acids and polypeptides. Still other transgenic organisms of the invention exhibit complete knockouts or point mutations of one or more of the A2M genes described herein.

Any technique known in the art is preferably used to introduce the polymorphic and/or mutant A2M transgene into animals to produce the founder lines of transgenic animals or to knock out or replace existing A2M genes. Such techniques include, but are not limited to pronuclear microinjection (Hoppe, P. C. and Wagner, T. E., 1989, U.S. Pat. No. 4,873,191); retrovirus mediated gene transfer into germ lines (Van der Putten et al., Proc. Natl. Acad. Sci., USA 82:6148-6152 (1985); gene targeting in embryonic stem cells (Thompson et al., Cell 56:313-321 (1989); electroporation of embryos (Lo, Mol Cell. Biol. 3:1803-1814 (1983); and sperm-mediated gene transfer (Lavitrano et al., Cell 57:717-723 (1989); etc. For a review of such techniques, see Gordon, Transgenic Animals, Intl. Rev. Cytol. 115:171-229 (1989), which is incorporated by reference herein in its entirety.

Aspects of the invention also concern transgenic animals that carry a polymorphic and/or mutant A2M transgene in all their cells, as well as animals that carry the transgene in some, but not all their cells, i.e., mosaic animals. The transgene can be integrated as a single transgene or in concatamers, e.g., head-to-head tandems or head-to-tail tandems. The transgene can also be selectively introduced into and activated in a particular cell type by following, for example, the teaching of Lasko et al. (Lasko, M. et al., Proc. Natl. Acad. Sci. USA 89: 6232-6236 (1992), herein expressly incorporated by reference in its entirety). The regulatory sequences required for such a cell-type specific activation will depend upon the particular cell type of interest, and will be apparent to those of skill in the art.

When it is desired that the polymorphic and/or mutant A2M gene transgene be integrated into the chromosomal site of the endogenous A2M gene, gene targeting is preferred. Briefly, when such a technique is to be utilized, vectors containing some nucleotide sequences homologous to the endogenous A2M gene are designed for the purpose of integrating, via homologous recombination with chromosomal sequences, into and disrupting the function of the nucleotide sequence of the endogenous A2M gene. The transgene can also be selectively introduced into a particular cell type, thus inactivating the endogenous A2M gene in only that cell type, by following, for example, the teaching of Gu et al. (Gu, et al., Science 265: 103-106 (1994), herein expressly incorporated by reference in its entirety). The regulatory sequences required for such a cell-type specific inactivation will depend upon the particular cell type of interest, and will be apparent to those of skill in the art.

Once transgenic animals have been generated, the expression of the recombinant A2M gene can be assayed utilizing standard techniques. Initial screening can be accomplished by Southern blot analysis or PCR techniques to analyze animal tissues to assay whether integration of the transgene has taken place. The level of mRNA expression of the transgene in the tissues of the transgenic animals can also be assessed using techniques which include, but are not limited to, Northern blot analysis of tissue samples obtained from the animal, in situ hybridization analysis, and RT-PCR. The section below describes antibodies of the invention and methods of making these molecules.

Cells and transgenic animals containing nucleic acids that include variant A2M gene or cDNA sequences as described herein have numerous uses. For example, such cells and animals can be used in methods of assessing candidate agents that modulate A2M activity and/or expression, and candidate therapeutic agents for the treatment of diseases, such as neurodegenerative diseases, e.g., AD. Such cells and animals can also be used to assess the effects of a particular variant of a polymorphism. For example, transgenic animals in which nucleic acid containing a particular variant of a polymorphism has been introduced may be analyzed for a particular phenotype. The transgenic animal may be one in which the wild-type gene or predominant allele may have been knocked out. RNA and/or protein is compared in the transgenic animal harboring the allelic variant with an animal harboring a different allele, e.g., a predominant or reference allele. For example, the variant may result in alterations of RNA levels or RNA stability or in increased or decreased synthesis of the associated protein and/or aberrant tissue distribution or intracellular localization of the associated protein, altered phosphorylation, glycosylation and/or altered activity of the protein. Furthermore, various molecular, cellular and organismal manifestations of a disease can be monitored. For example, to assess a polymorphism for an effect that may be related to Alzheimer's disease, certain characteristic features of the disease, such as APP gene products, particularly A protein, neurite plaques, deficits of memory and learning and neurodegeneration of specific systems of cells may be evaluated in a transgenic animal containing nucleic acid containing the polymorphism. Such analysis could also be performed in cultured cells into which the variant allele gene or portion thereof is introduced. If the host cell contains a different allele of the same gene, it is possible to replace the endogenous gene with the variant gene in the cell, if desired. These effects can be determined according to methods known in the art and as described below. Particular variants of a polymorphism can be assayed individually or in combination.

Antibodies Specific for Polymorphic and/or Mutant A2M Polypeptides

Following synthesis or expression and isolation or purification of the A2M protein or a portion thereof, the isolated or purified protein can be used to generate antibodies and tools for identifying agents that interact with polymorphic and/or mutant A2M polypeptides. Depending on the context, the term “antibodies” can encompass polyclonal, monoclonal, chimeric, single chain, Fab fragments and fragments produced by a Fab expression library. Antibodies that recognize polymorphic and/or mutant A2M polypeptides have many uses including, but not limited to, biotechnological applications, therapeutic/prophylactic applications, and diagnostic applications.

For the production of antibodies, various hosts including goats, rabbits, rats, mice, etc. can be immunized by injection with polymorphic and/or mutant A2M polypeptides, in particular, any portion, fragment or oligopeptide that retains immunogenic properties. Depending on the host species, various adjuvants can be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. BCG (Bacillus Calmette-Guerin) and Corynebacterium parvum are also potentially useful adjuvants.

Peptides used to induce specific antibodies can have an amino acid sequence consisting of at least three amino acids, and preferably at least 10 to 15 amino acids. Preferably, short stretches of amino acids encoding fragments of polymorphic and/or mutant A2M polypeptides containing one or more of the mutations described in Table 1 are fused with those of another protein such as keyhole limpet hemocyanin such that an antibody is produced against the chimeric molecule. While antibodies capable of specifically recognizing polymorphic and/or mutant A2M polypeptides can be generated by injecting synthetic 3-mer, 10-mer, and 15-mer peptides that correspond to a protein sequence of polymorphic and/or mutant A2M polypeptides into mice, a more diverse set of antibodies can be generated by using recombinant polymorphic and/or mutant A2M polypeptides.

To generate antibodies to polymorphic and/or mutant A2M polypeptides, substantially pure polypeptides are isolated from a transfected or transformed cell. The concentration of the polypeptide in the final preparation is adjusted, for example, by concentration on an Amicon filter device, to the level of a few micrograms/ml. Monoclonal or polyclonal antibody to the polypeptide of interest can then be prepared as follows:

Monoclonal antibodies to polymorphic and/or mutant A2M polypeptides can be prepared using any technique that provides for the production of antibody molecules by continuous cell lines in culture. These include, but are not limited to, the hybridoma technique originally described by Koehler and Milstein (Nature 256:495-497 (1975), the human B-cell hybridoma technique (Kosbor et al. Immunol Today 4:72 (1983); Cote et al Proc Natl Acad Sci 80:2026-2030 (1983), and the EBV-hybridoma technique Cole et al. Monoclonal Antibodies and Cancer Therapy, Alan R. Liss Inc, New York N.Y., pp 77-96 (1985), all of which are hereby incorporated by reference in their entireties. In addition, techniques developed for the production of “chimeric antibodies”, the splicing of mouse antibody genes to human antibody genes to obtain a molecule with appropriate antigen specificity and biological activity can be used. (Morrison et al. Proc Natl Acad Sci 81:6851-6855 (1984); Neuberger et al. Nature 312:604-608(1984); Takeda et al. Nature 314:452-454(1985), all of which are hereby incorporated by reference in their entireties. Alternatively, techniques described for the production of single chain antibodies (U.S. Pat. No. 4,946,778) can be adapted to produce specific single chain antibodies, hereby incorporated by reference. Antibodies can also be produced by inducing in vivo production in the lymphocyte population or by screening recombinant immunoglobulin libraries or panels of highly specific binding reagents as disclosed in Orlandi et al., Proc Natl Acad Sci 86: 3833-3837 (1989), and Winter G. and Milstein C; Nature 349:293-299 (1991), all of which are hereby incorporated by reference in their entireties.

Antibody fragments that contain specific binding sites for polymorphic and/or mutant A2M polypeptides can also be generated. For example, such fragments include, but are not limited to, the F(ab′)₂ fragments that can be produced by pepsin digestion of the antibody molecule and the Fab fragments that can be generated by reducing the disulfide bridges of the F(ab′)₂ fragments. Alternatively, Fab expression libraries can be constructed to allow rapid and easy identification of monoclonal Fab fragments with the desired specificity. (Huse W. D. et al. Science 256:1275-1281 (1989)).

By one approach, monoclonal antibodies to polymorphic and/or mutant A2M polypeptides are made as follows. Briefly, a mouse is repetitively inoculated with a few micrograms of the selected protein or peptides derived therefrom over a period of a few weeks. The mouse is then sacrificed, and the antibody producing cells of the spleen isolated. The spleen cells are fused in the presence of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, E., Meth. Enzymol. 70:419 (1980), and derivative methods thereof. Selected positive clones can be expanded and their monoclonal antibody product harvested for use. Detailed procedures for monoclonal antibody production are described in Davis, L. et al. Basic Methods in Molecular Biology Elsevier, N.Y. Section 21-2, herein expressly incorporated by reference in its entirety.

Polyclonal antiserum containing antibodies to heterogenous epitopes of a single protein can be prepared by immunizing suitable animals with the expressed protein or peptides derived therefrom described above, which can be unmodified or modified to enhance immunogenicity. Effective polyclonal antibody production is affected by many factors related both to the antigen and the host species. For example, small molecules tend to be less immunogenic than others and can require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al. J. Clin. Endocrinol. Metab. 33:988-991 (1971), herein expressly incorporated by reference in its entirety.

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973). Plateau concentration of antibody is usually in the range of 0.1 to 0.2 mg/ml of serum (about 12 μM). Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980). Antibody preparations prepared according to either protocol are useful in quantitative immunoassays that determine concentrations of antigen-bearing substances in biological samples; they are also used semi-quantitatively or qualitatively (e.g., in diagnostic embodiments that identify the presence of polymorphic and/or mutant A2M polypeptides in biological samples). In the discussion that follows, several methods of molecular modeling and rational drug design are described. These techniques can be applied to identify molecules that interact with polymorphic and/or mutant A2M polypeptides and, thereby modulate their function.

Diagnostic Embodiments

Generally, the diagnostics of the invention can be classified according to whether the embodiment is a nucleic acid or protein-based assay. Some diagnostic assays detect mutations or polymorphisms in A2M nucleic acids or A2M proteins, which contribute to or place individuals at risk of acquiring neuropathies, such as AD. Other diagnostic assays identify and distinguish defects in A2M activities by detecting a level of polymorphic and/or mutant A2M RNA or A2M protein in a tested subject that resembles the level of polymorphic and/or mutant A2M RNA or A2M protein in a subject suffering from a neuropathy (e.g., AD) or by detecting a level of RNA or protein in a tested subject that is different than a subject not suffering from a disease.

Additionally, the manufacture of kits that incorporate the reagents and methods described in the following embodiments so as to allow for the rapid detection and identification of individuals at risk of acquiring a neuropathy, such as AD, are contemplated. The diagnostic kits can include a nucleic acid probe or an antibody or combinations thereof, which specifically detect a polymorphic and/or mutant A2M polypeptide or nucleic acid or a nucleic acid probe or an antibody or combinations thereof, which can be used to determine the level of RNA or protein expression of one or more polymorphic and/or mutant A2M nucleic acids or polypeptides. The detection component of these kits will typically be supplied in combination with one or more of the following reagents. A support capable of absorbing or otherwise binding DNA, RNA, or protein will often be supplied. Available supports include membranes of nitrocellulose, nylon or derivatized nylon that can be characterized by bearing an array of positively charged substituents. One or more restriction enzymes, control reagents, buffers, amplification enzymes, and non-human polynucleotides like calf-thymus or salmon-sperm DNA can be supplied in these kits.

Useful nucleic acid-based diagnostic techniques include, but are not limited to, direct DNA sequencing, Southern Blot analysis, single-stranded confirmation analysis (SSCA), RNAse protection assay, dot blot analysis, nucleic acid amplification, and combinations of these approaches. The starting point for these analysis is isolated or purified nucleic acid from a biological sample. If the diagnostic assay is designed to determine the presence of a polymorphic and/or mutant A2M nucleic acid, any source of DNA including, but not limited to hair, cheek cells and blood can be used as a biological sample. The nucleic acid is extracted from the sample and can be amplified by a DNA amplification technique such as the Polymerase Chain Reaction (PCR) using primers that correspond to regions flanking DNA recognized as a SNP and/or mutation in the A2M gene (See Table 1).

Once a sufficient amount of DNA is obtained from an individual to be tested, several methods can be used to detect a polymorphism and/or mutation. Direct DNA sequencing, either manual sequencing or automated fluorescent sequencing can detect such sequence variations. Another approach is the single-stranded confirmation polymorphism assay (SSCA) (Orita et al., Proc. Natl. Acad. Sci. USA 86:2776-2770 (1989), herein incorporated by reference). This method, however, does not detect all sequence changes, especially if the DNA fragment size is greater than 200 base pairs, but can be optimized to detect most DNA sequence variation.

The reduced detection sensitivity is a disadvantage, but the increased throughput possible with SSCA makes it an attractive, viable alternative to direct sequencing for mutation detection. The fragments that have shifted mobility on SSCA gels are then sequenced to determine the exact nature of the DNA sequence variation. Other approaches based on the detection of mismatches between the two complimentary DNA strands include clamped denaturing gel electrophoresis (CDGE) (Sheffield et al., Am. J. Hum. Genet. 49:699-706 (1991)), heteroduplex analysis (HA) (White et al., Genomics 12:301-306 (1992)), and chemical mismatch cleavage (CMC) (Grompe et al., Proc. Natl. Acad. Sci. USA 86:5855-5892 (1989), all of which, including the references contained therein, are hereby expressly incorporated by reference in their entireties). A review of currently available methods of detecting DNA sequence variation can be found in Grompe, Nature Genetics 5:111-117 (1993).

Seven well-known nucleic acid-based methods for confirming the presence of a polymorphism are described below. Provided for exemplary purposes only and not intended to limit any aspect of the invention, these methods include:

(1) single-stranded confirmation analysis (SSCA) (Orita et al.);

(2) denaturing gradient gel electrophoresis (DGGE) (Wartell et al., Nucl. Acids Res. 18:2699-2705 (1990) and Sheffield et al., Proc. Natl. Acad. Sci. USA 86:232-236 (1989)), both references herein incorporated by reference;

(3) RNAse protection assays (Finkelstein et al., Genomics 7:167-172 (1990) and Kinszler et al., Science 251:1366-1370 (1991)) both references herein incorporated by reference;

(4) the use of proteins which recognize nucleotide mismatches, such as the E. coli mutS protein (Modrich, Ann. Rev. Genet. 25:229-253 (1991), herein incorporated by reference;

(5) allele-specific PCR (Rano and Kidd, Nucl. Acids Res. 17:8392 (1989), herein incorporated by reference), which involves the use of primers that hybridize at their 3′ ends to a polymorphism and, if the polymorphism is not present, an amplification product is not observed; and

(6) Amplification Refractory Mutation System (ARMS), as disclosed in European Patent Application Publication No. 0332435 and in Newton et al., Nucl. Acids Res. 17:2503-2516 (1989), both references herein incorporated by reference; and

(7) temporal temperature gradient gel electrophoresis (TTGE), as described by Bio-Rad in U.S./E.G. Bulletin 2103, herein incorporated by reference.

In SSCA, DGGE, TTGE, and RNAse protection assay, a new electrophoretic band appears when the polymorphism is present. SSCA and TTGE detect a band that migrates differentially because the sequence change causes a difference in single-strand, intramolecular base pairing, which is detectable electrophoretically. RNAse protection involves cleavage of the mutant polynucleotide into two or more smaller fragments. DGGE detects differences in migration rates of sequences using a denaturing gradient gel. In an allele-specific oligonucleotide assay (ASOs) (Conner et al., Proc. Natl. Acad. Sci. USA 80:278-282 (1983)), an oligonucleotide is designed that detects a specific sequence, and an assay is performed by detecting the presence or absence of a hybridization signal. In the mutS assay, the protein binds only to sequences that contain a nucleotide mismatch in a heteroduplex between polymorphic and non-polymorphic sequences. Mismatches, in this sense of the word refers to hybridized nucleic acid duplexes in which the two strands are not 100% complementary. The lack of total homology results from the presence of one or more polymorphisms in an amplicon obtained from a biological sample, for example, that has been hybridized to a non-polymorphic strand. Mismatched detection can be used to detect point mutations in DNA or in an mRNA. While these techniques are less sensitive than sequencing, they are easily performed on a large number of biological samples and are amenable to array technology.

In some embodiments, nucleic acid probes that differentiate polynucleotides encoding wild type A2M from polymorphic and/or mutant A2M are attached to a support in an ordered array, wherein the nucleic acid probes are attached to distinct regions of the support that do not overlap with each other. Preferably, such an ordered array is designed to be “addressable” where the distinct locations of the probe are recorded and can be accessed as part of an assay procedure. These probes are joined to a support in different known locations. The knowledge of the precise location of each nucleic acid probe makes these “addressable” arrays particularly useful in binding assays. The nucleic acids from a preparation of several biological samples are then labeled by conventional approaches (e.g., radioactivity or fluorescence) and the labeled samples are applied to the array under conditions that permit hybridization.

If a nucleic acid in the samples hybridizes to a probe on the array, then a signal will be detected at a position on the support that corresponds to the location of the hybrid. Since the identity of each labeled sample is known and the region of the support on which the labeled sample was applied is known, an identification of the presence of the polymorphic variant can be rapidly determined. These approaches are easily automated using technology known to those of skill in the art of high throughput diagnostic or detection analysis.

Additionally, an opposite approach to that presented above can be employed. Nucleic acids present in biological samples can be disposed on a support so as to create an addressable array. Preferably, the samples are disposed on the support at known positions that do not overlap. The presence of nucleic acids having a desired polymorphism in each sample is determined by applying labeled nucleic acid probes that complement nucleic acids that encode the polymorphism and detecting the presence of a signal at locations on the array that correspond to the positions at which the biological samples were disposed. Because the identity of the biological sample and its position on the array is known, the identification of the polymorphic variant can be rapidly determined. These approaches are also easily automated using technology known to those of skill in the art of high throughput diagnostic analysis.

Any addressable array technology known in the art can be employed with this aspect of the invention. One particular embodiment of polynucleotide arrays is known as Genechips™, and has been generally described in U.S. Pat. No. 5,143,854; PCT publications WO 90/15070 and 92/10092. These arrays are generally produced using mechanical synthesis methods or light directed synthesis methods, which incorporate a combination of photolithographic methods and solid phase oligonucleotide synthesis. (Fodor et al., Science, 251:767-777, (1991)). The immobilization of arrays of oligonucleotides on solid supports has been rendered possible by the development of a technology generally identified as “Very Large Scale Immobilized Polymer Synthesis” (VLSPIS™) in which, typically, probes are immobilized in a high density array on a solid surface of a chip. Examples of VLSPIS™ technologies are provided in U.S. Pat. Nos. 5,143,854 and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and diagnostic information. Examples of such presentation strategies are disclosed in PCT Publications WO 94/12305, WO 94/11530, WO 97/29212, and WO 97/31256, all of which are hereby incorporated by reference in their entireties.

A wide variety of labels and conjugation techniques are known by those skilled in the art and can be used in various nucleic acid assays. There are several ways to produce labeled nucleic acids for hybridization or PCR including, but not limited to, oligolabeling, nick translation, end-labeling, or PCR amplification using a labeled nucleotide. Alternatively, a nucleic acid encoding a polymorphic and/or mutant A2M polypeptide can be cloned into a vector for the production of an mRNA probe. Such vectors are known in the art, are commercially available, and can be used to synthesize RNA probes in vitro by addition of an appropriate RNA polymerase such as T7, T3 or SP6 and labeled nucleotides. A number of companies such as Pharmacia Biotech (Piscataway N.J.), Promega (Madison Wis.), and U.S. Biochemical Corp (Cleveland Ohio) supply commercial kits and protocols for these procedures. Suitable reporter molecules or labels include those radionuclides, enzymes, fluorescent, chemiluminescent, or chromogenic agents, as well as, substrates, cofactors, inhibitors, magnetic particles and the like.

The RNAse protection method, briefly described above, is an example of a mismatch cleavage technique that is amenable to array technology. Preferably, the method involves the use of a labeled riboprobe that is complementary to polymorphic and/or mutant A2M nucleic acid sequences selected from the group consisting of 6i, 12i.1, 12i.2, 12e, 14e, 14i.1, 14i.2, 17i.1, 20e, 20i, 21i, 28i and 30e. The riboprobe and either mRNA or DNA isolated and amplified from a biological sample are annealed (hybridized) and subsequently digested with the enzyme RNAse A, which is able to detect mismatches in a duplex RNAse structure. If a mismatch is detected by RNAse A, the polymorphic variant is not present in the sample and the enzyme cleaves at the site of the mismatch and destroys the riboprobe. Thus, when the annealed RNA is separated on a electrophoretic gel matrix, if a mismatch has been detected and cleaved by RNAse A, an RNA product will be seen which is much smaller than the full length duplex RNA for the riboprobe and the mRNA or DNA.

Complements to the riboprobe can also be dispersed on an array and stringently probed with the products from the Rnase A digestion after denaturing any remaining hybrids. In this case, if a mismatch is detected and probe destroyed by Rnase A, the complements on the array will not anneal with the degraded RNA under stringent conditions. In a similar fashion, DNA probes can be used to detect mismatches, through enzymatic or chemical cleavage. See, e.g., Cotton, et al., Proc. Natl. Acad. Sci. USA 85:4397 (1988); Shenk et al., Proc. Natl. Acad. Sci. USA 72:989 (1975); and Novack et al., Proc. Natl. Acad. Sci. USA 83:586 (1986). Mismatches can also be detected by shifts in the electrophoretic ability of mismatched duplexes relative to matched duplexes. (See, e.g., Cariello, Human Genetics 42:726 (1988), herein incorporated by reference). With any of the techniques described above, the mRNA or DNA from a tested organism that corresponds to regions of an A2M gene having a polymorphism selected from the group consisting of 6i, 12i.1, 12i.2, 12e, 14e, 14i.1, 14i.2, 17i.1, 20e, 20i, 21i, 28i and 30e can be amplified by PCR before hybridization.

The presence of polymorphic and/or mutant A2M polypeptides in a protein sample can also be detected by using conventional assays. For example, antibodies immunoreactive with a polymorphic and/or mutant A2M polypeptide can be used to screen patient biological samples to determine if said patients are at risk of acquiring AD or have a predilection to acquire AD. Additionally, antibodies that differentiate the wild type A2M from polymorphic and/or mutant A2M polypeptides can be used to determine that an organism does not have a risk of acquiring AD or a predilection to acquire AD.

In preferred embodiments, antibodies are used to immunoprecipitate the polymorphic and/or mutant A2M polypeptides from solution or are used to react with the polymorphic and/or mutant A2M polypeptides on Western or Immunoblots. Favored diagnostic embodiments also include enzyme-linked immunosorbant assays (ELISA), radioimmunoassays (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference. Other embodiments employ aspects of the immune-strip technology disclosed in U.S. Pat. Nos. 5,290,678; 5,604,105; 5,710,008; 5,744,358; and 5,747,274, herein incorporated by reference.

In another preferred protein-based diagnostic, antibodies of the invention are attached to a support in an ordered array wherein a plurality of antibodies are attached to distinct regions of the support that do not overlap with each other. As with the nucleic acid-based arrays, the protein-based arrays are ordered arrays that are designed to be “addressable” such that the distinct locations are recorded and can be accessed as part of an assay procedure. These probes are joined to a support in different known locations. The knowledge of the precise location of each probe makes these “addressable” arrays particularly useful in binding assays. For example, an addressable array can comprise a support having several regions to which are joined a plurality of antibody probes that specifically recognize a particular A2M and differentiate the polymorphic and/or mutant A2M polypeptides from wild type A2M.

Proteins are obtained from biological samples and are labeled by conventional approaches (e.g., radioactivity, colorimetrically, or fluorescently). The labeled samples are then applied to the array under conditions that permit binding. If a protein in the sample binds to an antibody probe on the array, then a signal will be detected at a position on the support that corresponds to the location of the antibody-protein complex. Since the identity of each labeled sample is known and the region of the support on which the labeled sample was applied is known, an identification of the presence, concentration, and/or expression level can be rapidly determined. That is, by employing labeled standards of a known concentration of polymorphic and/or mutant A2M polypeptide or wild-type A2M, an investigator can accurately determine the protein concentration of the particular A2M in a tested sample and can also assess the expression level of the A2M. Conventional methods in densitometry can also be used to more accurately determine the concentration or expression level of the A2M. These approaches are easily automated using technology known to those of skill in the art of high throughput diagnostic analysis.

In another embodiment, an opposite approach to that presented above can be employed. Proteins present in biological samples can be disposed on a support so as to create an addressable array. Preferably, the protein samples are disposed on the support at known positions that do not overlap. The presence of a protein encoding a polymorphic and/or mutant A2M polypeptide in each sample is then determined by applying labeled antibody probes that recognize epitopes specific for the polymorphic and/or mutant A2M polypeptide. Because the identity of the biological sample and its position on the array is known, an identification of the presence, concentration, and/or expression level of a particular polymorphism can be rapidly determined.

That is, by employing labeled standards of a known concentration of polymorphic and/or mutant A2M polypeptides, an investigator can accurately determine the concentration of A2M in a sample and from this information can assess the expression level of the particular form of A2M. Conventional methods in densitometry can also be used to more accurately determine the concentration or expression level of the A2M. These approaches are also easily automated using technology known to those of skill in the art of high throughput diagnostic analysis. As detailed above, any addressable array technology known in the art can be employed with this aspect of the invention and display the protein arrays on the chips in an attempt to maximize antibody binding patterns and diagnostic information.

As discussed above, the presence or detection of one or more of the mutations and/or polymorphisms provided in Table 1 can provide a diagnosis that the tested subject is at risk of acquiring AD or has a predilection to acquire AD. Additional embodiments include the preparation of diagnostic kits comprising detection components, such as antibodies, specific for one or more of the particular polymorphic variants of A2M or A2M described herein. The detection component will typically be supplied in combination with one or more of the following reagents. A support capable of absorbing or otherwise binding RNA or protein will often be supplied. Available supports for this purpose include, but are not limited to, membranes of nitrocellulose, nylon or derivatized nylon that can be characterized by bearing an array of positively charged substituents, and Genechips™ or their equivalents. One or more enzymes, such as Reverse Transcriptase and/or Taq polymerase, can be furnished in the kit, as can dNTPs, buffers, or non-human polynucleotides like calf-thymus or salmon-sperm DNA. Results from the kit assays can be interpreted by a healthcare provider or a diagnostic laboratory. Alternatively, diagnostic kits are manufactured and sold to private individuals for self-diagnosis.

In addition to diagnosing disease according to the presence or absence of a polymorphic and/or mutant A2M nucleic acid or A2M polypeptide, some diseases may result from skewed levels of wild-type A2M as compared to polymorphic and/or mutant A2M. By monitoring the level of expression of specific A2M polypeptides, for example, a diagnosis can be made or a disease state can be identified. Similarly, by determining ratios of the level of expression of various A2M polypeptides a prognosis of health or disease can be made. The levels of expression of different types of A2M in various healthy individuals, as well as, individuals suffering from AD can be determined, for example. These values can be recorded in a database and can be compared to values obtained from tested individuals. Additionally, the ratios or patterns of expression of various A2M polypeptides from both healthy and diseased individuals is recorded in a database. These analyses are referred to as “disease state profiles” and by comparing one disease state profile (e.g. from a healthy or diseased individual) to a disease state profile from a tested individual, a clinician can rapidly diagnose the presence or absence of disease. .

The nucleic acid and protein-based diagnostic techniques described above can be used to detect the level or amount or ratio of expression of a particular A2M RNAs or A2M proteins in a tissue. Through quantitative Northern hybridizations, In situ analysis, immunohistochemistry, ELISA, genechip array technology, PCR, and Western blots, for example, the amount or level of expression of RNA or protein for a particular A2M (wild-type or mutant) can be rapidly determined and from this information ratios of A2M expression can be ascertained. Preferably, the expression levels of A2M genes having one or more of a polymorphism and/or mutation selected from the group consisting of 6i, 12i.1, 12i.2, 12e, 14e, 14i.1, 14i.2, 17i.1, 20e, 20i, 21i, 28i and 30e are measured to determine the ratios.

Once the levels of various A2M polypeptides or nucleic acids are determined, the information can be recorded onto a computer readable media, such as a hard drive, floppy disk, DVD drive, zip drive, etc. After recording and the generation of a database comprising the levels of expression of the various A2M polypeptides or nucleic acids studied, a comparing program is used which compares the levels of expression of the various A2M polypeptides or nucleic acids so as to create a ratio of expression. The following section describes the preparation of pharmaceuticals having polymorphic and/or mutant A2M polypeptides or binding partners, which can be administered to organisms in need to modulate A2M activities.

Pharmacogenomics

It is likely that subjects having one or more different allelic variants of the A2M gene will respond differently to drugs to treat associated diseases or disorders. For example, alleles of the A2M gene that associate with neurodegenerative disease will be useful alone or in conjunction with other genes associated with the development of neurodegenerative disease (e.g., APOE4) to predict a subject's response, either positive or negative, to a therapeutic drug. Multiplex primer extension assays or microarrays comprising probes for specific alleles are useful formats for determining drug response. A correlation between drug responses and specific alleles or combinations of alleles (haplotypes) of the A2M gene and other genes that associate with disease can be shown, for example, by clinical studies wherein the response, either positive or negative, to specific drugs of subjects having different allelic variants of polymorphic regions of the A2M gene alone or in combination with allelic variants of other genes are compared. Such studies can also be performed using animal models, such as mice having various alleles and in which, e.g., the endogenous uPA gene has been inactivated such as by a knock-out mutation. Test drugs are then administered to the mice having different alleles and the response of the different mice to a specific compound is compared. Accordingly, assays, microarrays and kits are provided for determining the drug which will be best suited for treating a specific disease or condition in a subject based on the individual's genotype. For example, it will be possible to select drugs which will be devoid of toxicity, or have the lowest level of toxicity possible for treating a subject having a disease or condition, e.g., neurodegenerative disease or Alzheimer's disease.

For example, therapeutic agents for treatment of neurodegenerative disease that can be genetically profiled include, but are not limited to, ALCAR, Alpha-tocopherol (Vitamin E), Ampalex, AN-1792 (AIP-001), Cerebrolysin, Daposone, Donepezil (Aricept), ENA-713 (Exelon), Estrogen replacement therapy, Galanthamine (Reminyl), Ginkgo Biloba extract, Huperzine A, Ibuprofen, Lipitor, Naproxen, Nefiracetam, Neotrofin, Memantine, Phenserine, Rofecoxib, Selegiline (Eldepryl), Tacrine (Cognex), Xanomeline (skin patch), Resperidone (Risperidol™), Neuroleptics, Benzodiazepenes, Valproate, Serotonin reuptake inhibitors (SRIs), Beta and Gamma Secretase Inhibitors, CX-516 (Ampalex), Statins and AF-102B (Evoxac).

Other therapeutic agents for treatment of neurodegenerative disease include those that are neuroprotective. Drugs with anti-oxidative properties, e.g., flupirtine, N-acetylcysteine, idebenone, melatonin, and also novel dopamine agonists (ropinirole and pramipexole) have been shown to protect neuronal cells from apoptosis and thus have been suggested for treating neurodegenerative disorders like AD or PD. Also, free radical scavengers, calcium channel blockers and modulators of certain signal transduction pathways that might protect neurons from downstream effects of the accumulation of A-Beta intracellularly and/or extracellularly. Also, other agents like non-steroidal anti-inflammatory drugs (NSAIDs) partly inhibit cyclooxygenase (COX) expression, as well as having a positive influence on the clinical expression of AD. Distinct cytokines, growth factors and related drug candidates, e.g., nerve growth factor (NGF), or members of the transforming growth factor-beta (TGF-beta) superfamily, like growth and differentiation factor 5 (GDF-5), are shown to protect tyrosine hydroxylase or dopaminergic neurons from apoptosis. CRIB (cellular replacement by immunoisolatory biocapsule) is a gene therapeutical approach for human NGF secretion, which has been shown to protect cholinergic neurons from cell death when implanted in the brain ((2000) Expert Opin Investig Drugs 9(4):747-64).

Provided herein is a method for predicting a response of a subject to an agent used to treat an A2M-mediated disease which includes a step of determining in nucleic acid obtained from the subject the identity of nucleotide(s) at one or more polymorphisms of an A2M gene that occur at positions corresponding to 6i, 12i.1, 12i.2, 12e, 14e, 14i.1, 14i.2, 17i.1, 20e, 20i, 21i, 28i, and 30e, wherein the presence or absence of a particular nucleotide(s) at the one or more polymorphisms, individually and/or in combination, is indicative of an increased or decreased likelihood that the treatment will be effective. Also provided are methods for predicting a response of a subject to an agent used to treat a neurodegenerative disease or disorder which include a step of determining in nucleic acid obtained from the subject, the identity of nucleotide(s) at one or more polymorphisms of an A2M gene that occur at positions corresponding to 6i, 12i.1, 12i.2, 12e, 14e, 14i.1, 14i.2, 17i.1, 20e, 20i, 21i, 28i, and 30e, wherein the presence or absence of a particular nucleotide(s) at the one or more polymorphisms, individually and/or in combination, is indicative of an increased or decreased likelihood that the treatment will be effective.

Also provided are any of the above methods wherein the neurodegenerative disease or disorder is Alzheimer's disease. In particular methods, the neurodegenerative disease or disorder is Alzheimer's disease wherein the age of onset is greater than or equal to about 50 years, or greater than or equal to about 60 years, or greater than or equal to about 65 years.

Also provided are any of the above methods which include a step of determining the identity of a nucleotide(s) at a position corresponding to the position of at least one polymorphism of at least one different gene, wherein the different gene is associated with a neurodegenerative disease or disorder. For example, the at least one different gene can be APOE4.

As set forth above, the ability to predict whether a person will respond to a particular therapeutic agent or drug is useful, among other things, for matching particular drug treatments to particular patient population to thereby eliminate from a treatment protocol drugs that may be less efficacious in particular patients.

Provided herein is a computer-assisted method of identifying a proposed treatment for a disease, such as, for example, a neurodegenerative disease. The method involves the steps of (a) storing a database of biological data for a plurality of subjects, the biological data that is being stored include for each of the plurality of subjects (i) treatment type, (ii) the presence or absence of a particular nucleotide(s) at one or more polymorphisms of the A2M gene selected from the group consisting of 6i, 12i.1, 12i.2, 12e, 14e, 14i.1, 14i.2, 17i.1, 20e, 20i, 21i, 28i, and 30e, and (iii) at least one disease progression measure for the neurodegenerative disease (e.g., AD), or other disease, from which treatment efficacy may be determined; and then (b) querying the database to determine the dependence on the one or more polymorphisms of the effectiveness of a treatment type in treating the disease, to thereby identify a proposed treatment as an effective treatment for a subject carrying a particular polymorphism (or combination of polymorphisms) for the disease, such as AD. The polymorphisms entered into the database can also include previously known polymorphisms, including, for example, polymorphisms included in Table 2.

Any suitable disease progression measure can be used. For example, for neurodegenerative disease, measures of motor function, cognitive function, dementia and combinations thereof can be used as measures of disease progression. The measures can be scored in accordance with standard techniques for entry into the database. Measures can be taken at the initiation of the study, and then during the course of the study (that is, treatment of the group of patients with the experimental and control treatments), and the database can incorporate a plurality of these measures taken over time so that the presence, absence or rate of disease progression in particular individuals or groups of individuals may be assessed. The database can be queried for the effectiveness of a particular treatment in patients carrying any of a variety of polymorphisms, or combinations of polymorphisms, or who lack particular polymorphisms. Computer systems used to carry out these methods may be implemented as hardware, software, or both hardware and software. Systems that may be used to implement these methods are known and available. See, e.g., U.S. Pat. No. 6,108,635 and Eas, M. A.: A program for the meta-analysis of clinical trials, Computer Methods and Programs in Biomedicine, vol. 53, no. 3 (July 1997); D. Klinger and M. Jaffe, An Information Technology Architecture for Pharmaceutical Research and Development, 14^{th} Annual Symposium on Computer Applications in Medical Care, November 4-7, pp. 256-260 (Washington D.C., 1990); M. Rosenberg, “ClinAccess: An integrated client/server approach to clinical data management and regulatory approval,” Proc. Of the 21^{st} Annual SAS Users Group International Conference (Cary, N.C., Mar. 10-13, 1996). Querying of the database maybe carried out in accordance with known techniques such as regression analysis or other types of comparisons such as with simple normal or t-tests, or with non-parametric techniques. Such querying may be carried out prospectively or retrospectively on the database by any suitable means, but is generally done by statistical analysis in accordance with known techniques.

Rational Drug Design

Rational drug design involving polypeptides requires identifying and defining a first peptide with which the designed drug is to interact, and using the first target peptide to define the requirements for a second peptide. With such requirements defined, one can find or prepare an appropriate peptide or non-peptide that meets all or substantially all of the defined requirements. Thus, one goal of rational drug design is to produce structural or functional analogs of biologically active polypeptides of interest or of small molecules with which they interact (e.g., agonists, antagonists, null compounds) in order to fashion drugs that are, for example, more or less potent forms of the ligand. (See, e.g., Hodgson, Bio. Technology 9:19-21 (1991)). An example of rational drug design is shown in Erickson et al., Science 249:527-533 (1990). Combinatorial chemistry is the science of synthesizing and testing compounds for bioactivity en masse, instead of one by one, the aim being to discover drugs and materials more quickly and inexpensively than was formerly possible. Rational drug design and combinatorial chemistry have become more intimately related in recent years due to the development of approaches in computer-aided protein modeling and drug discovery. (See e.g., U.S. Pat. Nos. 4,908,773; 5,884,230; 5,873,052; 5,331,573; and 5,888,738).

The use of molecular modeling as a tool for rational drug design and combinatorial chemistry has dramatically increased due to the advent of computer graphics. Not only is it possible to view molecules on computer screens in three dimensions but it is also possible to examine the interactions of macromolecules such as enzymes and receptors and rationally design derivative molecules to test. (See Boorman, Chem. Eng. News 70:18-26 (1992). A vast amount of user-friendly software and hardware is now available and virtually all pharmaceutical companies have computer modeling groups devoted to rational drug design. Molecular Simulations Inc., for example, sells several sophisticated programs that allow a user to start from an amino acid sequence, build a two or three-dimensional model of the protein or polypeptide, compare it to other two and three-dimensional models, and analyze the interactions of compounds, drugs, and peptides with a three dimensional model in real time. Accordingly, in some embodiments of the invention, software is used to compare regions of polymorphic and/or mutant A2M polypeptides and molecules that interact with polymorphic and/or mutant A2M polypeptides (collectively referred to as “binding partners”) with other molecules, such as peptides, peptidomimetics, and chemicals, so that therapeutic interactions can be predicted and designed. (See Schneider, Genetic Engineering News December: page 20 (1998), Tempczyk et al., Molecular Simulations Inc. Solutions April (1997) and Butenhof, Molecular Simulations Inc. Case Notes (August 1998) for a discussion of molecular modeling).

For example, the protein sequence of a polymorphic and/or mutant A2M polypeptide or binding partner, or domains of these molecules (or nucleic acid sequence encoding these polypeptides or both), can be entered onto a computer readable medium for recording and manipulation. It will be appreciated by those skilled in the art that a computer readable medium having these sequences can interface with software that converts or manipulates the sequences to obtain structural and functional information, such as protein models. That is, the functionality of a software program that converts or manipulates these sequences includes the ability to compare these sequences to other sequences or structures of molecules that are present on publicly and commercially available databases so as to conduct rational drug design.

The polymorphic and/or mutant A2M polypeptide or binding partner polypeptide or nucleic acid sequence or both can be stored, recorded, and manipulated on any medium that can be read and accessed by a computer. As used herein, the words “recorded” and “stored” refer to a process for storing information on computer readable medium. A skilled artisan can readily adopt any of the presently known methods for recording information on a computer readable medium to generate manufactures comprising the nucleotide or polypeptide sequence information of this embodiment. A variety of data storage structures are available to a skilled artisan for creating a computer readable medium having recorded thereon a nucleotide or polypeptide sequence. The choice of the data storage structure will generally be based on the component chosen to access the stored information. Computer readable media include magnetically readable media, optically readable media, or electronically readable media. For example, the computer readable media can be a hard disc, a floppy disc, a magnetic tape, zip disk, CD-ROM, DVD-ROM, RAM, or ROM as well as other types of other media known to those skilled in the art. The computer readable media on which the sequence information is stored can be in a personal computer, a network, a server or other computer systems known to those skilled in the art.

Embodiments of the invention utilize computer-based systems that contain the sequence information described herein and convert this information into other types of usable information (e.g., protein models for rational drug design). The term “a computer-based system” refers to the hardware, software, and any database used to analyze an polymorphic and/or mutant A2M or a binding partner (nucleic acid or polypeptide sequence or both), or fragments of these biomolecules so as to construct models or to conduct rational drug design. The computer-based system preferably includes the storage media described above, and a processor for accessing and manipulating the sequence data. The hardware of the computer-based systems of this embodiment comprise a central processing unit (CPU) and a database. A skilled artisan can readily appreciate that any one of the currently available computer-based systems are suitable.

In one particular embodiment, the computer system includes a processor connected to a bus that is connected to a main memory (preferably implemented as RAM) and a variety of secondary storage devices, such as a hard drive and removable medium storage device. The removable medium storage device can represent, for example, a floppy disk drive, a DVD drive, an optical disk drive, a compact disk drive, a magnetic tape drive, etc. A removable storage medium, such as a floppy disk, a compact disk, a magnetic tape, etc. containing control logic and/or data recorded therein can be inserted into the removable storage device. The computer system includes appropriate software for reading the control logic and/or the data from the removable medium storage device once inserted in the removable medium storage device. The polymorphic and/or mutant A2M or binding partner (nucleic acid or polypeptide sequence or both) can be stored in a well known manner in the main memory, any of the secondary storage devices, and/or a removable storage medium. Software for accessing and processing these sequences (such as search tools, compare tools, and modeling tools etc.) reside in main memory during execution.

As used herein, “a database” refers to memory that can store a polymorphic and/or mutant A2M or binding partner nucleotide or polypeptide sequence information, protein model information, information on other peptides, chemicals, peptidomimetics, and other agents that interact with polymorphic and/or mutant A2M polypeptides, and values or results from functional assays. Additionally, a “database” refers to a memory access component that can access manufactures having recorded thereon polymorphic and/or mutant A2M or binding partner nucleotide or polypeptide sequence information, protein model information, information on other peptides, chemicals, peptidomimetics, and other agents that interact with polymorphic and/or mutant A2M polypeptides, and values or results from functional assays. In other embodiments, a database stores a “polymorphic and/or mutant A2M polypeptide functional profile” comprising the values and results (e.g., ability to associate with a receptor, amyloid β, a protease, zinc, or the ability to form a tetramer) from one or more “A2M functional assays”, as described herein or known in the art, and relationships between these values or results. The sequence data and values or results from these functional assays can be stored and manipulated in a variety of data processor programs in a variety of formats. For example, the sequence data can be stored as text in a word processing file, such as Microsoft WORD or WORDPERFECT, an ASCII file, a html file, or a pdf file in a variety of database programs familiar to those of skill in the art, such as DB2, SYBASE, or ORACLE.

A “search program” refers to one or more programs that are implemented on the computer-based system to compare a polymorphic and/or mutant A2M or binding partner (nucleotide or polypeptide sequence) with other nucleotide or polypeptide sequences and agents including but not limited to peptides, peptidomimetics, and chemicals stored within a database. A search program also refers to one or more programs that compare one or more protein models to several protein models that exist in a database and one or more protein models to several peptides, peptidomimetics, and chemicals that exist in a database. A search program is used, for example, to compare one polymorphic and/or mutant A2M functional profile to one or more polymorphic and/or mutant A2M functional profiles that are present in a database so as to determine an appropriate treatment protocol, for example. Still further, a search program can be used to compare values or results from A2M functional assays and agents that modulate A2M-mediated activities.

A “retrieval program” refers to one or more programs that can be implemented on the computer-based system to identify peptides, peptidomimetics, and chemicals that interact with a polymorphic and/or mutant A2M polypeptide sequence, or a polymorphic and/or mutant A2M polypeptide model stored in a database. Further, a retrieval program is used to identify a specific agent that modulates A2M-mediated activities to a desired set of values, results, or profile. That is, a retrieval program can also be used to obtain “a binding partner profile” that is composed of a chemical structure, nucleic acid sequence, or polypeptide sequence or model of an agent that interacts with a polymorphic and/or mutant A2M polypeptide and, thereby modulates (inhibits or enhances) an A2M activity, such as binding to a receptor, amyloid β, a protease, zinc, or tetramer formation. Further, a binding partner profile can have one or more symbols that represent these molecules and/or models, an identifier that represents one or more agents including, but not limited to peptides and peptidomimetics (referred to collectively as “peptide agents”) and chemicals, and a value or result from a functional assay.

As a starting point to rational drug design, a two or three dimensional model of a polypeptide of interest is created (e.g., polymorphic and/or mutant A2M polypeptide, or a binding partner, such as the LRP receptor, amyloid β, a protease, or an antibody). In the past, the three-dimensional structure of proteins has been determined in a number of ways. Perhaps the best known way of determining protein structure involves the use of x-ray crystallography. A general review of this technique can be found in Van Holde, K. E. Physical Biochemistry, Prentice-Hall, N.J. pp. 221-239 (1971). Using this technique, it is possible to elucidate three-dimensional structure with good precision. Additionally, protein structure can be determined through the use of techniques of neutron diffraction, or by nuclear magnetic resonance (NMR). (See, e.g., Moore, W. J., Physical Chemistry, 4^{th} Edition, Prentice-Hall, N.J. (1972)).

Alternatively, protein models of a polypeptide of interest can be constructed using computer-based protein modeling techniques. By one approach, the protein folding problem is solved by finding target sequences that are most compatible with profiles representing the structural environments of the residues in known three-dimensional protein structures. (See, e.g., U.S. Pat. No. 5,436,850). In another technique, the known three-dimensional structures of proteins in a given family are superimposed to define the structurally conserved regions in that family. This protein modeling technique also uses the known three-dimensional structure of a homologous protein to approximate the structure of a polypeptide of interest. (See e.g., U.S. Pat. Nos. 5,557,535; 5,884,230; and 5,873,052). Conventional homology modeling techniques have been used routinely to build models of proteases and antibodies. (Sowdhamini et al., Protein Engineering 10:207, 215 (1997)). Comparative approaches can also be used to develop three-dimensional protein models when the protein of interest has poor sequence identity to template proteins. In some cases, proteins fold into similar three-dimensional structures despite having very weak sequence identities. For example, the three-dimensional structures of a number of helical cytokines fold in similar three-dimensional topology in spite of weak sequence homology.

The recent development of threading methods and “fuzzy” approaches now enables the identification of likely folding patterns and functional protein domains in a number of situations where the structural relatedness between target and template(s) is not detectable at the sequence level. By one method, fold recognition is performed using Multiple Sequence Threading (MST) and structural equivalences are deduced from the threading output using the distance geometry program DRAGON that constructs a low resolution model. A full-atom representation is then constructed using a molecular modeling package such as QUANTA.

According to this 3-step approach, candidate templates are first identified by using the novel fold recognition algorithm MST, which is capable of performing simultaneous threading of multiple aligned sequences onto one or more 3-D structures. In a second step, the structural equivalences obtained from the MST output are converted into interresidue distance restraints and fed into the distance geometry program DRAGON, together with auxiliary information obtained from secondary structure predictions. The program combines the restraints in an unbiased manner and rapidly generates a large number of low resolution model confirmations. In a third step, these low resolution model confirmations are converted into full-atom models and organized to energy minimization using the molecular modeling package QUANTA. (See e.g., Aszódi et al., Proteins:Structure, Function, and Genetics, Supplement 1:38-42 (1997)).

In a preferred approach, the commercially available “Insight II 98” program (Molecular Simulations Inc.) and accompanying modules are used to create a two and/or three dimensional model of a polypeptide of interest from an amino acid sequence. Insight II is a three-dimensional graphics program that can interface with several modules that perform numerous structural analysis and enable real-time rational drug design and combinatorial chemistry. Modules such as Builder, Biopolymer, Consensus, and Converter, for example, allow one to rapidly create a two dimensional or three dimensional model of a polypeptide, carbohydrate, nucleic acid, chemical or combinations of the foregoing from their sequence or structure. The modeling tools associated with Insight II support many different data file formats including Brookhaven and Cambridge databases; AMPAC/MOPAC and QCPE programs; Molecular Design Limited Molfile and SD files, Sybel Mol2 files, VRML, and Pict files.

Additionally, the techniques described above can be supplemented with techniques in molecular biology to design models of the protein of interest. For example, a polypeptide of interest can be analyzed by an alanine scan (Wells, Methods in Enzymol. 202:390-411 (1991)) or other types of site-directed mutagenesis analysis. In alanine scan, each amino acid residue of the polypeptide of interest is sequentially replaced by alanine in a step-wise fashion (i.e., only one alanine point mutation is incorporated per molecule starting at position #1 and proceeding through the entire molecule), and the effect of the mutation on the peptide's activity in a functional assay is determined. Each of the amino acid residues of the peptide is analyzed in this manner and the regions important for A2M activities, are identified. These functionally important regions can be recorded on a computer readable medium, stored in a database in a computer system, and a search program can be employed to generate a protein model of the functionally important regions.

Once a model of the polypeptide of interest is created, a candidate binding partner can be identified and manufactured as follows. First, a molecular model of one or more molecules that are known to interact with A2M or portions thereof are created using one of the techniques discussed above or as known in the art. Next, chemical libraries and databases are searched for molecules similar in structure to the known molecule. That is, a search can be made of a three dimensional data base for non-peptide (organic) structures (e.g., non-peptide analogs, and/or dipeptide analogs) having three dimensional similarity to the known structure of the target compound. See, e.g., the Cambridge Crystal Structure Data Base, Crystallographic Data Center, Lensfield Road, Cambridge, CB2 1EW, England; and Allen, F. H., et al., Acta Crystallogr., B35: 2331-2339 (1979). The identified candidate binding partners that interact with A2M can then be analyzed in a functional assay (e.g., binding assays with amyloid β, the LRP receptor, zinc, protease, or tetramer formation) and new molecules can be modeled after the candidate binding partners that produce a desirable response. Preferably, these interactions are studied with both wild-type A2M and polymorphic and/or mutant A2M polypeptides. By cycling in this fashion, libraries of molecules that interact with A2M, preferably polymorphic and/or mutant A2M polypeptides, and produce a desirable or optimal response in a functional assay can be selected.

It is noted that search algorithms for three dimensional data base comparisons are available in the literature. See, e.g., Cooper, et al., J. Comput.-Aided Mol. Design, 3: 253-259 (1989) and references cited therein; Brent, et al., J. Comput.-Aided Mol. Design, 2: 311-310 (1988) and references cited therein. Commercial software for such searches is also available from vendors such as Day Light Information Systems, Inc., Irvine, Calif. 92714, and Molecular Design Limited, 2132 Faralton Drive, San Leandro, Calif. 94577. The searching is done in a systematic fashion by simulating or synthesizing analogs having a substitute moiety at every residue level. Preferably, care is taken that replacement of portions of the backbone does not disturb the tertiary structure and that the side chain substitutions are compatible to retain the receptor substrate interactions.

By another approach, protein models of binding partners that interact with A2M, preferably polymorphic and/or mutant A2M polypeptides, can be made by the methods described above and these models can be used to predict the interaction of new molecules. Once a model of a binding partner is identified, the active sites or regions of interaction can be identified. Such active sites might typically be ligand binding sites. The active site can be identified using methods known in the art including, for example, from the amino acid sequences of peptides, from the nucleotide sequences of nucleic acids, or from study of complexes of the wild-type and/or polymorphic and/or mutant A2M polypeptides with a ligand. In the latter case, chemical or X-ray crystallographic methods can be used to find the active site by finding where on the wild-type and/or polymorphic and/or mutant A2M polypeptides the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures can be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method can be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site of the known binding partner, either experimentally, by modeling, or by a combination, candidate binding partners can be identified by searching databases containing compounds along with information on their molecular structure. Such a search seeks compounds having structures that match the determined active site structure and that interact with the groups defining the active site. Such a search can be manual, but is preferably computer assisted. One program that allows for such analysis is Insight II having the Ludi module. Further, the Ludi/ACD module allows a user access to over 65,000 commercially available drug candidates (MDL's Available Chemicals Directory) and provides the ability to screen these compounds for interactions with the protein of interest.

Alternatively, these methods can be used to identify improved binding partners from an already known binding partner. The composition of the known binding partner can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

A number of articles review computer modeling of drugs interactive with specific-proteins, such as Rotivinen, et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236:125-140 and 141-162; and, with respect to a model receptor for nucleic acid components, Askew, et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific for the modulation of A2M activities.

Many more computer programs and databases can be used with embodiments of the invention to identify new binding partners that modulate A2M function. The following list is intended not to limit the invention but to provide guidance to programs and databases that are useful with the approaches discussed above. The programs and databases that can be used include, but are not limited to: MacPattem (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, J. Mol. Biol. 215: 403 (1990), herein incorporated by reference), FASTA (Pearson and Lipman, Proc. Natl. Acad. Sci. USA, 85: 2444 (1988), herein incorporated by reference), Catalyst (Molecular Simulations Inc.), Catalyst/SHAPE (Molecular Simulations Inc.), Cerius². DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), Modeller 4 (Sali and Blundell J. Mol. Biol. 234:217-241 (1997)), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), Biopendium (Inpharmatica), SBdBase (Structural Bioinformatics), the EMBL/Swissprotein database, the MDL Available Chemicals Directory database, the MDL Drug Data Report data base, the Comprehensive Medicinal Chemistry database, Derwent's World Drug Index database, and the BioByteMasterFile database. Many other programs and data bases would be apparent to one of skill in the art given the present disclosure.

Once candidate binding partners have been identified, desirably, they are analyzed in a functional assay. Further cycles of modeling and functional assays can be employed to more narrowly define the parameters needed in a binding partner. Each binding partner and its response in a functional assay can be recorded on a computer readable media and a database or library of binding partners and respective responses in a functional assay can be generated. These databases or libraries can be used by researchers to identify important differences between active and inactive molecules so that compound libraries are enriched for binding partners that have favorable characteristics. The section below describes several A2M functional assays that can be used to characterize A2M interactions with candidate binding partners.

A2M Characterization Assays

The term “A2M characterization assay” or “A2M functional assay” or “functional assay” the results of which can be recorded as a value in a “A2M functional profile”, include assays that directly or indirectly evaluate the presence of an A2M nucleic acid or protein in a cell and the ability of a particular type of A2M polypeptide, in particular polymorphic and/or mutant A2M polypeptides, to associate with a receptor, a protease, amyloid β, zinc, or to form a tetramer.

Some functional assays involve binding assays that utilize multimeric agents. One form of multimeric agent concerns a manufacture comprising a polymorphic and/or mutant A2M polypeptide disposed on a support. These multimeric agents provide the polypeptide in such a form or in such a way that a sufficient affinity for its ligand is achieved. A multimeric agent having an polymorphic and/or mutant A2M polypeptide is obtained by joining the desired polypeptide to a macromolecular support. A “support” can be a termed a carrier, a protein, a resin, a cell membrane, or any macromolecular structure used to join or immobilize such molecules. Solid supports include, but are not limited to, the walls of wells of a reaction tray, test tubes, polystyrene beads, magnetic beads, nitrocellulose strips, membranes, microparticles such as latex particles, animal cells, Duracyte®, artificial cells, and others. A polymorphic and/or mutant A2M polypeptide can also be joined to inorganic carriers, such as silicon oxide material (e.g., silica gel, zeolite, diatomaceous earth or aminated glass) by, for example, a covalent linkage through a hydroxy, carboxy or amino group and a reactive group on the carrier.

In several multimeric agents, the macromolecular support has a hydrophobic surface that interacts with a portion of the polymorphic and/or mutant A2M polypeptides by a hydrophobic non-covalent interaction. In some cases, the hydrophobic surface of the support is a polymer such as plastic or any other polymer in which hydrophobic groups have been linked such as polystyrene, polyethylene or polyvinyl. Additionally, polymorphic and/or mutant A2M polypeptides can be covalently bound to carriers including proteins and oligo/polysaccarides (e.g. cellulose, starch, glycogen, chitosane or aminated sepharose). In these later multimeric agents, a reactive group on the molecule, such as a hydroxy or an amino group, is used to join to a reactive group on the carrier so as to create the covalent bond. Additional multimeric agents comprise a support that has other reactive groups that are chemically activated so as to attach the polymorphic and/or mutant A2M polypeptides. For example, cyanogen bromide activated matrices, epoxy activated matrices, thio and thiopropyl gels, nitrophenyl chloroformate and N-hydroxy succinimide chlorformate linkages, or oxirane acrylic supports are used. (Sigma).

Furthermore, in some embodiments, a liposome or lipid bilayer (natural or synthetic) is contemplated as a support and polymorphic and/or mutant A2M polypeptides, or binding partners are attached to the membrane surface or are incorporated into the membrane by techniques in liposome engineering. Carriers for use in the body, (i.e. for prophylactic or therapeutic applications) are desirably physiological, non-toxic and preferably, non-immunoresponsive. Suitable carriers for use in the body include poly-L-lysine, poly-D, L-alanine, liposomes, and Chromosorb® (Johns-Manville Products, Denver Colo.). Ligand conjugated Chromosorb® (Synsorb-Pk) has been tested in humans for the prevention of hemolytic-uremic syndrome and was reported as not presenting adverse reactions. (Armstrong et al. J. Infectious Diseases 171:1042-1045 (1995)).

The insertion of linkers, such as linkers (e.g., “λ linkers” engineered to resemble the flexible regions of λ phage) of an appropriate length between the polymorphic and/or mutant A2M polypeptides and the support are also contemplated so as to encourage greater flexibility and thereby overcome any steric hindrance that can be presented by the support. The determination of an appropriate length of linker that allows for an optimal cellular response or lack thereof, can be determined by screening the polymorphic and/or mutant A2M polypeptides with varying linkers in the assays detailed in the present disclosure.

A composite support comprising more than one type of polymorphic and/or mutant A2M polypeptides is also envisioned. A “composite support” can be a carrier, a resin, or any macromolecular structure used to attach or immobilize two or more different binding partners or polymorphic and/or mutant A2M polypeptides. In some embodiments, a liposome or lipid bilayer (natural or synthetic) is contemplated for use in constructing a composite support and polymorphic and/or mutant A2M polypeptides or binding partners are attached to the membrane surface or are incorporated into the membrane using techniques in liposome engineering.

As above, the insertion of linkers, such as λ linkers, of an appropriate length between the polymorphic and/or mutant A2M polypeptides or binding partner and the support is also contemplated so as to encourage greater flexibility in the molecule and thereby overcome any steric hindrance that can occur. The determination of an appropriate length of linker that allows for an optimal cellular response or lack thereof, can be determined by screening the polymorphic and/or mutant A2M polypeptides or binding partners with varying linkers in the assays detailed in the present disclosure.

In other embodiments of the invention, the multimeric and composite supports discussed above can have attached multimerized polymorphic and/or mutant A2M polypeptides, or binding partners so as to create a “multimerized-multimeric support” and a “multimerized-composite support”, respectively. A multimerized ligand can, for example, be obtained by coupling two or more binding partners in tandem using conventional techniques in molecular biology. The multimerized form of the polymorphic and/or mutant A2M polypeptides, or binding partner can be advantageous for many applications because of the ability to obtain an agent with a higher affinity for A2M, for example. The incorporation of linkers or spacers, such as flexible λ linkers, between the individual domains that make-up the multimerized agent can also be advantageous for some embodiments. The insertion of λ linkers of an appropriate length between protein binding domains, for example, can encourage greater flexibility in the molecule and can overcome steric hindrance. Similarly, the insertion of linkers between the multimerized binding partner or polymorphic and/or mutant A2M polypeptides and the support can encourage greater flexibility and limit steric hindrance presented by the support. The determination of an appropriate length of linker can be determined by screening the polymorphic and/or mutant A2M polypeptides and binding partners with varying linkers in the assays detailed in this disclosure.

Thus, several approaches to identify agents that interact with a polymorphic and/or mutant A2M polypeptide, employ a polymorphic and/or mutant A2M polypeptide joined to a support. Once the support-bound polypeptide is obtained, for example, candidate binding partners are contacted to the support-bound polypeptide and an association is determined directly (e.g., by using labeled binding partner) or indirectly (e.g., by using a labeled antibody directed to the binding partner). Candidate binding partners are identified as binding partners by virtue of the association with the support-bound polypeptide. The properties of the binding partners are analyzed and derivatives are made using rational drug design and combinatorial chemistry. Candidate binding partners can be obtained from random chemical or peptide libraries but, preferably, are rationally selected. For example, monoclonal antibodies that bind to polymorphic and/or mutant A2M polypeptides can be created and the nucleic acids encoding the VH and VL domains of the antibodies can be sequenced. These sequences can then be used to synthesize peptides that bind to the polymorphic and/or mutant A2M polypeptides. Further, peptidomimetics corresponding to these sequences can be created. These molecules can then be used as candidate binding partners.

Additionally, a cell based approach can be used to characterize polymorphic and/or mutant A2M polypeptides or to rapidly identify binding partners that interact with said polypeptides and, thereby, modulate A2M activities. Preferably, molecules identified in the support-bound A2M assay described above are used in the cell based approach, however, randomly generated compounds can also be used.

Many A2M characterization assays take advantage of techniques in molecular biology that are employed to discover protein:protein interactions. One method that detects protein-protein interactions in vivo, the two-hybrid system, is described in detail for illustration only and not by way of limitation. Other similar assays that can be can be adapted to identify binding partners include:

(1) the two-hybrid systems (Field & Song, Nature 340:245-246 (1989); Chien et al., Proc. Natl. Acad. Sci. USA 88:9578-9582 (1991); and Young K H, Biol. Reprod. 58:302-311 (1998), all references herein expressly incorporated by reference);

(2) reverse two-hybrid system (Leanna & Hannink, Nucl. Acid Res. 24:3341-3347 (1996), herein incorporated by reference);

(3) repressed transactivator system (Sadowski et al., U.S. Pat. No. 5,885,779), herein incorporated by reference);

(4) phage display (Lowman H B, Annu. Rev. Biophys. Biomol. Struct. 26:401-424 (1997), herein incorporated by reference); and

(5) GST/HIS pull down assays, mutant operators (Granger et al., WO 98/01879) and the like (See also Mathis G., Clin. Chem. 41:139-147 (1995); Lam K. S. Anticancer Drug Res., 12:145-167 (1997); and Phizicky et al., Microbiol. Rev. 59:94-123 (1995), all references herein expressly incorporated by reference).

An adaptation of the system described by Chien et al., 1991, Proc. Natl. Acad. Sci. USA, 88:9578-9582, herein incorporated by reference), which is commercially available from Clontech (Palo Alto, Calif.) is as follows. Plasmids are constructed that encode two hybrid proteins: one plasmid consists of nucleotides encoding the DNA-binding domain of a transcription activator protein fused to a nucleotide sequence encoding a polymorphic and/or mutant A2M polypeptide, and the other plasmid consists of nucleotides encoding the transcription activator protein's activation domain fused to a cDNA encoding an unknown protein that has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology can be used to screen activation domain libraries for proteins that interact with the “bait” gene product. By way of example, and not by way of limitation, polymorphic and/or mutant A2M polypeptides can be used as the bait gene product. Total genomic or cDNA sequences are fused to the DNA encoding an activation domain. This library and a plasmid encoding a hybrid of a bait gene encoding the polymorphic and/or mutant A2M polypeptide fused to the DNA-binding domain are cotransformed into a yeast reporter strain, and the resulting transformants are screened for those that express the reporter gene. For example, and not by way of limitation, a bait gene sequence encoding a polymorphic and/or mutant A2M polypeptide can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait polymorphic and/or mutant A2M polypeptides are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait polymorphic and/or mutant A2M gene-GAL4 fusion plasmid into a yeast strain which contains a lacZ gene driven by a promoter which contains GAL4 activation sequence. A cDNA encoded protein, fused to GAL4 transcriptional activation domain, that interacts with bait A2M gene product will reconstitute an active GAL4 protein and thereby drive expression of the lacZ gene. Colonies that express lacZ can be detected and the cDNA can then be purified from these strains, and used to produce and isolate the binding partner by techniques routinely practiced in the art. The examples below describe preferred A2M characterization assays.

Pharmaceutical Preparations and Methods of Administration

The polymorphic and/or mutant A2M nucleic acids and polypeptides and their binding partners are suitable for incorporation into pharmaceuticals that treat or prevent neuropathies, such as AD. These pharmacologically active compounds can be processed in accordance with conventional methods of galenic pharmacy to produce medicinal agents for administration to organisms, e.g., plants, insects, mold, yeast, animals, and mammals including humans. The active ingredients can be incorporated into a pharmaceutical product with and without modification. Further, the manufacture of pharmaceuticals or therapeutic agents that deliver the pharmacologically active compounds of this invention by several routes are aspects of the invention. For example, and not by way of limitation, DNA, RNA, and viral vectors having sequence encoding the polymorphic and/or mutant A2M polypeptides, binding partners, or fragments thereof are used with embodiments. Nucleic acids encoding polymorphic and/or mutant A2M polypeptides or binding partners can be administered alone or in combination with other active ingredients.

The compounds of this invention can be employed in admixture with conventional excipients, i.e., pharmaceutically acceptable organic or inorganic carrier substances suitable for parenteral, enteral (e.g., oral) or topical application that do not deleteriously react with the pharmacologically active ingredients of this invention. Suitable pharmaceutically acceptable carriers include, but are not limited to, water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Many more suitable vehicles are described in Remmington's Pharmaceutical Sciences, 15th Edition, Easton: Mack Publishing Company, pages 1405-1412 and 1461-1487(1975) and The National Formulary XIV, 14th Edition, Washington, American Pharmaceutical Association (1975), herein incorporated by reference. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The effective dose and method of administration of a particular pharmaceutical formulation having polymorphic and/or mutant A2M polypeptides or nucleic acids or binding partners, or fragments thereof can vary based on the individual needs of the patient and the treatment or preventative measure sought. Therapeutic efficacy and toxicity of such compounds can be determined by standard pharmaceutical procedures in cell cultures or experimental animals, e.g., ED50 (the dose therapeutically effective in 50% of the population). The data obtained from these assays is then used in formulating a range of dosage for use with other organisms, including humans. The dosage of such compounds lies preferably within a range of circulating concentrations that include the ED50 with no toxicity. The dosage varies within this range depending upon type of polymorphic and/or mutant A2M polypeptide or nucleic acid or binding partner, or fragment thereof, the dosage form employed, sensitivity of the organism, and the route of administration.

Normal dosage amounts of various polymorphic and/or mutant A2M polypeptide or nucleic acid or binding partner, or fragment thereof can vary from any number between approximately 1 to 100,000 micrograms, up to a total dose of about 10 grams, depending upon the route of administration. Desirable dosages include, for example, 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5, 6 g, 7 g, 8 g, 9 g, and 10 g.

In some embodiments, the dose of polymorphic and/or mutant A2M polypeptide or nucleic acid or binding partner, or fragment thereof preferably produces a tissue or blood concentration or both from approximately any number between 0.1 μM to 500 mM. Desirable doses produce a tissue or blood concentration or both of about any number between 1 to 800 μM. Preferable doses produce a tissue or blood concentration of greater than about any number between 10 μM to about 500 μM. Preferable doses are, for example, the amount of active ingredient required to achieve a tissue or blood concentration or both of 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 120 μM, 130 μM, 140 μM, 145 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration of greater than 800 μM are not preferred, they can be used with some embodiments of the invention. A constant infusion of the polymorphic and/or mutant A2M polypeptide or nucleic acid or binding partner, or fragment thereof can also be provided so as to maintain a stable concentration in the tissues as measured by blood levels.

The exact dosage is chosen by the individual physician in view of the patient to be treated. Dosage and administration are adjusted to provide sufficient levels of the active moiety or to maintain the desired effect. Additional factors that can be taken into account include the severity of the disease, age of the organism, and weight or size of the organism; diet, time and frequency of administration, drug combination(s), reaction sensitivities, and tolerance/response to therapy. Short acting pharmaceutical compositions are administered daily whereas long acting pharmaceutical compositions are administered every 2, 3 to 4 days, every week, or once every two weeks. Depending on half-life and clearance rate of the particular formulation, the pharmaceutical compositions of the invention are administered once, twice, three, four, five, six, seven, eight, nine, ten or more times per day.

Routes of administration of the pharmaceuticals of the invention include, but are not limited to, topical, transdermal, parenteral, gastrointestinal, transbronchial, and transalveolar. Transdermal administration is accomplished by application of a cream, rinse, gel, etc. capable of allowing the pharmacologically active compounds to penetrate the skin. Parenteral routes of administration include, but are not limited to, electrical or direct injection such as direct injection into a central venous line, intravenous, intramuscular, intraperitoneal, intradermal, or subcutaneous injection. Gastrointestinal routes of administration include, but are not limited to, ingestion and rectal. Transbronchial and transalveolar routes of administration include, but are not limited to, inhalation, either via the mouth or intranasally.

Compositions having the pharmacologically active compounds of this invention that are suitable for transdermal or topical administration include, but are not limited to, pharmaceutically acceptable suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference. Examples of suitable transdermal devices are described, for instance, in U.S. Pat. No. 4,818,540 issued Apr. 4, 1989 to Chinen, et al., herein incorporated by reference.

Compositions having the pharmacologically active compounds of this invention that are suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, intradermal, or subcutaneous injection.

Compositions having the pharmacologically active compounds of this invention that are suitable for transbronchial and transalveolar administration include, but not limited to, various types of aerosols for inhalation. Devices suitable for transbronchial and transalveolar administration of these are also embodiments. Such devices include, but are not limited to, atomizers and vaporizers. Many forms of currently available atomizers and vaporizers can be readily adapted to deliver compositions having the pharmacologically active compounds of the invention.

Compositions having the pharmacologically active compounds of this invention that are suitable for gastrointestinal administration include, but not limited to, pharmaceutically acceptable powders, pills or liquids for ingestion and suppositories for rectal administration. Due to the ease of use, gastrointestinal administration, particularly oral, is a preferred embodiment. Once the pharmaceutical comprising the polymorphic and/or mutant A2M polypeptide or nucleic acid or binding partner, or fragment thereof has been obtained, it can be administered to a organism in need to treat or prevent a neuropathy, such as AD.

Having now generally described the invention, the following examples are offered to illustrate, but not to limit the claimed invention.

### EXAMPLES

The nucleic acid embodiments of the invention include isolated or purified nucleic acids comprising, consisting essentially of, or consisting of an A2M gene (e.g., SEQ ID NO: 1) with one or more of the SNPs and/or mutations described in Table 1. Other embodiments include isolated or purified nucleic acids comprising, consisting essentially of, or consisting of an A2M gene having at least one SNP and/or mutation described in Table 1 along with other SNPs, such as those described in Table 2. Still other embodiments relate to isolated or purified nucleic acid fragments of the A2M gene which include at least one of the SNPs described in Table 1. Such fragments can range in length from at least 10, at least 15, at least 20, at least 25, at least 30, at least 40, at least 50, at least 75, at least 100, a least 150, at least 200, at least 250, at least 300, at least 400, at least 500, at least 750, at least 1000, at least 2500, at least 5000, at least 7500, at least 10,000, at least 20,000, at least 30,000, at least 40,000, at least 50,000 or greater than 50,000 nucleotides and include both exons and introns of the A2M gene. Isolated or purified nucleic acid fragments of the A2M gene having at least one SNP and/or mutation described in Table 1 along with other SNPs, such as those described in Table 2, are also contemplated. Such fragments can range in length from at least 10, at least 15, at least 20, at least 25, at least 30, at least 40, at least 50, at least 75, at least 100, a least 150, at least 200, at least 250, at least 300, at least 400, at least 500, at least 750, at least 1000, at least 2500, at least 5000, at least 7500, at least 10,000, at least 20,000, at least 30,000, at least 40,000, at least 50,000 or greater than 50,000 nucleotides and include both exons and introns of the A2M gene. Other embodiments of the present invention include fragments of the A2M gene, wherein the fragments contains at least 9, at least 16, or at least 18 consecutive nucleotides of the polymorphic or mutant A2M gene but including at least one of the SNPs and/or mutations in Table 1. Isolated or purified nucleic acids that are complementary to said A2M nucleic acids and fragments thereof are also embodiments. Some embodiments also concern genomic DNA, RNA, and cDNA corresponding to polymorphic and/or mutant A2M genes, described herein. Accordingly, in some contexts, the term “polymorphic and/or mutant A2M nucleic acids” refers not only to the full-length polymorphic and/or mutant A2M nucleic acids (e.g., SEQ ID NOS: 1) but also to fragments of these molecules at least 9, at least 16, or at least 18 nucleotides in length but containing at least one of the SNPs and/or mutations identified in Table 1, nucleic acids that are complementary to said full-length sequences and fragments thereof, and genomic DNA, RNA, and cDNA corresponding to said sequences.

The discovery of SNPs and/or mutations in the A2M gene was made while analyzing the sequences of the A2M gene obtained from patients suffering from AD. The approaches used in these experiments is described in EXAMPLE 1.

### Example 1

### Methods of Identifying SNPs and Other Mutations in the A2M Gene

The following protocol that was used to identify the SNPs and/or mutations described herein in patients from the National Institute of Mental Health (NIMH) AD Genetics Initiative Sample. However, it will be appreciated that this protocol has general applicability to any human subject.

The A2M gene was identified as a candidate gene linked to AD based both on its known function and available linkage data. Sample sets of DNA showing strong linkage disequilibrium and/or association in the A2M region were chosen for further study.

The genomic DNA sequence of the A2M gene was obtained as a part of the draft sequence of chromosome 12 from a Human Genome Project information database located at the University of California Santa Cruz available at genome.ucsc.edu. The full-length A2M coding sequence (SEQ ID NO: 2) and A2M protein (SEQ ID NO: 9) sequences were also obtained. The coordinates of publicly available SNPs in the A2M gene were obtained from bio.chip.org. The program SNPer (available at bio.chip.org) was used to place the publicly available SNPs in relation to the exons of the A2M gene. Exon positions generated by SNPer were verified by comparing the cDNA sequence (SEQ ID NO: 2) to the genomic database at the NCBI using (Basic Local Alignment Search Tool) BLASTN with the default filter (Altschul, et al. (1990) J. Mol. Biol. 215:403-410). Alternatively, the A2M cDNA sequence was queried against the High Throughput Genomic Sequence (HTGS) database using BLASTN.

Subsequent to exon verification, specific regions of the A2M gene were selected for sequencing. Regions selected for sequencing were as follows: (1) a region beginning approximately 1000 base pairs upstream of the nucleic acid sequence corresponding to the start codon and extending about 150-200 base pairs beyond last nucleotide of the first exon; (2) a region beginning approximately 150-200 base pairs upstream of the nucleic acid sequence corresponding to the beginning of the least exon of the A2M gene and extending about 700 base pairs beyond last nucleotide of this exon; and (3) a nucleic acid region surrounding each exon which begins approximately 150-200 base pairs upstream and ends approximately 150-200 base pairs downstream of each remaining exon.

Within the selected regions, 500-800 base pair fragments were amplified by using amplification primers flanking specific regions of interest (forward and reverse primers). In general, primers used for amplification ranged from 20 to 24 nucleotides and had an annealing temperature between 54-60° C. Amplification was performed using about 30 ng of human genomic DNA, 5 μmol of each primer, and HotStarTaq Mix (Qiagen). Thermocycling was initiated by heating for 15 minutes at 95° C. followed by 35 cycles of (a) 94° C. for 30 seconds; (b) primer annealing temperature for 45 seconds; and (c) 72° C. for 1 minute. The cycling was followed by a final 7 minute extension at 72° C. Subsequent to thermocycling, PCR products were purified then quantitated.

Both strands of each amplified fragment were sequenced using sequencing primers complementary to a region near the 3′-end of each strand. Approximately, 3.2 pmol of sequencing primer and 12 ng of amplified fragment were added to sequencing buffer including Big Dye Terminator Mix (Applied Biosystems—ABI) according to the manufacturer's instructions. Thermocycling included 30 cycles of (a) 96° C. for 10 seconds; (b) 50° C. for 5 seconds; and (c) 60° C. for 4 minutes. Reaction products were purified using CentriSep 96 well plates (Princeton Separations) according to manufacturer's instructions. Data was collected from purified reaction products using an ABI 3700 DNA Analyzer.

Using the above amplification and sequencing protocol, several SNPs and/or mutations were found in the A2M gene, including both exon and intron regions, in individuals having AD. These results are set out in Table 1 herein.

In view of the fact that the presence of one or more of SNPs and/or mutations in an individual can present a risk that the individual will acquire AD, it is contemplated that the SNPs and/or mutations described in Table 1 (i.e., 6i, 12i.1, 12i.2, 12e, 14e, 14i.1, 14i.2, 17i.1, 20e, 20i, 21i, 28i and 30e) can be indicative for altered risk for AD. As a preliminary evaluation of the risk associated with possessing one or more of these SNPs, an association analysis in families and individuals having AD was performed. That is, the nucleotide identities at the position of one or more of SNPs and/or mutations included in Table 1 (i.e., 6i, 12i.1, 12i.2, 12e, 14e, 14i.1, 14i.2, 17i.1, 20e, 20i, 21i, 28i and 30e) in individuals and families with AD were determined and tested by both single SNP association analyses and haplotype analyses. EXAMPLE 2 describes these experiments.

### Example 2

### Association of A2M SNPs and Haplotypes with Alzheimer's Disease

The polymorphisms listed in Table 1 can be detected from biological samples provided by families having members afflicted with AD using the methods described below as well as methods known to those having ordinary skill in the art. Furthermore, association of one or more polymorphisms listed in Table 1 with an altered risk of AD can be determined using the methods described below as well as those described in U.S. Pat. No. 6,265,546, the disclosure of which is incorporated herein by reference in its entirety, and those methods known to those having ordinary skill in the relevant art. As described in Example 1, for each of the polymorphisms listed in Table 1, the A2M-1 allele corresponds to the allele represented in SEQ ID NO: 1. The A2M-2 allele corresponds to an allele having the polymorphic change (nucleotide substitution or mutation) as indicated in column 3 of Table 1 at the sequence position specified in column 2 of Table 1 (the positions and nucleotides affected by each polymorphism and/or mutation are also provided in the FIGURE).

1. To test for a link between the polymorphisms described herein and AD, samples from families having members afflicted with AD were used. An example of an appropriate population is the National Institute of Mental Health (NIMH) Genetics Initiative AD sample, a large sample of affected sibling pairs and other small families with AD. It should be noted, however, that any population of families having members meeting the criteria described below can be used for association and haplotype analyses.

Participants in the NIMH sample were recruited from local memory disorder clinics, nursing homes, and the surrounding communities with the only requirement for inclusion in the sample being that each family member include at least two living blood relatives with memory problems. They were evaluated following a standardized protocol (Blacker, D., et al., Arch. Neurol. 51:1198-1204 (1994)) to assure that they met NINCDS/ADRDA criteria for Probable AD (or in the case of secondary probands, Possible AD) (McKhann, G., et al., Neurology 34:939-944 (1984)), or research pathological criteria for Definite AD (Khachaturian, Z., Arch. Neurol. 42:1005 (1985)). Among the affected individuals, 142 (22.2%) had autopsy confirmation of the diagnosis of AD. Unaffected relatives, generally siblings, were included when they were available and willing to participate.

There were a total of 239 unaffected subjects from 131 families (45.6%). An additional 22 study subjects with blood available who had unclear phenotypes were considered phenotype unknown, as were 5 unaffected subjects with unknown ages, and 19 unaffected subjects below 50 years of age (primarily children of affected participants). There were a total of 639 individuals affected with AD, from 286 families. The majority of the affected individuals were sibling pairs (202 families, 71%), but there were 46 larger sibships (16%), and 38 families with other structures (13%; e.g., parent-child, first cousin, avuncular, extended). All subjects (or, for significantly cognitively impaired individuals, their legal guardian or caregiver with power of attorney) gave informed consent.

The full NIMH sample can be used in the descriptive statistics for genotype counts and allele frequencies, for the analyses of age of onset in affected individuals, and for all of the genetic linkage analyses (except ASPEX, which uses sibships only). However, because the Mantel-Haenzel test, conditional logistic regression, and Sibship Disequalibrium Test and EV-FBAT depend on comparisons of closely related affected and unaffected individuals, they are performed on a subsample including all families in which there is at least one affected and at least one unaffected sibling with A2M data available: 104 families with 217 affected and 181 unaffected siblings.

In order to avoid examining very early onset AD, which appears to have a distinct genetic etiology (Blacker, D. & Tanzi, R. E., Arch Neurol 55:294-296 (1998)), only those families in which all examined affected individuals experienced the onset of AD at age 50 of later are included. Although Late Onset Alzheimer's Disease (LOAD) is conventionally identified based on onset after age 60, families with onsets between 50 and 60 are included because onset in this decade is only partly explained by the known AD genes. Age of onset is determined based on an interview with a knowledgeable informant and review of medical records.

The polymorphisms described herein can be manually genotyped according to, for example, the protocol described in Matthijs et al. (Matthijs, G., & Marynen, P., Nuc. Acid Res. 19:5102 (1991)). Alternatively, an appropriate fragment of the A2M gene corresponding to the region of a polymorphism and/or mutation described herein is amplified and sequenced using the methods described in Example 1.

In one example, manual genotyping is carried out using a 96-well microtiter dish format as follows. Three to 10 nanograms of human DNA is mixed with a reaction buffer, deoxynucleotide mix (e.g. for a poly-[dGdT]STR, the final concentration is 200 mM each of dATP, dCTP, and dTTP; and 2 mM dGTP), 1 mCi alpha-³²PdGTP or ³³P-dGTP, 15 pM of each flanking primer and 0.25 units of Taq polymerase in a total volume of 10 μL. The reaction are denatured at 94° C. for 4 minutes, followed by 25-30 cycles of 1 minute denaturing at 94° C., 0.5-1 minute annealing (variable temperature, usually 55-65° C.) and extension for I minute at 72° C. Forty-eight (48) experimental and two control (for standardization of size) samples are loaded on a gel at one time, thereby increasing the amount of information per gel. Whenever possible (e.g., if maker background is sufficiently low) multiple markers (two to four markers) are multiplexed, or are temporally staggered (30-45 minutes) two to three mm on a single gel. Allele sizes for CEPH individuals 1331-01 and 1331-02 are used as standards. In the rare event that no standards are available for a marker, an initial gel is run, which includes a sequencing ladder, to determine allele sizes in these individuals. Two μL of sample are mixed with loading dye and size-fractionated on a 6% denaturing polyacrylamide gel. The gels are then dried and placed on X-ray film for 2-24 hrs. at −80° C. and read by two independent readers.

It will be appreciated that the manual geneotyping method described above is only one method that is available for detecting specific alleles at polymorphic loci. Several other methods that are useful for detecting specific alleles at polymorphic loci, in particular human polymorphic loci. The preferred method for detecting a particular polymorphism, depends on the nature of the polymorphism. Several methods of determining the presence or absence of allelic variants of a gene are provided below. Methods that are useful are not limited to those described below, but include all available methods.

Generally, these methods are based in sequence-specific polynucleotides, oligonucleotides, probes and primers. Any method known to those of skill in the art for detecting a specific nucleotide within a nucleic acid sequence or for determining the identity of a specific nucleotide in a nucleic acid sequence is applicable to the methods of determining the presence or absence of an allelic variant of these genes on chromosome 12. Such methods include, but are not limited to, techniques utilizing nucleic acid hybridization of sequence-specific probes, nucleic acid sequencing, selective amplification, analysis of restriction enzyme digests of the nucleic acid, cleavage of mismatched heteroduplexes of nucleic acid and probe, alterations of electrophoretic mobility, primer specific extension, oligonucleotide ligation assay and single-stranded conformation polymorphism analysis. In particular, primer extension reactions that specifically terminate by incorporating a dideoxynucleotide are useful for detection. Several such general nucleic acid detection assays are known (see, e.g., U.S. Pat. No. 6,030,778).

Any cell type or tissue may be utilized to obtain nucleic acid samples, e.g., bodily fluid such as blood or saliva, dry samples such as hair or skin.

a. Primer Extension-Based Methods

Several primer extension-based methods for determining the identity of a particular nucleotide in a nucleic acid sequence have been reported (see, e.g., PCT Application Nos. PCT/US96/03651 (WO 96/29431), PCT/US97/20444 (WO 98/20166), PCT/US97/20194 (WO 98/20019), PCT/US91/00046 (WO91/13075), and U.S. Pat. Nos. 5,547,835, 5,605,798, 5,622,824, 5,691,141, 5,872,003, 5,851,765, 5,856,092, 5,900,481, 6,043,031, 6,133,436 and 6,197,498.) In general, a primer is prepared that specifically hybridizes adjacent to a polymorphic site in a particular nucleic acid molecule. The primer is then extended in the presence of one or more dideoxynucleotides, typically with at least one of the dideoxynucleotides being the complement of the nucleotide that is polymorphic at the site. The primer and/or the dideoxynucleotides may be labeled to facilitate a determination of primer extension and identity of the extended nucleotide.

A preferred method of genotyping or determining the presence of an allelic variant two-dye fluorescence polarization detected single base extension (FP-SBE (12)) on an LJL-Biosystems Criterion Analyst AD (Molecular Devices, Sunnyvale, Calif.). PCR primers are designed to yield products between 200-400 bp in length, and are used at a final concentration of 100-300 nM (Invitrogen Corp., Carlsbad, Calif.) along with Taq polymerase (0.25 U/reaction; Qiagen, Valencia, Calif. and Roche, Indianapolis, Ind.) and dNTPs (2.5 uM/rxn; Amersham-Pharmacia, Piscataway, N.J.). All PCR reactions are performed from −10 ng of DNA. General PCR thermo-cycling conditions are as follows: initial denaturation 3 minutes at 94EC, followed by 30-35 cycles of denaturation at 94EC for 45 seconds, primer-specific annealing temperature (see below) for 45 seconds, and product extension at 72EC for 1 minute. Final extension at 72EC for six minutes. PCR products can be visualized on 2% agarose-gels to confirm a single product of the correct size. PCR primers and unincorporated dNTPs can be degraded by adding exonuclease I (Exol, 0.1-0.15 U/reaction; New England Biolabs, Beverly, Mass.) and shrimp alkaline phosphatase (SAP, 1U/reaction; Roche, Indianapolis, Ind.) to the PCR reactions and incubating for 1 hour at 37EC, followed by 15 minutes at 95EC to inactivate the enzymes. The single base extension step is performed by directly adding SBE primer (100 nM; Invitrogen Corp., Carlsbad, Calif.), Thermosequenase (0.4 U/reaction; Amersham-Pharmacia, Piscataway, N.J.), and the appropriate mixture of R110-ddNTP, TAMRA-ddNTP (3 uM; NEN, Boston, Mass.), and all four unlabeled ddNTPs (22 or 25 uM; Amersham-Pharmacia, Piscataway, N.J.) to the Exol/SAP treated PCR product. Acycloprime-FP SNP detection kits (G/A)(Perkin-Elmer, Boston, Mass.) may also be used for the SBE reaction. Incorporation of the SNP specific fluorescent ddNTP is achieved by subjecting samples to 35 cycles of 94EC for 15 seconds and 55EC for 30 seconds. The length of the SBE primers are designed to yield a melting temperature Tₘ of 62-64EC. Fluorescent ddNTP incorporation is detected using the Analyst™ AD System (Molecular Devices, Sunnyvale, Calif.) and measuring fluorescent polarization for R110 (excitation at 490 nm, emission at 520 nm) and TAMRA (excitation at 550 nm, emission at 580 nm). Genotypes are called manually or automatically using the manufacturer's software (‘Allelecaller vers. 1.0’, Molecular Devices, Sunnyvale, Calif.). In view of the polymorphic regions provided herein, SNP specific PCR primers (5′ to 3′ sequences), annealing temperature, product length, SBE primer sequence, SNP location and reference sequence position, can readily be determined by those of skill in the art using well-known methods.

b. Polymorphism-Specific Probe Hybridization

Another detection method is allele specific hybridization using probes overlapping the polymorphic site and having about 5, 10, 15, 20, 25, or 30 nucleotides around the polymorphic region. The probes can contain naturally occurring or modified nucleotides (see U.S. Pat. No. 6,156,501). For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163; Saiki et al. (1989) Proc. Natl Acad. Sci U.S.A. 86:6230; and Wallace et al. (1979) Nucl. Acids Res. 6:3543). Such allele specific oligonucleotide hybridization techniques may be used for the simultaneous detection of several nucleotide changes in different polymorphic regions. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid. In a preferred embodiment, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix, Santa Clara, Calif.). Mutation detection analysis using these chips comprising oligonucleotides, also termed “DNA probe arrays” is described e.g., in Cronin et al. (1996) Human Mutation 7:244 and in Kozal et al. (1996) Nature Medicine 2:753. In one embodiment, a chip includes all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

C. Nucleic Acid Amplification-Based Methods

In other detection methods, it is necessary to first amplify at least a portion of a gene prior to identifying the allelic variant. Amplification can be performed, e.g., by PCR and/or LCR, according to methods known in the art. In one embodiment, genomic DNA of a cell is exposed to two PCR primers and amplification is performed for a number of cycles sufficient to produce the required amount of amplified DNA. In another embodiment, the primers are located between 150 and 350 base pairs apart.

Alternative amplification methods include: self sustained sequence replication (Guatelli, J. C. et al. (1990) Proc. Natl. Acad. Sci. U.S.A. 87:1874-1878), transcriptional amplification system (Kwoh, D. Y. et al. (1989) Proc. Natl. Acad. Sci. U.S.A. 86:1173-1177), Q-Beta Replicase (Lizardi, P. M. et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

Alternatively, allele specific amplification technology, which depends on selective PCR amplification may be used in conjunction with the alleles provided herein. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 1 7:2503). In addition it may be desirable to introduce a restriction site in the region of the mutation to create cleavage-based detection (Gasparini et al. (1992) Mol. Cell Probes 6:1).

d. Nucleic Acid Sequencing-Based Methods

Any of a variety of sequencing reactions known in the art can be used to directly sequence at least a portion of a gene and to detect allelic variants, e.g., mutations, by comparing the sequence of the sample sequence with the corresponding wild-type (control) sequence. Exemplary sequencing reactions include those based on techniques developed by Maxam and Gilbert (1977) Proc. Natl. Acad. Sci. U.S.A. 74:560) or Sanger et al. (1977) Proc. Natl. Acad. Sci 74:5463. It is also contemplated that any of a variety of automated sequencing procedures may be used when performing the subject assays ((1995) Biotechniques 19:448), including sequencing by mass spectrometry (see, for example, U.S. Pat. Nos. 5,547,835, 5,691,141, and International PCT Application No. PCT/US94/00193 (WO 94/16101), entitled “DNA Sequencing by Mass Spectrometry” by H. Koster; U.S. Pat. Nos. 5,547,835, 5,622,824, 5,851,765, 5,872,003, 6,074,823, 6,140,053 and International PCT Application No. PCT/US94/02938 (WO 94/21822), entitled “DNA Sequencing by Mass Spectrometry Via Exonuclease Degradation” by H. Koster, and U.S. Pat. Nos. 5,605,798, 6,043,031, 6,197,498, and International Patent Application No. PCT/US96/03651 (WO 96/29431) entitled “DNA Diagnostics Based on Mass Spectrometry” by H. Koster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track sequencing or an equivalent, e.g., where only one nucleotide is detected, can be carried out. Other sequencing methods are known (see, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”).

e. Restriction Enzyme Digest Analysis

In some cases, the presence of a specific allele in nucleic acid, particularly DNA, from a subject can be shown by restriction enzyme analysis. For example, a specific nucleotide polymorphism can result in a nucleotide sequence containing a restriction site which is absent from the nucleotide sequence of another allelic variant.

f. Mismatch Cleavage

Protection from cleavage agents, such as, but not limited to, a nuclease, hydroxylamine or osmium tetroxide and with piperidine, can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an allelic variant with a sample nucleic acid, e.g, RNA or DNA, obtained from a tissue sample. The double-stranded duplexes are treated with an agent, which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they differ (see, for example, Cotton et al. (1988) Proc. Natl Acad Sci U.S.A. 85:4397; Saleeba et al. (1992) Methods Enzymod. 217:286-295). The control or sample nucleic acid is labeled for detection.

g. Electrophoretic Mobility Alterations

In other embodiments, alteration in electrophoretic mobility is used to identify the type of allelic variant of a gene of interest. For example, single-strand conformation polymorphism (SSCP) may be used to detect differences in electrophoretic mobility between mutant and wild type nucleic acids (Orita et al. (1989) Proc. Natl. Acad. Sci. U.S.A. 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another embodiment, the subject method uses heteroduplex analysis to separate double stranded heteroduplex molecules on the basis of changes in electrophoretic mobility (Keen et al. (1991) Trends Genet 7:5).

h. Polyacrylamide Gel Electrophoresis

In yet another embodiment, the identity of an allelic variant of a polymorphic region of an gene is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

i. Oligonucleotide Ligation Assay (OLA)

In another embodiment, identification of the allelic variant is carried out using an oligonucleotide ligation assay (OLA), as described, e.g., in U.S. Pat. No. 4,998,617 and in Landegren, U. et al. (1988) Science 241:1077-1080. The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. U.S.A. 87:8923-8927). In this method, PCR is used to achieve the exponential amplification of target DNA, which is then detected using OLA.

Several techniques based on this OLA method have been developed and can be used to detect specific allelic variants of a polymorphic region of a gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucl. Acids Res. 24:3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

j. SNP Detection Methods

Several methods have been developed to facilitate the analysis of single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment, a solution-based method for determining the identity of the nucleotide of a polymorphic site is employed (Cohen, D. et al. (French Patent 2,650,840; PCT Application No. WO91/02087)). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

k. Genetic Bit Analysis

An alternative method, known as Genetic Bit Analysis or GBA™ is described by Goelet, et al. (U.S. Pat. No. 6,004,744, PCT Application No. 92/15712). The method of Goelet, et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO91/02087), the method of Goelet, et al. is preferably a heterogeneous phase assay, in which the primer or the target molecule is immobilized to a solid phase.

l. Other Primer-Guided Nucleotide Incorporation Procedures

Other primer-guided nucleotide incorporation procedures for assaying polymorphic sites in DNA have been described (Komher, J. S. et al. (1989) Nucl. Acids Res. 17:7779-7784; Sokolov, B. P. (1990) Nucl. Acids Res. 18:3671; Syvanen, A. C., et al. (1990) Genomics 8:684-692, Kuppuswamy, M. N. et al. (1991) Proc. Natl. Acad. Sci. (U.S.A.) 88:1143-1147; Prezant, T. R. et al. (1992) Hum. Mutat. 1: 159-164; Ugozzoli, L. et al. (1992) GATA 9:107-112; Nyren, P. et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al. (1993) Amer. J. Hum. Genet. 52:46-59).

For determining the identity of the allelic variant of a polymorphic region located in the coding region of a gene, yet other methods than those described above can be used. For example, identification of an allelic variant which encodes a mutated protein can be performed by using an antibody specifically recognizing the mutant protein in, e.g., immunohistochemistry or immunoprecipitation. Binding assays are known in the art and involve, e.g., obtaining cells from a subject, and performing binding experiments with a labeled lipid, to determine whether binding to the mutated form of the protein differs from binding to the wild-type protein.

m. Molecular Structure Determination

If a polymorphic region is located in an exon, either in a coding or non-coding region of the gene, the identity of the allelic variant can be determined by determining the molecular structure of the mRNA, pre-mRNA, or cDNA. The molecular structure can be determined using any of the above described methods for determining the molecular structure of the genomic DNA, e.g., sequencing and single-strand conformation polymorphism.

n. Mass Spectrometric Methods

Nucleic acids can also be analyzed by detection methods and protocols, particularly those that rely on mass spectrometry (see, e.g., U.S. Pat. Nos. 5,605,798, 6,043,031, 6,197,498, and International Patent Application No. WO 96/29431, International PCT Application No. WO 98/20019).

Multiplex methods allow for the simultaneous detection of more than one polymorphic region in a particular gene. This is the preferred method for carrying out haplotype analysis of allelic variants of a gene.

Multiplexing can be achieved by several different methodologies. For example, several mutations can be simultaneously detected on one target sequence by employing corresponding detector (probe) molecules (e.g., oligonucleotides or oligonucleotide mimetics). Variations in additions to those set forth herein will be apparent to the skilled artisan.

A different multiplex detection format is one in which differentiation is accomplished by employing different specific capture sequences which are position-specifically immobilized on a flat surface (e.g., a ‘chip array’).

o. Other Methods

Additional methods of analyzing nucleic acids include amplification-based methods including polymerase chain reaction (PCR), ligase chain reaction (LCR), mini-PCR, rolling circle amplification, autocatalytic methods, such as those using QJ replicase, TAS, 3SR, and any other suitable method known to those of skill in the art.

Other methods for analysis and identification and detection of polymorphisms, include but are not limited to, allele specific probes, Southern analyses, and other such analyses.

Five groups of statistical analyses can be used to explore the relationship between A2M and AD in study families. First, the A2M genotype and allele frequencies for affected and unaffected individuals are calculated. Second, stratified on families, Mantel-Haenzel odds ratios (see Mantel, H. & Haenszel, W. J. Natl. Cancer Inst. 22:719-748 (1959), the disclosure of which is incorporated by reference in its entirety) are calculated for the effect of possessing an allele for each polymorphism and/or mutation described herein on altering the risk for AD, and conditional logistic regression, conditioning on family, is used to control for the effect of APOE-ε4. Third, association for each polymorphism and/or mutation described herein is tested for using the Sibship Disequilibrium Test (SDT) of Horvath and Laird (Horvath, S. & Laird, N., Am. J. Hum. Genet. 63:1886-1897 (1998), the disclosure of which is incorporated by reference in its entirety), a variation of the Transmission Disequilibrium Test (TDT) that is able to detect linkage and association in the absence of parental data or the FBAT or EV-FBAT developed by Rabinowitz and Laird (Rabinowitz, D & Laird, N., Hum. Hered. 50:211-23 (2000), the disclosure of which is incorporated by reference in its entirety). Fourth, a variety of techniques are used to assess whether any A2M effect occurs via a change in age of onset. Fifth, several genetic association methods can be used to assess the relationship between A2M and AD, and whether any allelic association might be related to the recent report of linkage to centromeric markers on chromosome 12. Wherever possible, APOE-ε4 effects are controlled for by stratification or by including APOE-ε4 as a covariate in multivariate analyses. Except as otherwise noted, the analyses reported here can be performed using statistical analysis software such as, the SAS statistical analysis package (SAS Institute, SAS Program Guide, Version 6, Cary, N.C. (1989)).

For all types of analysis, allele frequencies are computed from the data, but rare alleles can be adjusted up to a frequency of 0.01 (with a compensatory small decrease in the frequency of the most common alleles) in order to minimize the possibility of a false positive result. All analyses are repeated using the uncorrected frequencies.

For descriptive purposes, A2M genotype counts and allele frequencies are examined in affected and unaffected subjects in study families. Unaffected individuals in AD families are not genetically independent of their affected relatives, of course, and thus would be expected to show higher frequencies of AD-associated alleles compared to the general population. However, given an increased risk of AD with a given allele, its frequencies would be expected to be higher among affected individuals than among their unaffected relatives. However, since these frequencies are pooled across families, they are neither as accurate nor as powerful an indicator of genetic association as the SDT.

A2M genotype counts and allele frequencies for each polymorphism described herein are reported separately for primary and secondary probands, with primary probands serving as the primary subject population, and secondary probands as a confirmation sample. Allele frequencies in the probands are compared to those for unaffected individuals based on the oldest unaffected individuals from each of the 105 families in which one or more unaffected subjects with A2M data is available. In addition, the analyses are repeated using an unaffected sample that had passed through a majority of the age of risk, the “stringent” unaffecteds, those who are at least as old as the age of onset of the latest-onsetting affected family member, again selecting the oldest such individual in each family. Because age of onset is correlated in families (Farrer, L. A., et al., Neurology 40:395-403 (1990)), using onset ages in the subjects' own families is preferable to setting an arbitrary cutoff.

Initial genotype counts and allele frequencies for each polymorphism and/or mutation described herein are determined (Matthijs, G., Marynen, P., Nuc. Acid. Res. 19:5102 (1991)) in primary probands, secondary probands, unaffected individuals (oldest in family), and “stringent” unaffecteds, (those who have reached the onset age of the latest-onsetting affected, again using the oldest such individual), stratified on individual APOE dose.

Mantel-Haenzel odds ratios (see Mantel, H. & Haenszel, W. J. Natl. Cancer Inst. 22:719-748 (1959), the disclosure of which is incorporated by reference in its entirety) can be calculated for the odds of being affected given the possession of at least one allele of a polymorphism described herein. These analyses are preformed stratified on family using n-to-m matching, so all members of a sibship can be used and intercorrelations among siblings can be taken into account. Spielman and Ewens (Spielman, R. S., and Ewens, W. J. Am. J. Hum. Genet. 62:450-458 (1998)) have suggested the use of a similar analysis to test for linkage. The analyses are performed first using all unaffected siblings, and then only the stringent unaffected siblings.

Conditional logistic regression is used to control the Mantel-Haenzel odds ratio for the effect of APOE-ε4 on AD risk. Here, the outcome is disease status of each sibling, conditioning on family using an n-to-m matching paradigm, and including APOE-ε4/ε4 homozygosity as a covariate, along with a term for the interaction between APOE-ε4 and A2M alleles of polymorphisms described herein. Like the Mantel-Haenzel odds ratio, conditional logistic regression is a standard method for analysis of data from matched sets, and can control for clustering of genotypes within families of arbitrary size. These analyses are performed using the PHREG procedure in SAS (SAS Institute, SAS Program Guide, Version 6, Cary N.C. (1989)). These analyses are repeated using only the “stringent” unaffected siblings (those who were as least as old as the onset age of the oldest-onsetting affected sibling) in order to minimize the effect of misclassification of unaffected siblings. These analyses can also be performed coding APOE-ε4 as gene dosage, and including a term for the possession of an APOE-2 allele, previously shown to decrease disease risk (Corder, E. H., et al., Nat. Genet. 7:180-184 (1994); Farrer, L. A., et al., JAMA 278;1349-1356 (1997)).

Mantel-Haenzel odds ratios and p-values for the association of A2M alleles for each polymorphism described herein with risk of AD will be greater than 2 and less than 0.05, respectively. Conditional logistic regression analyses, which allow for the calculation of Mantel-Haenzel odds ratios adjusted for the effect of APOE-ε4 on AD risk, are also expected to generate statistically significant p-values (less than 0.05) for association of A2M alleles for each polymorphism described herein with risk of AD. Interaction between A2M alleles for each polymorphism described herein and APOE-ε4 are not expected to be statistically significant.

The Sibship Disequilibrium Test (SDT) (Horvath, S. & Laird, N., Am. J. Hum. Genet. 63:1886-1897 (1998), the disclosure of which is incorporated by reference in its entirety) is a non-parametric sign test developed for use with sibling pedigree data that compares the average number of candidate alleles between affected and unaffected siblings. The SDT is similar to the S-TDT, a recently developed test that also does not require parental data (Spielman, R. S., and Ewens, W. J., Am. J. Hum. Genet. (Suppl.) 53:363 (1993) the disclosure of which is incorporated herein by reference in its entirety), but has the advantage of being able to detect association in sibships of an arbitrary size. Like the TDT, S-TDT, and other family-based association tests, the SDT offers the advantage of not being susceptible to errors due to admixture. Another advantage of these methods is that misclassification of affection status (e.g., due to the unaffected siblings not having passed through the age of risk) decreases the power of the test, but does not lead to invalid results. The SDT can test for both linkage and linkage disequilibrium; it can only detect linkage disequilibrium in the presence of linkage, hence there is no confounding due to admixture. The null hypothesis of the SDT is that Θ=½ (no linkage) or δ=0 (no disequilibrium), i.e., H₀:δ(Θ−½)=0. The SDT program (for several platforms) and documentation may be found at ftp://sph70-57.harvard.edu/XDT/.

Because the SDT does not require parental data, and can use all information from sibships of arbitrary size, it is well-suited to the analysis of the NIMH AD data. Before using it to detect novel AD genes, the SDT is validated with the known AD gene APOE-ε4 in the sample. For example, in an examination of 150 sibships with 286 affected and 242 unaffected individuals from the sample, the SDT was able to detect not only the deleterious APOE-ε4 effect but also the more difficult to detect APOE-2 protective effect (Farrer, L. A., et al., JAMA 278:1349-1356 (1997); Corder, E. H., et al., Nature Genet. 7:180-184 (1994)) not previously detected in these data (Blacker, D., et al., Neurology 48:139-147 (1997)).

The primary analysis of the association of A2M polymorphisms with AD examines the probability of passing along an A2M polymorphic allele as a function of affection status. In order to increase the likelihood of correct classification of unaffected status, the analyses are repeated including only “stringent” unaffected siblings, those who were at least as old as the latest on setting affected siblings, a sample of 60 families. In addition, in order to assess whether the effect differed in different APOE genotypes persists in individuals with similar APOE genotypes, the analyses are repeated within strata defined by matching affected and unaffected siblings for APOE-ε4 gene dose. To provide further validation of the SDT, the Sibling TDT (Spielman, R. S. and Ewens, W. J., Am. J. Hum. Genet. 62:450-458 (1998), the disclosure of which is incorporated herein by reference in its entirety) (S-TDT) is applied.

The SDT Z values and p-values for the association of A2M alleles for each polymorphism described herein with risk of AD will be greater than 2 and less than 0.05, respectively. The SDT values are expected to be confirmed by the S-TDT.

The general approach to family-based examinations described by Rabinowitz and Laird (Rabinowitz, D & Laird, N., Hum. Hered. 50:211-23 (2000), the disclosure of which is incorporated by reference in its entirety) (FBAT and EV-FBAT) can also be used to test the association between the A2M alleles of the polymorphisms described herein and risk of AD. This approach is based on computing p-values by comparing test statistics for association to their conditional distributions given the minimal sufficient statistic under the null hypothesis for the genetic model, sampling plan and population admixture. The approach can be applied with any test statistic, so any kind of phenotype and multi-allelic markers may be examined, and covariates may be included in analyses. By virtue of the conditioning, the approach results in correct type I error probabilities regardless of population admixture, the true genetic model and the sampling strategy. The EV-FBAT test statistics and p-values for the association of A2M alleles for each polymorphism described herein with risk of AD will be greater than 2 and less than 0.05, respectively.

In order to see if A2M effects appear to operate via changes in age of onset, affected individuals are examined according to A2M genotype, stratifying on or controlling for the powerful effect of APOE-ε4. First, this is examined graphically using Kaplan Meier curves including all affected and unaffected individuals, first stratifying on A2M genotype alone, and then on A2M risk allele carrier status for each polymorphism describe herein and APOE-ε4 dose. Second, the mean ages of onset of primary and secondary probands are compared by A2M genotype overall, and stratified on APOE-ε4 gene dose. Third, analysis of variance (performed separately for primary and secondary probands) is used, including first only A2M genotype (defined as any 2 vs. none), then only APOE genotype (defined as APOE-ε4 gene dose or APOE-ε4/ε4 vs. not), then both, and then both plus an interaction term.

Analyses of haplotypes that are associated with AD can be performed using software such as TRANSMIT version 2.5 (Clayton, (1999) Am. J. Hum. Genet. 65: 1170-1177, see also Clayton et al., (1999) Am. J. Hum. Genet. 65: 1161-1169, the disclosures of which are incorporated herein by reference in their entireties). This approach is a generalization of the TDT and uses an expectation-maximization (EM) algorithm to reconstruct haplotypes with missing parental genotypes. Nominal global p-values are estimated using the empirical variance function.

For all types of analyses, allele frequencies are computed from the data, but rare alleles are adjusted up to a frequency of 0.01 (with a compensatory small decrease in the frequency of the most common alleles) in order to minimize the possibility of a false positive result. All analyses are repeated using the uncorrected frequencies.

The association analysis and haplotype analysis can be performed for the SNPs and/or mutations described herein using the methodology employed in U.S. Pat. Nos. 6,265,546; 6,090,620; 6,201,107; or 6,303,307; all of which are hereby expressly incorporated by reference in their entireties. The p-values for the association of haplotypes, which include A2M alleles for polymorphism and/or mutations described herein, with risk of AD will be less than 0.05.

SNP 18i (the site of a five base pair deletion of the sequence ACCAT located 1 base pair upstream of exon 18, see the FIGURE) and 24e polymorphism (site of a nucleotide substitution of A to G at nucleotide position 145 within exon 24 which results in an isoleucine to valine substitution in the A2M polypeptide (SEQ ID NO: 9) at amino acid position 1000, see the FIGURE) were examined for association with AD using some of the above-described methods. Specifically, the Sibling TDT described by Spielman and Ewens and the EV-FBAT described by Rabinowitz and Laird were determined. For 18i the population sample size was 76 and for 24e the sample size was 110. The p-value for the association of the 18i deletion with AD was 0.0002 using EVA-BAT and 0.0015 using S-TDT whereas the p-value for the association of the 24e polymorphism with AD was 0.09 using EV-FBAT and 0.14 using S-TDT. Accordingly, the A2M-2 allele of 18i (pentanucleotide deletion) showed strong statistical significance for association with AD and the A2M-1 allele of 24e (A) displayed a trend for association.

The 21i polymorphism described herein was tested for association with AD using the Sibling TDT and EV-FBAT as above. The population that was sampled has an effective size of 92 individuals. The frequency of the minor allele in this population was 0.22. The p-value calculated using the S-TDT was 0.001 whereas the p-value calculated using the EV-FBAT was 0.004. Each of these values are statistically significant and provide evidence that the 21i polymorphism, specifically the T allele, is associated with an increased risk of incurring AD.

Table 3 displays the results of similar analyses that were performed for 21i from other sample populations and for 12e. In particular, Table 3 lists the size of the population of AD patients sampled for each SNP and/or mutation and the frequency of the minor allele in that population. The p-values (based on EV-FBAT statistics) for each of these SNPs and/or mutations samples are also provided in Table 3. In some cases, the population was made up entirely of affected individuals over the age of 65. In these cases, a separate p-value is included that represents the significance of the association of the examined SNP and/or mutation with the development of Late Onset AD (LOAD). EVA-BAT-based p-values that are less than or equal to 0.05 indicate statistical significance. Additionally, for each SNP and/or mutation that was investigated, Table 3 provides an odds ratio (OR) and the corresponding 95% confidence interval, which describes the association with AD for both heterozygous and homozygous genotypesThe values shown in Table 3 for 12e are statistically significant and provide evidence that the 12e polymorphism, specifically the T allele, is associated with an increased risk of incurring AD.

TABLE 3
Genetic Association of Individual SNPs and/or Mutations with Alzheimer's Disease

| | | | | Odds Ratio (95% | Odds Ratio (95% |
|---|---|---|---|---|---|
| | | Minor | | Confidence | Confidence |
| SNP/ | Sample | Allele | p-value | Interval) for a | Interval) for two |
| Mutation | Size | Frequency | (EV-FBAT) | single risk^{a} allele | risk^{a} alleles |
| 12e | 37 | 0.06 | 0.0009 | 3.62 (1.79, 7.34) | 12.9 (0.94, 176) |
| 12e | 39 | 0.07 | 0.0018 | 3.18 (1.69,5.99) | 11.6 (0.88, 154) |
| 12e | 31* | 0.07 | 0.0031* | ND | ND |
| 21i | 92 | 0.22 | 0.004 | 2.00 (1.34, 3.02) | 4.01 (1.27, 11.8) |
| 21i | 71 | 0.17 | 0.041 | 1.72 (1.16,2.56) | 1.84 (0.55, 6.11) |
| 21i | 50* | 0.17 | 0.0039* | ND | ND |

*All individuals sampled were over the age of 65.
ND Not determined.
^{a}risk allele is T for 12e, T for 21i

Individual polymorphisms were also analyzed by FBAT-EV taking into account whether unaffected phenotype information was included and whether the sample was the total sample (1439 individuals from 437 families; all sampled affecteds had onset ages> or =50 years) or the late-onset stratum (all sampled affecteds had onset> or =65 years). By this analysis, the 18i deletion polymorphism is associated in the total sample (Pₙₒₘᵢₙₐₗ=0.02 for affecteds only, and 0.0059 with unaffected phenotypes included) and more strongly associated in the late-onset sample (Pₙₒₘᵢₙₐₗ=0.0033 for affecteds only, and 0.0023 with unaffected phenotypes included). The exon 24 nonsynonmous SNP (24e; Val 1000 Ile) displays a trend towards association in most analyses, and reaches significance in the late-onset stratum when unaffected phenotypes are included in the analysis (Pₙₒₘᵢₙₐₗ=0.037). Significant nominal association results were obtained for the synonymous SNP found in exon 12 (12e) in the total sample (Pₙₒₘᵢₙₐₗ=0.0018 for affecteds, only and 0.00080 with unaffected phenotypes included), with slightly less significant results in the late-onset stratum. Polymorphism 21i was significantly associated in the total sample (Pₙₒₘᵢₙₐₗ=0.041 for affecteds only, and 0.019 with unaffected phenotypes included), with more significant results in the late-onset stratum. Polymorphisms 14i.1 and rs1805654 (in intron 28, see FIGURE) gave significant evidence of association in the late-onset stratum when the unaffected phenotypes were included (Pₙₒₘᵢₙₐₗ=0.043 for 14i.1, and 0.037 for rs1805654), and the polymorphism 6i displayed a trend towards association in the same setting (Pₙₒₘᵢₙₐₗ=0.067).

For the polymorphisms showing at least a trend toward association by FBAT, odds ratios (ORs) for their effect on AD risk were calculated using conditional logistic regression, and are given in Table 4. The 95% confidence intervals (CIs) are provided to give an idea of the precision of these estimates, but it should be noted that these CIs are slightly too narrow because standard errors are slightly underestimated in this setting. Carriers of the 12e “T” allele have a 3-fold increase in risk (OR=3.27, 95% CI=[1.74, 6.16]). For the 18i “deletion” and the 21i “A” allele, the increase in risk is almost 2-fold (For 18i: OR=1.79, 95% CI=[1.21, 2.63]; for 21i: OR=1.73, 95% CI=[1.17,2.56]). Two copies of the 14i.1 “insertion” or 24e “A” or rs1805654 “G” allele might be protective, or viewed alternatively, being a carrier of the other allele could actually increase risk for AD.

TABLE 4
Odds Ratio from Conditional Logistic Regression

| | OR (95% CI) | | |
|---|---|---|---|
| | Carrier | Genotypic | |
| Poly. | Any 2^{a} | 12^{a} | 22^{a} |
| 6i | 1.61 (0.94, 2.75) | 1.68 (0.97, 2.90) | 1.43 (0.78, 2.61) |
| 12e | 3.48 (1.82, 6.67) | 3.38 (1.76, 6.74) | 12.21 (0.91, 164) |
| 14i.1 | 1.85 (1.08, 3.15) | 1.92 (1.11, 3.31) | 1.64 (0.89, 3.00) |
| 18i | 1.86 (1.24, 2.79) | 1.82 (1.21, 2.74) | 3.07 (0.98, 9.60) |
| 21i | 1.78 (1.19, 2.70) | 1.78 (1.18, 2.69) | 1.86 (0.56, 6.22) |
| 24e | 1.97 (1.16, 3.35) | 2.02 (1.18, 3.47) | 1.81 (0.99, 3.31) |
| rs1805654 | 1.81 (1.05, 3.15) | 1.85 (1.06, 3.23) | 1.72 (0.92, 3.21) |

^{a}Allele “2” is defined as the risk allele. The risk alleles are: 7i (C), 12e (T), 15i (insertion), 18i (deletion), 21i (T), 24e (A), rs1805654 (G).

Haplotype analyses were performed for groups of either five or six SNPs and/or mutations described in Table 1. The nominal p-value for each haplotype as calculated using TRANSMIT ver 2.5 is provided below in Table 5. In some cases, the population was made up entirely of affected individuals over the age of 65. In these cases, a separate p-value is included that represents the significance of the association of the examined SNP and/or mutation with the development of Late Onset AD (LOAD). Nominal p-values that are less than or equal to 0.05 indicate statistical significance.

TABLE 5
Association of Haplotypes with Alzheimer's Disease

| Haplotype | Nominal p-value |
|---|---|
| 6i, 12e, 14i.1, 18i, 20e | 0.07 |
| 6i, 12e, 14i.1, 18i, 21i | 0.0032 |
| 6i, 12e, 14i.1, 18i, 21i* | 0.060 |
| 12e, 14i.1, 18i, 21i, 24e | 0.0031 |
| 12e, 14i.1, 18i, 21i, 24e* | 0.033 |
| 14i.1, 18i, 20e, 21i, 24e | 0.040 |
| 18i, 20e, 21i, 24e, rs1805654 | 0.0016 |
| 6i, 12e, 14i.1, 18i, 21i, 24e | 0.00023 |
| 6i, 12e, 14i.1, 18i, 21i, 24e* | 0.014 |

*All individuals samples were over the age of 65.

The results demonstrate that haplotypes that include polymorphisms of the A2M gene provided herein associate with risk for AD. Furthermore, the results indicate that at least a few of the tested haplotypes can be associated with an increased risk of LOAD. The nucleotide identities of the haplotypes are the three most common combinations of genotypes as determined in the NIMH sample set using the TRANSMIT analysis program. Thus, in methods provided herein which include genotyping an individual for the polymorphisms included in the haplotypes, a step can be determining the identity of the nucleotide(s) to see if it is consistent with any of these three most common haplotypes.

The seven polymorphisms listed in Table 4, as well as the upstream polymorphism A2M_{—}1us (rs226380; see FIGURE), were grouped into haplotypes for further analysis using a haplotype analysis test within the FBAT package.

Combining all eight polymorphisms in one analysis revealed a trend for association in the total sample (P_{global,nominal}=0.08) and nominally significant association in the late-onset families (P_{global,nominal}=0.015). To explore which part of the gene contributes most to this overall association, a “sliding window” approach was employed, where each set of five consecutive polymorphisms was tested for association with AD. In these analyses, the strongest association signals were observed in the 3′ portion of the gene, i.e., in the last two adjacent windows: ([12e, 14i.1, 18i, 21i, 24e], P_{global,nominal}=0.046 [total] and 0.011 [late]; and [14i.1, 18i, 21i, 24e, rs1805654], P_{global,nominal}=0.028 [total] and 0.0036 [late]). These windows also contain, respectively, three (12e, 18i and 21i) and two (18i and 21i) of the individually most significantly associated polymorphisms, and the results for specific haplotype alleles are consistent with this (Table 6). Association was also observed in the late stratum in the haplotype [1us, 6i, 12e, 14i.1, 18i] (P_{global,nominal}=0.018).

Table 6 shows the alleles in significantly associated haplotypes and haplotype statistics.

TABLE 6

| A2M Polymorphisms^{a} | | | | | | | | HaplotypeStatistics | | |
|---|---|---|---|---|---|---|---|---|---|---|
| 1us | 6i | 12e | 14i.1 | 18i | 21i | 24e | rs1805654 | Strata | Freq. | P-value |

| | | | | | | | | | | | |
|---|---|---|---|---|---|---|---|---|---|---|---|
| G | C | C | ins | del | (R) | | | | Total | 0.12 | 0.19 |
| | | | | | | | | | Late | 0.12 | 0.031 |
| G | C | T | ins | del | (R) | | | | Total | 0.04 | 0.062 |
| | | | | | | | | | Late | 0.046 | 0.037 |
| T | C | T | ins | ins | (R) | | | | Total | 0.007 | 0.043 |
| | | | | | | | | | Late | 0.008 | 0.055 |
| | C | T | ins | ins | A | (R) | | | Total | 0.009 | 0.035 |
| | | | | | | | | | Late | 0.010 | 0.045 |
| | | T | ins | ins | A | A | (R) | | Total | 0.007 | 0.050 |
| | | | | | | | | | Late | 0.009 | 0.070 |
| | | | del | ins | A | G | A | (P) | Total | 0.30 | 0.12 |
| | | | | | | | | | Late | 0.29 | 0.039 |
| | | | ins | del | T | A | G | (R) | Total | 0.15 | 0.036 |
| | | | | | | | | | Late | 0.15 | 0.010 |

^{a}Specific bases for each polymorphism are given. Ins = Insertion, Del = deletion. “(R)” adjacent to a haplotype indicates that it appears to confer risk, i.e., observed transmissions exceed expected transmissions; “(P)” indicates protective, i.e., observed transmissions are less than expected.

It will be appreciated that other haplotypes which include one or more of the SNPs and/or mutations described in Table 1 in combination with SNPs and/or mutations that are described in Table 2 are likely to be implicated with an increased risk of AD.

### Example 3

### Screening Potential Therapeutics by Analyzing Clearance of Aβ by Polymorphic A2M

The activation of polymorphic and/or mutant A2M (A2M) by Aβ (amyloid β) can be detected by monitoring the LRP-mediated clearance of Aβ. HE 293 cells expressing LRP (LRP:TCRζ chimera) are seeded in 384 well microplates and grown in DMEM. HEK 293 cells not expressing LRP (IL-2:TCRζ chimeras) are used as negative controls. To each well is added 5, 20, 50 or 100 μg of test compound in DMEM. After an hour incubation at 37° C., unlabeled Aβ and polymorphic A2M from the media and extracts of the transfected cells are added. Unlabeled Aβ together with wildtype A2M (Sigma) are also tested as a positive control. After 3 days, the supernatant is removed from each well and Aβ levels are determined by ELISA.

To monitor the clearance of Aβ by ELISA, each well of the microplate is blocked with 200 μL of 1% BSA in Tris buffered saline pH 7.4 (TBS) for 1 hour. After the incubation, the supernatant is removed and each well is washed three times with 200 μL of TBS containing 0.1% Tween-20. 50 μL of a 1:3000 dilution of Aβ1-12 alkaline phosphatase conjugated monoclonal antibody 436 in TBS containing 1% BSA is added to each well and the microplate is incubated at room temperature for 1 hour. After the incubation, the supernatant is removed and each well is washed as described above. 50 μL of CDP-Star (Sapphire) luminescence substrate is added to each well and the plate is incubated in the dark for 5 minutes. The luminescence of each well is then quantitated using an ABI TR717 luminometer.

Compounds that enhance the binding of Aβto A2M promote the subsequent clearance of A2M/Aβ complexes from the medium via LRP. Accordingly, decreased luminescence indicates compounds that enhance the binding of Aβ to A2M.

### Example 4

### Screening Potential Therapeutics by Analyzing the Binding of Polymorphic A2M to Cells Expressing LRP

To screen for therapeutic compounds capable of modulating the binding of polymorphic A2M to LRP, A2M from the media and extracts of the transfected cells are labeled with ¹²⁵I then treated with 5, 20, 50 or 100 μg of test compound in Tris/HCl or sodium phosphate buffer at 37° C. for 2 hours. Untreated polymorphic A2M and wildtype A2M labeled with ¹²⁵I are used as controls. A2M can be labeled with ¹²⁵I using kit for radiolabeling proteins obtainable from Pierce according to the manufacturer's instructions.

HEK 293 cells expressing LRP (LRP:TCRζ chimera) and HEK 293 cells lacking LRP (IL-2:TCRζ chimeras) are seeded in 96 well microplates and grown for 18 hours in DMEM. Subsequent to growth, the cells are washed with 0.2 mL DMEM then pre-incubated for 30 minutes with 0.2 mL of assay medium comprising DMEM, 1.5% BSA, and 20 mM Hepes at pH 7.4. After the pre-incubation, the assay medium is removed and about 0.1 pmol of the ¹²⁵I-labeled A2M samples described above are added to duplicate wells in 0.1 mL of assay medium. To control for nonspecific background, wells to which no cells are added and wells to which no compounds are added are also included. Additional controls for binding specificity include wells to which 100-fold excess cold wildtype A2M or cold receptor associated protein (RAP) is added. Both RAP and cold wildtype A2M act inhibitors of labeled A2M binding.

After a 1 hour incubation at 4° C., the media layer is removed and the cells are washed twice with 1 mL of isotonic phosphate buffered saline (PBS). The cell layer is then solubilized using 0.5 mL of 10 N NaOH. The cell-bound ¹²⁵I-labeled A2M is quantified using a gamma counter.

### Example 5

### Screening Potential Therapeutics by Analyzing the Internalization and Degradation of Polymorphic A2M

To screen for therapeutic compounds capable of promoting the internalization and degradation of polymorphic A2M, A2M from the media and extracts of the transfected cells are labeled with ¹²⁵I then treated with 5, 20, 50 or 100 μg of test compound in Tris/HCl or sodium phosphate buffer at 37° C. for 2 hours. Untreated polymorphic A2M and wildtype A2M labeled with ¹²⁵I are used as controls. A2M can be labeled with an ¹²⁵I labeling kit for radiolabeling proteins obtainable from commercial suppliers, according to the manufacturer's instructions.

HEK 293 cells expressing LRP (LRP:TCRζ chimera) and HEK 293 cells lacking LRP (IL-2:TCRζ chimeras) are seeded in 48 well microplate and grown for 10 days in DMEM. Subsequent to growth, the cells are washed with 1 mL DMEM then pre-incubated for 30 minutes with 0.5 mL of assay medium comprising DMEM, 1.5% BSA, and 20 mM Hepes at pH 7.4. After the pre-incubation, the assay medium is removed and about 0.1 pmol of the ¹²⁵I-labeled A2M samples described above are added to duplicate wells in 0.4 mL of assay medium. To control for nonspecific background, wells to which no cells are added and wells to which no compounds are added are also included. Additional controls for binding specificity include wells to which 100-fold excess cold wildtype A2M or cold receptor associated protein (RAP) is added. Both RAP and cold wildtype A2M act as inhibitors of labeled A2M binding.

After a 2 hour incubation at 37° C., the media layer is removed and added to 50% trichloro acetic acid (TCA). The nondegraded material in the sample is precipitated by centrifugation at 14,000 g. The amount of degraded material present in each sample is determined by counting 0.3 mL using a gamma counter. The cell layer is washed twice with 1 mL of isotonic phosphate buffered saline (PBS). The cell layer is then solubilized using 0.3 mL of 10 N NaOH. This layer represents the cell-bound and internalized ¹²⁵I-labeled A2M is quantified using a gamma counter.

### Example 6

### Screening Potential Therapeutics by Analyzing Aβ Binding of Polymorphic A2M

To screen for therapeutic compounds capable of modulating the ability of polymorphic A2M to bind Aβ, A2M from the media and extracts of the transfected cells are treated with 5, 20, 50 or 100 μg of test compound in Tris/HCl or sodium phosphate buffer at 37° C. for 2 hours. Untreated A2M and untreated A2M that has been activated with methylamine are used as controls.

One method of detecting the binding of Aβ to A2M is through an assay based on gel-filtration chromatography. A second method is by immunoblot analysis. Both of these methods have been used successfully by other investigators to investigate Aβ binding to wild type and variant A2M(Narita, M., et al., J. Neurochem. 69:1904-1911 (1997); Du, Y., et al., J. Neurochem. 69:299-305 (1997)).

For the gel-filtration assay, Aβ1-42 is iodinated with ¹²⁵I, following the procedure of Narita et al. (Narita, M., et al., J. Neurochem. 69:1904-1911 (1997)). ¹²⁵I-Aβ (5 nmol) then is incubated separately with treated and untreated A2M samples as well as treated and untreated A2M samples that have been activated with methylamine according to the method described above. Activated A2M (Sigma) is also incubated with ¹²⁵I-Aβ as a positive control. A ten fold molar excess of Aβ is used and the samples are incubated in 25 mM Tris-HCl, 150 mM NaCl, pH 7.4 for two hours at 37° C. Controls containing only ¹²⁵I-Aβ are also incubated. The A2M/¹²⁵I-Aβ complex is then separated from unbound ¹²⁵I-Aβ using a Superose 6 gel-filtration column (0.7×20 cm) under the control of an FPLC (Pharmacia). 25 MM Tris-HCl, 150 mM NaCl, pH 7.4 are used to equilibrate the column and elute the samples. Using a flow rate of 0.05 ml/minute, 200 μL fractions are collected. Having standardized the column with molecular weight markers ranging from 1000 kD to 4 kD, A2M/¹²⁵I-Aβ fractions are counted in a γ counter to determine the elution profile of ¹²⁵I-Aβ. If treated samples of A2M bind ¹²⁵I-Aβ, ¹²⁵I-Aβ can be detected by gamma counter at two peaks, one corresponding to the molecular weight of the A2M/¹²⁵I-Aβ complex (about 724 kD depending on the polymorphism), and one corresponding to the molecular weight of unbound ¹²⁵I-Aβ (4.5 kD).

In some embodiments of the present invention, immunoblotting may be performed. For example, immunoblotting may be used to confirm the results of the gel-filtration analysis. In immunoblot experiments, unlabeled Aβ with A2M samples as described above. After incubation, the samples are electrophoresed on a 5% SDS-PAGE, under non-reducing conditions, and transferred to polyvinyl difluoride nitrocellulose membrane (Immobilon-P). Two membranes having parallel samples are then probed with polyclonal anti-A2M IgG and monoclonal anti-Aβ IgG. Immunoreactive proteins are visualized using ECL and peroxidase conjugated anti-rabbit IgG. Molecular mass markers are used to determine if the immunoreactive proteins from the anti-A2M and anti-Aβ blots for corresponding lanes display the same mobility. If the immunoreactive proteins display the same mobility then it will be concluded that Aβ binds the A2M sample.

### Example 7

### Screening Potential Therapeutics by Analyzing the Activation of Polymorphic A2M

To screen for therapeutic compounds capable of activating polymorphic A2M, unactivated tetrameric A2M from the media and extracts of the transfected cells is treated with 5, 20, 50 or 100 μg of test compound in Tris/HCl or sodium phosphate buffer at 37° C. for 2 hours. Untreated unactivated A2M, and untreated A2M activated with methylamine or trypsin are used as controls. For example, A2M positive controls can be activated by stirring A2M in a solution of 100 mM methylamine at room temperature in the dark for 30 minutes. The methylamine solution is then exchanged for Tris buffer using a desalting column according to the manufacturer's instructions. After the incubation with the test compounds, the activation of A2M can be determined by methods such as ELISA assay or gel mobility shift analysis.

An analysis of A2M activation by ELISA is as follows. Microtiter plates are incubated for 2 hours at 37° C. with 50 μl of LRP (10 μg)/well, and then rinsed with deionized water. The plates are then filled with blocking buffer and rinsed. 50 μl of treated A2M, untreated unactivated A2M, or untreated A2M activated with methylamine or trypsin is added to each well and incubated for 2 hours at room temperature. After rinsing, 50 μl anti-A2M IgG conjugated with MUP in blocking buffer is added to the wells and incubated for 2 hours at room temperature. After rinsing, MUP substrate is added to the wells, and incubated for 1 hour at room temperature. The amount of A2M bound is quantitated with a spectrofluorometer with a 365 nm excitation filter and 450 nm emission filter.

Alternatively, the activation of A2M can be monitored using a gel shift assay. Activation of A2M increases its electrophoretic mobility on a native polyacrylamide gel. To determine electrophoretic mobility, the A2M samples that were incubated with test compounds and A2M activated and unactivated controls are run on a native 3-8% polyacrylamide gel (Novex) at 75 V for a sufficient time to allow separation of activated and unactivated forms. The gel is then stained with Colloidal Blue using that procedure recommended by Novex. Activation of A2M by test compounds can be determined by comparing the electrophoretic mobility of activated and unactivated controls with the electrophoretic mobility of A2M incubated with test compounds.

### Example 8

### Screening Potential Therapeutics by Analyzing Multimer Formation of Polymorphic A2M

To screen for therapeutic compounds capable of modulating the ability of polymorphic A2M to form multimers, A2M from the media and extracts of the transfected cells is treated with 5, 20, 50 or 100 μg of test compound in Tris/HCl or sodium phosphate buffer at 37° C. for 2 hours. Untreated A2M and wildtype A2M are used as a control.

To assess the ability of the test compound to modulate tetramer formation, treated and untreated A2M samples are run on a native 3-8% polyacrylamide gel (Novex) under nonreducing conditions, at 75 V for a sufficient time to allow separation of the tetramer from other multimeric forms. 10 μL of prestained molecular weight markers (BioRad) are also run. The proteins are then transferred from the gel to a polyvinyl difluoride nitrocellulose membrane (Immobilon-P) by electroblotting at 100 V for 1 hour. The A2M samples are then detected with polyclonal A2M antibody (Sigma) using standard Western blotting techniques known to those of ordinary skill in the art. An A2M sample treated with a compound capable of inducing tetramer formation produces a band at 720 kD.

The ability of the test compound to modulate dimer formation can also be determined using the above method except treated and untreated A2M samples are run on a denaturing 3-8% polyacrylamide gel (Novex) under nonreducing conditions, at 75 V for a sufficient time to allow separation of the dimer from monomers. An A2M sample treated with a compound capable of inducing dimer formation produces a band at 360 kD. Monomeric A2M produces a band at 180 kD. In the disclosure below, several diagnostic embodiments of the invention are described.

Although the invention has been described with reference to embodiments and examples, it should be understood that various modifications can be made without departing from the spirit of the invention. Accordingly, the invention is limited only by the following claims.

All references cited herein are hereby expressly incorporated by reference in their entireties. Where reference is made to a uniform resource locator (URL) or other such identifier or address, it is understood that such identifiers can change and particular information on the internet can be added, removed, or supplemented, but equivalent information can be found by searching the internet. Reference thereto evidences the availability and public dissemination of such information.

What is claimed is:

1. A method for identifying a polymorphism or combination of polymorphisms associated with an A2M-mediated disease or disorder, comprising testing one or more polymorphisms in an A2M gene individually and/or in combinations for genetic association with an A2M-mediated disease or disorder, wherein the one or more polymorphisms is/are selected from the group consisting of 6i, 12i.1, 12i.2, 12e, 14e, 14i.1, 14i.2, 17i.1, 20e, 20i, 21i, 28i and 30e.

2. A method for identifying a polymorphism or combination of polymorphisms associated with a neurodegenerative disease or disorder, comprising testing one or more polymorphisms in an A2M gene individually and/or in combinations for genetic association with a neurodegenerative disease or disorder, wherein the one or more polymorphisms is/are selected from the group consisting of 6i, 12i.1, 12i.2, 12e, 14e, 14i.1, 14i.2, 17i.1, 20e, 20i, 21i, 28i and 30e.

3. The method of claim 1, wherein the nucleotide at 6i is C or A, the nucleotide at 12i.1 is C or G, the nucleotide at 12i.2 is A or T, the nucleotide at 12e is C or T, the nucleotide at 14e is T or C, the nucleotide at 14i.1 is no insertion or insertion of AAG, the nucleotide at 14i.2 is A or C, the nucleotide at 17i.1 is C or G, the nucleotide at 20e is C or T, the nucleotide at 20i is C or G, the nucleotide at 21i is T or A, the nucleotide at 28i is T or G and the nucleotide at 30e is T or C, or the complementary nucleotide thereof.

4. The method of claim 2, wherein the nucleotide at 6i is C or A, the nucleotide at 12i.1 is C or G, the nucleotide at 12i.2 is A or T, the nucleotide at 12e is C or T, the nucleotide at 14e is T or C, the nucleotide at 14i.1 is no insertion or insertion of AAG, the nucleotide at 14i.2 is A or C, the nucleotide at 17i.1 is C or G, the nucleotide at 20e is C or T, the nucleotide at 20i is C or G, the nucleotide at 21i is T or A, the nucleotide at 28i is T or G and the nucleotide at 30e is T or C, or the complementary nucleotide thereof.

5. The method of claim 2, wherein the disease is Alzheimer's disease.

6. A method of genotyping a cell comprising:
obtaining from an individual a biological sample containing an alpha-2-macroglobulin nucleic acid or portion thereof; and
determining the identity of one or more nucleotides in said alpha-2-macroglobulin nucleic acid or portion thereof wherein said one or more nucleotides are located at a position selected from the group consisting of 6i, 12i.1, 12i.2, 12e, 14e, 14i.1, 14i.2, 17i.1, 20e, 20i, 21i, 28i and 30e.

7. The method of claim 6, wherein said alpha-2-macroglobulin nucleic acid is genomic DNA.

8. The method of claim 6, wherein said alpha-2-macroglobulin nucleic acid is RNA.

9. The method of claim 6, comprising determining the identity of one or more nucleotides at a position selected from the group consisting of 6i, 12e, and 14i.1.

10. The method of claim 9, further comprising determining the identity of one or more nucleotides at a position selected from the group consisting of 18i and 1us.

11. The method of claim 10, comprising determining the identity of one or more nucleotides at each of positions 1us, 6i, 12e, 14i.1 and 18i.

12. The method of claim 6, comprising determining the identity of one or more nucleotides at a position selected from the group consisting of 6i, 12e, 14i.1 and 20e.

13. The method of claim 12, further comprising determining the identity of one or more nucleotides at position 18i.

14. The method of claim 6, comprising determining the identity of one or more nucleotides at a position selected from the group consisting of 6i, 12e, 14i.1 and 21i.

15. The method of claim 14, further comprising determining the identity of one or more nucleotides at a position selected from the group consisting of 18i and 24e.

16. The method of claim 15, comprising determining the identity of one or more nucleotides at each of positions 6i, 12e, 14i.1, 18i and 21i.

17. The method of claim 6, comprising determining the identity of one or more nucleotides at a position selected from the group consisting of 12e, 14i.1 and 21i.

18. The method of claim 17, further comprising determining the identity of one or more nucleotides at a position selected from the group consisting of 18i and 24e.

19. The method of claim 18, comprising determining the identity of one or more nucleotides at each of positions 12e, 14i.1, 18i, 21i and 24e.

20. The method of claim 6, comprising determining the identity of one or more nucleotides at a position selected from the group consisting of 14i.1, 20e and 21i.

21. The method of claim 20, further comprising determining the identity of one or more nucleotides at a position selected from the group consisting of 18i and 24e.

22. The method of claim 6, comprising determining the identity of one or more nucleotides at a position selected from the group consisting of 20e and 21i.

23. The method of claim 22, further comprising determining the identity of one or more nucleotides at a position selected from the group consisting of 18i, 24e and rs1805654.

24. The method of claim 6, comprising determining the identity of one or more nucleotides at a position selected from the group consisting of 14i.1 and 21i.

25. The method of claim 24, further comprising determining the identity of one or more nucleotides at a position selected from the group consisting of 18i, 24e and rs1805654.

26. The method of claim 25, comprising determining the identity of one or more nucleotides at each of positions 14i.1, 18i, 21i, 24e and rs1805654.

27. A method of genotyping a cell comprising:
obtaining from an individual a biological sample containing an alpha-2-macroglobulin polypeptide or portion thereof; and
determining the identity of one or more amino acids in said alpha-2-macroglobulin polypeptide or portion thereof wherein said one or more amino acids are located at a position selected from the group consisting of 14e, 20e and 30e.

28. A method of identifying a subject at risk for Alzheimer's Disease, said method comprising:
obtaining from said subject a biological sample containing an alpha-2-macroglobulin nucleic acid or portion thereof; and
determining the presence or absence of one or more polymorphisms or mutations in said alpha-2-macroglobulin nucleic acid or portion thereof wherein said one or more polymorphisms or mutations occur at a position selected from the group consisting of 6i, 12i.1, 12i.2, 12e, 14e, 14i.1, 14i.2, 17i.1, 20e, 20i, 21i, 28i and 30e.

29. The method of claim 28, wherein said alpha-2-macroglobulin nucleic acid is genomic DNA.

30. The method of claim 28, wherein said alpha-2-macroglobulin nucleic acid is RNA.

31. The method of claim 28, wherein the nucleotide at 6i is C or A, the nucleotide at 12i.1 is C or G, the nucleotide at 12i.2 is A or T, the nucleotide at 12e is C or T, the nucleotide at 14e is T or C, the nucleotide at 14i.1 is no insertion or insertion of AAG, the nucleotide at 14i.2 is A or C, the nucleotide at 17i.1 is C or G, the nucleotide at 20e is C or T, the nucleotide at 20i is C or G, the nucleotide at 21i is T or A, the nucleotide at 28i is T or G and the nucleotide at 30e is T or C, or the complementary nucleotide thereof.

32. The method of claim 28, comprising determining the presence or absence of one or more polymorphisms at a position selected from the group consisting of 6i, 12e, and 14i.1.

33. The method of claim 32, further comprising determining the presence or absence of one or more polymorphisms at a position selected from the group consisting of 18i and 1us.

34. The method of claim 33, comprising determining the presence or absence of one or more polymorphisms at each of positions 1us, 6i, 12e, 14i.1 and 18i.

35. The method of claim 34, wherein the nucleotide at position 1us is G, the nucleotide at position 6i is C, the nucleotide at position 12e is C, the nucleotide at position 14i.1 is insertion of AAG, the nucleotide at position 18i is a pentanucleotide deletion, or the complementary nucleotide thereof.

36. The method of claim 35, wherein the nucleotide at position 1us is G, the nucleotide at position 6i is C, the nucleotide at position 12e is T, the nucleotide at position 14i.1 is insertion of AAG, the nucleotide at position 18i is a pentanucleotide deletion, or the complementary nucleotide thereof.

37. The method of claim 35, wherein the nucleotide at position 1us is T, the nucleotide at position 6i is C, the nucleotide at position 12e is T, the nucleotide at position 14i.1 is insertion of AAG, the nucleotide at position 18i is no deletion, or the complementary nucleotide thereof.

38. The method of claim 28, comprising determining the presence or absence of one or more polymorphisms at a position selected from the group consisting of 6i, 12e, 14i.1 and 20e.

39. The method of claim 38, further comprising determining the presence or absence of one or more polymorphisms at position 18i.

40. The method of claim 28, comprising determining the presence or absence of one or more polymorphisms at a position selected from the group consisting of 6i, 12e, 14i.1 and 21i.

41. The method of claim 40, further comprising determining the presence or absence of one or more polymorphisms at a position selected from the group consisting of 18i and 24e.

42. The method of claim 41, comprising determining the presence or absence of one or more polymorphisms at each of positions 6i, 12e, 14i.1, 18i and 21i.

43. The method of claim 42, wherein the nucleotide at position 6i is C, the nucleotide at position 12e is T, the nucleotide at position 14i.1 is insertion of AAG, the nucleotide at position 18i is no deletion, and the nucleotide at position 21i is A, or the complementary nucleotide thereof.

44. The method of claim 28, comprising determining the presence or absence of one or more polymorphisms at a position selected from the group consisting of 12e, 14i.1 and 21i.

45. The method of claim 44, further comprising determining the presence or absence of one or more polymorphisms at a position selected from the group consisting of 18i and 24e.

46. The method of claim 45, comprising determining the presence or absence of one or more polymorphisms at each of positions 12e, 14i.1, 18i, 21i and 24e.

47. The method of claim 46, wherein the nucleotide at position 12e is T, the nucleotide at position 14i.1 is insertion of AAG, the nucleotide at position 18i is no deletion, the nucleotide at position 21i is A, and the nucleotide at position 24e is A, or the complementary nucleotide thereof.

48. The method of claim 28, comprising determining the presence or absence of one or more polymorphisms at a position selected from the group consisting of 14i.1, 20e and 21i.

49. The method of claim 48, further comprising determining the presence or absence of one or more polymorphisms at a position selected from the group consisting of 18i and 24e.

50. The method of claim 28, comprising determining the presence or absence of one or more polymorphisms at a position selected from the group consisting of 20e and 21i.

51. The method of claim 50, further comprising determining the presence or absence of one or more polymorphisms at a position selected from the group consisting of 18i, 24e and rs1805654.

52. The method of claim 28, comprising determining the presence or absence of one or more polymorphisms at a position selected from the group consisting of 14i.1 and 21i.

53. The method of claim 52, further comprising determining the presence or absence of one or more polymorphisms at a position selected from the group consisting of 18i, 24e and rs1805654.

54. The method of claim 53, comprising determining the identity of one or more nucleotides at each of positions 14i.1, 18i, 21i, 24e and rs1805654.

55. The method of claim 54, wherein the nucleotide at position 14i.1 is insertion of AAG, the nucleotide at position 18i is a pentanucleotide deletion, the nucleotide at position 21i is T, the nucleotide at position 24e is A, and the nucleotide at position rs1805654 is G, or the complementary nucleotide thereof.

56. The method of claim 28, comprising determining the presence or absence of one or more polymorphisms at a position selected from the group consisting of 12e, 14i.1, and 21i.

57. The method of claim 56, wherein the nucleotide at position 12e is T, or the complement thereof, the nucleotide at position 14i.1 is AAG insertion, or the complement thereof, and the nucleotide at position 21i is T.

58. A method of identifying a subject at risk for Alzheimer's Disease, said method comprising:
obtaining from said subject a biological sample containing an alpha-2-macroglobulin polypeptide or portion thereof; and
determining the presence or absence of one or more polymorphisms or mutations in said alpha-2-macroglobulin polypeptide or portion thereof wherein said one or more polymorphisms or mutations occur at a position selected from the group consisting of 14e, 20e and 30e.

59. A method of identifying a compound that modulates an alpha-2-macroglobulin activity comprising:
providing a plurality of cells that express the LRP receptor;
contacting said cells with a candidate compound;
contacting said cells with an alpha-2-macroglobulin polypeptide comprising at least one polymorphism or mutation having a position selected from the group consisting of 14e, 20e, and 30e; and
identifying a compound that modulates an alpha-2-macroglobulin activity.

60. The method of claim 59, wherein said alpha-2-macroglobulin activity is an interaction of said alpha-2-macroglobulin polypeptide with the LRP receptor.

61. The method of claim 59, wherein said alpha-2-macroglobulin activity is the degradation of said alpha-2-macroglobulin polypeptide.

62. The method of claim 59, wherein said alpha-2-macroglobulin activity is a protease inhibitor activity.

63. The method of claim 59, wherein said alpha-2-macroglobulin activity is the clearance of said alpha-2-macroglobulin polypeptide.

64. The method of claim 59, wherein said cells are contacted with an alpha-2-macroglobulin polypeptide in the presence of amyloid β.

65. The method of claim 64, wherein said alpha-2-macroglobulin activity is an interaction of amyloid b or said alpha-2-macroglobulin polypeptide with the LRP receptor.

66. The method of claim 65, wherein said alpha-2-macroglobulin mediates clearance of amyloid β.

67. A method of identifying a compound that modulates an alpha-2-macroglobulin activity comprising:
providing an alpha-2-macroglobulin polypeptide comprising at least one of the polymorphisms or mutations having a position selected from the group consisting of 14e, 20e, and 30e;
contacting said alpha-2-macroglobulin polypeptide with said compound;
contacting said alpha-2-macroglobulin polypeptide with methylamine; and
identifying a compound that modulates an alpha-2-macroglobulin activity by detecting a modulation in the activation of said alpha-2-macroglobulin polypeptide.

68. A method of identifying a compound that modulates an alpha-2-macroglobulin activity comprising:
providing an alpha-2-macroglobulin polypeptide comprising at least one of the polymorphisms or mutations having a position selected from the group consisting of 14e, 20e, and 30e;
contacting said alpha-2-macroglobulin polypeptide with said compound;
contacting said alpha-2-macroglobulin polypeptide with amyloid b; and
identifying a compound that modulates an alpha-2-macroglobulin activity by detecting a modulation in the formation of a complex of amyloid β and said alpha-2-macroglobulin polypeptide.

69. A method of making a pharmaceutical comprising:
identifying a compound by the method of claim 59 incorporating said compound into a pharmaceutical.

70. A purified or isolated nucleic acid comprising an alpha-2-macroglobulin sequence having a polymorphism or mutation at a position selected from the group consisting of 6i, 12i.1, 12i.2, 12e, 14e, 14i.1, 14i.2, 17i.1, 20e, 20i, 21i, 28i and 30e, wherein the nucleotide or nucleotide sequence at said position is other than an A2M-1.

71. The purified or isolated nucleic acid of claim 70, wherein said alpha-2-macroglobulin sequence is SEQ ID NO: 1 or a sequence complementary thereto.

72. The purified or isolated nucleic acid of claim 71, wherein the nucleotide or nucleotide sequence at said position is A2M-2.

73. The purified or isolated nucleic acid of claim 70, wherein said alpha-2-macroglobulin sequence is selected from the group consisting of SEQ ID NOS: 2-8 and said polymorphism or mutation is at a position selected from the group consisting of 14e, 20e and 30e.

74. The purified or isolated nucleic acid of claim 73, wherein the nucleotide or nucleotide sequence at said position is A2M-2.

75. The purified or isolated nucleic acid comprising a fragment of at least 16 consecutive nucleotides of SEQ ID NO: 1 having a polymorphism or mutation at a position selected from the group consisting of 6i, 12i.1, 12i.2, 12e, 14e, 14i.1, 14i.2, 17i.1, 20e, 20i, 21i, 28i and 30e, wherein the nucleotide or nucleotide at said position is other than an A2M-1 or a sequence complementary thereto.

76. The purified or isolated nucleic acid of claim 75, wherein the nucleotide or nucleotide sequence at said position is A2M-2.

77. A purified or isolated polypeptide comprising an amino acid sequence selected from the group consisting of SEQ ID NOS: 9-15 having a polymorphism or mutation at a position selected from the group consisting of 14e, 20e and 30e, wherein the amino acid at said position is other than A2M-1.

78. The purified or isolated polypeptide of claim 77, wherein the amino acid at said position is A2M-2.

79. A purified or isolated polypeptide comprising a fragment of an amino acid sequence selected from the group consisting of SEQ ID NOS: 9-15 having a polymorphism or mutation at a position selected from the group consisting of 14e, 20e and 30e, wherein the amino acid mutation at said position is other than A2M-1.

80. The purified or isolated polypeptide of claim 79, wherein the amino acid at said position is A2M-2.

81. A recombinant vector comprising the nucleic acid of claim 75.

82. A cultured cell comprising the nucleic acid of claim 75.

83. A cultured cell comprising the polypeptide of claim 79.

84. A cultured cell comprising the recombinant vector of claim 81.

85. An isolated or purified antibody that specifically binds to the polypeptide of any one of claim 79.

86. The antibody of claim 85, wherein said antibody is monoclonal.

87. A method of expressing an alpha-2-macroglobulin polypeptide comprising:
providing a construct comprising a promoter operably linked to an alpha-2-macroglobulin nucleic acid having a polymorphism or mutation at a position selected from the group consisting of 14e, 20e and 30e, wherein the nucleotide at said position is other than an A2M-1; and
expressing said alpha-2-macroglobulin from said construct.

88. The method of claim 87, wherein said nucleotide at said position is A2M2.


SEQUENCE LISTING (15 sequences)

SEQ ID NO: 1 - DNA, 88624 bp, Homo sapiens
tctttgcatccaatactccaacttctctgtggctgaccaaagaattggcacctatcttgccagtcaggtagttctgatgggtccagcacagactggctgcctgggggagaaagacagcattgatttgaagtggtgaacactataactcccctagctcatcacaaaacaagcagacaagaaccacagcttcctgcttctccctgagaagagaaaggattgttagaatctcccacaacctccaacaaggctgattgataggaaccttctcctatacaagactagtctgtgaagaatgggagaggtgccttcctttgtctaatgcagaggcaacaacacagagagtcaaagaaaatgaagaattaggcaaagatattcctttaaagaggaacaaaatacattctagaaattaacactaatgaaatggaattatgtgatttactttatggagaattcaaaataattctcataaagatgctcactgaagtcaaaagaacaatgtatgagcagtgagaatttcaacaaaaccacaaaaagtatcaaaaggtaccaagcagaaatcattgagctgaagaacacagtaacttaaaaattcactataagagttcaatagcaaactagataaagcagaagaaaagatcagttaatttgaacaccagtcattggaagtagttcagtcagaagagaaaaaaagacaaagaaataaaaagtgtagaaaacctaaggaacttatgtagcaccatcaaattgaccattatacaaattatgagagtcagaaaaggagaatagaaagagaaagaaagaaaaaacttattcatagaaataatgactaaaaccttctcaacctgaaaaaggaaatggaatccaggttcaaaaataactaagtaagatgaacccaatgaaatccacataaaaatacataatcattaaattatcaaaagtaaaagagaattttcaaagcaataagagaacagtgacttgtaagatagacaagatgcctgataagatgatcagctggtttttcagcagaaatttgcagtccagaaggcagtgaaattattcacagtggtaaaataatacaaacctgctaaccaagaatactatacctgggaaacctgtccatcaaaaatggaggagtaataaagactttctcagacaaacgaaagctgagggagttcatcacctctagatttgtcttaccagaaatgctaaagagagtttttcaactgaaagaaaaggacactaaacagcaacacaatatcaagagaaggtatgaaactgattggcaaaggcaaatataaagaaaaacacatgatactgtattactgtaatgctagtaagtcacttttacttccagttaaaagttaaaagagaaaagtattaaaaataacaaactaaaatatgttttaaaacataaaatagatatcaattttcacaaaaataaagtgtgtagggacagatgataaagggcaaagtttttgtatgtgattaaactgaagttgttatcagcttaaaacagactgctataactacaagatattttgtgtagactccaaggtaaccccaaaaagtctataaaagttacacaaaagacagagatttaaaaatcaaagtatattggtacacaaaaaaacaataaaacacaaggaagacaggaagagaggaaaagacagacaaaataattacaaggctaacataaaacaactaacatggcaaaaataaatcttcccctatcaacaattactttaaatgcaaattgagtaaacctcccaatgaaaacacatatagtgactgaaaagttaaaaaaacagacccaaatatattctatatacaaggtacttactttagatttaagaacacacataggctgaaagtgaagggatgaaaaagatatcccatgcaaatggtaatcacaagagagtaggtgacagggcaggagtatcatcatcttggacaagcactggcattttaaagttccccttaatcaaaaactgccccaaagggcattggcctaatggctaacgtcagcatgaccataaaccacaaatgacatctctgaccagaaacattccaacacgaaaataaaccctccccgaccagagatatgcctgccccaagataacctcccctccggccagagagatgtcagccccaagataactttccctctgaccagagacattccaaccccacaataaacttctcctccacacagaaacattccaagcctgtgataagctctctcaccctaaaacccttaaatactcttagtctgtaagagagagtggtcctgactaaaattggccagaagcccctctcaggtttattctccaaaataaacctgtctttgactgttgagccactaatcgtgtttctttcctctttctttaactcttacatttggtgccaaaacccaggacgggtgttgtgggtagaggctctcttgcaacccaggaagcagtgggcagtggcagctcatcccactggatcctgagagtctctggccaaccaccccatcttgcctcttacttcacttttcaagtgatttacatgagcaggacaactaacctgaagggaactgtgaggctcaggctggggctactctccagtgggctctcagagtcctgagacctgaccacttctgaccacccacagtgggtattttgctctctaacacttgtcccctcctcctccctcatcctcactttcctttctctctgtctctctctgtctctctctctctctctctcttcctcatgtggctctggttcaagaggccctttgccaattccaactggaacatccaatattggacactaatccagccaactggtaagatctgccttcccctgactttctcatggtacccgggaaagtcaggtatgccatcctgatcctcagaggaccagtgggactaggctagaagaaatcttggggacacccagtttcttctcagcttaattgttctctttagaaagaggattctgggtctctgtcttttgtctggggacacctacaacaaaaacagacaccctaggcttcttcttaccagtccacataggtgctcaacaatccaaaattcctatgtcctctccactgagctgtctccttcacagccttgccaaacttggcttatgggaagcataaagccaaagtgtttggctttttattgcaacgtggccgggccccagtgcaaattagataatgacagctaatggcccgaaaatggcacctttgatttttcaaattctcagggaccttgacaactttataaccaggaacagcaaatggcaggaggttcttgcctgaatattcaggctttcttctacctaagatcctgaccctccctgtgtcaagcttgcacccctcatgaaatccttcttaataaaaccctccccaggtttctccttcctctgaaattccttccttctaaaccccttcctctgaaaccccttttgaccctacagatgaatgccctctctatcctcatccccctggatccacttcttgcctgtccgaaccctcagccccaaactccactcttcctccttctccacctgttactcattcaaaaactgcttcaaccagtcaaaccacctctgcctttctcctactctgggaagtggctagggtcgaaggtattgcttgtgttcatgtccctttctccatgtctgatttgtcacagattaaacagcaccttggatctttctctgaaaatccctctcattatcacagggaattcctgcacataacccaatcctttaatttaacttggtatattatttacataattctaacctcaaccctcacccctgatgaaaaagagcactcagcttaactaaaatcgatgtccaagctatgagtatattcaaaggcctttatgtttttctcttcataaatcttgttttcctggaaaaggttttttcccagtcaactgaattacttttctccattctttcttgccactcttggtgcaggtattaaagaccctaaaatgacttctggtggcctgggattccttgggaaaacagaaaagttgccacaaatcccatttggggaaaaacctttgttttcgttgtggaacccctggaattagaggtaaataagtacctctcaaaatctgtctttgtctctcagctttacttgtttattaggccctggaaattattttcctagccctgttcttaaagggcctcacccaaaggccaataatccaattggaaaattagaaaaaaaatcttataactactggattttcttctggttgtctgtgtggctatatatgtgttaggtgtgcaatgtctatttaaaaagctctaattgactggcctaagaaaaataagtgcttaaatcaaatatttttagaggaaaagtaaaagctatgggacctttcagttcacgtgactttaatctttaaaacttactggcacagtaaaattagaaatgttttaagagttgccagcatacatttttgtttgcatttattaatcaagcaatttcatacttatctctgccaaatactattaggtgtcaaaatttggcatagagactacaaaactataactcagcccaaacagaataatctttgcttgtgtaattttttaataaatgaaacattaatattggtttaataaagatagcttcatcttgaactatttagtgaaataccctaacttctaattttgtggccttaggcagtctagtgcacagacatgaaggaagtttgctttgggaaaggactgttatcatctttgatattaaagaaaagagaatttatacaaaaaagaatcatatatggtaaattcctgtcctgaagtaaattaactagttgtttaaagagagggatgtttacaacaaagtcgaggcatgtcagagactgtccatgtaagtcatgaaaaaatttataaaagggaatttatgcaagaaatgttgtacaatttaaaagtgattaggactcctgaatgctttataaaatgccatataactcttagctgtacaacttgcctgctttgcagctaggtaagacctaggacacatggagttaaatgctggaataagtcggaccttatctgaacttctgtctgggtcctaggctctccacctagtacataattaaaatcccaaacttaccaacaaaagtaaaggttgctaaaagttaacagtgtaacatgtatttaagactattgaaaaaacagtttacatatacttttggtaaaaagattataaggaggcatgagaatgtggatttttacctagattaaaaggttaaagaattgttttaagttgaataaaataaaaatgaaggtttaagcaagttttggaaggttaattgtaaaggaaattctgtgtgtaaatatattggctaaagttgaagaagtatcatccagtttttctgtaaactgacattaaaataaaagcacagtgggtttggtttctcttaaagcactaacctgctctttaacaaaaattataaagggttaaaaagggtctatagaaatcttaccttatggtcaaacattaaaatcgggtaaatgtatctacaaggttctattaaaaattgagtttaacattagtagcacactaatataaaggtttagcttatttggtataaaatcatacaggaagcattgtcaaatataaaatggtgtttggctttctttgggctatatttgcatacatatgttattggtatgtgttccaaagttataagagactcctatatttctgatatatcttagtgtacgttatcagtaataattataattgttatgttaaaatattgcatgccacaaaggtaacagatattcttgtcaattgtaactttatggctactataaaactttttgtcatccataaacaattgttgtcttgtttttggtcccctagagactgaagtaatcttttttactttttgagtatatttaagttatggcaatatagttatttccatcagtgcaataagaatctgttttattttgtaacagaacatgatttgaaaaactggttattttaccaaggctttgactggagaggtgtgctgtcctttaaggaatcaaacttgacttatggagccaataaaactctcgggaaactggcctcatattttatgtgcacagtccctgtacagggtttctgacctgtggtaagtaaagaatgtcactttctgacaggccagtacccccaagttatcttggaacctcaggaggagaggaattcacccaactcataggtatttaatggtacaattccatgactgggctcagctttaaaaggccttatctcagattccttctatggaacaaaattccatcaatgccagtttaaaaggcctaggtaacaaataattattcttgctgcactgtatgcaaataattaaaccaagtataataatgcaaaccattcctaccatgatttattttttaataacggttactggcagaaaataacacgtggccctttccaaacatgtgcctctgcctctcattaggtaaggaatgttgcttctatctcaaccaattgggccgagtaagaaacactgctaaataacttaaagaaagggccaaagagctaagggaattccaaaacaaccaaatggattcttgatttgggaaaaaaaccatagcatgggtcatcccattcctgggcccccccctactactatgcctaggactaatgttcttaccctgcctaattaatcttttccagagatttttaactgacaggatcatggccatttcacagacaactacccaaaaactgccccaaagggcatcagcctaatggctaatgtcagcatgaccataaaccacaaatgacatctctgaccagaaacattccaacacgaaaataaacccctccccagccagagacatgcccatcccaagataacctcctctccggccagagagatgcagccccaagataacctcccctctgaccagagacattccaaccccacaataaacttctcctccacacagaaacattccaagcctgtgataagctctcttaccctaaaacccttaaatactcttagtctgtaagagagagtggtcctgaccaaaaattggtcagaatcccctctcaggtttattctccaaaataaacctgtctttcactgttgagccatttttcatgtttctttcctctttctttaactcttacaggaggagtggctacacttatatcagataaaatagactgagttaaaaactgttacaagagaccaaaagggaaattttataatgataaaagggtcaattcaacaggaatatataacaattacaattatatatgcatccaacagtagaacacctaaatatataaagcaaacattggaaaaacagaaaagagaaatagggagcaataaaataatagtaggaaacttcaatactatttgcaataatggatacatcattcagacagaaatctgtatccataagaaaacagcagacttgaataacacaatagaccaaatggaccaaacagacatatgcagaatattccaccaaacagcagctgaatacatactcttctcatgtgcactcagatccttccccaggataagtcacatattaagtcacaaaataagttataacaaatttagaaagattgaaatcacaccaagtgtctttctggccacaacaaaattaaactataaatcaataacagaaggaagactggaaaaataaaaaatacatagatattaaacaacagactcttgaaaagtcattgagtgaaagaaggaatcaagaaggaatttaaagtgccttaatacaaatgaaaacaaaaataaaaaacactaaaattatggtattcagcaaaagcactattaagaaggaggtttactgtgataaatacctacattaacaaagaacaaagatctaaaataatcaacccaaatttatacttcaagaggctagaaaaagaacaaactaagctcaaaattagcagagaaagaaataacaaaaattagagaagaaataaataaaacaagaaaagaaaaaaatcaaagaggctcagtgggttttttgaaaaaaataattaacacacccttatttagactaaaaaaaaagaaatgaaagaagatacattataactgctcttttagaaataaaaatgatcataagtgaacaaatatgtcaactaatcgaataacctagaagaattgtgtaaattcccaaatacacaaaacatgtcaaaagtaaaaatcaagaaagtttgaacagacctatcattagtatggagatttaatgaataacaaaaatccttctaacaaataaaaacccaggatcagatagcttcacaggtagattctaatacacatttttttaaaaaagtgccaatcagtctcaaatgcttccaaaaagtgagagaacacttccaaactcattttataaggccagtagcacactgctaccaaagccagacaaggacactacaagaaaaaaaaaatgacaggccaatatctctgatgaatatagatgcaaaaaatattttaaaaatattaggaaatagaatccaacagcacattaaagggatcatacatcatgaccaagtagtatttattcccaggatgcaaggatggttcagtatgtataaatttgtaccacattcacagaataaagggaaaaaaaatcacataattatataaatacaagcagagaaagcatttgacaaaattcaacattctttcatgttaaaaactctcaagaaactataaataggagagtatctcaacataatacaggctatatatgaaaggcccatagataatatcagactcaacggtgaaaagttgaaagcttttcctttaagaacaggagcaaggcaatgatgcccactcttgccacttttattcaatatagaaccaaagccctagccagaacagttaggtaagaaaaataaattaaagcctaccaaatcagaaaggaagagataaactttttcctgtttgctgatgccattatattatgtattaaaaatcccaaaggctccattttaaaaaactgttaaaactaatacacaaatacagtaagattgcaagctacaaaatcaacttacaaaaatcagttgcatttctatacactagcaataaactctgaaaaggaaattaagacaacaatcccatttacaatagcacaaaaaagaattaaatacttaaggaaaaacttttccaaggaagtgaaagacctgtgtctggaaacaaaaacattgatgaaagaaattaagacacaattaaataaaaatatataccatgttcattgattggaagatttactattgtcaaaataaccataatatcaaaagcaatctatagattcaatgcaacccctgtcaaaatcacattggtattgtttaaaaaaatagaaaaggaaatcctaaaatttatagggaatgagaaaacaccacaaataacaaaatcaatcttgagaaagaagaagaaagctggaggactcacacttcctaatttcaaaatttagtacaaaaccacagtaatcaaaacagtatggtgctggcataaagacagataaacatcaatgacagaatagagatctcaggaataaatgcacccataaaaggtcaactggtctttgacaagggtaccaagaatacactagggggaatggatagtcccttcaacaaatggtgtggagaaaactgtatatccataagcaaaacaataaaatttgatgtttatcttacaccatacacaagaattaactcaaagtggattaaagacataaaagtaaggcctgaaactgtaaaactgatataagaaaaaataaaagacatgctttatgatcttggtcttggcaatgatttcttggatatgacaccaaaatcacagacaacgaaaacaaaaacaaataagttgaactatgtcaaactggaaagctcttgcaaagcaaaggaataatcaacaaagtgaaaagacaacatatggaatggtagaaaatatttgcaaaccatgtgtctgacaaggggttgctatccaaaacatataagcaactcctacaactcaactcaacagcaaaaaaactaataacatgactttaaaatgggcaaggatctgagtagacatctttcaaaagaaaacatacaaatggccaataggtatatgaaaaaatgctcaatgccactaatcagggaaatgcaaatcaaaaccacaatgagatatcgcttcacacatgtcaggatgcctattatcaaataaaagacaacaagtggttggcaaagatgtggagaaaactggaatctttgtatactgttggtgttaatgcaaaatggtgcaactgctatggaaacatggtgtatatattcatctatatttgtatatatgtatatgcatattatatatacatgcatatatatatgcacacatatatgcacacacacatgcacaatggaatattgccttttttaaatgccaagataagaaacaatttattacagaagaaaaatttctcatccaaaatatagaaatcaatacaactttgccacaatcaatatacacgaactgtacaaatgtatacccattcataatttaccaaataaaagatgattaacaaagttcacaaaatagatgaaaatacttttacccaggaaagttacaaaccagacctccaatttctaaaatagaagtttactcagtcttagaaaactacaagctagcaaatgtacgtagagctggctggtgccaacaccacagttgaaacagtctttctaagggtctttttaaaaacccgttgccatggcagattctggtcacttgctactttcaaggtcaaaaacacaatacaaagtctgaccattttcccaggtcatgtttgctagcttgtctttatgtacatttagaaatatttgctaggtaaaagtcttgtcgtaaaatttccagtactactatgtttaaaacgttgagctcccctattgagctgccaaaaaggtaaacaataattttcaagtgtgatagttcaaattcctctgcgagatctactacagagaaaggttctttgacatacggattttctttaaaggaattgatgtaaaaatttaagtatgtctgggagaagctgaaatcactctaggacttcactccctagcaaataaagtgatcatttacttggactcataggctattaaattattgaaagatactgtacaaactatggcactgtcacttttaaaaaaatgtttaccactctatcttgtgccggatcttcacagctgtgacatggtttaaattccataatccatccccaataggagcccacccaaagccaaaatcaaatttatccatgcactataagatgatccatcttaacctgatacagtcatcatactgtagtttttggaagggctggttctgcccaagagaaattcgtccttacagtttattcagctgtctaccatttgtatgtcggtgctgttttgagtgctaccccctgctggtggggctttcatacagcacacagatggagccatcttctccaattctgcaggacagacatctcataggttgaggtgagcgtgagtccaacccagagtgtgagttcacttgggaaaagcttgaacagctcctgactgctcggtccaatccactgtgctgcctgtccaggggatccatttcatggttgatgcgaatacaaagataacttgatcttttgtatggcttttctgggaatcagtgatgtttataatgttctgtcagcagttcctgcaggctgtggctgaaggtctgcagctgttgcccgttcgtgagccctttgctgtggagaaacttggagacgaaggacatggaggcggccatctcgcctaccctggtggtggctctggggtagaagggatgcaaggaggcagaggtggggcggcccgggaacgccggggctcggctgcgctgcctcagcagctgagcagcccccagggcttcctcacagagcagagggctgacaggaagaaagggtgtgaggggcagacagctacttttttctttcattagagtaaaaaagttattttctagacaggaagaggcagaagagaggaagagaggagcgatggcggtttggttacatggctaggacctccccagctgcctccacccctggctgcagagcctctgaagatcccaacagttgcctttccagcaccgaagggcgaacttctttaaaaagaagaaaattctgtatacgacaaaactaatgaatcttgaggacattatgctcagtgaaataagtcacagaaagacaaatactgcatgattccacttacatgatttatctaaaacagccaaattcatagaatcaaagagtaaaacagtggttatcaggggctaaactgaagagggaacggggagtttgtaatcaatggacataaaatttcagtgaagcaaggtaaataagctctggagatttgctctacaatgttgtacttatagtcgacaataatatattgtacacttacaatttgttaaaagggagatctcatgttaaatgttcttaccacaataaaataaaatttttaaaagaaaaaagacacctggtgtctttttcttgtagcaaccgaagtaaatagagatgtatgggtgtataggcattagtttgattttctcttacgaagaggtctggattatagcctccatcttgagaccggagtcaagcacttgaggtggggaatgatggagagattggaagtggcattcttctccacaacaccatgggttctgtagtcccttgtgcaattctgcaattctgccatgatagttattgagaccaagacaggcaaagtctaaccagggaaagaagagttctgtagacttccttccaattctaccaaggttgatattgagagcaggcttcttgccagcagcattaaacccaacaatctatacttcctaggacaatctctacctttgatttctgtaaaataacacttttctgtttttcttctgaggcccctcctccacagaccagtttctggatatcttcttctttatcagatctctaaatgttgagattcttctagatttagatctatgctgcttgttctttgcactctacattttcttcattcatttctacaacttttaatagtgtgtataggtagctcccagattgctattttcaccgtgattctcttgcctcatacttttcccctctttggtgatgatgtgtcagcacattggccctctctctaatcttgaatactttatttctgtctggggagtttagcacatgcttccctctgcctataatgctgtataacttgccctctccctttttgcaaggataaattcccagcttaaatgccatatcataagagaggtcttgactgaatgcacaatctatgttggatctccctggatgttcagggaagaaaagaaacatttttttctttcacagcatttatcatcatatggaattaccttatttaatttatttgtttatatactttgatctgtattcttccacaggaatgtataatccccaatggcaggaacatgtggtttttgttaaccacagaacagtatctgatacaaaatatgcattaaaattcttgtgaactgactaaataatatccatctctccacttgattgctttacttgccacacatccccagtacctaattcaagagcttggctcattgtcagcctttaaatattgactgaacccaattaatcacttttttctttaacttctgaactctgtgcccagctccctacttgaatagcttcactggatatctcaaaagaacctcaaattcaatatgtctactttccctatctgttattagcaacaaacccagcacttgccctagtaggaaatctacaatcatcctttacttatctctaaaatgaaaaaaaaacccacacctttttcataaagttttgagttcactaagtgaaataagcaaggtgcctatcacaatgcctggcatacagtatctataaatattagtttagctataccagttttcattcattcaaagaggcacatcttttcacatctctgaaatcaagatgtgttttgtgatcagtagtgtttcataattggatatacatctaatcattaatcaggtggaaattttttctttaaatactttattagaaaattgtgtcctttacctcaatggtgcttcagatgtgataatttgcagtaacacaacttctctctccaatgtgcccatatatattaaagtagttattaggtcacatcacgtacatttgaaatccatattttttctcactcttcccactgttgtaatcttactttgggccctcattttcttccaactgaacactgaattagccccctgaatggtcttcctgctgactatattgcccttctccaatgtgtgtgtcatattcatgccagagtaatcatgtttctctcatgcctaactcacttcattatctacacaactgcctgcagaatggaaaccagattctttcacatggcacacagggctctgactctctctctctttagcctaatccctaagccacagtcaagttgacctcctttctgtacttgcagatgacttaacactgtatgttgtttcacaacccatgtcgtttatcacactgacctatcttgaaaatattttgtatcagctggaatccctctggcaataaataacagaaaagcaactagcagctgcttaaacaaataggaaagtccataggttggtgttccaaggttgatagaattgcccaataatgctgatgaggaccaaggcccattttatctttcagacttccttaacatgcagaccttcatccccctgcttgttactttgctgtctcaagatggtcattgaaatattaaatatcacatctattttccattaagtaagaacaaggaaggacattcaggggtatgcccactacaactcttctttttaagaaagcaaatgtttcccataagccctacccagcagttttctacttatatctcattgcccaaacagcaccatagtcactctggtttgaagatgcctagagagagggatgttgtgggtagtttagccaatgagcagtgtctgccacagtctgcttggaaaatatcttctcattttttgtgagattcaactcaagagttatatcttagaaatttagaaatgtgtatatatatgtatataaactatatatgtatataaattatattgatatttatataagcatacataaaacacaaatatataaacgtatatatagtatgtaccctttcatcattaactgaatatttatcaaacacctgtacacagtgcgtgctaggtactgttctaggcactgggaatacagcaatgaacaacatcttatcctttgtggaatttacatcctagtgtttaccctttgatctaactttttcacctctagtaacttaccttacagactatacctcaaaaacaaggatgctcattgaagtgataactagaataacaaaaatctgattaaatatcactatgggattgattataatatacataaaattataaaatgtaattattcaaattacctcttaatagagatatactaacatggaaagctgtccccaacatatattttaatttaaataatggaagaccaaaatgtatagtaaactctcacttttaaaaagaaaatccatccccaaattcatacggaatctcgaggaatctgaaatagccaaaacaactttggaagaagaataaaggtattagactcacacttcctgatttcaaagcatatacaaagacacaatgatcaaagcagtgtggcactggcataaagacagacaaataaaccatcgtaatagaataaagagccgagaaataagcctgtgtgtatatggtcaaatgatcttcaacaagggtgccaagaccactcattgagaaaatgatagtctcctcaacaaacggtgttgggaacaatggaaatgaaagttaaacctttattttacactatctacaaaatttaacttataatatattaaagacataagcataagacctaaaactatgaaatttctagaagaaaacagacaggataatctcatgacatggggtttgtcaatgacttcttagatatgacaccaaaagcacaggtaacaaaagcaaaaaaagataaatgggactacatcaaacttgaaaacatttgtgcatcaaaggacacaatcaaaagagtgaagggcaacatacaaaatgggaaaaaaaaatttacaaagaatatatttagaagttattatccacaatatataaagaactcccacaactaaacaatgaaaaaaaaatcaaataactagattttaaagtgtgcaaaatatttgaatagatatttctctaaagaatatatacaaatggctaataaacattgaaaaggtgctcaacattactaatcaccagagaaatgcaaatcaaaatcacaatgacatactacttcacatctgtgaggatggctgctataaaaaaaaaacagaaagtaacaagtgttggcaaggatggggacaaattgaaacccttgtgcgcagttggtgggattgtaaaatggtttaactgctatggaaaacaccgtcaagtttcctcaaaaaattcaaatagaactaccatatgatccaggaattttacttatgggtgtatatccaaaagaattgaaaacatggtcttgaagagatatttatataccacagtcatagcactagtcccaataaccaagaagtaaaagcgagccaaatgtccatcaatagaagaatgaatacatacaatcaaatattattcagccttaaaaagaaatgaaattctgatacatgctgcaacatggatgaactttgaagaaattttgctaagtaaaataagctagtcacaaaatgacaaatactgtattatttcacctatatgaagtatccaagccaattcataaaaacagaaagaagaacgctggttaccagggacagagaagaggagaaaggggaagtgtttaataggttgtttagtagttatagagtttcagatttgcaagatataaaagctcggaaaatctgtctcacaacaatgtgtatatactttacactactaaactgtacatttaaaaatggttcaccaggcgcggtggctcacgcctgtaatcccagcattttgggaggccgacgcgggtggatcacaaggtcaggagatctagaccatcctggctaacacggtgaaatcccgtctctactaaaaatacaaaaaaagtagccgggcgtggtggcgggcgcctgtagtcccagctactcctgaacccgggaggcggagtttgcagtgagcagaacgcgccactgcactctagcctgggcgacagagtgaaactccgtctcgggaaaaaaaaatgggtaaaatgggaaaaaggaaaacccatacctgcatcttcatatatattattatatacaataatactgaaaatactttcagtttatgaatttggtgggtagctcacacattactttatattttcttcatacttatatattttataataagtatacattactttttatagtcagagaaagattagaaaaaatttaaaaaattacaaggtactttcttctctatctctctcctcaaatagtaatgaccatttatttgtacttatgtagtcaccaacagaatttatatgtattgaatgaaataataaatgtatcatcaactagtgataatgaatgccttcaataaaaatttatttatatttagcaccaaattaagaaattaatttaatatagttcttcatttaatgaataccgattgttattaagtgcctaggatatgcccgatgtgcctgtgtaggttttgagtaataaacatggttatttcttgaaactatcttttgatgaagaaagagaaaaagagaaagagagaaagaaagaggttatggctagctggtaacattcaaaagcaagccacttcatgaaaccccattacaagtcaagatagtatagtccaggtgtgtagtactctggtggtctggttttatcatagttagagcctaaatacatagcaaagagaacagatttcacatatttcaaaaacatgtaacagcgagtatgaaatacccgcagacccagtcaccccactccagcttctgccagagaaaactggtgtaagaaaaactggtttggaagttaattgcattcttccccccacccccttgggccataaaattttattagcaggtaaggggaagagccaggagtaagggtccctcccttcccatcccctacccaggatcctccctctgaaggagagcagaagcccaggagcctgccactcagcggaccttgcaggtgggcaggttgctgtaaggatctttctggcgctcgagcaggatgcggtcggcagtgatctcctgctcaggcctcctctggcagtcagcacacacattgcccagctggacttcggcaccgctgctcagagcctggatccgcaccagctgggcctcaacgcaggcctccgttcctgcagtacggtcttccaatgcggttttcatgctgagctgcgactgcagctcaatctcaagaccccggagggtacgccaccggtccgcgactcggacttgtgcatctggagctgctccgtgtggccagcgacctcccagctcagatcctcagtctggctggtgaccaggcttcagcatcttgggcatctgctcagttatgaccgcatactggcttcacgtgttgcccgggatcttggcaagaccaatgcctggagcagaatccacctccacactgacctggccacccgcagcactgatttcctcgttgtggttcttctccaggtgatccagctcctcctgcaggccttggatgtgcgtcttcaggtcagccctggccggggtcagctcatcgggcgccctgcgcaggccttgatgtctgcctccaagctccttcgcagagactgctccatctcagacttggttcggaagtgatctgcagccaggcggcgggtgtcaatctgcaagacaatccgggagttctcaatggtgacaccaagaatcttgtcccgcaggtcttcaatggtcttgaagtagtggctgtaattgccggggccccggggcccagtcgcagatcttcaccttcagctcacccggcctcctccagagcccgcaccttgtccaggtaggaaccaagtggtcgtggatgttctgcgtggtgatcttggtgcccgtcagcagcctgtcggacctgcagaggacgctggcatcgccgctgccatagccccagaggaggatgagggcacaaagtgggtggaggacacagacaggctacggcttcggagccccagtggatgctgggctctaggaaggcgcccccgactccaaagtgcacggagccgctgcccagcccccaaaggactaagtggccaatgactggcgaaagctgcaggaagttagggtgaggcagagaacggttcaaggagctgcaatcggcaggaggaatcggaaattttaattgcattattaccaattataagaatttttgtgctatgagtgggttttgtgctatgaggggacccaaaaaagggctgataaaacaccaaaaccactggtgaggagaacttaagagggaaccaggcttcaagctcctaaactcaccatggactgccacagaatttaaagcacagtccatattcctcagaaagctaaccaagcccaccactgggtgtcctaccaaaggccgttccctggcattattctagacagaacaacccagacggcaaaaatgtactgctgtggagagatgaatcaattcctaggaggactggacagacattctcaaaagtttgttggaaagatagcagagaagagaagaatgaggtgctttaataaagggataggaagaaggtgagaacaaggaactgagattaggagctgagtgtgtccatgacagaccgcctagaaagaatgtagtgtctagagcttttactcatagaccgcgggacaaggtatatttaccatatagctctgtattttccctcttgtgttataatgttttctgtaattaattactctaacccttttagcaaagtctaatacacttcagcattgtctcgtgatgatatacagactaaatggacaggtgagtaaaagaaaaaattaaggcgtcctcactggtgggagcagaatcccaggcagagaactatgcacagaagtttcaggcagtgatcagctgaaagtcagattgcagtccattgtattccatggtggagacaagaacagggagggagcacccatacttaaaagaaacttctaggcttggagcagtatggtattttatacacatcacctctgttagtcaagctttggatgggggctagatagcagcccactgaaagacctggagtccctgtattcttaattgctatgtcggaaagttccatatttagaaatcagatgggcagcaggtaagttaggcattcttctgaaaaaaattaagcctctaccaggactctcgcttggtttataaaatgagccacagaagaaacagcaaactcaagttttcctctcaaaaagtcctgtgtcacttgaaaagttgcttcatacacattgtgtcctaaatatgaaatctaaagttgttactgttttgaaataaagtttcaagagaatatattttatataacattcaaatgagttatagtaaaaatgctaggtcttaagagattcaccggatgaaataacattctccacttcagaaaatgaaactagtacaatattaaatactgtgattaataataatctctaatatttctacatacttttgctcctaacagttgagttatatgctcaccctgtatgattgtggtatattctgttatgccagttgcattattgtaattataaatttgaggaataaagtacacgaacttataaaatataaacatggccaggcacagtggctcacgcctgtaatcccagcactttgggaggccgaggtgggtggatcacaagctcaggagtttgagaccagcctgcccaatatggtgaaaccccgtctctactaaaaatacaaaaattagccaggcatggtggtgagcacctgttgtcccagctacttgggaggctgaggcaggagaatcatttgaacccgggaggtggaggttgcagtgagccaagattgtgccactgcactccagcctgggcgttagagcaagattccatctcaaaaaacaaacaaacaaacaaacaaaaaatatacatacacgcactattttaaaactcagtttttatctaaaacctagattaaaagtctttggaaagagtccatggagaggaatacgttaaaaatgccattgaagccattctgcaatgtataatatttcagaatgacatgttgcacacaatgtctacataatttttcgtcaattgaaaataatgtgatcaattggattaaaaatatatcattaactcatgaatttaaatatatttaatgagcttaagttgattgtattttctgtatttcttaaactcaaggtattacctagtgctttgaggtgtcgattttctggccagaaacttctgcggctggcgacaagctcttgtcctgcatccaggaagaatgacaggaagaatgaggtacacgaacaagtgaagggtgcacaataagaatgaggtacccagacaagtgaaggacaagatgaagatgagctttactaaattttagaacagctcagaggagacccacagtgggtagctcctctccgtaggcaggtcttcccaacatctactgctctcagcagagaggaggccctggagcgggtgctccttgctcctctctgcactgtagtcccgaagtctctgcaggtctctgaagctctcagcagagagggtagttcctctgtgcagctggttgtcccatcgtctcctgctattagcagagagggcagcttctctctgcaactggtcttcctgtccctccatcctctctcttgctctgcctgagcccgaggcttttatggggtggaggaaatgcctgctgattgatccatgggcagccataggcaggccaaaggaggcaccacaagtcctagggactgactgccgggatcccaaccttcaggtcctccctggcctgaaggtggggccttacaggggacctgcccccttctgcccaggagtctgcctgcctcccgctgccattcatggcccctgggctcagccccaagattggagcaggctctgggagaggagaaaggccaggcaatgggagcagacacccctgagcctgcagggtcgagagaaggggagggtcttcccggctctcgagggtgcaggctgcagagatgcctggacctgcacctgtgagggtgccgcagctgcaccagggaatctcccgcgcagccaactgggaatgggcaggtctcccgcttgtcccgggctctctctggctcagtggagcagtaggcccaggtctgcagccactggtcggggtgctgcagccgcacggggagtgtagatcttgcctgctcccagccccttccaagagcacagggaggctcagatccacagccgcagtttgggcggggctcctacctgctccatagagcaggaggccgcggtctgcagcctcggtttgggcagctgcagcggcaccggggagctctggccccaactcagaagggacggagctcccaccggctccactgcagccaacattatggcagcagcggctaccatcaatggtatcccatctttggcaagtggaaacatcttaatgaatttcccgtttgccccgagaatcctcttgtctctgatcctaaggtaacatcacatacatgtctgttacactaggattagagacaagttctgtttagaaataactccaagaacagcttttatattttattttcacattgaaaatcagtcagatttgctccagcctcaaagaatgtgtttactaaaattaaatgaatgctggcagggagctgcactttttttttctaaataggaaatgggttaagggcggcagctgagtcctttcgacataaccctaatagtctttggcagcatcctttctgtttggtaggataagatattctaggctcatcttgcattttccgcgaaaaaaaaaaagccattgaattaggcatagaggaaattactataaagtttaagggataatgtaaaggatctaggaaaatactctaattgcttcacaaacaaggaagaaactgaaactagaaatgtagtcaattagtattgctgatgtgctttatgcaagaaagatagtatatttgttctctcttatctgcagcttcactttctgtggtttcagttacccttggtcatccagggtccaaaaatattaaatggaaaattccagaaataaacgattcataaattttcaattgtgtgccattctgagtggtgtgataaaatcttgacacccgggatatgaatcattcctttctccagcgtatccatgctgtatatgctgctgttattccccctaacccccaccccagtcacttagtagctatctcaatactgcagggcttatgttcaaataatccttattttacttaataatggtcccaaagctcaagagtagtgatgttggcatattgttataattgttcttctattatcagttttgttaatctcttactaagcctaatttataaattaaacttcatcctaagtatgtatgtataggaaaaaacatagtatatatagggttaggtactatctgtggtttcaggtgtccactggggatcttggaacatatgcccctcggataaggggaactactatatacaagattaaataaggtcacttaaaattcatttataaaattatgtcctaaaatatttgtaccagtaacaagatatattcaaattattttatctaataaaaattaacatgttttattttctaataaaagttaaaatatgttagcagtctataccacttctttaattacatggaaaaaattacattatgatgtctaattaataaatagttatttataattactgccaaagtcaaaaaccagtttgatacttacatcttaaataccatgcctttttatacccttttctttataaataacttgtaatttaaccttcaaattatactgcattactttaaattaaataatgagagtacgtatgtataacttgctttgaaaataaatgaattatgaaagctacactatttaaaacttgagaactttcaataataccttatacaatatattccataattactttttaataaatttttataatattactgtaaattgtgggtttagggaccgacagttcaactattagcaaaataatcttttagcataacaaaaattggaacatctaaatctcttagaacttgtcttagcccatttatatagtaagtactacgtaattgctagctgtcattacctgttactgttgtagctattgttgttagcctcattatcatcagcatcatctgagtaattaagtaaagttggcaaacaactttatctttcctacttttaaaatttaaccccagtgtttgctcccaaactaagtagaactctctaaaatgaaagttctgacactggaaagttccacagcgaacatgagcactacagtgaacaaaggggctcctatggttaagggtcatgaaccaaccttgtgcaaacccacacacaaacacgcatacacttattttctgttcatcaaaggaaaagtaatcctgtaacatttcccccaccttgcttcctattccaaaataaactgcagatgaagattaaaatctaaacacaaatacttaatgtaggaaaaaatgcaggataatatttttacaatttcagtatgcatatgcagaaggatttcttaatcagaaaataaagatacagtataaacaaaaagaaagttaaacaaagataaattataagcaacagggttcctacaagtgacaatgatgctgaacatcaccagtaatcatgaacttgaaaaagaaaagacaaaatagatcatgctcccctccccatcagattggtaaaattttaaatgagtgctgggaaaatatatacacataagcattgctagtgagaaggtaagttggcattttagaaagttgatgagatagtatctataaatttgaaatgtatatacatagcaaattcacaatggggacagattcattatacaaaaatacttcggcatgccccaaagtataaatatgttaggacagccattgaattctattgtttgtagtaaattgttttagtccaaacactaattcctctgtagcaaacataggatctaataaaatggattatgtgtggaaatcagtcctctttagaaacctaaaggaccaagtgtatcctgattaaaaagataaaacgctttctttctttctttttgtttttgtttttttgtttgtttgtttcgagacagaggctcgctctgttgccaggctggagtgcagtggcgtgatctcggctcactgcaacctctgcctcccgggtttaagcgattctcgtgcatcagtctcccgtgcagctgggactacaggcgcacgccaccacacccagctaatttttgtagtttaagtagagacggggtttcaccatgttggccaggatggtctcaatctcttgacctcatgatccacctgcctcagtctcccaaagtgctttttgataattttgagaaatgatggaggcatattagaatgaaaacaacctgaggatgtgcttttatctttgtatattcaaatattttttctcattaaaaagcagaaagtccgggtatgatggttcatgcctgtaaccctaacactttgcggggccgagataggaagatcccgtgaggccaggactttgaggctagcctgagcaacatggtaggaccctgtctccataaaaagcttaagaaaaaaattagcggggcgtggtggagtgcacctgtagtcttagctatttgggaggctgagatgggaggatcagttgagcctaggagttcaaggctgcactgagctatgatctaaccactgtactccagcctgggcaacagagcaagaccctgtctctgaaaaaaaaaaatacacacacacacacacacacacacacacacacgttagtgggatagcacaaatgagaaaaactctgctctttgatcactgagtacatctctgtagatatatatttccttcactgcagattttgcccaagatacttcgtcaaagacaaagccagtacaccctctaatagggtgaatatggttatgccacctactgagcttgtttttgatactagttaatatgtaaccagatgaaattgtcattatcgtcactgtcaggactatgggaagcttaagtgttctcttttcaaggacaatgtgcgctaactgtacaattggtacaattaaataagttatattcagttcctgggaagcactatagcaatacaaggagaaaatttgattctatttatttttgttaaggcccacctacctcctaatcctaatttctctcatttcccaaatattccttgtttgttcttactgttatgtgttttcctgtattttgctcttctactttcttttccatggactatctttttcccttcctttttttcgctctacccctttacctcagctttctagcagtatttgctaaatacttcaaaactgtatagaactggttcaaattgtgtgctcccttttctgtcaagaacttgctactcaggtaacccaattggtgatttttcctggaaacactgatggatgctgttcctatagcgaaacccagaacagagatgaaatagatgtcatcctcagccattagcattcaaactataaaaattaatttacactggtatagtaaggatcagaatgtcaaagctgtgttacacctagcatcttgtatgaaactaccccattaaggtgagaccacagatattattgccccactattggcatgaaagctgaggctcagagcagttaactgagttacccaggaccacacagctaagttagaagtagggctcaggtgtcctggcaactaactggtccagttattttttctctcaagctcgttttccctctcctaaagaataggaggctctgtcgtggtgaaaggcgattttagtaatactttcctttttatctgtgattataatgaatgcggcatctctcccattaaggatcattcctccacccacattcttaatacatctgctgcatgcatccttcagagacctccctctgggatcatcccttctcactccaaaaagctcaacttctcccctgtcatttgtacctcccactcagcatttttagaagcaatatttcattcaaacttattcaagtttatttccacctaaagaaatattcctttcaccctggcatctccgtcaggtactgctctgttgtttttctccccttcagacaaactgccaaactggctctagttcctcacattccccatcaccctcagcaagcttctgccccacaccggcactgaaacagctgaatcccaatgtccttgtccttaaacccagcagaaaaaaaaaatcaatcaattatttgatttcacagcggcacttgacatgggtagccaggaatttatcaatgacaacctttacagatcatctttgtaatttatcatgaggcatcaaatgaatgctattaacattaatccctcctattttaagtcattaatccaagtaaatgctcacttatttctagcgtcttagaaaccatttaaattatgttacattatgaatcaatacattataaaattataccatcatttgtaataattttttaaaatgttgtgtgctattaacattgatgccttggtataaagtcatgatcattctggtctagtagcaatcttctattgactattctcttactaaagcggtcccttccgtgggactcagagacctcacactctcctgcctgtgtttcttcctctctaattggcccttcttgctccacttgggtgctcctgcccattgcctagacaagagcattccctgtaactctgtcttgggctctttttctcttttcatcaacatcttctacgtgggtattatcatccatttccatggcatcagcttgcccaataaactgataaatccatagtctctataagtacagcagatctcatcaagctagtggcattcagactgctttaactttaaccaaaaataagggattttgtacatgttcaataagcagttcccactgtgacactgtaatcacattttcacaattgtgacctaggacacttagagtaaaggatacagatgattgagacagaaatagtgacaaagaaaaataaggttaggatatagattttaatgctgtaacagacctcaaaatacaatggcttaactaagagaatgcatttctctgtcacataaaggtcccaactggcgtagacttttgatgactcaagggctcaggctgtgcctggtttgtggttctgccttccttaacacatggcttccatctgatgagctacagcagtacctatcactagtcagcatgtccacattccagcctgggcaaggaagaaaggggaagcgcagaactgtacccttccttttttaagtcatgaactgaaagttgcatgtatcacttccacttgccctccagtcaccagaacttagtcatatgccatacccagcttcaagggagtgggttaaaaacatagaagtcaactaggcagtctgcacccagcaaaggatcgggagttctattattaaagcagaattggagaagtggtaacaggaaacaaccaccagcctctgctgcatgtatatgaaacagatgtttcccaaatcactattctcacttattctgtctgatacactgtattttttattatattctctttcattttttaaaatcctggtcatgactcacagggcatgatgttacaacccacttagatgctaacaccataatctgaaaaatattacctatattatgtctaatattggccacttgaagtatggctagcctaaattgatctatgttgtaagtataaaattcacaccagcttgtgaaaacaaattatgaaaaaaaagtctttaagatatcattaacaattttatattggctaaatgttgaaatgatcatattttggatatattggattaaataaaatacactattaaaattaatttaatgtttctctttatgtggttactagaaaatttaaaatttaaaattacacagggcgatcacattctatttctagtagaccacactgctgtaagctcaagattcaaatgtcaaactcctgtgaatattaatacgtgaatatcccacaagcacttactccatcttcccaaccctcagcccttctgtcctccttctgctcccaccaatctgtgtttcttctgtttcactcacccagctaaaggcaacacaattcactccgtgacgagccaggaaaatggaaagacacattttcctttattcctcacattgatatattcactgagcactataattacctcttaaatatgatataaatctgcaagctcttttcaataccaccacaaattccatagttcaaaatgccatcagctttcacctatattattacaccagctcccatctggtcttcctgcatcctggatcacctctttctagctgccctttcaaatttcaataagagcaagctttccaggaaacaaacctgaagtcaatccactgagtactcctctgaataccttaatattgttgacaaattcctttctgatttgaagtatcagaaaggaatatttcctccataccaaatagttttcatttcatgcatgtgccgtgattcttctccctcctttgcatctgtcattcgttatgcttagaaagctcttttcatctctttgttcttcgagacaaccactactcatacttcagagcttaatttacattttgctttccctcaaaatttttttaaaaggttccaggtctgggttatgtgctctcttatgtgctcccagagcatcctgaacttctgcaataatatgtttggctactgtattttatacagtagttttatattgtattttatactgtattttatacagtaggtgttatattgtattttatacagtagttgtttttctgtctgtttttgccccaacaagaatgtaaaatctttaagtgcctgttttcatacttatttgaccaccctatctctagaatcttgcatgatgtctagccctagtaggatcaaaaaatacttacaaagcaactgaatagctacatgaatagatggatgaataaatgcatgggtggatggatggattaatgaaatcatttatatgacttaaagtttgcagaggagtatcatatttggaaggcagtaaggaagtctgtgtagtcgatggtaaaggcaattgggaagtttgttaggcacaataggtcaaaatttgtttttgaagtcctgttacttcacgtttctttgtttcactttcttaaaacaggaaactcttttctatgatcattcttccagggcctggctcttcatctgcaacccagtaatatccctaatgtcaaaaagctactggtttaattcgtgccattttcaaagaggactactgaattctgatgtggcttcaaacatttaggttaggcatatctaatggagaacttgcagccacactgacttgtagtgaaatatctattttgagcctgcccagtgttgcttaaattgtagttttccttgccagctattcatacaagagatgtgagaagcaccataaaaggcgttgtgaggagttgtgggggagtgagggagagaagaggttgaaaagcttattagctgctgtacggtaaaagtgagctcttacgggaatgggaatgtagttttagccctccagggattctatttagcccgccaggaattaaccttgactataaataggccatcaatgacctttccagagaatgttcagagacctcaactttgtttagagatcttgtgtgggtggaacttcctgtttgcacacagagcagcataaagcccagttgctttgggaagtgtttgggaccagatggattgtagggagtagggtacaatacagtctgttctcctccagctccttctttctgcaacatggggaagaacaaactccttcatccaagtctggttcttctcctcttggtcctcctgcccacagacgcctcagtctctggaaaaccgtgagttccacacagagagcgtgaagcatgaacctagagtccttcatttattgcagatttttctttatatcattcctttttctttcctatgatactgtcatcttcttatctctaagattccttccagattttacaaatctagtttactcattacttgcttacttttaatcattcttccccaactctctgaagctctaatatgcaaagccttcctaaggggtgtcagaaatttttagctttttaaaagaataaattttagatattcacattcatattgatctacttgagaccatgctatttatcttttcttatttcctctttctcaagggtccattttctattttataaaaataaagacaattctctcccacaaccaaacatggaacaatgccctggagtataaaaatctatagagtgccaaataaaggaacaatttgaaatactggtgttgatattgaaaaagcaagggactctaatgtcagaagagaaatccttttgcagatgaggtggtgatgaattctttgtttcaacacaactgaaggaggaactgaaggaaataccagctgatgagtgatgagaagggattcttgataatagagtactaggtgatttttggcatgtaatgcagaagttgcaagaagtggtaacaatgatgcaattgttttacctgccatttatttacttttatgtgagccattcttcttagcacttatagctacacaaaacaaaaatagtaacagaattaatgttgtttaattcttgcaatccatggatgcataaattcactgggggaaaaaacagctcatcattctcattaaagatgtgcttcaaaagtattttaattttatatctaatatgtatgaatcatactttgtatttattttgttttgatcagttatatacaagtatttttgaacatagctcagtcagaaggaaatgtttaatatttataaatttatggttacattctatttaaaagaggagttaaagttaaatttacctacccacatatgttacatatatatgtatttatgtatatgtattcatatatgtatatatgtgaacataagtatacatacgtatatgtatagatgcttgacaataaagaagtaagaataattcacaacattttttgaaatataaaaatttaggataaatttctgtatggtaattggcatggaaattcaaattcaaaaaggaaaaaagaagagaaagatattaaatatcagaccattaaaagaattttttaatgtacttttaaatagtgatagtaggtatcttatactacagtgtttattattcatgagaaaattgtaaaagtaatctaagtattaatttaaaatatcatcaaaaataatatcttttgctattacttaaaatcatgataaaaatatgtttacttgaaaatatgtaaggagtgcacagagtccaaaaattattttaggagttctgtgagcaaaaatgtataaaaactacagggttgatcttaaattacatgtcagggtactgagaaagtttctgtactgcacatgagttaccaaggtctaaagtcaatcaccagaggaccatttttggatggagccattgtctaatcatgagctgaaaggcaaatatttaaaatgcaaacatccatggttaggtaacactcttaagaccttattagctgcttaccacaactgagactgtgaagtaatggctcactttctttgaggctcagattccatatctgtggagtggaagcgagtgcttaccatacaattttcacagggctgctgaatgtgtgtatgtatgtatgtgtgtatatatattttaatgaaaattctataatttgattagtttttgtaatgtccgcatgactgagagcttgcttactttttacagcaacttgaaggtaaaaatagattttacaacatgaacaaatgtaactacatatttttatttgaattcagatgttcacaaattgttccttaaagtgaagcatgcctacaagttttaatctgtttaagacctacctcaagtaaaatgttcactgccatggcatgtgagggaaaagggaaataattcttatgcatggccttcaacggccaaatttcatgctcatcagtacatcttctcttggtgtagaactgatgatgataattatgatgatggaaaaaagtgctgttgatagcaatgcctctcttccttcactttcctctaactgaaccgtctcattcccaggcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtgagtgtggctgtttgacttaatatacttggttcttttagtcagggtcatagggatctagtattctgtcagatgaggctttgggaggttggtaagaactgcaggaaggaatccaaatgtagcaaactaagtatagaaattaaggagcaatgcatgactctccagccatgggagacaaatatttacaggcaaactaaaagtcagcttaataatcacatagaaaccctattagccagaaaggaaaaaaaaaaaaagaaaatgtgacttcttaaaattaagatggaaaaaatattaataaagcaaactagaccaaggctcaacatgagccacccgtggtggactgacaggaacacatcactccacactacttctaagagtaaaaattgtagaaattaccctgagcaaaggtgtaatatctcttcagagttgtccacatgtgagatgcttacagcctagtgtcagtaaaatacggggtttttttccatagcctgtaaaacatgcttcgaccatgcccttttagcatgtaatatcagcctggtaaagctcagcgtataattgaatcaagactgtttctgtgagctgtatgaaggtgtgaatctcacttacaacctctcttactgatttattttcctccaccttgtgtcctgtctccccatttgtaaaatggcagaagtgattcctgtccatgccaactttccctgctgagaacaagggagtgaagttaagtaagaagggcaagatctccagggaagagtgcaacagcaaataggggaggagacctgcgggtgctgaatggggtttcaggatcggtcctgtatttcaggtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacaggtatgaagaagcctacagacaggacaacttcaaaaaggaaagatcttcttcccctggatgttccccaggcaaagttcctataatcttggttccttaatagcttgtcttaccagcctacaggcctactttgggtttgggggctcatgaaaaatatttctgtttcagtgaaatttcgtgttgtctccatggatgaaaactttcaccccctgaatgagttggtgagttttctattatctacataaaatgattgtctgtataaacaggctgggaacctgttttttgtgctgagggaagaccagggagaggaagaatctggtatcattaacagtaacttctggcattacaacagcacaagatcctaatctaaaacatcattccaggtaaagaaagtaggtaattctttctgtcttggtgctggtactcagtcagttgtcacacaattaaatttacttttcggatggttcttaattaggacaattagaaagatacattcaatagcagacacagaaaaatcctcaaagaacctaagctcaaaaaacattttaaagatttagattttttctatacacatctaccaaaatcttcactataaaggaaagtcagggtaattaatttgttcctcaagactaactcttggtatctgtgataagaaacagttctttctattgtaatgcagacatcaacccaaagtcttcatttttctctcccaaattaacttcttcacattttcttatctcaaaaagaggcaactcttctcttctagctccaaagacaaaagattatggcctagttcttgtttctctttctctcatacccacatccacttcactggaaaatcatgttggcttaaaatatattcagactatttcttatcatctgaactactgctgcaagctagtcctagtcaatgtcatctctaaataagatcattacaataaccttcaaagtggtctcccagcttctactttcactcctctgtctaaaatgaggcaccacacccatcactctgtcagcctaacttggctttgtttttctatttgcacttaccaccattctatacgtattgatttttaaaaatctgcctatttctattataacataagctccattaaaacatggtttattgttcttttgtgcgtggttatattctcaatacctagaatgatacacagcgcatgagaaggtacttaataaagattagtttttaaaaatgaataaacattcatagtagcttcttatcaatgtttattcatttttaaaagtaatatttattaaactaaatttattaatgaaatatgtccattcctcctcattcttagaactacgtaagaattctatgacccatcttaatgattacatctgaacatatttacttatagttacataagtgtttatttctcaacctgtaaaattgcatagcaaaggattgatcttcactaggatgctataagcacttaagacatgttaatccatttttagtaaatggcactttacatgtatatttgttgctgaaggcctagtaagttcttaacattatttattaattgcttaaaatagattaatgaaaagttctataaaatttaatctggaatatttctgtatttcactatagagggaattattctatatgaaactaatttagattttttaaactttttattgtttttaatttttgtggaaacatagcaggtatatatatttatgggttacatgagatattttgatacaggcatgcagtgcatgataatgattatggatgataatcattatcattatccatatcattatccaatcatggataatgattattatcatgcattgcatgcctgtatcaataaatggagtacccatcccctcaaacatttattctttgtgttacgaacaaatccaattatacttttagttatttttaaatgtacaattaaattattttttactatactcaccctgttatgctagcaaatactagctgctttgcttataaatgagatttaagaatatttgaaaataattataaacttcttttttcttttgtctttcagattccactagtatacattcaggtaagcaacatgaaacattccatattaaaaggaaagcaatacatatagggaaaatgttcttatttcagaggtttttacaatatctcagaaacttgtcattaaaggagaagccttcaaactcccatagagctagatggctataactcatctcctctactcaccctttactactaccccatttgacctttttgtagataacttagggtttccatagatatcttttattagctccaatgctccaggtgcttttgtaagttataattaatttactcatataggatcccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttggtatggatcatgaaaagtcatcaagcattatttttcttcatatttaaactcttaggtcctggaatttaagttcatttggagtctttccatttcccatgggtgacattgggcttggagtagaattaattacacctaagtccaatgaggacatcagtgatctgtgaataggacttcacatagcttcgttattttctgtagcaatatttaataccaacccccaaaattaaaacattcttgttttaatggagttttccataattaattaagcacacagtgatctctcatagtccctcaactgaaatcttcatttgagaggaggatagataagaatagattggagagcagagctaccttttcagagccctaaaatattattagggaactgttacagggaacctgaaaataggaattcccccaaagttgaaaaccaatcaccaaccttctttatcaccaatcaacagttcttcccaagtttgaagtacaagtaacagtgccaaagataatcaccatcttggaagaagagatgaatgtatcagtgtgtggcctgtgagttcattttttaaaaatcttttgtgggggattatttaaaagagactcaccatttgggatattttaactactctctccgggagcagtggcaacacaaaaattttaagtgctttgacagcatcctcatctgtagaatgttattctcctgttgcttttctatttttattttctttcacgttcttatcagtattattctatcatgaggtaagaaactgtttctagagaggttatgctaataggattgattctgaaagtgacaaaactgcacacacacacacacacaaaatgggaagggtggatagtgttgaagggtattggttctgccttaaccaaaaataaccaaacgtatattagggagataattaacacatggctataggggaaattcaatcatcaacagatcatttacctgaccttgcatgcttactggaaaaatcacttagattcagaatttgtagagataggcatacaactgaaatctcatctaactcactagtccacctaaggcgggtctacatacctgtctgcactgacatttttacgaagagactgaacgtgatttctcaagacagtctgttccaaccttgaaaagtcttgtttcctggatcttatgttcccatccatggtggcacacagtagcagtagcaggaagagcaggtatagtcctgtccaaagatcacacatgtaatacacctactattcagctaggcccctgctccagttctgtctcacagtgatggccaagttcctggttcctcataactggtgtgatcttgcaaatagacatacagaggacattctcaaataggaaaggagacaaatgcacttggaaacccagcaattgtctatgacatatttacgacacaatcccacttttgtaaaaaagtaaacaggcccggcgtggtggctcacgcctgtaatcccagcactttgggaggctgaggtgggcagatcacgaggtcaggagatcaagaccatcctggctaacatggtgaaaccccgtctctactaaaaaatacaaaaaattagccaggcatggtggccagcgcctgtagtcccagctacttgggaagctgaggcaggagaatggcatgaacccaggaggtggaggttgcagtgagccaagatcgcaccactgcactccagcctgggcgacagagcgagactccatctcaaaaataaaataaaataaaataaaataaaataaaataaaataaacaagtgaaactcgatatagagatggctgcacatggggagaggatgatggaagaagagacactatcgtactaatagtttgtattcctaggtagacttgagggcgtttgggttttttttgttctgttttgttttgtttgtttcgtcttgctggtattgtttctcttttaagaacgggtttaagttttataataagaaagacttttttaagataaaactaaaaaaaaacgaggaaaaaaaagaaatgataaaagaagaatgtaaatttcagcatgttgcaatggagatttctataagagccattagtgactcttgtcttcaatattgtgtgagcccagcagagagcagagggaagtacagacagggaatatactgtagctaaaggggagatataaatagctagaaaatggggaggttcagtgtctgctctgattcctttgggaatgttctcatgacagatacacatatgggaagcctgtccctggacatgtgactgtgagcatttgcagaaagtatagtgacgcttccgactgccacggtgaagattcacaggctttctgtgagaaattcagtggacaggtaggttgaacactattttttctagagaatagcgataaaggcattgttgaaaagcagtgagttgcagcatttttctgacgcaggaagagaacaatctagaagagaattccatgttggctattgtaatttttcaaaaaaaatcatgaacttagcacaatgggaattatttatttctcgtaattgcccattgtgagtgtttcagaacgatagacactgagccatctaaagcctccatgggcattcacttctacaaaggaaggaaaaaaccatacacctcttaattgccttagctggccaggccatcagttctgctctctctgtgaacaagaacctatcacatggccccaccaagatgccagagagttgacaaacacagtccccatatagaaggccgcttcccagccacagctgaatattatggaggaggaaccagactttgatgaggagttctaatggtcaagagcagatgtactgtgtatttcaaaatagcaagtggacaggacttgaactatttccaacacatagaaatgatacatacttgagttggtaggcaccctaaatcccgcgatgtgatcattacacattctctgcatgtaacaaaatatctcgtgtaccccataaatatgtataaatattatgcatcctttgtacaaaaaaaattactcctcaaattttaagacatttctatcgcaatatatctagggaatttcagataccaaggaatacatctgtcaattatacatgagatattgttgtgaaatttaatatttagttgctagagaatatttattgtgtcctcgtcatagaaaatgcctacatgatgttgtccccccacaaaaatcacaccggttatctgaccactgatctatttagtataatatgcattatatttttgtattttattttctcccttcttagctaaacagccatggctgcttctatcagcaagtaaaaaccaaggtcttccagctgaagaggaaggagtatgaaatgaaacttcacactgaggcccagatccaagaagaaggaacaggtttgtgtactacatgggtataagagaaaacacaacaggcattgattttcttagccaaataatgaattgtaagttggggggaggtgatagaattttagagacatcattcttcccaaaaataacagatttttttctcttttttcagtggtggaattgactggaaggcagtccagtgaaatcacaagaaccataaccaaactctcatttgtgaaagtggactcacactttcgacagggaattcccttctttgggcaggtggagtattttccagttcactcatcaacccatgtactgttacctaattagcacaatagttatggtttgtgctaaaaccatgcctggttaatgttatcatttaatataaccaaaagtataaaatatcaccaaggcttgattagtataaccaaaggtataaaactacataaaaatagatttattcttctgtaaatttgtgtatgaaatgtatgtaattatcctaaggccttattaaaattagtagagttttccccccttcttttgaaacagcattgtacaagtcactcaatctccttctcatggtttaccaggcagtctcaggtttttatgacattttctcacaagaatctcaaaattcatgctgaccagtttcattacgatgctaacactaactttgtatggaaacaggtgggtaggtggttttaatttttattttgaagtattaaagattctacaataatgtttatttcatggatagtatatttacactatttttctataacaagtatatttctaaaacagtgaacatggggtaaaacactgctatttaaggttctcagatttttgaattatgaattttcatgttatctaccaaaaaaatcttctcttacaatttttctgttgtcagcagatgtgcaaatggatctttatgactctagaagtctgagatcacagttacatgtgcctcaatgtgtatttactgtgtatcattttcttaatgtaaatgttacaggatttcagctatgaagggtgtaaaagaggcatagactcaacaaagtggagtacattctagaaggcttggttatgtcatgacaccaaaatgtattcaacttcctaaaatgaaagtgtagctcatttgtaaatttctcagaaagatagtgtactttgtaagtacattttattgctgttaaatatcctatttgtcataagactctctagctagaagaaaatcagaattatgtgcctattcttgtctttgtacatgcagccaacatttcactggcaaacttagattttgtaaatcaattatgagtactgatgggggtcagcctattgttcttcctgctaccaactgtagcttatactgaaaaagaattgccacctttacaatctgaactggactactcaactgccaatacaatattagcagtaaattagatttggcaacttatttaatactgtgtgcctcttttcaattttatatttttcacatgggaagtgtgtcataaaatctgcctaccttccaaaaggctgaagatctaggttgagaagcagagaccatgtctaaggcaactggagaaacacacaggacaatgtattggcaattgtttacttgtgcacttatgagacttcagacactaatctataggaagttaatggtccactccaaaataggtgtttggggcacaaataaatttagttaatagattaatagattaaaatatttcattatcatattactctagtgctctgtgactctctaagagttatatataataatacacagcacttccaaaatatctagaagacatttttcaggtcactcttgttattatccctatcactctctatcccttcacactgctctgtttttcttcttaggattgattactactaaattagtgtatgttatgtatatatttatttagcatctatctctttcactagacagtaagctctgtgatggaagaaacttcgttttttttcactgctgtgtcctcagtgcctggaaccatgttcaacatagaggaggcactaaaaaatgtgataaatgaatatgtttggtgcctattagttattaccaatataactaatccacagcttttaatcttcaggtgcgcctagtagatgggaaaggcgtccctataccaaataaagtcatattcatcagaggaaatgaagcaaactattactccaatgctaccacggatgagcatggccttgtacagttctctatcaacaccaccaatgttatgggtacctctcttactgttagggtaagtttggaaagaaattaccaatgacatgaagtagccttggaaacaaggttgcaacctaagggtgagaaaatttccaaactgtgtctagttctatggagagaaaaaaactagcaattagaaaccgattgaaggttaacttttttaaagtttatgaaaagaaggcagtatattgtgattaaaagtgcgggttagactttagcagtgttgctgggaacgtaaaatggcggagccactatgaaaaacagtatagtagttcctgaaaaaattaaaaaatagaattaccaaatgatccagtaatcctacttctggacatatattcaaaagaatcgaaaacggggtctcaaagagctatttgcacacccgtgttcatagccgcactattcacaatagctgagagatcgaggctacccaaatgtccatcaagggatgaacaggtaaacaaaatgtggtatataaatacaacagaatattatgcagcctgagaagggaagaaaatcctgtcacatgctacagcatgaatgatccttgaggacgttatggtaagtgaaataagctagtcacaaaaagaccaatactgtatgattcacgtacatggggtttctaaagtagtcaaagtcatagaaacagaaaacaggatggtggttgccaagggctagggaaagagagaaatggggaattcctcttattggtattcaggtttagttttacaagatgaaaagttctggagatctgttgcacaacagatatacttaatactaccaaactgtacactaaagaataatgaagatggtaattttatgttgtgtgtgtgtgtgtgtttataataacttttttgaaaagtgtgaattccagaatctgaatcagataaagtggttaaaaatactggctcaaccctattttaaggaattaactaaaacctctgtgcttcatttccttatctgtaaaatgattgcaatactaacacctgactcttggggtggttgtgaagattaagtgaaatcatacatgttgaatcacttagtaagccccctttaactgttagatacttttactataaaagccaattctaacataattagcatttagttttaaatatatatactccaaaaattattaccttactttattttgtcttgctattctaattttctccagatgacctgttcataccaatataagttattcggtatagcatgcagtcctctattcatcaagagagatgtagacatgtctatagatacatggatatacaatacatatttaatatatattacataatcatggtaatggaaaacgcctgcctctttagagttttaccttgttatagagaaataaataatacattttatttatttagctttaatagatggcataaaagcacctccctctaatttgaatgtttactcatttcaaaaagtgtcttgaatgctttccacgtgccagcactgtgctagtcttgacaatgaaattattatttctacctgttccctgtttggggtgatcatatatgtgttgtcaccaaaatatacattaagatatgaacaaaatgttttgctcaaggattcactagtgctattgggagctggggatgaaggtagaggaagcctgaaaggagctaaggaataacttctacaggaagaaaagttggaaatataacccaaatagggcctctgaggtattagaatccagattgcagggagcattaaaaatagtcagcctaggccaggcacggtagctcacgcctgtaatcccagcactttgggaggctgaggtgggtggattgcgtgaggccaggagatcgagacgagcctaggcaacgtggtgaaaccctgtctctacaaaatatacaaaaaattagctgggtggggtggcatgcacctgtagtcctagcaactcaggaggctgactgggaggatcacttgagccccagaggcagagattgcactgagccaagattgtgccactgtactccagcatgggcgccagagcaaaaccctgtctcaaaaaaataataataatagcaatcagcctaagatagcccagaagaggtagacttcagctaattcatcagctcagctcttaaaccaatgctttctaccaatgtcttctcagacccctaaggttacaatattttatttattcataatacccattcaaaatccaccctaaaagaggcagctgctttctgaaagcacagttcttccatttctagagattatttactctcctcaatgaagtttcatagctccagtgtctctaattgcacaggtaaagcagtcaaagaaatttcaagcaagctaatcagagcaaaggatgcctccttttatgctcttaagaaatataaatctcaatcccaggaggctctgcagtgtaaagtcacaaagcatgcctacatttgaagcagagaaacaaaatcaggggtccttctcccacttttcattgtggaacaaaagatttctagctactagttaaggtaggacagtaaacttacgtagttttgtgagaacattaatctttatgacgtataatctaaaaataatataatttttctaattcattaggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttctattatctggtgagaagggaggttactgcgttgacttcactgtagacaaaagctctctgtggagcaagtaatcatgaagctctttagatgtcattacttcaacttcttatccatgttccttttgaaagtttgatttctcttgaggtgaattattgccggcagggactcaataaaacaagtatattgaagtgagactgaagcgtgttctctctggcacatttaatttctttttatttccttttttgcagataatggcaaagggaggcattgtccgaactgggactcatggactgcttgtgaagcaggaagacagtgagtatttccatcatctctgcattgctgccccattctgacccattcagccttacaccacggaagtatcagaatactttccctttttttccataggtttttgggggaacaggtggtgtttggtcacatgaatgaataaattctttggtagtgatttccgagattttggtgcacccatcacccgagcagtatacattgtacccaatttgcagtcttttatccctcacccccttccacccttccccctgaatccacaaagtccattgtatcattcttatgcctttgcatcctcataacttagctcccacttcctaatagcttagctgccacttcctaatagcttagctcctacttctgagtgagaagatatgatgtttggttttccattcctgagctgggaatatcaggaatactttcaagtaacgagagaccatttccatctaatattaattaaaagaaagttgagggagggaaaacaataatggtttttttctcaccctttatcaaatatgtgttgtcttttttttcagcctattttctttgcgttatttaaaatattggtgtgggccagctatgatggcttacgcctgtaatcccagtactttgggagaacgggatgggaggatctcttgaggtcaggagtttgagaccagcctggtcaacatagtgagaccccatctctataaaagtaaataaataaataaataaataaataagaacaaagggggaaaaataaaatacataaaatattgttgtgtataatctatgcttattccagatttttaaaccataaatattcttgaacctcttctcaaaatgagaattgtgttagagaatggggtggagaaaatgtacgatttcaaggtatgttgtatataccaggtcttgtgctgagtcatttatgtcatttatattattataaatatcttttttccctttcaagatagttatgatttatgaagcattaaattgggtacttctaccgactgaagcggactcttgccatgagcaaatatgtcataatataaacctttgaaggttgctcctagtctagtagactctaaaagataacttgctagaaaagtatcagtaatctactctctcattagttcttgtagcaacacagaagcaccatagcagactgaaggaaacttaatgcaacctctaaagaatctacataggcatcaaaaaataacttctgaaggagcatggtgttcctcttatggagaaagaaccaaatattggctcatagaaggttaaagagcatgtagcatgaatacaatgtgttgaactcttactcttacatctcataacttcaggttctatttccctcttctagtatgaggcctagatcaaatggtagtgaattggatgtcttcatacccttaactagcatcagaacattaggtgggtgagctgaagcttaaagctggtgaaaaatcaacagagtatatgatttatgagaggagagagggctttgatagggtttttttctaaagctagagtccttagaggtaatcatccatttgttacatctactgtttattatgtctgttacatctactaatggaaatagtcattaacctatgatggcttcctctcattctctttgtgccccagtgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtgtgtgttttagatcataaaatcttcaacatgtaaaactagaagttactattgttatcatttgttttacacatgtgaacattagggccaaaagggttaaacaaattccccagagacattcagcaaggtagtggcagagtcacactaagaagcagaatcacttgattccttatacaaaactctcaaacttctcctagcagtgcctcttaccaattacacagttcagagtatgttattcccttcttctatgatgaaacgttgggaaatatgtaagcaagtttttaagactactaaggagccaaaagaaaaatgcaagagcacctagacccatgcatgtcccaccacaattaatgagcaccttgccgtgtatcaaagataaaaatggcatttcatgactagtaatttataagaataattagaaataattctactggctcaagtgattcttgagaatgaaagagaagtgtagggaacaaaaagcttcaggattgacacaatgccaaccctccacgaagtcaagcagagtggttatacattgtacatgaggaacactctgcaaatgcgagtgaatgcatgggtaaacctggaaatttgcttttctgaccttttgttccaatttcacaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtgagttcctggcagcctcaggaatcaagaagggccgtgccaggggctcagagcaaggaaaaatgactggataagtaggataagatcaattaaaattatatttatctaggaattaattgctttgagctctcttgtgggctttcattggtaaggaattatatatatatatatatataactactcacttctgcagttaaaaaaataaacaaaatacaatatattgaatcaaataaaagaatttttaaaaggacaaaatgtgatgaaaattaacaaccaaaaaaggactcgttattcagacacgttaagcttcttgctcaagagcacatagcaatacaaattcaaatcttctgattgtgtcaggacatccgtatatgcaaggctggagagaatacagagcagattgtgaaaagtgctatatgagaagtgcagttatgcaaaataaaagaaaggattaactgagcaaccggggaagccgttcgaaaattatatatttaaaatgtaaaaagaccatacagtacccaaagtccttaaaatcccagagctccatgcaaccagtaataggagttgtcaaactagtttcaacatttcaaaaagcctaacaaaagtgatacatatacctgcactgaggctatacaggctaaatgaaatatcagatttccgtttttataagaattatggctgggcctggtggctcacgcctgtaatcccaacactgggaggccaaggcaggcagataacttgagatcaggagtttgagaccagtctggccaatataatgaaaccccgtatctactaaaaatacaaaaattagtcagacgtggtggcgggcgcctgtcatcccagctactcaggaggctgaggcaggagaatcacttgaacccaggaggcagaggttgagtgagccgagatcgtgccactgcactccagcctgggcaatagagcaagacttggtgtcaaaaaaaaaaaaaaaaaaagaattacatcagttgaaataattgctgtcccaagctacacataatttctgagggaacatatgtattatctctgaggaacaaaacttagggaaattgaactactatataattgacattgataactgaactctttgaaaatatggagctaatagaagaaataccaaaaggatatgctgaattggaacaacaaaaactagttaatagcatgtactaatagtagtttgctcacatcttcaaatttttaatatgagaccaattgtaataacctcttactgaagctttttctactaccataatttaacaaagagaaatttatatttttattttcctgaaatagaaacttataaaaaaatgtttcttacttgtttgtccttctaatgggattttaactgaaaatattagaatacttctaaaggaagcatcaagtacatttcaacagtaggatctcaagaggatatgtgggaaaataatatacatgttttatacttttataatataattttataattctcaaagaatttttcaaattataacaaccctgatgggtagataaggcagacagtcctatttattaataataaagcagaagctttcagggatttagtagtagacctgagttaaaacaccaactcttctcaagctttgattagtgtcttaccaatgaaacactttgctgctactagtgtaggtcattcattcaacacatttatttaatgcccactgtgttctaggtattatactaagtgctagtagagatcaagcagtgagctactggaaagataaaaatgtatgtctcatggaacttacattgtctgtcccatagatgagacagacaataattatgcaatatgccacaataaaagcagggagaggaaatgagaaatgttaagatactttgagaaagtgtctaatttcatcacaccactcactttgctcatctgttcttgtcaatcagttttaaactcctacgaatataatgcaatgtaactatcacaatttttatgtctgtcttacttctcaccttcaaatggacttgaaagcatcatgcctagaattttacggttaaagttgtatgtattatatgaagatctggagcattttgtttccactaataatacctaagaaaatgccatcgtgtcctgtggagagaggatattcctattcgtgtgcctgtttagaacatgcacccattaactttgctatatactgagtcagttgctcaccacaagataagcacaaaactatcatttccttctatcatctcaaagctttgtgcaatgtcacaaatacagcagacctcgatttttcaattaataaagttttatttcattccagtgttgagtctagtggtggcctctgaactgtgtaacgaagtagtacttagtacttagatgagtacttagatggagtgtttggtttttcctaaaattgttaaacatcttcaaaatgaaaacactgtgtcaagaaaatgatccataccctctataaatcatcaaagcaatgagagcgctcaaagaaagacggatgttcattattcctgttcttttctccttgaacttaaaaaatgtcacaaaggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccgggaagcggagcttgcagtgagccgagattgcgccactgcagtccgcggtccggcctgggcgacagagcgagactccgtctcaaaaaaaaaaaaaaaaaaaaaaaaaaaagtcacaaataagtttgcctttttgtctttcgtatttgtacaggtttacaacctgctaccagaaaaggacctcactggcttccctgggcctttgaatgaccaggacaatgaagactgcatcaatcgtcataatgtctatattaatggaatcacatatactccagtatcaagtacaaatgaaaaggatatgtacagcttcctagaggtaaactccttatgttgcagatggtctgatcttaagcttcttaaaatattacacatggaaaagagtctgtatttgaatgccttcatgtcctagttgagggtaatgggatataaagaggtaagtggcttctctactaatagcagcagatctgcgaaaagctgctgaactaagctggaactttttggagtattattcaagttttcttttttcacaggaattaattgctctgtgatgtttattaaaatcacatatacaaaataattactgttgaaagagtttaatgaaaagaataaaattacatccttaagtatataaatatcccttctacagatgtgaatgttaaaagtttaatttaagttaactggattgcttgtcaaattcaataaaaagaagtagctacctatgtatattttataatatatatttactgattagatgataattttctttgcaggacatgggcttaaaggcattcaccaactcaaagattcgtaaacccaaaatgtgtccacagcttcaacagtatgaaatgcatggacctgaaggtctacgtgtaggtttttatggtaaacaaaaaattaataaatatatattgcctaatatattcaccaaattttaaattttttaaaagatacaatgtgacaaaaattaacaaacaaaaaggacatgtgagtcatacatattaaggttattgctcaaggtcatatagtaatataaactcaaattctagtaaatggaggtacatgtgttaggctgaaaggaaagagaaagtttccaagcgtaggattagtgtaaacagaaatagaaatgttcacacaacaacactacattctccatcagtcaggtaaaaaagctgttcaacttccccaaaacatcagccaatatttatgttggaaccacacacattcatcaatgacatgagctacttctttgatgaactgaatagtaatcaagcatttatttatagtcccatattgtcaaatcatgattgaggataagttgagtacagagaaaagagggtcaaaaatcaaggaaagcatttaaggagaatcagttggctctatggaattcactatgaagcctaccgcatattttattatttataaattatattgtataatcctttatcagtaagatatatagtaaatttatgcatgtataggtatatatatatatatatatatatatatatatatatgcacaatttttttttccagagagctggttgtgaaatattgaccagcacacccttaatagaaggtgaatagagtgaaggaaactaactgtaatcttctgacacgatagagaataaaaagtctttattattattaaacttttccctcctgtaaactgtttctcaaagccactgtcataacatgtgtgtcagtttttccttgctcctgcagaggagcacaccaatggaaacttggattcctgcccctcttcacggccttgtgtcataacactctccacttagcacagcggcagcagccacataggactccacagagtccttgtgccccacagacaatccaagcctctgtctgccaaagtctctatagcctctgcttatgaccctcctcccccagctcactcctagcttattcttgtttttttttgttgtttgtttgtttgtttgtttgtttgagacgaagtctcactctgtcgctcaggtcgcccaggctggagtgcagtggcgccgtctcggcctcggcctctcaaagtgctgggattacaggcatgagccaccgcgcccggcctctcctggcttgttaagtcaaccaacatatttcctcctcaaagaagctttccttgatgacccaagcgccctctcctccttcctctctatttcatcagacgatttgttttttgttttttttttttggtggcaactattacattctctcataagctttatctgtatgtttattgtaatgtcttcttcctcactcaccatagagtcagatgtaatgggaagaggccatgcacgcctggtgcatgttgaagagcctcacacggagaccgtacgaaagtacttccctgagacatggatctgggatttggtggtggtaaagtaagtaacttcctgcatatgcaatatgcaacaatagaggtccctgactattttcaactctttgctagtttttctgtttttattgttttatatgtttatgatgtacaacatgatgttttgatatatatatatacacatacacatagtgaaatgattactacagtcaagcaaattaacacatccattagctcacattgttacctatttttgtgtgcatagcaagcacacctaaaatctatcctcttagccaagtttcagtatccaatacagtattatcaactgcagtcctcatgctgtactttagatttctaaatttattcatcctacgtaacttcaactttgtaccctttgacctgcttcttcccatccctgcaaccccaaccacctttctactctctgttcctatgtattcaacttcttttagcatccacatacaagtaagatcacgcagtacttgtcctgtgtctggcttatttcacttagcataatctcccccaggttcatccatgttgtcacaagtggcaggatcttcttccacttaaggctgaataatattccattacgcatagccacaatttatttatccattcacccagacacttaggttgtttttatatcttggcaactgtgaataatgctgtaatgaacaccaaaacacatatatctctacaaggtgcttatttcatttcctttgggagtatacccagaagagggatttctgggacatatggtagtttccattttaaaatttttgaggtatctccatactgttttccataatggctgtggcaatgtacatttgcaccaaggggagtgaacaaattccttccagagagacactggaaacagagttttatccgtgaaacaagccagtggggcaggtggagagaaagagccggaatggccatatactccctttcaggctcctggagtcttgtttacttctccctttcactcccagatgcaggctgtttagaagcccaacccttaggagacagccagaaatgggagattttgcctgttctctctgtattgagccaaggggtggtgggggcgggtagccactggcattgctcaaaggcctatttaaaaccacctctgtgttcactgtggtctagggagactcctgattgcagagctccatctactcctggagctaggtgatttaggagccagacccttaggtacgagctgtaaacattggggtgcttgatgcatagacaaattattcccaggggtatgttcagacctggttttatccgtggggcgagccaggggaaggaggcatgggaagtgcccttactgcttttcagccttcctgtaagtctgtcgtttccctgctccttctgcttcccagtgcaggctagttagaagcccaacactcagtcagcaactgataaagtgggtagacgaagcccttccagggagaaactgggagctgttagctaatttttcagtactgaagtacttctttcaaacgcattgctaatttcaggagatgtttaacataaatacatcagctaagaagtcttactaatctaattgcatcagagctaaaaaattttgtgcaaatttaattctaagatttccagaaaatgggcataaggacctaatacaaccaaggactgcacagattgcctgtaagagatccctcactggttagcaatcctgagttaaatacagattcagtcaggcccactatacacaatagtagtagaatttaaaattataaagcagccctcagtgaaatagcattttagagaagagaacttaagaacaatctcaaactgcatgttaaatttataactatattgtctgtaaaagattatgctacaattctgatatactacaattaaaaacagttggaagaaaaggacttatattcccatctcaatccttgattatactctccattattggtatttctattgagtgtttttaagtcatggcagtagaatcattcctagggattctctccctagaaaggatgtatttaactgcttactttctgttcttcacttacactcctctccagctcagcaggtgtggctgaggtaggagtaacagtccctgacaccatcaccgagtggaaggcaggggccttctgcctgtctgaagatgctggacttggtatctcttccactgcctctctccgagccttccagcccttctttgtggagctcacaatgccttactctgtgattcgtggagaggccttcacactcaaggccacggtcctaaactaccttcccaaatgcatccgggtaaggatctctttcctaaattaaatacaaggtagccatcaagtaaattaaaagttgcattcctaggaatactgcaaactcttgtatgcaaaatatgcttactagatattcatgattgtaaaagttacaggttttgagatctccaaataccaaattgcaccataaagcaagtttctagcccttataacccttcaaagttagtatttgtgtggtatgaagaattctgacaggggtgacaaaagtcagtactctattctcatgacagattctacaaggtttcaacctctacgatctcatatatttaacttttcgtagctcattcattatattaaacctaattttaaaagtcgtttgtgagcatcttacctttgctgaaaccataactgtttataagtcttgtatcctctgccgggagatccgggggaatggtcaaagttccagaccaaagaggtagagcagcatgccatcattacctttcccttcctctggtcccatcatgtgaaagagcaggttgcttccaaaataactcagatttacctgtgtaaatctgatacattaagatccacttaaatatatttcaggtacttgatcttcatttatatcatctttaatgtgaggcaacctgaaatctaaccaattcctcaagatgctttacttcaaacccattctccctctgttctccttccctctctactccctttcttatgtgtggtttcaggtcagtgtgcagctggaagcctctcccgccttcctagctgtcccagtggagaaggaacaagcgcctcactgcatctgtgcaaacgggcggcaaactgtgtcctgggcagtaaccccaaagtcattaggtgagcaaaaaactgctagagataattctctactcaaagattgtatatggcagtgggaaccttatattgagtgctacttccttcaggaaaagaccactagatgctgcgatttttttcctttgccttttattctaagatgcctacaaggatatcctcaacatctccaccttgaattctcagtatcattcacctctcatttgcatgtttccgttcctgcttctgtgttttaataaaacaaaagtttacagagcattgaacatttctaaatcttgagtttggaggcatggaggaaggggaagatgctattcatttctactggccttttttttcaggaaatgtgaatttcactgtgagcgcagaggcactagagtctcaagagctgtgtgggactgaggtgccttcagttcctgaacacggaaggaaagacacagtcatcaagcctctgttggttgaagtaagtaaacctaaataatatatagtccacaataatatataatatatgtgggtaatataataatatatggatattttataatattattctcatgtatctctctgtcctatctctctcttgatttactttctgttttgttgggggtttttgtttttgtttttgaggcagagtcttgctctgtcatccaggctggagtgcagtggcaggatctctgctcactgcaacctccgcctcctgggttcaagcaattctcgtgcctcagcctcctgagtagctgggattacaggtgtgcaccaccacgcccagctagtttttgtatttttagtagagacaggatttcaccacgttggccaggctggtctcgaactcctggcctcaagtgatctgcccacctcagcttcccaaagtgctgggattataggtgtgagccaccatgcacagcctccctttgatttactttcttaatttttccttcatttgttcatgcatcgaactacctcctacgtatattgcttatatgtacagaattttcttagataatacagttcaaatccttctcttcactatccaaatatctgtggtccctccattaaaacacatgttctgaaggtcagtccattctcactagcttttctttcttttacctaaagcctgaaggactagagaaggaaacaacattcaactccctactttgtccatcaggtaagagtcaaccatcataatttaaaaaacattaaagtctaacatttaaagttcaaagaacatttatatattattcctacactttctctgtgatctaagacctgaagcaccatcaatgcatttgacaaatgtggaaaatagttcttaggaaggccaagtaatttgatcagaatatccctaggcctgcattctgagtcttgatcttttgcagcacctgtgcaaacaccaaatgactttctgaccagtgtatggtatgggcataggtagaaagtgggtagaatcaaaattaatattaccaaaagggatgtttccttaaataattaataatgcaaactatggacggctgaatttagggcattctaacactgagttttacatagccaacagtatttgataacgggattgctatttcccaaaggaaaagttgtcatggcctttaccattattgtcatattaatatctgtttgatgcctatcccgtacctaatgccctatcaaacatttgagaaggaactgaagaaacttacaggaaaaatttaatacactaagaaatttatcagcacaatgcattctcaccccaaaccaacattgaatcaacatcatacataggttcattgcctttctctgactacctacaaatttagtatgtttttcgtactaaatactttatctattcatctgttgccaagatgtaacacataaaatgtaccctaaaaacataacttccttgtcatttagccttatttctacatttaagtgaactgattacctatcattcaatccttttatcatgacttctccgtttctgagttactcattttgatgtatctcttaagtgtaagggctaatcatcaaatagttttactaaatttcattttaattaccaacataatcaaatgtgcctacctaattttacaaaaatatattcttctttaaaaaaaaaaacagaacatcacattaaaggttaatgtcacccccctgaacatttttcagtactttgccatccatttatctttagaaataatgtgtgtagatgtatatgtttgtggatgtgtgatttacatataataaactgtataagtttcattctataaatcactgtttgtttttcactcagcatcctgtcttggagatttacctatgttaaattgtagatctaggtctttccttggaattgcttttaagcctataatataaatacatcacaattctgcttagtgttttagtcttcctatattggttttttcaactattcactagctttaaaaaattagttagttaattataataagagcctcttaatgaacatatgcaagtacagctagggtagatccaaaatgttaaattcctgagtcagagagcatatgcatagatatgcatagttttgtttggttggttgttgttgttgttctttaattacattgtaaactgaccatttataattgtatatatatacagcatacaaagtgatgttatgatttatgaataaaatgtgaaataattaaatcaagctaacctgaaatacttatgttttgtggtgggaacatttgaaattctctaagcaagtttgaaacataaaatacactattattaactatattcaccatgctgtgcaatagatcccaaaaagaaaaaaatgtattccttctgtctgagactttgtgtcccttgaacaccaccttccttttactccagcttcatcctccataaccaccattctactctctgctcctgtgaatttgaatgttttagcttccacatacaaatgagaacatgcaatatttgttttcctatacctggcttatttcacataacataatctcctccagatttaatcatgctgccataaatagcagaatgttcttgttttttaaaatggaatggaattctatgtgtatataccaaattttctttatctgttcatctgttgatgacacttatgattccataactagacatcagtaatttgttagggattacattcaatatgtagattgctttgggtagtgtggacattttaacagtattaattcttccaatccatgaacattgtattttttttcatttatttgtgttctctttgatttctttcatcagtgttttataattttcactgtacatttcacctccttgattagatttatttctacatattgtttatagctattgtaaatgggattgtttttatttctttctcaatcattcattgttagtgaacagaaaatactactgatttttatgtgttaattttgtatcttgcaactttattgcattcatttataagttcttgcagccctttggtgaagtcttttgagcttccaatatataagataatgtcatcaccaacagtgaaaattttacttcttccttgtcaatttggatatttttcatttctttttcatgtttgattgctcttgctgctacttccagtgctactttgaaaataaatggtggcagtgggtatccttgtcttgttccagatcttaaaggaaagtctttcaattttccactgttaaatatgtaagctataggtttatcatacatgccctttattgtgttgaggaacattgcttgtatatctaatttggtgagagttttatcataaaagagcattgaattctgtcaaatactttttctccatctaacaagatgatggtatggtttttacccttcattctgtaaatgtaatgtatcacatttattgatatgcatatgttgaacaatttttgcatctcagggataaatcccacttgactaggtagatgatccttttactgtattgttggatttagtctgctagtttatttcgtgtggttggttggttggtttagttttttaagtgatgagcttttgctgtgttccccaggctggacttgaactcctgagttcaagcaatcctaccacctcagccacccacaggtgtatgccaccatgcttagctgctatgctagtattttattgaggatttttgcatctatattcatcaagaatattggtctgtaattcttttttgtaatgtctttttatgacattggtatcagggtaatgcttgcctcataaaatgagcttgaaagtattccttcctcttccagcttttggcagagtttgagaaggattggtattcattctttttaaatgataagtagaacctagcagtgaagccaacagttattaggcttttctttaatggaaaactttttattactgattcaatctctttactcattatttgtcagttcagattttctatttcttcatgattcagtcttggtagtatgtatatgtctaggaatgcattcatttcttctagatcatccaatttattggtgtatacttatgcataatagtctcttatgatcctttgtatttctgtggtatcagccataatttctctttcatttctgattttatatatttaaggcctccctcttttttcttagctaacctagctaaaagtttttgtctgtctttttaaaaaaacaattcagtttcattgatcttttgtattctttttctagtctctatttgatttatttctgctctaatctttattattttcctttcttctgccaattttgagcttactttgttcttcttttcctacttctctgaggaatatcattagtatctttattggaaatttttcttcttttttggtgtttattgttataagctttcctcttagaattgcttttactttatgctatgttttgttatgctccatttccatgttcatttgtcttaagatatttttgaatttccttttaaatttctttattgacccattggctgttcaggagcatgtcatttaattttcatatatttgtgaattttttctaattcctcctgttactcatttctagttttcatagtattgtggtcagaaaagatacttgatacgatgaaacgatttcagtcttcttaaatttgctaagacttgttttgtgggctaaaatatgatctatcttggagaatgtttcttgtgtgcttgagatgaaatgttctgtatgtatccattaggtctatttgatctaaagtgttggtcaagttcaacgttttcttattaattttctttctggataatctatccatttttaaaagtgagatgttgaaattccctgatattactgcattgcaacatatctctcccttcaacctttaatatttgttttatatatttaggtgctccaatgttggatatgtatagatttacaattgttatatccttttgatgaattgacccttttatcattatataatgttctcctttgtctctttgtacagtttttgactttaagtctgttttgttgaatataagtatagctacccctgctctcttttagtccccatttacctagaacatctttttccttctcttcactttcagtctatgtgtgtccttaaaaattaggtgagtctcttgtaattagcatatgtttcggtcctgtttttttaaatccattcagtcactttatgtcttttaaatggggaatttagtccatttgcattcaaggtaattattaatttaaaaatgacttggtactaccattttgttgttttctggttgttttgcttctttgttcctcttttgctgtcttcctttgtggtctgatgttctgtagtggtatgatttgaatcttttaaaatttttgttctgtgcttctattaaagatttttgccttgctgttactatggggtttacagtcaatttcaagctgataacaacttaactttgcattctttcactcccccacacacattttatgtcgttgatgtcagaatttacatattttgtaatgtgtatttattgacaatttatttttagctatgcttgttattaatattttgtcttttaacccttgtactagagataaaattgctttaaataccatcattacagtcatagagtattttgaatatggctctatattacttataccattaaattttgtgcttttgtgtttttgtattattaattaggggccttttgtttcagcttaaagaactcccttcagtaattcctgaaggcaggcctaatgttgacgactcccttagcttttagtttgtctgggaatgttttatttctccctcatttctgaaagacagctttgctggatgaagaattcttgattccatgttgtttttatttttgttttccttcagtactttgaatatattattccactctccctcagcctgccgggttactgctaaaaatccatggatagttgtattggaattcctttgtatgtgatatgtttctttatcaccttctgcttttcagaatttttttttgtctttgatttttgatagtttaattattgtgtcttagtgagcagttctttcatttgaatttcactggagacctctgtgcctcctgtacttggatgctagcatctatcccctaattagggaagttttcagcccttactgcttttttttttttctgattctatactcttttttttcattattgctttaaatgtgctttatagtctctttcttcttcttctggactttctttaatgcaaaggtttgatttcatgatgatgtcccataatttccataggctttcttcattcttttgtctttctgctcttctgcctggataattccaaatactctatctttgagctcactgattcttctgcttgatcaagtctgctgttgagcttactttgaatttttaattttagtcattgtattctttatttccaggatttctatttggtttctttttgattgtttctatttattttttattttacaatattagctaagttgcagataattgtttctatttcttttttttttatactttaagttctagggtacatgtgcacaatgtgcaggtttattacatatgtatacatgtgccatgttggtgtgctgcacccattaactcgtcatttacattagctatatctcctaatgctatccctcccccatcccctcaccccacaacaggccccggtgtgtgatgttccccttcctgtgtccaagtgttctcattgttcaattcccacctatgagtgagaacatgcggtgtttgtttttctgtccttgcaatagtttgctgagaatgatggtttccagcttcatccatgtcccaggaaacaacaggtgctggagaggatgtggagaaataggaacacttttacactgttggtgggactataaactagttcaaccactgtggaagtcaatgtggcgattcctcagggatctagaactagaaatacatttgacccagccatcccattactgggtatatacccaaaggattataaatcatgctgctataaagacacatgcacacatatgtttattgtggcactattcacaatcgtttctatgtcaaacttctcagtttgttggtgtattgttttgcaaatttcatttaattttgtatttatatattcttgtagtccactgaatttcttcaagaggattattctgaattctttgtcagtgatttcatagatctttatttctatgaggtcaattttttgagcttggccagtttcttttggaggtgtcatcattccttgattcttcataatcctgtgtccttgcattatttgtgcatttgaggagaaagccacttcttctggtttttataggtattctttggcagggataaaggtttgctatttagtctagcctataattctggaaagatcagttggtgacaaccttgagcaggcagagttttcatgggttccctagttggctgggccactgcctttgctcttatgtttggtagggccactggttgggccttgctctctggcaagatcactgttttgtctctgctatctggtggagctgctggctgggtactacaatggcctctggtcaggccagtcacaagatgtgttgcctggctggatgattctgctatttgggacctgaagttaggcagggtcacaatccgggctgtgaggttaggtagagttgttgcttgggatgggcagaaataaatactatacttcttagatgtgcataatagaggattgctaccccacccttgtgaatggagccatggagtgaggttttggctgagttgagctaccctttagactcccaggtcaagcatatttaacccctacacttctatgaaatacacagaggtggtgtctgctacctgggtggggtcactggcataacctctgaagctgggcttacagactggccatctggaaactcaagctaggttgaacttcccaacatgcttctgaaagtgaccagctcagttttgcagatgggctatgcagttggctggtatctctgaatgggtgccatagctggcagaaacacagaagcactaccaaaatccacatgctggtcactgtgagctctgtccttctttgtttctacctgacctcattacttcctgtgttcccggtgaaatgagaccagagtgggcttcctgagaagtgtctttgaatacttgagaatcttgatgtctacccctggttctcttccccgctgtagaaactgtgaccccagggaactcctctctatctggcattgtgctaacctaaaggagtgggaacaatgacatggtcaaagtgagaccattcttcctactcttctaatttgtcttcactcagttctatgaacaatgtaggtgtcctagacttgtttccaagtattggggttttcaaaatagattttctgatctgtggatagcagctagttggactttctgtggagggaggaagatcctgagactttctagtccatcatcttgctttattctgaattttaattgaactacaaaacgaaaatcctcctctttattacctaaatgcatttatacttccaccaggatacatttccatagtgttatatttgccatcatctgttaccatcaaagtttttaattttaatttttgccaaaaatttagaaaaaaaaattttgctgttgttttaatttatattttcttaattacaaggatgaccttatttttgcatgtttattaattgcctttataatctttggctatttgtcttttgagtagtttttttctgactcagttgtatgacactaattctttatctgttgaatatgttgcagatattttctttcacttggtcatttttttaactttgtttatggcatctgtttttttacaaaagttttaacattaattttatgagaaagggaagataactgctacatttttcatttgtatataattcaccaatactaaaattgtagtaaatgtatgttcatcagtagcagttattttattttcagtgagtcaagcattttattttgcttagccatttgtcctttaactatgcttatggcttttttttaagaaacattttaatatgaattttatgaggaagggattcagtatgaaaataaataccatacttctctcattttcatttcatataatttaccaagattaaaatggaagtaaatgtatgttcatgagtagtaattattttattttcaatgcatcaaatactgttcgtcttcacttccttaccctcaattttctaggttttccataaaaatactatctttgtatatgaaatttgaagaaagaacatagcattattatagaattcaggaccttttgtgggtaattttacttatgtatacttatagggctttgttgttggtgttttctccatacaactgttgagtaaggaagttggtggtgggaactaaatagatcatcttgtgataaccgtcttgtgtcagccatcagatgacagcaactgaatcacaacatcaccaggctcttacaatttgttgtcttatttggcatgcgattctacataaattactgaaaagatcattgaagaagaaattctgaaaatcacaggaaaccagtagcccatttttaagatatttatatattactgttgtattaaaggcggacaacttttcaggaggagtttaggtataaggcatagtcctagcttctgggtcatagagctgtttagaaagatataatgcagaaataattttcatatgtctgatttgcttatttctctaggtggtgaggtttctgaagaattatccctgaaactgccaccaaatgtggtagaagaatctgcccgagcttctgtctcagttttgggtgagtctccagcccctagtggatccgggcattaacagcttctattatactatttttatttcccataaatatttactaaaaataatactataattttaacttctttcttctcttcttctttgggcttgtttattgctttcaatcatacttctatccctggaagaatcatccttcctaaaaattctcaatttctaagctcaactaattatttctgcttaatgactttgatagatgataatctccaagctttatgacttcccatctctcccattctctaggagacatattaggctctgccatgcaaaacacacaaaatcttctccagatgccctatggctgtggagagcagaatatggtcctctttgctcctaacatctatgtactggattatctaaatgaaacacagcagcttactccagagatcaagtccaaggccattggctatctcaacactggtgagtgattacttgagtaagggaaaacttgaatgttatttcaactggatttcccagtaggtttcagttacttatgaatattatgatacattagcttagctcactatgatagctgctatgatagttaatttcaaggaaactatccactctccaacctccaataaaatatttaaggctcagaaactcctaatctatgacaacaaaatttaagaaatgtcacaagagaagccaaggtacttttagtaatttctccaccctcagcatgcacattaatccattgtgctgtttcgttaatcttcctttccaggttaccagagacagttgaactacaaacactatgatggctcctacagcacctttggggagcgatatggcaggaaccagggcaacacctggtaaggaaagaacaattttttgagcttctttttgtgtgccagctcttttacatgtattacctcaattatattcacagcaacactatcagatatgtattatcagaccgatggtttgttatactagataaatccaccaagattagcaaggtaatcagaagaaaacctgatatccaaatacatgttatgttaggcttgtttccaaaatggatcctattaataatgtaccaaggttttctttctgaaatggctattctttctaaagtagctaccataaccatgagttttaaaatgatattgccagtgaacatatataacttccagataaaccatgttaacttcagcttatattgtcacattctaagtcattcagcttgacttggaatgaattcattaataagaggaaacaattgagaaggaaacagtaatataaaacatttttttaaatccctaaagtaaagcaatattaaaatttactgcatgtaagagctgcatgtgagaagattctgtcatctgcagaaggaaatctctaaagataagagagatttaaagccttactcaagtaactaacaaaaataagtacattcaaattacttgaatgtaaatttgttcaaccattgtggaagacagtatggcgattcttcaaggatctagaaccagaaataccatttgacctagtaatcccattactgggtatatacccaaaggaatataaatcattctactataatgacacatgcacatgtatgtttatcgcggcactatttacaataacaaagtcatggaactaacccaaatgctcatcaatgacagactggataaagaaaatgtggtacatatacatcatggaatactatgcagcaataaaaagaaatgaaatcatgtcctttgcagggacatggatgaagctggaagccatcagcctcagcaaactaacacaggaacagaaaaccaaacaccacatttctcactcataagtgggagttaagcaatgagaacacacggacacagggacaggaacaacacacaccagggcctgttgggaggtgtggggtgacgggagggaactaagcggatgggtcaataggtgcaagaaaccaccatggcacacgtatacttatgtaacaaacctgcacgttctgcacatgtatctcggaactaaaataaaattaaatatactaagactccctgtggcaaagagagagttagcaaggaaatactacatctagcagattaatcaggcagactaaagattaatcaaggagataagctctctaagtacacaagaattttgttagctaactcacatcatatgaagcctgttgctgtgaagtggttataaaaccattttgacaacataaacatcatgattgcttcctccctggtcaggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaaggtgaatcattctggagctagttttgatttgtccattatgatatctgcaaggatgaggataggaagtgataatgtgaaaaattctaagggaaagcctcagaggaaaataaaacctggatggcaccaaaaaagaggggatagaacaaaagttgattgtgatactttgccctatagggatggatatgggtaaggatgaattccatgacacagcagaatagaaagaactaatcaatagcattctcagaagttgaattattcagatctctctctcgtattcacagggaggagtagaagatgaagtgaccctctccgcctatatcaccatcgcccttctggagattcctctcacagtcactgtaggtaccaccccattcctctgctgaaggagagttctggatgcaatgaaactgctgacctgctgtctgaaatactatcctattaaaagcaaagcatcagctttctttctatgcaatgccagtgcttcccagatctacagagaatttggtcagcccattaagaaaggtttaaattttcccagtaattcccctaggctatttaccaccaccactcaaaaaagaatcttaaagatgtatcttttgaatgtgagaataacagataaaaataatattatatctattgataagaatgaggaatcgttggaaaaatgcgtttgaaaaacttctgtgctgtgatccgtgtatttgcctgggaatgctaatatgcctgtttacatagcttagttcccttcttgttctgccttcacagcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaaggtgagagcacacctgagatccttctcctggcccatcctctgtatcaagaactgcatggcaaaaatccctcactcctacctcctgtgatccctgtctcctctcttcttttctatatatcatatatattttgtccatattgcatcttataaaatctaggatttcttaatcaaatcagaaatcagaagacaagaggccgtgcagatgcttctcaattacgatggggttatatcctgacaaactcattgtaaagtctaaaaaatcttaagtgggaccattgtaagtcagggaccatctctatagtatgctggtaagaagagcattctctggagactagctccaaaatgtgctacctatgtgagcttgggaaagtcattaacttccttgtgtttcagttccttcatcagtaaaatggggataataatagtatttacctcacagagctgttgtaataaatgaattggtacacgtaaaacacttagtagagtacatgtcacatagcaaatcctataaaagtactagttattacaattaacatatcagttctcaatatatgcccaacccttacctggtacattatataaccttaaacataagaaaataatcatggaagtaactccttgaatgaattctggtattttaagcccatttcataagaccaataatgttgaccaatctactcatattcacacagtacttctacatataccatggtctatatgagcggttgaagaaatagaaaataaaatgcaaatcacaagatgtccattaaaacagtctacctttttcctttgacagccattaattcttcttaaaatgtattgagaaatattttataatagatatacaaaagggcataagctataactagaaaacactgtacaactctctcatagattaagaaatagaaaattaccgacatgggaaaaataaatcccttgtgtatcactaccacctccagaggcaatcattatcctgaatttgtcgttacaattccatggatttccttatatttttgctgcatatgtatccctaactaatatttagaatcttcacatgtgtttcatcttgagagaaacaagatttttatttctctcttattcaagaaacaagagaaacatttttgaatatttcagcagcttagttttttgtttgtctgctttttattttctaagttcaacactatgttgatgaagacccctatacaagtatgtgcagagtcagctcattaattttcactgctgcataatatattacagtctataaattagtcataatttacacatcgagtttctcctcatggattttttttatgttttgctattaaaaaaaatgctgcaatgaatactcatgtgcctgttttcttgtgcatctgtgtttctccaaattatgctttgagaagcataatgactgattagtgggctaagcacatcttccccattgctgaatattgccaaagagcagttggcttcccacagcagtgtatattggttcccattgtttcacatccatgtcagcctttggtatttcaaggattactgtattttttttttcaatttaatgagtagaaactctactatttcatgtgatctgtgatcctcacaagaaactgataaagacacactttataggaaatgtaacaaactccagctatagtctaatataacatcataaataggaaatagccagactcaatgagacaactgctgtgccatttctttctcccaccaacccgactatagcaacgatttgaaaacatagataggcataggcttctgactccagcatcaatatctgccttagctgggctaaaacacaccaaattcagatttacatgaagggaaaagcatctacatacagtacaggggattataatgggcatgcaattctcatttcagcactggcttgggtactttcaccttgaattaaatataaatatgtaggcacttataaatatctttttctcatctttaagacaactctgtccattgggagcgccctcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggttggtgatttgccaaaaccttttatttcaccttcaggtagcaaaagatttgaatgaaaaagaaacaaacacatccaagaagaaaaaaatacagatgacagtaacttgaaatgaggaaaagttttcagtatccaaggataatggaaataaaagcaaatcaaagtcaaagagggccaaaaggaaatgctcagaatcccggcaccccatcgctgtgttattatccatctcctatttcccataacaacactgccttcctcaagcagcagtggagcaccagcagaatgaaggagatgtctcctgccattctcctgaaagctctagggtctctttcaaactgttcaaaggaactctactcaaaatccaacaacctctcctcgcaaatctctccattcttaggtcccctttaataggcttttctcaaaactacacattttgtgcttcccctattcaccttttttttttttttttttttaagacagagtcttgctctgtcacctaggctggaatgcagtggtgcaatctcggctcactgcaacctccatctcccaggttcaagcgattctagtgcctcagcctcccaagtatctaggattacagtcatgtgcaatcatgcctggctaatttttgtatttttagtagagacgaggttttgccatgttgcccaggctgatctcgaagtcctgagctcaggcaatccatccgccttggcctcacaaagtgctaggattataggtgtgagccactgcgtccagccccctattcacctcttaatacacaaacatttattcatcaggagcataaagaactgtctttattcatccaacctcctaaatctagctatataaccatgtatctgaacaattcattgatatgtacacagcagaaagttttatcttcagagaattcggatgtttgcttatataccctaaaacggaaaaaatgtgacaaaatggcattccatcctatttccattgtattaatcttttatcatatgaatgaaaaaaactaagtaattttgttaaaggttatcattcatttattagaaacatattatttgaaggaggccaagcaggtttaatgttgttgaggatacataccagcagacattcactgggaacaggaaatcatccaataaaaagggaaagccaaataaaaatgtcattaaatccagaagataattataatactcatcttttatttcttttggagaaactgaagcatgactctgctcatggctgcaaagaaccttgggttctctccaggacactgacctcagcaactgagcaaagtttaatatgggagagagccagactgaactttgcttgagtggtggcagatatgagcatagttgtcaagaaagacatgttagcaaatagctgatgccaataactgattgccattcacatgttttccacattccatgtcccacatatacttacagagagaaaaggatcaattttctgataaataaaataaacatgtagggcatacagtccaaggtagatatgtgaatgttatggttcttcaactatctaaagattataatcaatcttgaaattacagctcctatatttaagtagtgagggaagtaggaaatcaaagtccctcacatgggtctttgaaaaatatctcagccctcaaagccttataatgcccaatgggttctctcactcatctgtctctaacaggacacagtggtggctctccatgctctgtccaaatatggagcagccacatttaccaggactgggaaggctgcacaggtgactatccagtcttcagggacattttccagcaaattccaagtggacaacaacaaccgcctgttactgcagcaggtctcattgccagagctgcctggggaatacagcatgaaagtgacaggagaaggatgtgtctacctccaggtgagactcttgggcaggtgaggacaggacagatgaggacagcagctgttctctctgagaagtcctaactcagaaaacaatgggacagatcagagaaagggttagggacgtggacaggaattctgggaaagggcaaaaaactgattttgtctttgatgttctatagacatccttgaaatacaatattctcccagaaaaggaagagttcccctttgctttaggagtgcagactctgcctcaaacttgtgatgaacccaaagcccacaccagcttccaaatctccctaagtgtcaggtaagaccttctgactctatcacctaatcctaagaataaccaccagtcttctttcgggaactcctctttaagtaaagcagtgcaacagtagatatttgcactattcacaaaaaatgcaatgtattctcttaagttgatataatttctcaatgatggggtttacattgtccatccaggatctactattgtgcaacctcattgtttaaagggtaataatttccctcaataacaactaagtaaatattacccattgcctctgacctgaattccttgttatgtaatgaaatcctatattattcttgctttattgaagatagagatgaagaattattgaaaagtttgaatagaaggaagtagtgactccttagttagaattcctactggcaataataaatctcaggttatatatgatataattaatttggggggaagatacacttatatgcatcaatatttaaatagctgcagatctgataaaaaactctctctccacaaacatattattacttggttggagatactattcaggaaaaaagttaggacaaaatacatgtaacaaataactggcacacatcaaaaagaatgagatcatgacctttgcaggaacatggatggagatggaggtcattatccttggcaaactagcacaggaatggaaaaccaaacactgcatgttctcatttgtaagtgggagctaaatgatgagaacatatggacacaaaaaggagaacaacagacaccagagcctacttgagggttaaggatgggaggagggagaagatcagaaaaaaacaacaattcagtgcaaaatttagtacccaagtgataaagtaatctgtacaccaaacccccatgacacgagtttacctatataacaaacctgcatgtgtacgcctgaacctaaaagttaagtatatatatatatatttttttcatttaatttggtgtatatatatgccaaaaaataaattaagcagtccaaatttcggatgcaaactctcggggacaagacgctaggtgtttctaagtgttttgttgaaagccagtgtttaagtaaacattataaattattgttgtttttgtaaataatgtagactgaaatttattattcataatatacatcattttgtcagctgaaagaaaataaaagtaaacaaataaataaaataactggcatagattagaggtcacaaacagcctgtgcatcacttgtagagctttcttaaaatgcagatcctcagccgggcgtggtggctcacgcctgtaatctcagcactttgggaggccaaggcgggcagattaccttaggtcgggagttcaagaccagcctgaccaacatggagaaaccccgtctgtactaaaaatacaaaattagtcggacgtggtggtgcatgcctgtaatcccagctactcgggaggctgaggcaggagaatcacttgaacccaggaggcggaggttgcggtgagccgaaatcatgccattgcactccagcctgggcaagaagagtgaaaaactccatcaaaaaaaaaaaatgcagatcctcagcccccatacactagacattctgattcatcaggtctagagtagggcctggtctctgtggctttaacaggcttcctaaaaattctacgcacaccacttttacaaaccactgggataagatatttgggaagacttacgtgtaccttttagagctgtggaatgcttaacatggacatagaagaagaaaatatttaaaaacacagaaaaccctaatcctttcctcccctggatcctcagttacacagggagccgctctgcctccaacatggcgatcgttgatgtgaagatggtctctggcttcattcccctgaagccaacagtgaaaatggtaggtttatcataaccccagactgccctattttatttaatgatgtatgtatccccagcataagacaatactaatatcaaaatactattaaagtcaatctctatcaaagccttatcctttttccagctcagaaatataatcacatgtgtttgtatgaatgctgaccatgtgcagagcactgtgctaggacccatgactacaagaaaaagattgtcagcaggttcctgcttttcaatttcttcttagcttagaattttgctaagaagataaaagatatgaacacgaaccagtgaaaaatatgaaaatgactgattggcataaactataagtattacagaagttaaaagaaaaatagagcaagcaaaacaggaaaaaaactccttatgaagaaatagaaactgaattgaactttgaaatatgagtaactaccaagtttaggatacttagctgtcttttcttcagataaataactttacacattagtcgtgtgttatactaatagtaaaccctttatgcctttcatttttaattgtattacattatatatttccttacaaaaagcatttgaagaattctaccctcagggttattttggcaatacaaagattttttctctggatcccccaggggtttcatctatttattaacatttgtggtatttcaattttcttcagcttgaaagatctaaccatgtgagccggacagaagtcagcagcaaccatgtcttgatttaccttgataaggtaagagaacttccagtctatttgcaaaaaaacgtagataataatcctctaagggaacatctgggaaggtaaatgcattttagaaacatcacttccatgctagaaatttgagaattctaatgttaactctaaaagaatgttcttctctcctttatttatatttcaccagggattacaggtagaaatggcttattatgatcttgggatatgaatattcctaaaatcccataagcaagaaatcttcacaaaatgtgtttattatgttgacaagttttttggatacccagtaatataaggaagtagcccttgtgattagtcaattattagttaattatcaacatactcaacaacaatatgaaagggaaaaaaaactgtcagtctccacaaggacttgaaccataaaataataagaccagttcaccagtaaaccaatctgattttatagatatgtgtggtaggagagtttgttcatgcataagttgatgggaattatagtttacaaattttatgaaacttaagcctgggaagatcaaccttttagatgcctctttgagtctacgcaagtattcctgcaagacagagaagtcaaactataccaaatctctggatattaaaaaatgaacacagttagtcatccaataaaaagtatatatcatttacccccatgaacagagctatgtattggcattgacagaggtatatgcgttatgttagttatttaagaaataatctggagaatttatcatcccctctgagagatttctgcacaatttaattaaggaccctatagtgtgctgtaggataataaagcttttcccccaaaaaacaggtgaatacttaaactaattcaaagagagaagaaagcttcctgaaaggtcatttaattgacttttgctttccaggtgtcaaatcagacactgagcttgttcttcacggttctgcaagatgtcccagtaagagatctgaaaccagccatagtgaaagtctatgattactacgagacgggtgagtgagagtgattttcacgtagaaatatttaattcctgatcacagaaattcaggtttaggagatgtgttggggttatttattacattaagtaattacattatcacttcattttgtctccatcaagtctgatgcccctctttttgtctcttatacatacattatagaaacaacctacattataaatttatcaactactaatacaaaacacctgtgggatatttagttcccttttcatcagataaatggactgtatgacaatatgagatttaagtaagtagaacatctgaagagtccttcaggagtttgggataaaagaatatataaaacactatatttgaaaggagaatataaggtagcaagcaacacatcagatgaatgatgcttatgtttctggtacaatactgttcttcccacaacaaactccttccttggcctgtatcccacagatgtttgctttctttctcacttcatgtaatgatttctggttttttgttggttttttttttttcagatgagtttgcaattgctgagtacaatgctccttgcagcaaaggtaagccactcacactcctccaaaaggcagtcagagctccttcagcttgccccccaaaccttctccttcataaaacgctgggtaaatatttgtcaaaaacatcaaattactcacactgcacattattatagaaaaacacatttattggagagggccgctgactctgtcaaacctcagagagtccataggattgcttatgggtaatgatttggaatagatttggtttcccactgtactgattaggtttccttgggcactatgctacccagaactaagggaaagaatactctctgctcatggagacccaaatctgtcttaattttttttctttccaatgtcacagatcttggaaatgcttgaagaccacaaggctgaaaagtgctttgctggagtcctgttctcagagctccacagaagacacgtgtttttgtatctttaaagacttgatgaataaacactttttctggtcaatgtctttccctgtttcctgttcattcaataaatatcattgtacatttccatatgattcccaatagaataccaagattaaacttaaaggaatcaagtgctgaaggacttcagaatacaaaaaaatgatacagtgatgtcggtctgagtaggcttcatgtaaggactgtggggaaagaagaaagtattgggttatgtactaggaaagtgtaaagtgtgtttggttatgggaataccctatgaaaaacccaaagggtgaatttttatgagaaaataaaagactgacttcaccagaaaagactttttacattaaaatgaagtagaatgaaatacaacattgaacatgtcatattgagaggcaagataattgggacttgacctgaattgggagtgatgtgtcctatgttacaccaaaatctgccactgatgagagtgatcagtcagttaacctggggtttcagattcaataatagatgagctgaaaataatgaagggaggattcatgcagaagcacgttttctcagaagaaggaatgtgtatgactcaaagtccaaataggagtattatattggatcatctttcttctggaactttgagccaggattaaaggatagctgtaaagtcaaggagatattctgatgcagaaatcagttctcacaacatctgattgatgtctgatgtctcacaacatctctttagtctatttttaaaatatataattttctttgcagtaagtattgcgacatatatttccattctatagaaggggaagcaaaacttcaggagtttttgaagtaggaaaggttaaagcaggaggattgagccaagagagtctgaggacaatcgtaggagtcctactcttcatttggcacaaaaatgacaatgcttagttaggcagaaggtgagtatggattgtataaactaagaactggaaaagactttgcagttcaaggaatccttagctctgtctccaggctagacaaaataagaaataaaagctatcacttctgtgtggtgcttatagaatagaattaacatatcagcattatgggatctttagggtgtcgctttcctggccagtctagtggcacctttgcctgagttttgctctgggcccactgggctgcttctgcccactcgcacttgctaccaacctggatcccggatccaagggagattgagacgggtggagcagaggggtgtgctaggggtgtgtgagcaagcgtggccactgtgcagtcacacacacaagctgctgcccaggttgggcagctccaggtgccagcacaggctctccatgaggtggctggaccaggcacacaacaaacagcttccccctggaccaggcgcatcacaagcagcttccaacagtggcactggcgaatgcagtgacgccaaccagggccccaaagagggagtcacagcccgggctcaaggagctcccaggtctgggcttctccgagggccagagctcttctctccctgtggggagcaaggggcatgttgcagccctgtttgtgttacagctcttttaaccttgctgtgcagctcctcagctcctgcatcaagcagacaagtggagaatgagacagatgaagaggagcattactgagcaatggaacagaaggataaggaagatgaagaggaaggagacctgcagttagtagctcatttccacagtaaggatgtcctttccacagcaagggtgtcccaacgagtgttcagcttctagcagaacggagaccctggagtggctggctcctctctgcaaacaggtcttcccattgagtgttcagctttcagcagagaggaggttctggaatgggtagtttctctccacaggtaggtcatccattgtcttcccatcctctcttccaatctagctgagtctgggggattttatgagcctcagagggaggaaatgcatgctgattggtccatgggcagccatgagtgggcccagggagcagcaccacaagttacctctctggtctgcaggcttcaagccctcaccagcttgagggtgggacttcactggggacccatccccttccacccaggaacctgtctgcctcctgctcccaggctgttcatgccaaggagcgcctgcaagtcagtgtccagctgtcttcagacccctctcagcctccctcccacgcttgttggtgcccaagttccaaagggggccgagacggcagggggctggcgtatcagcactgtcctgagcgtgtgcacgctcggccaggctgtgacagtacccaggctcggcccgaccttgctctgagttcagagtgggtgctaacagtggagagaagccaggcagccggagtaggcaccctggagcctgcagtgggcaggggactttgctgggcctctgagagcacagaaaatgtccacagccgcggcaaggtggctgcagctgcaccctgggagctcctgctccaccagttcggaaggggcggggctcctgcttgtccctggctcacctgctcctgagtgtgcaggtccggtggcgcctccttgcaggctgggctgatgggcgggggcggggggggggggggggaggaagggaatgttccaggtcctccctgggcccgggactgtgtccggggcagggatgacgtcgctgcaagttcttcccgtggccccggggctcaggggcagcccaggactctccctcgcccggctcacggccctgcctggggggcgcctccgggagcagatcacgagccctggggctcagccctcaggcgcgtctagctcggcggtcaccccagtgccgggaggaccctgaagacgcgccccaggcggccctactcagagcctcctcccaaggcccaggaatgcggcgctgtcggaggtgtgcgcggtggccacaccgctgtccgggtccccaaagcgggccccgctcccacttctcgccttggccccgaaccctgggtccagccccagcgctttgtgtgcgaacaccgctccgccccggacccagctccgccttggggcccctctctgcctgcccctccgtgcccgactacactgcttcccctccggcgggcgactcagcccggtccatcgtggcggcttccagggcggcaggctccgggagtactcccggggccggctccaaggactgttcccctcctccccactcccactccgcggcggcggcgggcgagagcggcgacatagggccagggtccggagcggtggaggctcctggccggggagcacgtcgccccacccggcaacgcgaggatggtggcggcgcagtcggctgctttggggtctcaaggcacaggggacgcgaggcacagatgtcccacagcagccactgcggctcccgcagctgctccgccgccgctgcccgcccctccctgctgcagctggcgtgatggcagcggcagctctggacgccccactgctgccaccatcagccttgtgaaataggtactaccttaacaaatgaaattgaagcagagacatgtaatttgcccaaagttactaagttagtgacaaagctagaattcaagaccaagaagtctagcttccatgctcttaacttccaaccatggtgacacctcaaacaacttcagacaaaaaggccaggagaaagtatatttcagagcttaataaacattataattagctgtcaaattaagtatcaagccagggcacagaacataaaagaaatcagagtatggctatgggaacaagacaacaggattataattttacctcttggttctagtttttttctttgttcatatggaaatcgttactgaaaaggtactttaaggatatgcttgttgcaaatcattagctgtatcactgaccaagagtgtttattcctgaaatactaactgattgcctactatctgccaggcacaatatcccatgctataatacaaaattaaacaaaataggattcctcccttagaaaaactcaccgcagagtaaaagaaaaagatacacatctgggtcattataatgatcaggtgctcaagctattcattgccccagtggactggagatacaatggccttctcaagttttggggtattacactcaaattcatatgtaatactggagaaaaggcctaattacaacttaaactggatttcccaccagcctggtggcatgggaatcttggaattaaaattacgtagaatttttaaaagtgatgtatcttctacatctgattttgtgaactgaagtttattctttccaggaaagcatatagatacacgacaggaaatgaaatggatacttgttggggtcagttttatgtatagtttgtattttattttgaaatatgatacactgctattctcttgcattttcttatatgtgactcaccactaaccctatattcccccatttcaggccagttggtcataagcatccatttgcctcagagaatactgggttgttatgacaagaatataaagttggaaagaaatagaatatttgagtctaccctgtaagaataaaaagaataaaaggggtttaaatttatttagaccctattgtttaatcaagaattctggccaggagcagtggttcatgcctataatcccaatgctttaggaggccaaggcaggaggatcatttgaggccaagagtttgagaccagcctgggcaaattattgctcgggaaaaaaaggtcttatttagtattttagtctttacaatgtttttttctattatgcaatattctctcaaatactttatgtccatccacgttgtctgagacatgccactttaacattctagttatgctgtagctgtcattttaccctaagccgttagtagtactgatccacataaaattgggctgtttaggtgtttaactgtttaaatgtataatatatctgatatatttatatattgtataaaaaatcacctaacacaatagatatttactatgtctgctataaatatatatcatataacatataacaatatattataaatgataatatactataatataaaatataataaatatattataaatgtacaatatatccgatataaatatataaatatgtcaactatattatacatataaatgtatatgtataatatatacatatatgtataatacaatgtatttattatgtatatatataaatgtatatgtataatatataaatgtataatatatctgatataaacatatcagatatattatccatctattgtgtcaggtgattctttatacaatatataaatatatcagatatattatccatctattgtgtcaggtgattttttatacaatatataaatatatcagatacattatccatctattgtgtcaggtgattttttatacaatatataaatatatcagatatattatccatctattgtgtcaggtgattttttatacaatatacaaatatatcagatatattatccatctattgtgtcaggtgattttttatacaatatataaatatatcagatacattatccatctattgtgtcaggtgattttttatacaatatataaatatatcagatatattatccatctattgtgtcaggtgattttttatacaatatataaatatatcagatatattatccatctattgtgtcaggtgattttttatacaatatataaatatatcagatatattacatatctattgtgtcaggtgatttcttatacgatatataaatatatcagatatattatccatctattgtgtcaggtgattttttatacactatataaatatatcagatatactatacagttcagcccatcaaagcaccatattttggggggttggtttccgtgtcccaacactagctacgaaaatattagctattagctacctataactcttcagtagtaaattcaagaaacgtaaagtaattctcttcattaagttcttgccttgtctactaaaaaaatggtcatcaccgatgtggacaatgaagacctgtggggttaaaagctctaactagtatgcctccaagattctttgattgcctgccatcatgatcgaagaataaataacttctttttcatcttatttatttattttttgtagagatagggtctcgctatgctgcccaggctggtctcaaactcctgggctcaagagatccttctacttaagcctctcaaagtgctggaattacaggggtgagtcaccacgactgaccattaataatttctttcaatgacactttaacataggctcattcatctttacctctaaagaaaagtctttctggtctttttaaaattatattttttggccaggcacaatggccaggtgcggtggctgacacctgtaatcctagcactttgggaggccaaggtaggaagattgcttgaggccaggagtgcaagaccaacctggcaaacatctggaaaacatagcaaggccccatctctattaaaaaaaaattaattctatttttctaagagaaaaaagattcccaattcaacaacacttttcaaaaactttatctggcagctactcaggagattgagatgggaggatcatatgaagcccaggaattcaaaaccagtgtgggcaacatagtgagatcctatctcataaaaaaataaaaaataaaaaaagctttacttggaatacaacccatgactctggttataaatacaaaattcttcaaattcatttaaaggaatttaatcctagcttctcggatgaaaaaaggaaataatattcacaatttgatccatcatcagtagacaagttaaatgtgtttcacaaaagcaagacatattaattaagcaaaatcatattcgagtaaccacaggaaatataaatatactgtctcttacctagagaaatcttatagtctaattgtgaagatagtcttcacgtgacgaaaaagatcatcattaatccaaaacatataagttataaagaagcgacatataccagcaattctaaaatctggttagcatcctttgtagaatttattttaaaatgcagatatccaggtctcatcaataaagatttaattaattatttttggggatgtgctcagacatctgcgttttttgttttttgttttcgtttttttgttttttgagatggagtctcactctgttgcccaggctggagtgcaatggcgcaatctcagctcactgcaacctctgctcccaggttcaagcaattcttctgcctcagcctccctaggagctggaactataggcgcccaccaccacgttgggctaacaggcatctatgtttttaatgaactctgtaggtggttctatcatgcagttagttttcagaaccattcacactgacagtaaaggctatttattcccagcagttgaaagaccactaaggacacaggaatagttagcaaagctacttaaagatgccagggctggggccgggtgcgatggctcacgcctgtaatcccagcactttgggaggccaaggtgggcagatcatgaggtcaggagatcgagaccatcctggctaacacaatgaagccccgtctctacaaacaaacaaacaaacaaacaaaatacaaaaaattagccggatgtggtggcgggcacctgtagtcccaactactcgggaggctgaggcagcagaatggcttgaacccaggaggcggagcttgcagtgagccgagatcacgccactgcactccagcctgggcgacagagcgaaactccatctcaaaaaaaaaaaaaaaaaaaaaaaaaagatgccagggctggctgggcacagtggctcacacctgtaaccccaacactttggtttgggaggccaaggcggatggattgcttgagttcaggggttcaagaccagcccaggaaacatggcaaaacctcatctctaccaaaaacacaaaaattagccgggcatagtggcatgcacctgtggtcccagctactcaggaggctgaggtgggaggatagctggagcctgggaagctgcagtgatcagtgatcatgtcaccacactccagcctcggtgacagagcaagaacctgtctcaacatacatacatgcatatataaaattaaacataaaaacaaaaataaataaagatgtcagggcttatgttgaaccttaactgagagcaagattcaaaagacactgaggcttatttttctttcttatatctatagttacacagggagctgtctaatcttggatgtatccaagttgatatctggttttatccattgaaacccacagtgaaaatggtaaataggtgctaggtgtttggatttttttaatccaatgtaagaataaaacaatggtatcctaataatgtcaaagcaacattggtcataatctaaggaaattgaattcatatagtaccaaatatatatttagcattgtgctaggtgctgatacattctagataaaaatattacacatgggtaaccaaaactgtcaaatgacatttcagggcaagatataattaagtaccaaaatcattggcatagtctttaagtactgtgaaatgtagagaaagctgagatgaatggcagtgtagagacagatggctttgcaaatcatctcagatgactagctatccaatgtgaggacacttctccctcaccttcaaacaaatgctaaagacgcctgttacttaatcatatgaatattcaatcttgtatctaatgtggtggtattcataatactctgtatatgttttcatcttactggacaagtgtcttcgaactcatttaaatgaatttaaccccagctttgtttatgtatagacttcttcaatctcatagtctatttgtctctttgtaccccacaggctttctttattcagtaacactggtgatttctcttattttccttagcttgaaagatctagccacgtgagcaggacagaagtgcacaacaaccatatcttgatttctgtggaccaggtggggcccctgccagccttgctagacagacccaggtgaacagtccttctaggggatctcatcaccaggcaagcacgtggtacgagaagagcagtcattaggaaggccatttggaaaagcacatcctctctgttcacgtgagatattttacatcctcattcctcatcgcaagcttcctgggatttggagtgtcacagacaagagggttgggggaggccagtaggtatggatttgtttatattaaaatgagcatatgaatatttatatgtttatattaaaacatatatgtttgtttatattacaatgagcatatgaatatttatatgtttatattaaaacatatatgtttgtttatattaaaatgagcatatgaatatttctgtatacttcagataaacattcttttccataaataagcttcatcatccagaagccatgttgaaagttggtaatcaaggataggaagtgtttccaagggttgtcagtgattaaatcaaccttaccttagcatacatgta

SEQ ID NO: 2 - DNA, 4530 bp, Homo sapiens
atggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggatcccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggaattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaatgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttctattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacaatgaagactgcatcaatcgtcataatgtctatattaatggaatcacatatactccagtatcaagtacaaatgaaaaggatatgtacagcttcctagaggacatgggcttaaaggcattcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtaaactcagcaggtgtggctgaggtaggagtaacagtccctgacaccatcaccgagtggaaggcaggggccttctgcctgtctgaagatgctggacttggtatctcttccactgcctctctccgagccttccagcccttctttgtggagctcacaatgccttactctgtgattcgtggagaggccttcacactcaaggccacggtcctaaactaccttcccaaatgcatccgggtcagtgtgcagctggaagcctctcccgccttcctagctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagagatcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcgccctcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagcagccacatttaccaggactgggaaggctgcacaggtgactatccagtcttcagggacattttccagcaaattccaagtggacaacaacaaccgcctgttactgcagcaggtctcattgccagagctgcctggggaatacagcatgaaagtgacaggagaaggatgtgtctacctccagacatccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctgaaaccagccatagtgaaagtctatgattactacgagacgggtgatttgcaattgctgagtacaatgctccttgcagcaaagatcttggaaatgcttgaagaccacaaggctgaaaagtgctttgctggagtcctgttctcagagctccacagaagacacgtgtttttgtatctttaaagacttgatgaataaacactttttctg

SEQ ID NO: 3 - DNA, 4577 bp, Homo sapiens
gctacaatccatctggtctcctccagctccttctttctgcaacatggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggatcccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggaattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaacgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttttattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaggcattcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtaaactcagcaggggtggctgaggtaggagtaacagtccctgacaccatcaccgagtggaaggcaggggccttctgcctgtctgaagatgctggacttggtatctcttccactgcctctctccgagccttccagcccttctttgtggagcttacaatgccttactctgtgattcgtggagaggccttcacactcaaggccacggtcctaaactaccttcccaaatgcatccgggtcagtgtgcagctggaagcctctcccgccttccttgctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcgccctcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagccgccacatttaccaggactgggaaggctgcacaggtgactatccagtcttcagggacattttccagcaaattccaagtggacaacaacaatcgcctgttactgcagcaggtctcattgccagagctgcctggggaatacagcatgaaagtgacaggagaaggatgtgtctacctccagacctccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctcaaaccagccatagtgaaagtctatgattactacgagacggatgagtttgcaatcgctgagtacaatgctccttgcagcaaagatcttggaaatgcttgaagaccacaaggctgaaaagtgctttgctggagtcctgttctctgagctccacagaagacacgtgtttttgtatctttaaagacttgatgaataaacactttttctggtc

SEQ ID NO: 4 - DNA, 2041 bp, Homo sapiens
cccgccttcctagctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagagatcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagacctgctggcctatgcttttgccctggcaggtaaccaggacaagaggaaggaagtactcaagtcacttaatgaggaagctgtgaagaaagacaactctgtccattgggagcgccctcagaaacccaaggcaccagtgggggatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagcagccacatttaccaggactgggaaggctgcacaggtgactatccagtcttcagggacattttccagcaaattccaagtggacaacaacaaccgcctgttactgcagcaggtctcattgccagagctgcctggggaatacagcatgaaagtgacaggagaaggatgtgtctacctccagacatccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctgaaaccagccatagtgaaagtctatgattactacgagacggatgagtttgcaattgctgagtacaatgctccttgcagcaaagatcttggaaatgcttgaagaccacaaggctgaaaagtgctttgctggagtcctgttctcagagctccacagaagacacgtgtttttgtatctttaaagacttgatgaataaacactttttctggtc

SEQ ID NO: 5 - DNA, 4577 bp, Homo sapiens; identical to SEQ ID NO: 3

SEQ ID NO: 6 - DNA, 256 bp, Homo sapiens
tatattttataatatatatttactgattagatgataattttctttgcaggacatgggcttaaaggcattcaccaactcaaagattcgtaaacccaaaatgtgtccacagcttcaacagtatgaaatgcatggacctgaaggtctacgtgtaggtttttatggtaaacaaaaaattaataaatatatattgcctaatatattcaccaaattttaaattttttaaaagatacaatgtgacaaaaattaacaaacaaaa

SEQ ID NO: 7 - DNA, 4576 bp, Homo sapiens
tacaatacagtctgttctcctccagctccttctttctgcaacatggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggatcccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggaattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaatgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttctattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacaatgaagactgcatcaatcgtcataatgtctatattaatggaatcacatatactccagtatcaagtacaaatgaaaaggatatgtacagcttcctagaggacatgggcttaaaggcattcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtaaactcagcaggtgtggctgaggtaggagtaacagtccctgacaccatcaccgagtggaaggcaggggccttctgcctgtctgaagatgctggacttggtatctcttccactgcctctctccgagccttccagcccttctttgtggagctcacaatgccttactctgtgattcgtggagaggccttcacactcaaggccacggtcctaaactaccttcccaaatgcatccgggtcagtgtgcagctggaagcctctcccgccttcctagctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagagatcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcgccctcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagcagccacatttaccaggactgggaaggctgcacaggtgactatccagtcttcagggacattttccagcaaattccaagtggacaacaacaaccgcctgttactgcagcaggtctcattgccagagctgcctggggaatacagcatgaaagtgacaggagaaggatgtgtctacctccagacatccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctgaaaccagccatagtgaaagtctatgattactacgagacggatgagtttgcaattgctgagtacaatgctccttgcagcaaagatcttggaaatgcttgaagaccacaaggctgaaaagtgctttgctggagtcctgttctcagagctccacagaagacacgtgtttttgtatctttaaagacttgatgaataaacactttttctggtc

SEQ ID NO: 8 - DNA, 6487 bp, Homo sapiens
gaattctattgtttgtagtaaattgttttagtccaaacactaattcctctgtagcaaacataggatctaataaaatggattatgtgtggaaatcagtcctctttagaaacctaaaggaccaagtgtatcctgattaaaaagataaaacgctttctttctttctttttgtttttgtttttttgtttgtttgtttcgagacagaggctcgctctgttgccaggctggagtgcagtggcgtgatctcggctcactgcaacctctgcctcccgggtttaagcgattctcgtgcatcagtctcccgtgcagctgggactacaggcgcacgcaccacacccagctaatttttgtagtttaagtagagacggggtttcaccatgttggccaggatggtctcaatctcttgacctcatgatccacctgcctcagtctcccaaagtgctttttgataattttgagaaatgatggaagcatattagaatgaaaacaacctgaggatgtgcttttatctttgtatattcaaatattttttctcattaaaaagcagaaagtccgggtatgatggttcatgcctgtaaccctaacactttgcggggccgagataggaagatcccgtgaggtcaggactttgaggctagcctgagcaacatggtaggaccctgtctccataaaaagcttaagaaaaaaattagcggggcgtggtggagtgcacctgtagtcttagctatttgggaggctgagatgggaggatcacttgagcctaggagttcaaggctgcactgagctatgatctaaccactgtactccagcctgggcaacagagcaagaccctgtctctgaaaaaaaaaaatacacacacacacacacacacacacacacacacacacacacatgttagtgggatagcacaaatgagaaaaactctgctctttgatcactgagtacatctctgtagatatatatttccttcactgcagattttgcccaagatacttcgtcaaagacaaagccagtacaccctctaatagggtgaatatggttatgccacctactgagcttgtttttgatactagttaatatgtaaccagatgaaattgtcattatcgtcactgtcaggactatgggaagcttaagtgttctcttttcaaggacaatgtgcgctaactgtacaattggtacaattaaataagttatattcagttcctgggaagcactatagcaatacaaggagaaaatttgattctatttatttttgttaaggcccacctacctcctaatcctaatttctctcatttcccaaatattccttgtttgttcttactgttatgtgttttcctgtattttgctcttctactttcttttccatggactatctttttcccttcctttttttcgctctacccctttacctcagctttctagcagtatttgctaaatacttcaaaactgtatagaactggttcaaattgtgtgctcccttttctgtcaagaacttgctactcaggtaacccaattggtgatttttcctggaaacactgatggatgctgttcctatagcgaaacccagaacagagatgaaatagatgtcatcctcagccattagcattcaaactataaaaattaatttacactggtatagtaaggatcagaatgtcaaagctgtgttacacctagcatcttgtatgaaactaccccattaaggtgagaccacagatattattgccccactattggcatgaaagctgaggctcagagcagttaactgagttacccaggaccacacagctaagttagaagtagggctcaggtgtcctggcaactaactggtccagttattttttctctcaagctcgttttccctctcctaaagaataggaggctctgtcgtggtgaaaggcgattttagtaatactttcctttttatctgtgattataatgaatgcggcatctctcccattaaggatcattcctccacccacattcttaatacatctgctgcatgcatccttcagagacctccctctgggatcatcccttctcactccaaaaagctcaacttctcccctgtcatttgtacctcccactcagcatttttagaagcaatatttcattcaaacttattcaagtttatttccacctaaagaaatattcctttcaccctggcatctccgtcaggtactgctctgttgtttttctccccttcagacaaactgccaaactggctctagttcctcacattccccatcaccctcagcaagcttctgccccacaccggcactgaaacagctgaatcccaatgtccttgtccttaaacccagcagaaaaaaaaaatcaatcaattatttgatttcacagcggcacttgacatgggtagccaggaatttatcaatgacaacctttacagatcatctttgtaatttatcatgaggcatcaaatgaatgctattaacattaatccctcctattttaagtcattaatccaagtaaatgctcacttatttctagcatcttagaaaccatttaaattatgttacattatgaatcaatacattataaaattataccatcatttgtaataattttttaaaatgttgtgtgctattaacattgatgccttggtataaagtcatgatcattctggtctagtagcaatcttctattgactattctcttactaaagcggtcccttccgtgggactcagagacctcacactctcctgcctgtgtttcttcctctctaattggcccttcttgctccacttgggtgctcctgcccattgcctagacaagagcattccctgtaactctgtcttgggctctttttctcttttcatcaacatcttctacgtgggtattatcatccatttccatggcatcagcttgcccaataaactgataaatccatagtctctataagtacagcagatctcatcaagctagtggcattcagactgctttaactttaaccaaaaataagggattttgtacatgttcaataagcagttcccactgtgacactgtaatcacattttcacaattgtgacctaggacacttagagtaaaggatacagatgattgagacagaaatagtgacaaagaaaaataaggttaggatatagattttaatgctgtaacagacctcaaaatacaatggcttaactaagagaatgcatttctctgtcacataaaggtcccaactggcgtagacttttgatgactcaagggctcaggctgtgcctggtttgtggttctgccttccttaacacatggcttccatctgatgagctacagcagtacctatcactagtcagcatgtccacattccagcctgggcaaggaagaaaggggaagcgcagaactgtacccttccttttttaagtcatgaactgaaagttgcatgtatcacttccacttgccctccagtcaccagaacttagtcatatgccatacccagcttcaagggagtgggttaaaaacatagaagtcaactaggcagtctgcacccagcaaaggatcgggagttctattattaaagcagaattggagaagtggtaacaggaaacaaccaccagcctctgctgcatgtatatgaaacagatgtttcccaaatcactattctcacttattctgtctgatacactgtattttttattatattctctttcattttttaaaatcctggtcatgactcacagggcatgatgttacaacccacttagatgctaacaccataatctgaaaaatattacctatattatgtctaatattggccacttgaagtatggctagcctaaattgatctatgttgtaagtataaaattcacaccagcttgtgaaaacaaattatgaaaaaaaagtctttaagatatcattaacaattttatattggctaaatgttgaaatgatcatattttggatatattggattaaataaaatacactattaaaattaatttaatgtttctctttatgtggttactagaaaatttaaaatttaaaattacacagggcgatcacattctatttctagtagaccacactgctgtaagctcaagattcaaatgtcaaactcctgtgaatattaatacgtgaatatcccacaagcacttactccatcttcccaaccctcagcccttctgtcctccttctgctcccaccaatctgtgtttcttctgtttcactcacccagctaaaggcaacacaattcactccgtgacgagccaggaaaatggaaagacacattttcctttattcctcacattgatatattcactgagcactataattacctcttaaatatgatataaatctgcaagctcttttcaataccaccacaaattccatagttcaaaatgccatcagctttcacctatattattacaccagctcccatctggtcttcctgcatcctggatcacctctttctagctgccctttcaaatttcaataagagcaagctttccaggaaacaaacctgaagtcaatccactgagtactcctctgaataccttaatattgttgacaaattcctttctgatttgaagtatcagaaaggaatatttcctccataccaaatagttttcatttcatgcatgtgccgtgattcttctccctcctttgcatctgtcattcgttatgcttagaaagctcttttcatctctttgttcttcgagacaaccactactcatacttcagagcttaatttacattttgctttccctcaaaatttttttaaaaggttccaggtctgggttatgtgctctcttatgtgctcccagagcatcctgaacttctgcaataatatgtttggctactgtattttatacagtagttttatattgtattttatacagtaggtgttatattgtattttatacagtagttgtttttctgtctgtttttgccccaacaagaatgtaaaatctttaagtgcctgttttcatacttatttgaccaccctatctctagaatcttgcatgatgtctagccctagtaggatcaaaaaatacttacaaagcaactgaatagctacatgaatagatggatgaataaatgcatgggtggatggatggattaatgaaatcatttatatgacttaaagtttgcagaggagtatcatatttggaaggcagtaaggaagtctgtgtagtcgatggtaaaggcaattgggaagtttgttaggcacaataggtcaaaatttgtttttgaagtcctgttacttcacgtttctttgtttcactttcttaaaacaggaaactcttttctatgatcattcttccagggcctggctcttcatctgcaacccagtaatatccctaatgtcaaaaagctactggtttaattcgtgccattttcaaagaggactactgaattctgatgtggcttcaaacatttaggttaggcatatctaatggagaacttgcagccacactgacttgtagtgaaatatctattttgagcctgcccagtgttgcttaaattgtagttttccttgccagctattcatacaagagatgtgagaagcaccataaaaggcgttgtgaggagttgtgggggagtgagggagagaagaggttgaaaagcttattagctgctgtacggtaaaagtgagctcttacgggaatgggaatgtagttttagccctccagggattctatttagcccgccaggaattaaccttgactataaataggccatcaatgacctttccagagaatgttcagagacctcaactttgtttagagatcttgtgtgggtggaacttcctgtttgcacacagagcagcataaagcccagttgctttgggaagtgtttgggaccagatggattgtagggagtagggtacaatacagtctggtctcctccagctccttctttctgcaacatggggaagaacaaactccttcatccaagtctggttcttctcctcttggtcctcctgcccacagacgcctcagtctctggaaaaccgtgagttccacacagagagcgtgaagcatgaacctagagtccttcatttattgcagatttttctttatatcattcctttttctttcctatgatactgtcatcttcttatctctaagattccttccagattttacaaatctagtttactcattacttgcttacttttaatcattcttccccaactctctgaagctctaatatgcaaagccttcctaaggggtgtcagaaatttttagctttttaaaagaataaattttagatattcacattcatattgatctacttgagaccatgctatttatcttttcttatttcctctttctcaagggtccattttctattttataaaaataaagacaattctctcccacaaccaaacatggaacaatgccctggagtataaaaatctatagagtgccaaataaaggaacaatttgaaatactggtgttgatattgaaaaagcaagggactctaatgtcagaagagaaatccttttgcagatgaggtggtgatgaattc

SEQ ID NO: 9 - protein, 1500 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETGDLQLLSTMLLAAKILEMLEDHKAEKCFAGVLFSELHRRHVFLYL

SEQ ID NO: 10 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEVKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 11 - protein, 643 aa, Homo sapiens
PAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKDLLAYAFALAGNQDKRKEVLKSLNEEAVKKDNSVHWERPQKPKAPVGDFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 12 - protein, 1474 aa, Homo sapiens; identical to SEQ ID NO: 10

SEQ ID NO: 13 - protein, 1474 aa, Homo sapiens; identical to SEQ ID NO: 10

SEQ ID NO: 14 - protein, 75 aa, Homo sapiens
DMGLKAFTNSKIRKPKMCPQLQQYEMHGPEGLRVGFYESDVMGRGHARLVHVEEPHTETVRKYFPETWIWDLVVV

SEQ ID NO: 15 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA